SEQUENCE LISTING (106 sequences)

SEQ ID NO: 1 - protein, 8 aa, synthetic construct
  REGION 1..8: note = Synthetic: HCDR1 (IMGT)
GYIFTSYY

SEQ ID NO: 2 - protein, 8 aa, synthetic construct
  REGION 1..8: note = Synthetic: HCDR2 (IMGT)
INPQTGKT

SEQ ID NO: 3 - protein, 13 aa, synthetic construct
  REGION 1..13: note = Synthetic: HCDR3 (IMGT)
AREYGNYNYPLDY

SEQ ID NO: 4 - protein, 6 aa, synthetic construct
  REGION 1..6: note = Synthetic: LCDR1 (IMGT)
ENQYGY

SEQ ID NO: 5 - skipped

SEQ ID NO: 6 - protein, 9 aa, synthetic construct
  REGION 1..9: note = Synthetic: LCDR3 (IMGT)
QHHLGTPYT

SEQ ID NO: 7 - protein, 5 aa, synthetic construct
  REGION 1..5: note = Synthetic: HCDR1 (Kabat)
SYYLH

SEQ ID NO: 8 - protein, 17 aa, synthetic construct
  REGION 1..17: note = Synthetic: HCDR2 (Kabat)
YINPQTGKTSYAQKFQG

SEQ ID NO: 9 - protein, 11 aa, synthetic construct
  REGION 1..11: note = Synthetic: HCDR3 (Kabat)
EYGNYNYPLDY

SEQ ID NO: 10 - protein, 11 aa, synthetic construct
  REGION 1..11: note = Synthetic: LCDR1 (Kabat)
RASENQYGYLA

SEQ ID NO: 11 - protein, 7 aa, synthetic construct
  REGION 1..7: note = Synthetic: LCDR2 (Kabat)
NYKNLVE

SEQ ID NO: 12 - protein, 7 aa, synthetic construct
  REGION 1..7: note = Synthetic: HCDR1 (Chothia)
GYIFTSY

SEQ ID NO: 13 - protein, 6 aa, synthetic construct
  REGION 1..6: note = Synthetic: HCDR2 (Chothia)
NPQTGK

SEQ ID NO: 14 - protein, 120 aa, synthetic construct
  REGION 1..120: note = Synthetic: VH
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSS

SEQ ID NO: 15 - protein, 107 aa, synthetic construct
  REGION 1..107: note = Synthetic: VL
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIK

SEQ ID NO: 16 - DNA, 360 bp, synthetic construct
  misc_feature 1..360: note = Synthetic: VH DNA
  source 1..360: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagc

SEQ ID NO: 17 - DNA, 321 bp, synthetic construct
  misc_feature 1..321: note = Synthetic: VL DNA
  source 1..321: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaa

SEQ ID NO: 18 - protein, 8 aa, synthetic construct
  REGION 1..8: note = Synthetic: HCDR1 (IMGT)
GFTFSSFG

SEQ ID NO: 19 - protein, 8 aa, synthetic construct
  REGION 1..8: note = Synthetic: HCDR2 (IMGT)
ISIGSDII

SEQ ID NO: 20 - protein, 13 aa, synthetic construct
  REGION 1..13: note = Synthetic: HCDR3 (IMGT)
TRRSYRSYWYFDV

SEQ ID NO: 21 - protein, 6 aa, synthetic construct
  REGION 1..6: note = Synthetic: LCDR1 (IMGT)
ENIYSY

SEQ ID NO: 22 - skipped

SEQ ID NO: 23 - protein, 9 aa, synthetic construct
  REGION 1..9: note = Synthetic: LCDR3 (IMGT)
QHHFLSPWM

SEQ ID NO: 24 - protein, 5 aa, synthetic construct
  REGION 1..5: note = Synthetic: HCDR1 (Kabat)
SFGMH

SEQ ID NO: 25 - protein, 17 aa, synthetic construct
  REGION 1..17: note = Synthetic: HCDR2 (Kabat)
YISIGSDIIYYADTVKG

SEQ ID NO: 26 - protein, 11 aa, synthetic construct
  REGION 1..11: note = Synthetic: HCDR3 (Kabat)
RSYRSYWYFDV

SEQ ID NO: 27 - protein, 11 aa, synthetic construct
  REGION 1..11: note = Synthetic: LCDR1 (Kabat)
RTSENIYSYLA

SEQ ID NO: 28 - protein, 7 aa, synthetic construct
  REGION 1..7: note = Synthetic: LCDR2 (Kabat)
NAKTLAE

SEQ ID NO: 29 - protein, 7 aa, synthetic construct
  REGION 1..7: note = Synthetic: HCDR1 (Chothia)
GFTFSSF

SEQ ID NO: 30 - protein, 6 aa, synthetic construct
  REGION 1..6: note = Synthetic: HCDR2 (Chothia)
SIGSDI

SEQ ID NO: 31 - protein, 120 aa, synthetic construct
  REGION 1..120: note = Synthetic: VH
DVQLVESGGGLVQPGGSRELSCAASGFTFSSFGMHWVRQAPERGLEWVAYISIGSDIIYYADTVKGRFTISRDNPKNTLFLQMTSLRSEDTAVYYCTRRSYRSYWYFDVWGAGTTVTVSS

SEQ ID NO: 32 - protein, 107 aa, synthetic construct
  REGION 1..107: note = Synthetic: VL
DIQMTQSPASLSASVGETVTITCRTSENIYSYLAWYQQKQGKSPHLLVYNAKTLAEGVPSRFSGSGSGTQFSLKIISLQPEDFGTYYCQHHFLSPWMFGGGTKLEIK

SEQ ID NO: 33 - DNA, 360 bp, synthetic construct
  misc_feature 1..360: note = Synthetic: VH DNA
  source 1..360: mol_type = other DNA
gatgtgcagctggtggagtctgggggaggcttagtgcagcctggagggtcccgggaactctcctgtgcagcctctggattcactttcagtagctttggaatgcactgggttcgtcaggctccagagagggggctggagtgggtcgcatacattagtattggcagtgatatcatctactatgcagacacagtgaagggccgattcaccatctccagagacaatcccaagaacaccctgttcctgcaaatgaccagtctgaggtctgaggacacggccgtgtattactgtacaagaaggtcttataggtcctactggtacttcgatgtctggggcgcagggaccacggtcaccgtctcctca

SEQ ID NO: 34 - DNA, 321 bp, synthetic construct
  misc_feature 1..321: note = Synthetic: VL DNA
  source 1..321: mol_type = other DNA
gacatccagatgactcagtctccagcctccctatctgcatctgtgggagaaactgtcaccatcacatgtcgaacaagtgagaatatttacagttatttagcatggtatcagcagaaacagggaaaatctcctcatctcctggtctataatgcaaaaaccttagcagagggtgtgccatcaaggttcagtggcagtggatcaggcacacagttttctctgaagatcatcagcctgcagcctgaagattttgggacttattactgtcagcatcattttcttagtccgtggatgttcggtggaggcaccaagctggaaatcaaa

SEQ ID NO: 35 - protein, 8 aa, synthetic construct
  REGION 1..8: note = Synthetic: HCDR1 (IMGT)
GYTFTDYN

SEQ ID NO: 36 - protein, 8 aa, synthetic construct
  REGION 1..8: note = Synthetic: HCDR2 (IMGT)
IGPNYGGT

SEQ ID NO: 37 - protein, 12 aa, synthetic construct
  REGION 1..12: note = Synthetic: HCDR3 (IMGT)
ARRGSIPRAVDY

SEQ ID NO: 38 - protein, 6 aa, synthetic construct
  REGION 1..6: note = Synthetic: LCDR1 (IMGT)
QDIHNY

SEQ ID NO: 39 - skipped

SEQ ID NO: 40 - protein, 9 aa, synthetic construct
  REGION 1..9: note = Synthetic: LCDR3 (IMGT)
LQYDEFPYT

SEQ ID NO: 41 - protein, 5 aa, synthetic construct
  REGION 1..5: note = Synthetic: HCDR1 (Kabat)
DYNID

SEQ ID NO: 42 - protein, 17 aa, synthetic construct
  REGION 1..17: note = Synthetic: HCDR2 (Kabat)
DIGPNYGGTIYNQKFKG

SEQ ID NO: 43 - protein, 10 aa, synthetic construct
  REGION 1..10: note = Synthetic: HCDR3 (Kabat)
RGSIPRAVDY

SEQ ID NO: 44 - protein, 11 aa, synthetic construct
  REGION 1..11: note = Synthetic: LCDR1 (Kabat)
KASQDIHNYLS

SEQ ID NO: 45 - protein, 7 aa, synthetic construct
  REGION 1..7: note = Synthetic: LCDR2 (Kabat)
RANRLVD

SEQ ID NO: 46 - protein, 7 aa, synthetic construct
  REGION 1..7: note = Synthetic: HCDR1 (Chothia)
GYTFTDY

SEQ ID NO: 47 - protein, 6 aa, synthetic construct
  REGION 1..6: note = Synthetic: HCDR2 (Chothia)
GPNYGG

SEQ ID NO: 48 - protein, 119 aa, synthetic construct
  REGION 1..119: note = Synthetic: VH
EVLLQQSGPELVKPGASVKIFCKASGYTFTDYNIDWVQQSHGKSLEWIGDIGPNYGGTIYNQKFKGKATLTVAKSSSTAYMELRSLTSEDTAVYYCARRGSIPRAVDYWGQGTSVTVSS

SEQ ID NO: 49 - protein, 107 aa, synthetic construct
  REGION 1..107: note = Synthetic: VL
DIKMTQSPSSMYASLGERVTITCKASQDIHNYLSWFQQKPGKSPKTLIYRANRLVDGVPSRFSGSGSGQDYSLTISSLEYEDMGIYYCLQYDEFPYTFGGGTKLEIK

SEQ ID NO: 50 - DNA, 357 bp, synthetic construct
  misc_feature 1..357: note = Synthetic: VH DNA
  source 1..357: mol_type = other DNA
gaggtcctgctgcaacagtctggacctgagctggtgaagcctggggcttcagtgaagattttctgtaaggcttctggatacacattcactgactacaacatagactgggtgcagcagagccatggaaagagccttgagtggattggagatattggtcctaattatggtggtactatctacaaccagaagttcaagggcaaggccacattgactgtagccaagtcctccagcacagcctacatggagctccgcagcctgacatctgaggacactgcagtctattactgtgcaagacgcggtagcatcccgagggctgtggactactggggtcaaggaacctcagtcaccgtctcctca

SEQ ID NO: 51 - DNA, 321 bp, synthetic construct
  misc_feature 1..321: note = Synthetic: VL DNA
  source 1..321: mol_type = other DNA
gacatcaagatgacccagtctccatcttccatgtatgcatctctaggagagagagtcactatcacttgcaaggcgagtcaggacattcataactatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaaataaaa

SEQ ID NO: 52 - protein, 702 aa, Homo sapiens
  REGION 1..702: note = misc_feature - full-length human CEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 53 - protein, 705 aa, synthetic construct
  REGION 1..705: note = Synthetic: Macaca CEA
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNSGAYACFVSNLATGRNNSIVKNISVSSGDSAPGSSGLSARATVGIIIGMLVGVALM

SEQ ID NO: 54 - protein, 344 aa, Homo sapiens
  REGION 1..344: note = misc_feature - full-length human CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 55 - protein, 693 aa, synthetic construct
  REGION 1..693: note = Synthetic: 1-687 of huCEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 56 - protein, 696 aa, unidentified
  REGION 1..696: note = 1-690 of monkey CEA
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNSGAYACFVSNLATGRNNSIVKNISVSSGDSAPGSSGLSARAHHHHHH

SEQ ID NO: 57 - protein, 326 aa, synthetic construct
  REGION 1..326: note = Synthetic: 1-320 of CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGHHHHHH

SEQ ID NO: 58 - protein, 115 aa, synthetic construct
  REGION 1..115: note = Synthetic: 1-78 of huCEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQN

SEQ ID NO: 59 - protein, 290 aa, synthetic construct
  REGION 1..290: note = Synthetic: 398-687 of huCEA
ELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGA

SEQ ID NO: 60 - protein, 411 aa, synthetic construct
  REGION 1..411: note = Synthetic: CEA-v
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 61 - protein, 243 aa, synthetic construct
  REGION 1..243: note = Synthetic: 1-273 of CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTIS

SEQ ID NO: 62 - protein, 92 aa, synthetic construct
  REGION 1..92: note = Synthetic: 596-687 of huCEA
PIISPPDSSYLSGANLNLSCHSASNPSPQYSWRINGIPQQHTQVLFIAKITPNNNGTYACFVSNLATGRNNSIVKSITVSASGTSPGLSAGA

SEQ ID NO: 63 - protein, 341 aa, synthetic construct
  REGION 1..341: note = Synthetic: CHIM
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 64 - protein, 464 aa, synthetic construct
  REGION 1..464: note = Synthetic: CEACAM1
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 65 - protein, 252 aa, synthetic construct
  REGION 1..252: note = Synthetic: CEACAM3
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 66 - protein, 265 aa, synthetic construct
  REGION 1..265: note = Synthetic: CEACAM7
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGIYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 67 - protein, 349 aa, synthetic construct
  REGION 1..349: note = Synthetic: CEACAM8
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 68 - DNA, 2109 bp, Homo sapiens
  misc_feature 1..2109: note = full-length human CEA DNA
  source 1..2109: mol_type = genomic DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 69 - DNA, 2118 bp, unidentified
  misc_feature 1..2118: note = monkey CEA DNA
  source 1..2118: mol_type = genomic DNA
atggggtctccctcagcccctcttcacagatggtgcatcccctggcagacgctcctgctcacagcctcacttctaaccttctggaacccgcccaccactgcccagctcactattgaatccaggccgttcaatgttgcagaggggaaggaggttcttctacttgcccacaatgtgtcccagaatctttttggctacatttggtacaagggagaaagagtggatgccagccgtcgaattggatcatgtgtaataagaactcaacaaattaccccagggcccgcacacagcggtcgagagacaatagacttcaatgcatccctgctgatccacaatgtcacccagagtgacacaggatcctacaccatacaagtcataaaggaagatcttgtgaatgaagaagcaactggccagttccgggtatacccggagctgcccaagccctacatctccagcaacaactccaaccccgtggaggacaaggatgctgtggccttaacctgtgaacctgagactcaggacacaacctacctgtggtgggtaaacaatcagagcctcccggtcagtcccaggctggagctgtccagtgacaacaggaccctcactgtattcaatattccaagaaatgacacaacatcctacaaatgtgaaacccagaacccagtgagtgtcagacgcagcgacccagtcaccctgaacgtcctctatggcccggatgcgcccaccatttcccctctaaacacaccttacagagcaggggaaaatctgaacctcacctgccacgcagcctctaacccaactgcacagtacttttggtttgtcaatgggacgttccagcaatccacacaagagctctttatacccaacatcaccgtgaataatagcggatcctatatgtgccaagcccataactcagccactggcctcaataggaccacagtcacggcgatcacagtctacgcggagctgcccaagccctacatcaccagcaacaactccaaccccatagaggacaaggatgctgtgaccttaacctgtgaacctgagactcaggacacaacctacctgtggtgggtaaacaatcagagcctctcggtcagttccaggctggagctgtccaatgacaacaggaccctcactgtattcaatattccaagaaacgacacaacgttctacgaatgtgagacccagaacccagtgagtgtcagacgcagcgacccagtcaccctgaatgtcctctatggcccggatgcgcccaccatttcccctctaaacacaccttacagagcaggggaaaatctgaacctctcctgccacgcagcctctaacccagctgcacagtactcttggtttgtcaatgggacgttccagcaatccacacaagagctctttatacccaacatcaccgtgaataatagcggatcctatatgtgccaagcccataactcagccactggcctcaataggaccacagtcacggcgatcacagtctatgtggagctgcccaagccctacatctccagcaacaactccaaccccatagaggacaaggatgctgtgaccttaacctgtgaacctgtggctgagaacacaacctacctgtggtgggtaaacaatcagagcctctcggtcagtcccaggctgcagctctccaatggcaacaggatcctcactctactcagtgtcacacggaatgacacaggaccctatgaatgtggaatccagaactcagagagtgcaaaacgcagtgacccagtcaccctgaatgtcacctatggcccagacacccccatcatatcccccccagacttgtcttaccgttcgggagcaaacctcaacctctcctgccactcggactctaacccatccccgcagtattcttggcttatcaatgggacactgcggcaacacacacaagttctctttatctccaaaatcacatcaaacaatagcggggcctatgcctgttttgtctctaacttggctaccggtcgcaataactccatagtcaagaacatctcagtctcctctggcgattcagcacctggaagttctggtctctcagctagggctactgtcggcatcataattggaatgctggttggggttgctctgatgtag

SEQ ID NO: 70 - DNA, 1035 bp, Homo sapiens
  misc_feature 1..1035: note = full-length human CEACAM6 DNA
  source 1..1035: mol_type = genomic DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatag

SEQ ID NO: 71 - DNA, 2079 bp, Homo sapiens
  misc_feature 1..2079: note = 1-687 of huCEA DNA
  source 1..2079: mol_type = genomic DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 72 - DNA, 2088 bp, unidentified
  misc_feature 1..2088: note = 1-690 of monkey CEA DNA
  source 1..2088: mol_type = genomic DNA
atggggtctccctcagcccctcttcacagatggtgcatcccctggcagacgctcctgctcacagcctcacttctaaccttctggaacccgcccaccactgcccagctcactattgaatccaggccgttcaatgttgcagaggggaaggaggttcttctacttgcccacaatgtgtcccagaatctttttggctacatttggtacaagggagaaagagtggatgccagccgtcgaattggatcatgtgtaataagaactcaacaaattaccccagggcccgcacacagcggtcgagagacaatagacttcaatgcatccctgctgatccacaatgtcacccagagtgacacaggatcctacaccatacaagtcataaaggaagatcttgtgaatgaagaagcaactggccagttccgggtatacccggagctgcccaagccctacatctccagcaacaactccaaccccgtggaggacaaggatgctgtggccttaacctgtgaacctgagactcaggacacaacctacctgtggtgggtaaacaatcagagcctcccggtcagtcccaggctggagctgtccagtgacaacaggaccctcactgtattcaatattccaagaaatgacacaacatcctacaaatgtgaaacccagaacccagtgagtgtcagacgcagcgacccagtcaccctgaacgtcctctatggcccggatgcgcccaccatttcccctctaaacacaccttacagagcaggggaaaatctgaacctcacctgccacgcagcctctaacccaactgcacagtacttttggtttgtcaatgggacgttccagcaatccacacaagagctctttatacccaacatcaccgtgaataatagcggatcctatatgtgccaagcccataactcagccactggcctcaataggaccacagtcacggcgatcacagtctacgcggagctgcccaagccctacatcaccagcaacaactccaaccccatagaggacaaggatgctgtgaccttaacctgtgaacctgagactcaggacacaacctacctgtggtgggtaaacaatcagagcctctcggtcagttccaggctggagctgtccaatgacaacaggaccctcactgtattcaatattccaagaaacgacacaacgttctacgaatgtgagacccagaacccagtgagtgtcagacgcagcgacccagtcaccctgaatgtcctctatggcccggatgcgcccaccatttcccctctaaacacaccttacagagcaggggaaaatctgaacctctcctgccacgcagcctctaacccagctgcacagtactcttggtttgtcaatgggacgttccagcaatccacacaagagctctttatacccaacatcaccgtgaataatagcggatcctatatgtgccaagcccataactcagccactggcctcaataggaccacagtcacggcgatcacagtctatgtggagctgcccaagccctacatctccagcaacaactccaaccccatagaggacaaggatgctgtgaccttaacctgtgaacctgtggctgagaacacaacctacctgtggtgggtaaacaatcagagcctctcggtcagtcccaggctgcagctctccaatggcaacaggatcctcactctactcagtgtcacacggaatgacacaggaccctatgaatgtggaatccagaactcagagagtgcaaaacgcagtgacccagtcaccctgaatgtcacctatggcccagacacccccatcatatcccccccagacttgtcttaccgttcgggagcaaacctcaacctctcctgccactcggactctaacccatccccgcagtattcttggcttatcaatgggacactgcggcaacacacacaagttctctttatctccaaaatcacatcaaacaatagcggggcctatgcctgttttgtctctaacttggctaccggtcgcaataactccatagtcaagaacatctcagtctcctctggcgattcagcacctggaagttctggtctctcagctagggctcatcaccatcaccatcac

SEQ ID NO: 73 - DNA, 978 bp, synthetic construct
  misc_feature 1..978: note = Synthetic: 1-320 of CEACAM6 DNA
  source 1..978: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggacatcaccatcaccatcac

SEQ ID NO: 74 - DNA, 345 bp, Homo sapiens
  misc_feature 1..345: note = 1-78 of huCEA DNA
  source 1..345: mol_type = genomic DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaat

SEQ ID NO: 75 - DNA, 870 bp, Homo sapiens
  misc_feature 1..870: note = 398-687 of huCEA DNA
  source 1..870: mol_type = genomic DNA
gaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcc

SEQ ID NO: 76 - DNA, 1233 bp, synthetic construct
  misc_feature 1..1233: note = Synthetic: CEA-v DNA
  source 1..1233: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 77 - DNA, 729 bp, synthetic construct
  misc_feature 1..729: note = Synthetic: 1-273 of CEACAM6 DNA
  source 1..729: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttcc

SEQ ID NO: 78 - DNA, 276 bp, synthetic construct
  misc_feature 1..276: note = Synthetic: 596-687 of huCEA DNA
  source 1..276: mol_type = other DNA
cccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcc

SEQ ID NO: 79 - DNA, 1023 bp, synthetic construct
  misc_feature 1..1023: note = Synthetic: CHIM DNA
  source 1..1023: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttcccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 80 - protein, 8 aa, synthetic construct
  REGION 1..8: note = Synthetic: HCDR1 (IMGT)
GYIFTSYW

SEQ ID NO: 81 - protein, 8 aa, synthetic construct
  REGION 1..8: note = Synthetic: HCDR2 (IMGT)
INPNTGYT

SEQ ID NO: 82 - protein, 6 aa, synthetic construct
  REGION 1..6: note = Synthetic: LCDR1 (IMGT)
ENIYGY

SEQ ID NO: 83 - skipped

SEQ ID NO: 84 - protein, 9 aa, synthetic construct
  REGION 1..9: note = Synthetic: LCDR3 (IMGT)
QHHYGTPYT

SEQ ID NO: 85 - protein, 120 aa, synthetic construct
  REGION 1..120: note = Synthetic: VH
QVQLQQSGAELAKSGASVKMSCKASGYIFTSYWLHWVKQRPGQGLEWIGYINPNTGYTNYSQKFKDKATLTADKSSSTAYMQLSSLTSEDSAVYYCAREYGNYNYPLDYWGQGTSVTVSS

SEQ ID NO: 86 - protein, 107 aa, synthetic construct
  REGION 1..107: note = Synthetic: VL
DIQMTQSPASLSASVGETVTITCRASENIYGYLAWYQQKQGKSPQLLVFNAKNLVEGVPSRFSGSGSGTQFSLKINSLQPEDFGSYYCQHHYGTPYTFGGGTKLEIK

SEQ ID NO: 87 - protein, 330 aa, synthetic construct [sequence omitted]

SEQ ID NO: 88 - protein, 107 aa, synthetic construct [sequence omitted]

SEQ ID NO: 89 - protein, 450 aa, synthetic construct
  REGION 1..450: note = Synthetic: BGA75921 heavy chain
QVQLVQSGAEVKKPGASVKVSCKASGYIFTSYWLHWVRQAPGQGLEWIGYINPNTGYTNYSQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, synthetic construct
  REGION 1..214: note = Synthetic: BGA75921 light chain
DIQMTQSPSSLSASVGDRVTITCRASENIYGYLAWYQQKPGKVPKLLIYNAKNLVEGVPSRFSGSGSGTDFTLTISSLQPEDVATYYCQHHYGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 120 aa, synthetic construct
  REGION 1..120: note = Synthetic: BGA75921 VH
QVQLVQSGAEVKKPGASVKVSCKASGYIFTSYWLHWVRQAPGQGLEWIGYINPNTGYTNYSQKFQGRVTMTRDTSTSTVYMELSSLRSEDTAVYYCAREYGNYNYPLDYWGQGTLVTVSS

SEQ ID NO: 92 - protein, 107 aa, synthetic construct
  REGION 1..107: note = Synthetic: BGA75921 VL
DIQMTQSPSSLSASVGDRVTITCRASENIYGYLAWYQQKPGKVPKLLIYNAKNLVEGVPSRFSGSGSGTDFTLTISSLQPEDVATYYCQHHYGTPYTFGQGTKVEIK

SEQ ID NO: 93 - protein, 107 aa, synthetic construct; identical to SEQ ID NO: 92
  REGION 1..107: note = Synthetic: BGA7592-1 VL

SEQ ID NO: 94 - protein, 120 aa, synthetic construct
  REGION 1..120: note = Synthetic: BGA7592-1F VH
QVQLVQSGAEVKKPGASVKVSCKASGYIFTSYWLHWVRQAPGQGLEWIGYINPQTGYTSYAQKFQGRVTMTRDTSTSTVYMELSSLRSEDTAVYYCAREYGNYNYPLDYWGQGTLVTVSS

SEQ ID NO: 95 - protein, 107 aa, synthetic construct
  REGION 1..107: note = Synthetic: BGA7592-1F-ph-L VL
DIQMTQSPSSLSASVGDRVTITCRASENQYGYLAWYQQKPGKVPKLLIYNFKNLVEGVPSRFSGSGSGTDFTLTISSLQPEDVATYYCQHHLGTPYTFGQGTKVEIK

SEQ ID NO: 96 - protein, 107 aa, synthetic construct
  REGION 1..107: note = Synthetic: BGA7592-1F-ph-M VL
DIQMTQSPSSLSASVGDRVTITCRASENQYGYLAWYQQKPGKVPKLLIYNTKNLVEGVPSRFSGSGSGTDFTLTISSLQPEDVATYYCQHHLGTPYTFGQGTKVEIK

SEQ ID NO: 97 - protein, 120 aa, synthetic construct
  REGION 1..120: note = Synthetic: VH
QVQLVQSGAEVKKPGASVKVSCKASGYIFTSYYLHWVRQAPGQGLEWIGYINPQTGYTSYAQKFQGRVTMTRDTSTSTVYMELSSLRSEDTAVYYCAREYGNYNYPLDYWGQGTLVTVSS

SEQ ID NO: 98 - DNA, 360 bp, synthetic construct
  misc_feature 1..360: note = Synthetic: VH DNA
  source 1..360: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggctataccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagc

SEQ ID NO: 99 - protein, 450 aa, synthetic construct
  REGION 1..450: note = Synthetic: BGA75921 K heavy chain
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 214 aa, synthetic construct
  REGION 1..214: note = Synthetic: BGA75921K light chain
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 4 aa, synthetic construct
  REGION 1..4: note = Synthetic: LCDR2
NAKN

SEQ ID NO: 102 - protein, 6 aa, synthetic construct
  REGION 1..6: note = Synthetic: HCDR1
YIFTSY

SEQ ID NO: 103 - protein, 13 aa, synthetic construct
  REGION 1..13: note = Synthetic: HCDR2
GYINPQTGYTSYA

SEQ ID NO: 104 - protein, 9 aa, synthetic construct
  REGION 1..9: note = Synthetic: HCDR3
EYGNYNYPL

SEQ ID NO: 105 - protein, 214 aa, synthetic construct
  REGION 1..214: note = Synthetic: Tusamitamab sequence LC
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 450 aa, synthetic construct
  REGION 1..450: note = Synthetic: Tusamitamab sequence HC
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0115677 A1
(43) Pub. Date: Apr. 10, 2025

(54) Anti-CEA Antibody Drug Conjugates and Methods of Use

(71) Applicant: BeiGene Switzerland GmbH, Basel (CH)

(72) Inventors: Charng-Sheng TSAI, Beijing (CN); Mei-Hsuan TSAI, Beijing (CN); Liang QU, Beijing (CN); Xiaodong WEI, Beijing (CN); Zewei WANG, Beijing (CN); Wei LUO, Beijing (CN); Maomao HE, Beijing (CN); Zhuo LI, Shanghai (CN)

(73) Assignee: BeiGene Switzerland GmbH, Basel (CH)

(21) Appl. No.: 18/940,225

(22) Filed: Nov. 7, 2024

Related U.S. Application Data
(63) Continuation of application No. PCT/IB23/61813, filed on Nov. 22, 2023.

(30) Foreign Application Priority Data
Nov. 24, 2022 (WO) PCT/CN22/134067
Jul. 12, 2023 (WO) PCT/CN23/107003

(51) Int. Cl. C07K 16/30; A61K 47/68; A61P 35/00

(52) U.S. Cl. CPC C07K 16/3007; A61K 47/68031; A61K 47/6853; A61P 35/00; C07K 2317/24; C07K 2317/33; C07K 2317/732; C07K 2317/92

(57) ABSTRACT

The present disclosure provides antibody drug conjugates comprising antibodies and antigen-binding fragments thereof that bind to human CEA and a linker-payload, a pharmaceutical composition comprising the anti-CEA antibody drug conjugate, and use of the anti-CEA antibody drug conjugate for treating a CEA-related diseases or disorders.

### CROSS-REFERENCE TO RELATED APPLICATIONS

This application is a continuation application of PCT Application No. PCT/IB2023/061813, filed Nov. 22, 2023, which claims the benefit of priority of PCT Application Nos. PCT/CN2022/134067, filed Nov. 24, 2022, entitled “Anti-CEA Antibody Drug Conjugates and Methods of Use,” and PCT/CN2023/107003, filed Jul. 12, 2023, entitled “Anti-CEA Antibody Drug Conjugates and Methods of Use,” which are hereby incorporated by reference in their entireties.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 20, 2023, is named “01368-0008-00PCT.xml” and is 156,334 bytes in size. The sequence listing contained in this .XML file is part of the specification and is hereby incorporated by reference herein in its entirety.

### FIELD OF THE DISCLOSURE

Disclosed herein are anti-CEA antibody drug conjugates (ADCs) comprising antibodies, or antigen-binding fragments thereof, that bind to human CEA, and are covalently linked to a growth-inhibiting agent, as well as therapeutic uses thereof.

### BACKGROUND

Antibody drug conjugates (ADCs) are chimeric molecules that combine antibody specificity to recognize and bind with high affinity to antigens such as tumor-associated antigens (TAA) with the potent enzymatic activity of a drug, e.g., toxin, to induce the death of target cells. Current ADCs present some therapeutic limitations, driving the need to develop new prototypes with optimized properties.

Carcinoembryonic antigen (CEA, also known as CEACAM5 or CD66e) is a glycoprotein with a molecular weight of about 70-100 kDa depending on the amount of glycosylation present. CEA is a TAA first described as an oncofetal protein in colorectal cancer. CEA is present at low levels in adult tissues from epithelial origin, such as colon, stomach, tongue, cervix, and prostate. CEA is restricted to the apical surface in non-tumor cells, but dislocated all over the cellular membrane in cancerous cells. CEA overexpression has been observed in many types of cancers, including colorectal cancer, pancreatic cancer, lung cancer, gastric cancer, hepatocellular carcinoma, breast cancer, and thyroid cancer. For example, CEA is found in the columnar epithelial and goblet cells of the colon. In tumors generated from these tissue types, CEA expression increases from the apical membrane to the cell surface and, once removed from the cell surface, enters into the bloodstream. CEA is constitutively released from tumor cells reaching detectable concentrations in peripheral blood such that CEA quantification has frequently been used to diagnosis cancer. Therefore, CEA is useful as a diagnostic tumor marker to determine the elevated levels of CEA in the blood of cancer patients in the prognosis and management of cancer.

CEA is also considered a useful tumor-associated antigen for targeted therapy. Retrovirus constructs that display an anti-CEA scFv to deliver a nitric oxide synthase (iNOS) gene to CEA-expressing cancer cells have been generated. Anti-CEA antibodies conjugated to radioisotopes have been used to demonstrate that radiation was directed specifically at the CEA-expressing tumor. The radioisotope approach has been extended to anti-CEA antibody drug conjugates (ADC), such as by conjugating an anti-CEA antibody to monomethyl auristatin E (MMAE).

However, one of the issues encountered for anti-CEA antibodies is that of cross-reactivity. CEA is highly homologous to other CEACAM family members. For example, human CEA shows 84% homology with CEACAM6, 77% homology with CEACAM8, and 73% identity with CEACAM1. There exists a need, therefore, for anti-CEA antibodies that are specific for CEA, and which do not significantly cross-react with human CEACAM1, human CEACAM6, human CEACAM7, or human CEACAM8. Moreover, a need remains for anti-CEA ADCs with highly potent anti-tumor activity combined with potent and non-cross-reactive anti-CEA targeting specificity.

### SUMMARY OF THE DISCLOSURE

The present disclosure encompasses at least the following embodiments.

The present disclosure is directed to antibody drug conjugates (ADCs) comprising an antibody or antigen-binding fragment (Ab) thereof, capable of specific binding to human CEA, and a cytotoxic agent (D).

In certain embodiments, the antibody or antigen-binding fragment (Ab) thereof comprises:
(i)
   three heavy chain CDRs:
   HCDR1 comprising an amino acid sequence as set forth in SEQ ID NO:24
   HCDR2 comprising an amino acid sequence as set forth in SEQ ID NO:25,
   HCDR3 comprising an amino acid sequence as set forth in SEQ ID NO:26, and
   three light chain CDRs:
   LCDR1 comprising an amino acid sequence as set forth in SEQ ID NO:27,
   LCDR2 comprising an amino acid sequence as set forth in SEQ ID NO:28,
   LCDR3 comprising an amino acid sequence as set forth in SEQ ID NO:23; or
(ii)
   three heavy chain CDRs:
   HCDR1 comprising an amino acid sequence as set forth in SEQ ID NO:7
   HCDR2 comprising an amino acid sequence as set forth in SEQ ID NO:8,
   HCDR3 comprising an amino acid sequence as set forth in SEQ ID NO:9, and
   three light chain CDRs:
   LCDR1 comprising an amino acid sequence as set forth in SEQ ID NO:10,
   LCDR2 comprising an amino acid sequence as set forth in SEQ ID NO:11,
   LCDR3 comprising an amino acid sequence as set forth in SEQ ID NO:6; or
(iii)
   three heavy chain CDRs:
   HCDR1 comprising an amino acid sequence as set forth in SEQ ID NO:41
   HCDR2 comprising an amino acid sequence as set forth in SEQ ID NO:42,
   HCDR3 comprising an amino acid sequence as set forth in SEQ ID NO:43, and
   three light chain CDRs:
   LCDR1 comprising an amino acid sequence as set forth in SEQ ID NO:4,
   LCDR2 comprising an amino acid sequence as set forth in SEQ ID NO:45,
   LCDR3 comprising an amino acid sequence as set forth in SEQ ID NO:40.

In certain embodiments, the present disclosure is directed to an antibody drug conjugate comprising the formula:

Ab-(C-L-(D)ₘ)ₙ

or a pharmaceutically acceptable salt, solvate, or hydrate thereof; wherein
Ab is the antibody or antigen-binding fragment thereof;
C is a conjugator,
L is a linker;
D is the cytotoxic agent;
m is an integer from 1 to 8; and
n is from 1 to 10.

In certain embodiments, m is 1. It will be appreciated that when m is 1, the antibody drug conjugate comprises (e.g., has) the formula: Ab-(C-L-D)ₙ.

In certain embodiments, C is a formula selected from (C-I), (C-Ia), (C-Ib), (C-II), (C-III), (C-IIIa), or (C-IV):

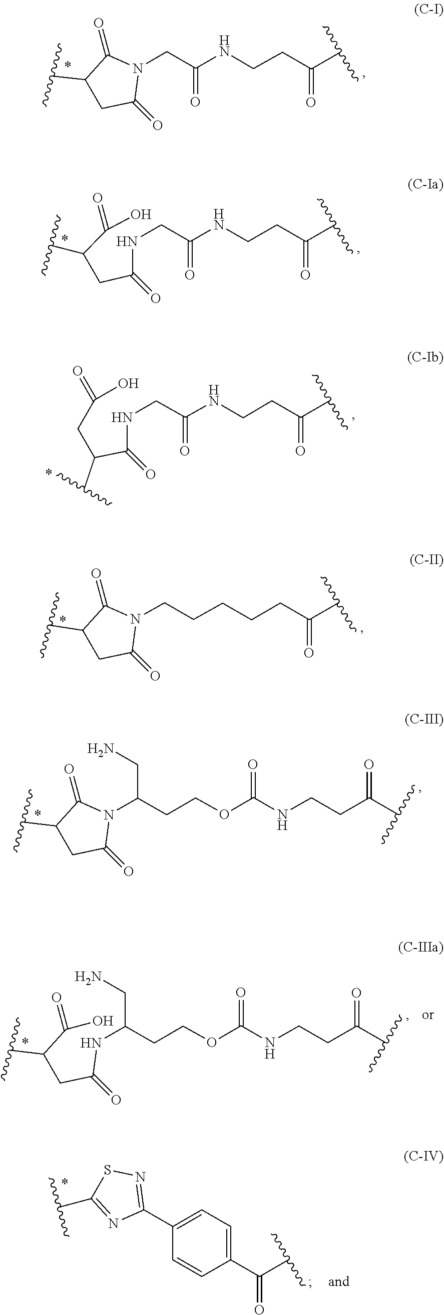

* marks the bond where C connects to Ab. In certain embodiments, C is a formula selected from:

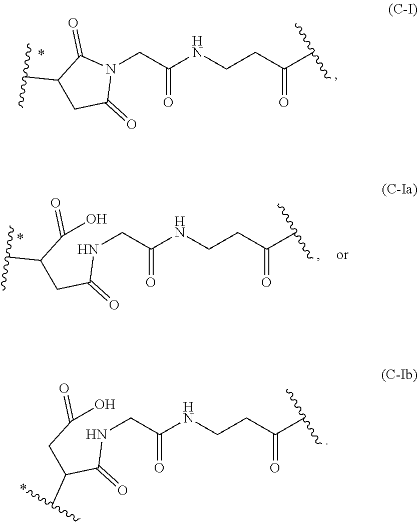

In certain embodiments, C is (C-Ic):

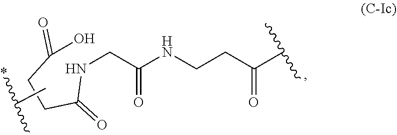

and * marks the bond where C connects to Ab.

In certain embodiments, C comprises (e.g., has) the following formula (C-I), (C-Ia), (C-II), (C-III), (C-IIIa), or (C-IV):

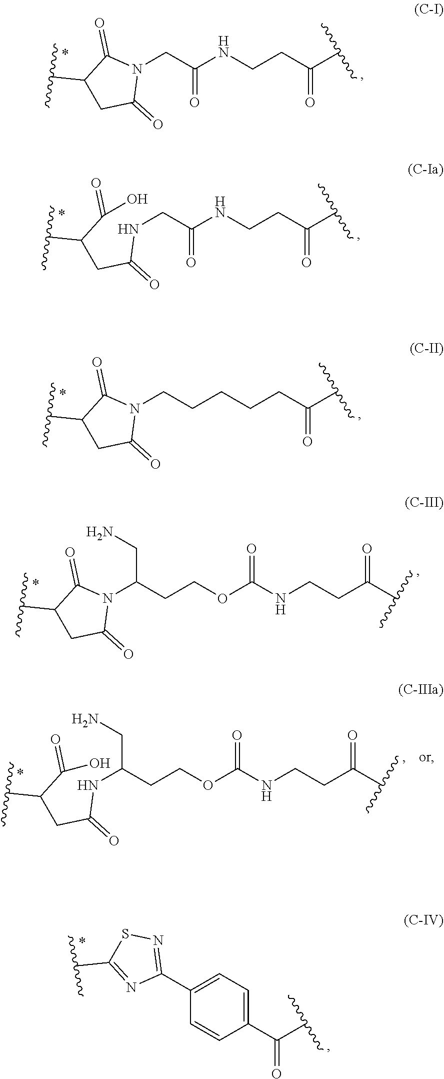 [SMILES: *C(=O)CCNC(=O)CN1C(=O)CC(*)C1=O ; *C(=O)CCNC(=O)CNC(=O)CC(*)C(=O)O ; *C(=O)CCCCCN1C(=O)CC(*)C1=O ; *C(=O)CCNC(=O)OCCC(CN)N1C(=O)CC(*)C1=O ; *C(=O)CCNC(=O)OCCC(CN)NC(=O)CC(*)C(=O)O ; *C(=O)c1ccc(-c2nsc(*)n2)cc1]

wherein * marks the bond where the conjugator connects to Ab.

In certain embodiments, L comprises (e.g., has) the following formula (L-I), (L-II), or (L-III):

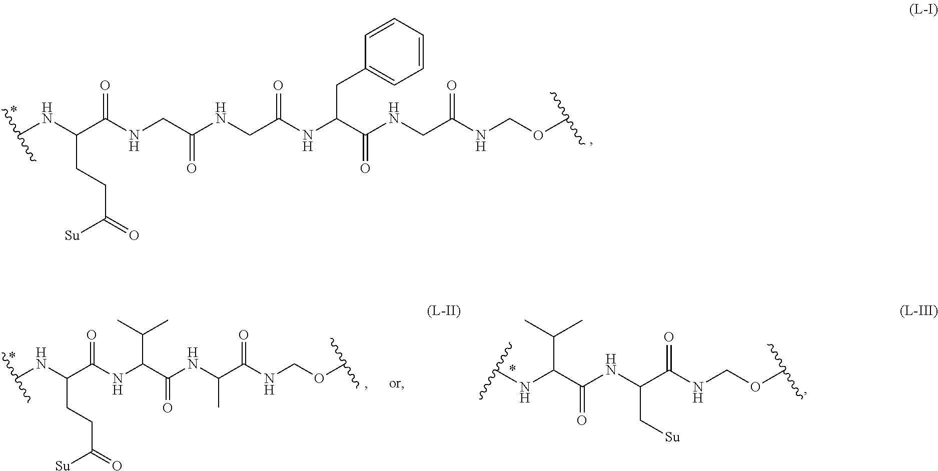

wherein Su is a hydrophilic residue; and
* marks the bond where the linker connects to the conjugator.

In certain embodiments, Su is

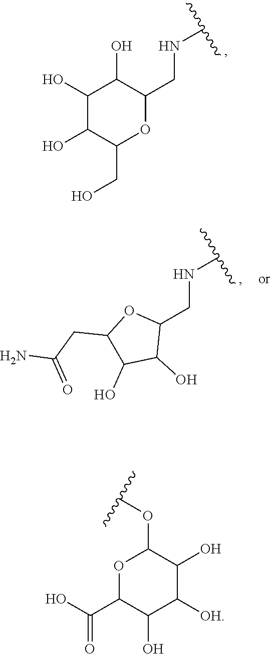 [SMILES: *NCC1OC(CO)C(O)C(O)C1O ; *NCC1OC(CC(N)=O)C(O)C1O ; *OC1OC(C(=O)O)C(O)C(O)C1O]

In certain embodiments, Su is

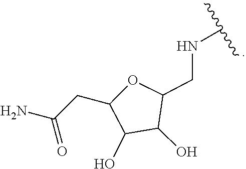 [SMILES: *NCC1OC(CC(N)=O)C(O)C1O]

In certain embodiments, Su is

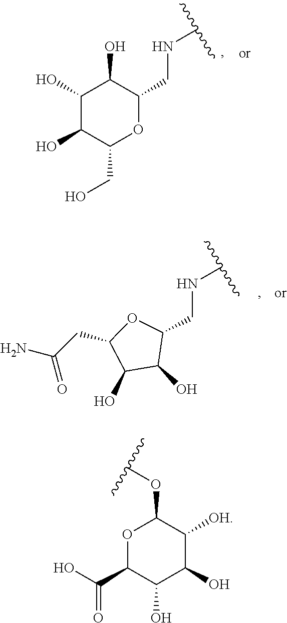 [SMILES: *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

In certain embodiments, Su is

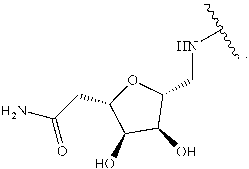 [SMILES: *NC[C@H]1O[C@@H](CC(N)=O)[C@H](O)[C@@H]1O]

In certain embodiments, L is

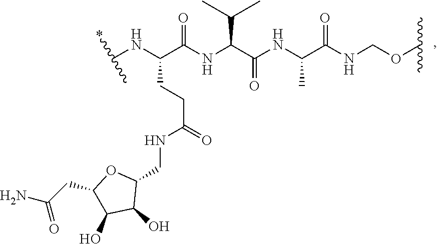

wherein * marks the bond where L connects to C.

In certain embodiments, the cytotoxic agent (D) is a topoisomerase inhibitor.

In certain embodiments, D is:

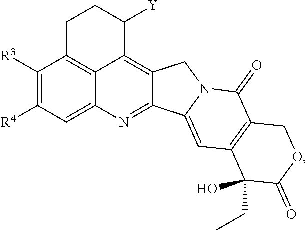

wherein values for the variables (e.g., Y, R³, R⁴) are as described herein.

In certain embodiments, D is of the following structural formula:

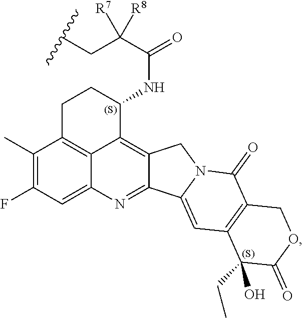

wherein values for the variables (e.g., R⁷, R⁸) are as described herein.

In certain embodiments, the cytotoxic agent (D) is

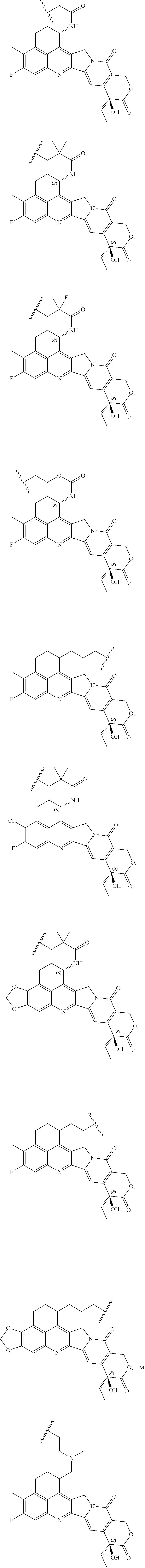

wherein values for the variables (e.g., R⁸, R⁸) are as described herein.

In certain embodiments, the cytotoxic agent (D) is

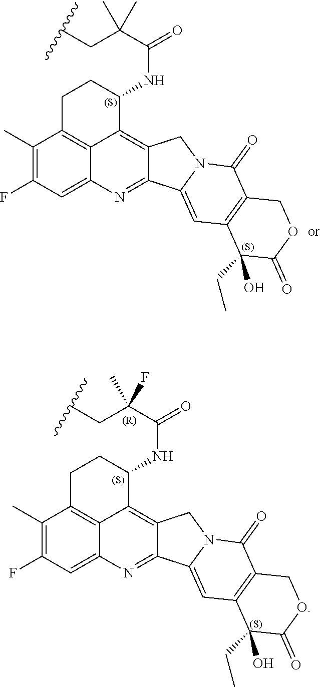

In certain embodiments, D is

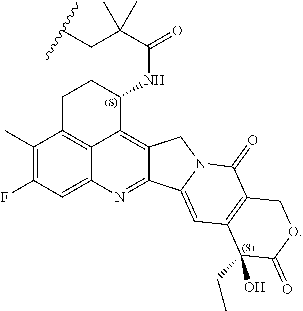

In certain embodiments, C-L-(D)ₘ is:

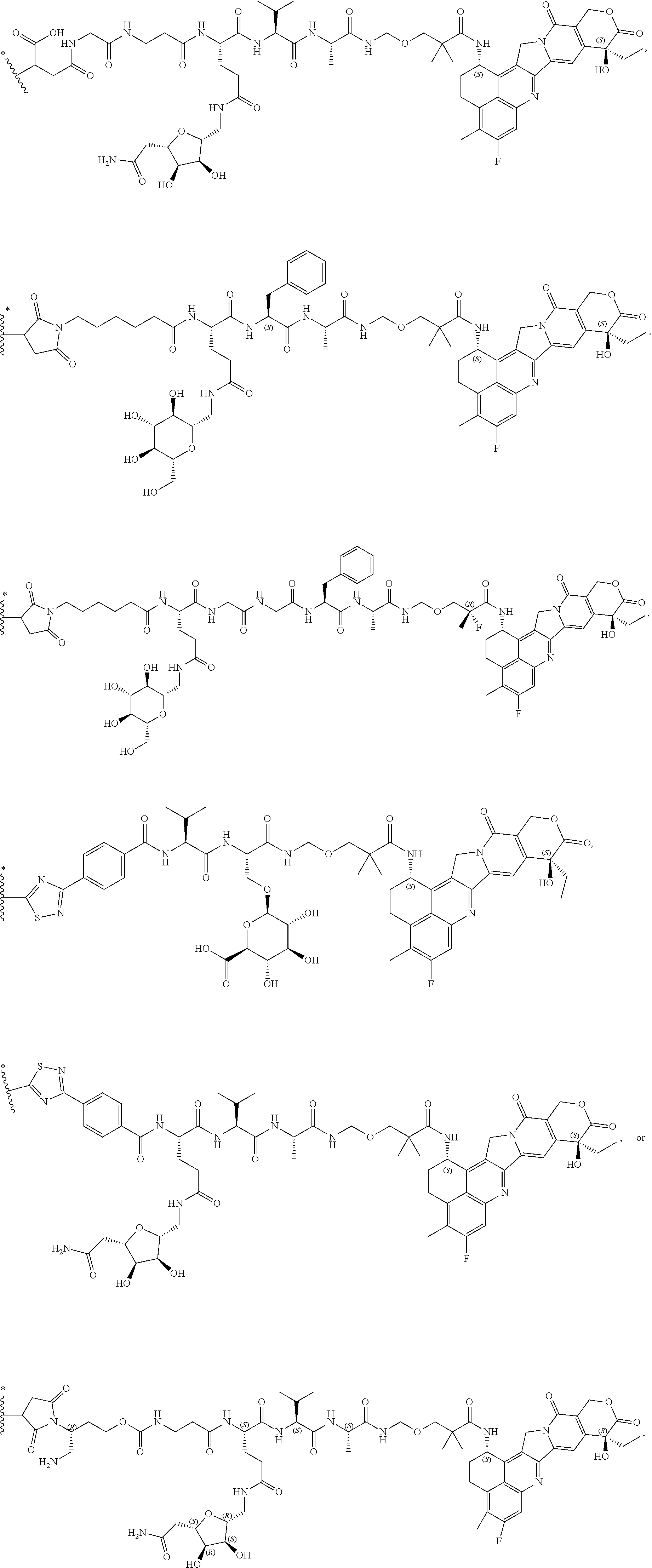

where * marks the bond where C connects to Ab.

In certain embodiments, C-L-(D)ₘ is:

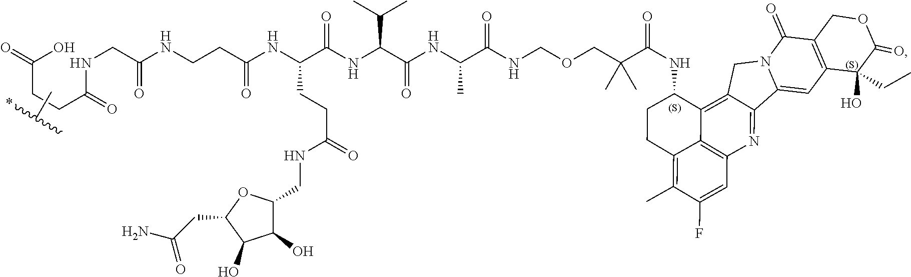

where * marks the bond where C connects to Ab.

In certain embodiments, C-L-(D)ₘ is:

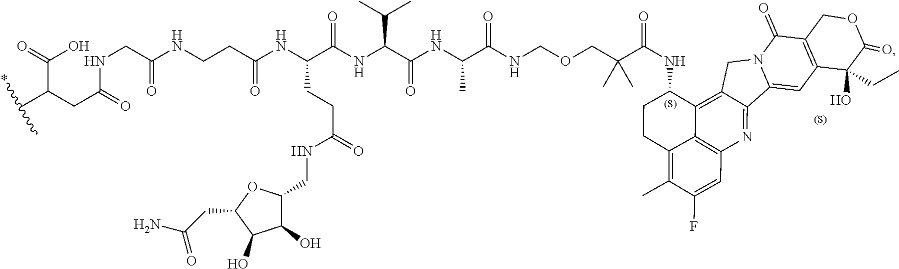

where * marks the bond where C connects to Ab.

In certain embodiments, C-L-(D)ₘ is:

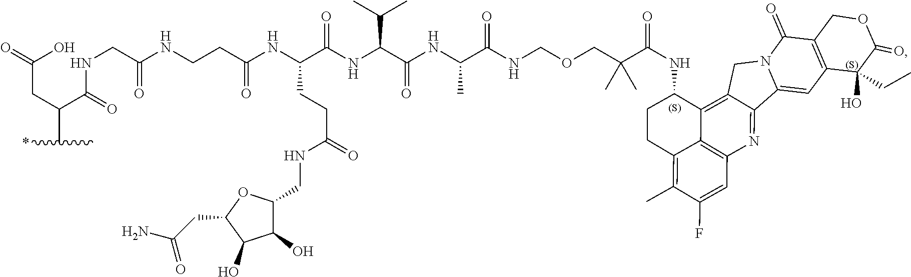

where * marks the bond where C connects to Ab.

In certain embodiments, the antibody drug conjugate is:

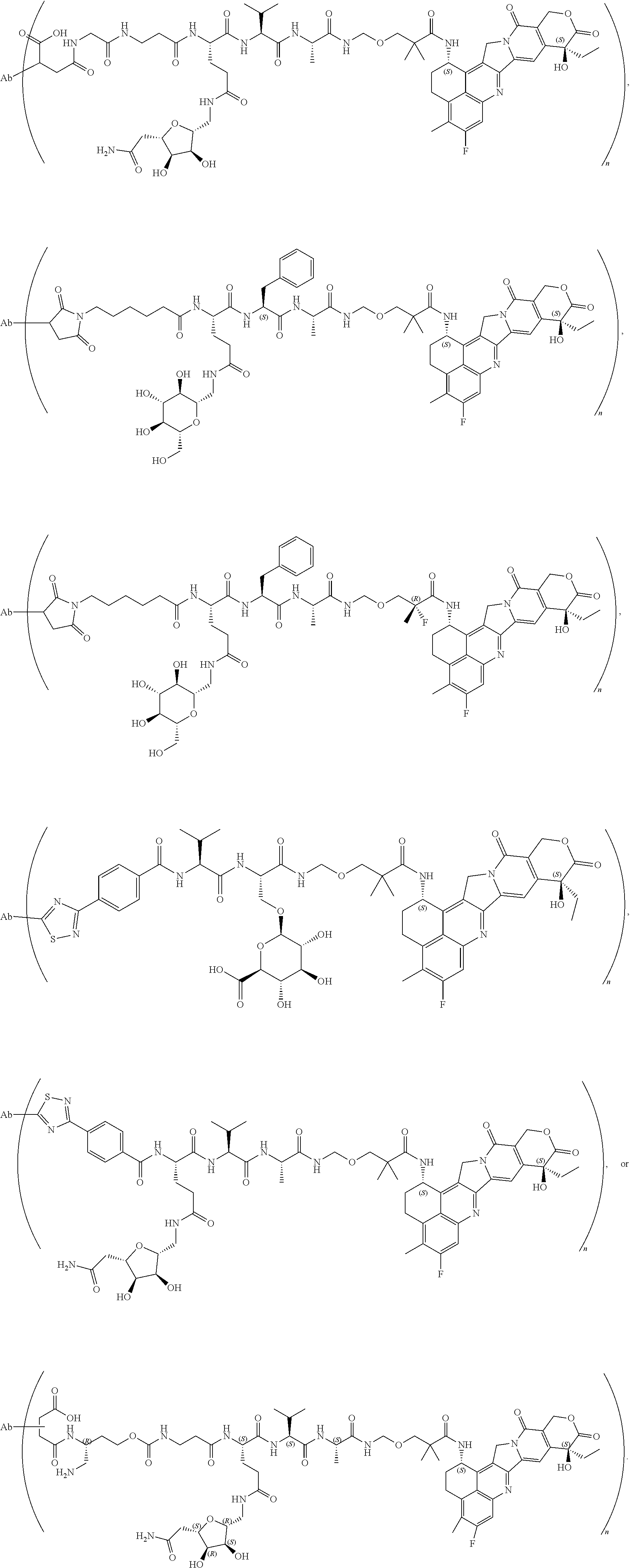

or a tautomer, pharmaceutically acceptable salt, solvate, or hydrate thereof, wherein Ab is an anti-CEA antibody or antigen-binding fragment thereof as described herein and n is as described herein, e.g., between 1 and 10, preferably about 7, 8, or 9.

In certain embodiments, the antibody drug conjugate is of the following formula:

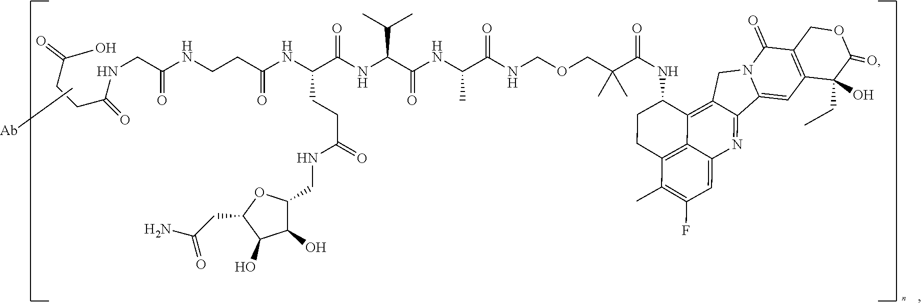

or a tautomer, pharmaceutically acceptable salt, solvate or hydrate thereof, wherein Ab and n are as described herein.

In certain embodiments, the antibody drug conjugate is of the following formula:

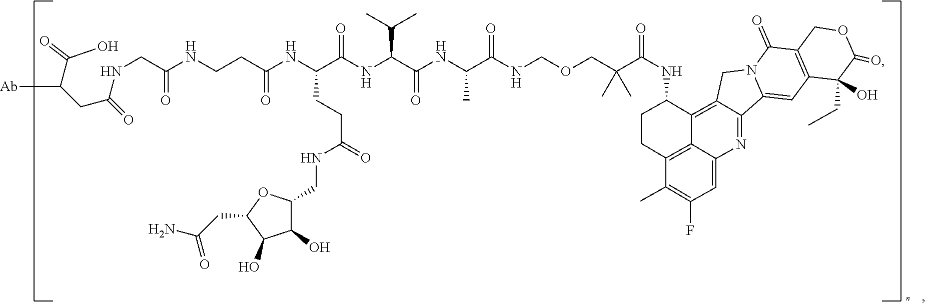

or a tautomer, pharmaceutically acceptable salt, solvate or hydrate thereof, wherein Ab and n are as described herein.

In certain embodiments, the antibody drug conjugate is of the following formula:

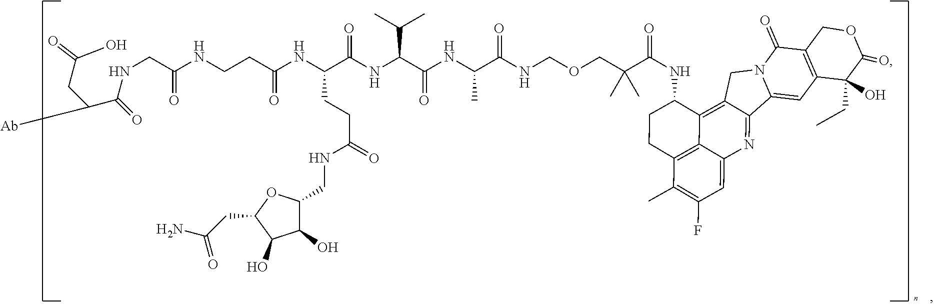

or a tautomer, pharmaceutically acceptable salt, solvate or hydrate thereof, wherein Ab and n are as described herein.

In certain embodiments, n is from 3 to 10, e.g., from 4 to 10, from 5 to 10, from 6 to 10, or from 7 to 9. In certain embodiments, n is about 8.

In certain embodiments, the present disclosure is directed to an antibody drug conjugate, comprising an anti-CEA antibody or antigen-binding fragment, wherein the antibody or antigen-binding fragment comprises:
(i) three heavy chain CDRs:
   HCDR1 comprising an amino acid sequence as set forth in SEQ ID NO:24,
   HCDR2 comprising an amino acid sequence as set forth in SEQ ID NO:25,
   HCDR3 comprising an amino acid sequence as set forth in SEQ ID NO:26, and
three light chain CDRs:
   LCDR1 comprising an amino acid sequence as set forth in SEQ ID NO:27,
   LCDR2 comprising an amino acid sequence as set forth in SEQ ID NO:28,
   LCDR3 comprising an amino acid sequence as set forth in SEQ ID NO:23; or
(ii) three heavy chain CDRs:
   HCDR1 comprising an amino acid sequence as set forth in SEQ ID NO:7,
   HCDR2 comprising an amino acid sequence as set forth in SEQ ID NO:8,
   HCDR3 comprising an amino acid sequence as set forth in SEQ ID NO:9, and
three light chain CDRs:
   LCDR1 comprising an amino acid sequence as set forth in SEQ ID NO:10,
   LCDR2 comprising an amino acid sequence as set forth in SEQ ID NO: 11,
   LCDR3 comprising an amino acid sequence as set forth in SEQ ID NO:6; or
(iii) three heavy chain CDRs:
   HCDR1 comprising an amino acid sequence as set forth in SEQ ID NO:41,
   HCDR2 comprising an amino acid sequence as set forth in SEQ ID NO:42,
   HCDR3 comprising an amino acid sequence as set forth in SEQ ID NO:43, and
three light chain CDRs:
   LCDR1 comprising an amino acid sequence as set forth in SEQ ID NO:44,
   LCDR2 comprising an amino acid sequence as set forth in SEQ ID NO:45,
   LCDR3 comprising an amino acid sequence as set forth in SEQ ID NO:40; or
(iv) a heavy chain variable region comprising SEQ ID NO:31, and a light chain variable region comprising SEQ ID NO:32;
(v) a heavy chain variable region comprising SEQ ID NO:48, and a light chain variable region comprising SEQ ID NO:49; or
(vi) a heavy chain variable region comprising SEQ ID NO:14, and a light chain variable region comprising SEQ ID NO:15; or
(vii) a heavy chain variable region comprising a sequence having at least 85%, 90%, 95%, 96%, 97%, 98%, or 99% identity to SEQ ID NO:31, and a light chain variable region comprising a sequence having at least 85%, 90%, 95%, 96%, 97%, 98%, or 99% identity to SEQ ID NO:32; or
(viii) a heavy chain variable region comprising a sequence having at least 85%, 90%, 95%, 96%, 97%, 98%, or 99% identity to SEQ ID NO:48, and a light chain variable region comprising a sequence having at least 85%, 90%, 95%, 96%, 97%, 98%, or 99% identity to SEQ ID NO:49; or
(ix) a heavy chain variable region comprising a sequence having at least 85%, 90%, 95%, 96%, 97%, 98%, or 99% identity to SEQ ID NO:14, and a light chain variable region comprising a sequence having at least 85%, 90%, 95%, 96%, 97%, 98%, or 99% identity to SEQ ID NO:15.

In certain embodiments, the antibody or antigen-binding fragment is a monoclonal antibody, a human engineered antibody, a single chain antibody (scFv), a Fab fragment, a Fab′ fragment, or a F(ab′)2 fragment.

In certain embodiments, the antibody or antigen-binding fragment comprises an scFv comprising a VH having the amino acid sequence of SEQ ID NO:14 and a VL having the amino acid sequence of SEQ ID NO:15.

In certain embodiments, the antibody or antigen-binding fragment comprises an scFv comprising a VH having the amino acid sequence of SEQ ID NO:31 and a VL having the amino acid sequence of SEQ ID NO:32.

In certain embodiments, the antibody or antigen-binding fragment comprises an scFv comprising a VH having the amino acid sequence of SEQ ID NO:48 and a VL having the amino acid sequence of SEQ ID NO:49.

In certain embodiments, the antibody or antigen-binding fragment comprises an scFv having the amino acid sequence of SEQ ID NO:14, SEQ ID NO:31, or SEQ ID NO:48.

In certain embodiments, the antibody or antigen-binding fragment thereof comprises a heavy chain constant region of the subclass of IgG1, IgG2, IgG3, or IgG4, and/or a light chain constant region of the type of kappa or lambda.

In certain embodiments, the antibody or antigen-binding fragment thereof comprises a heavy chain constant region of the subclass of IgG1, and a light chain constant region of the type of kappa.

In certain embodiments, the present disclosure is directed to an antibody drug conjugate of any one of the following formulas:

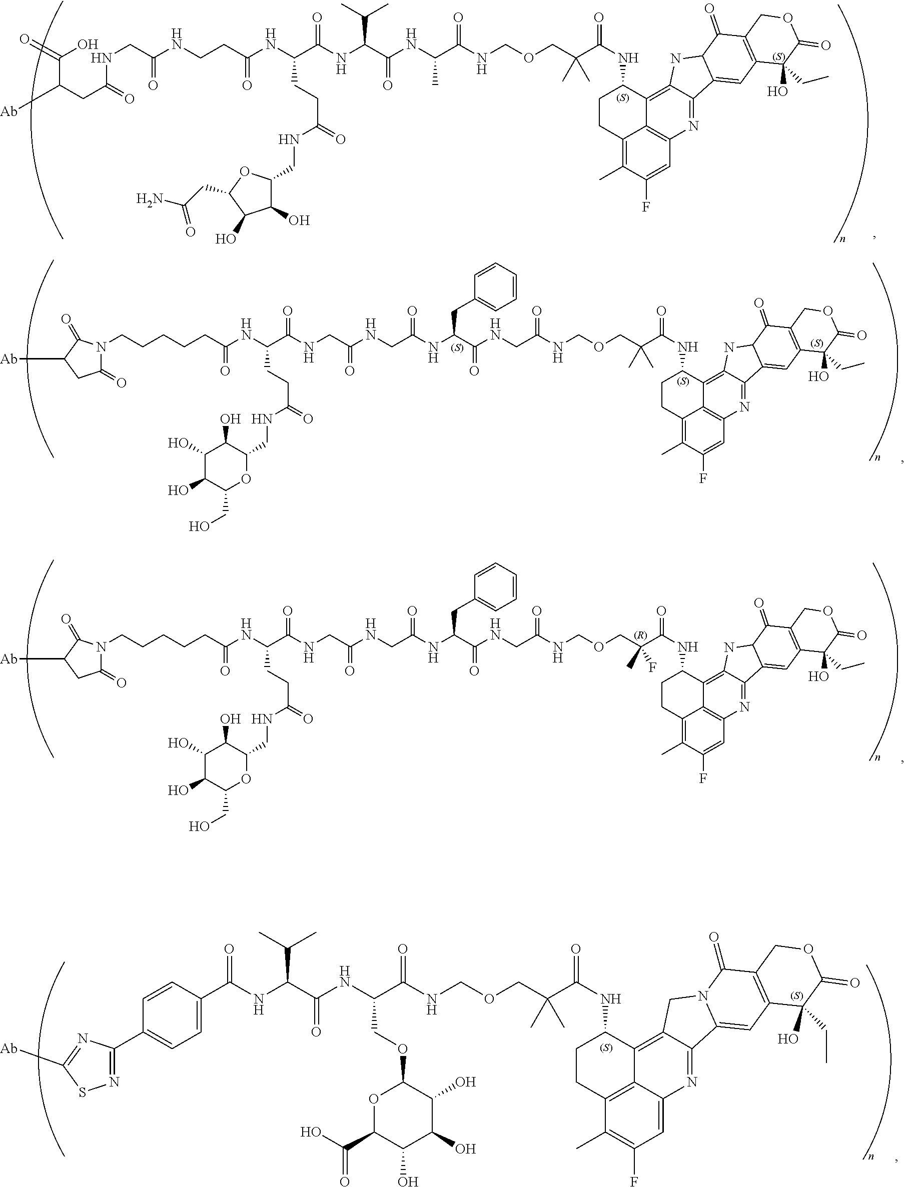

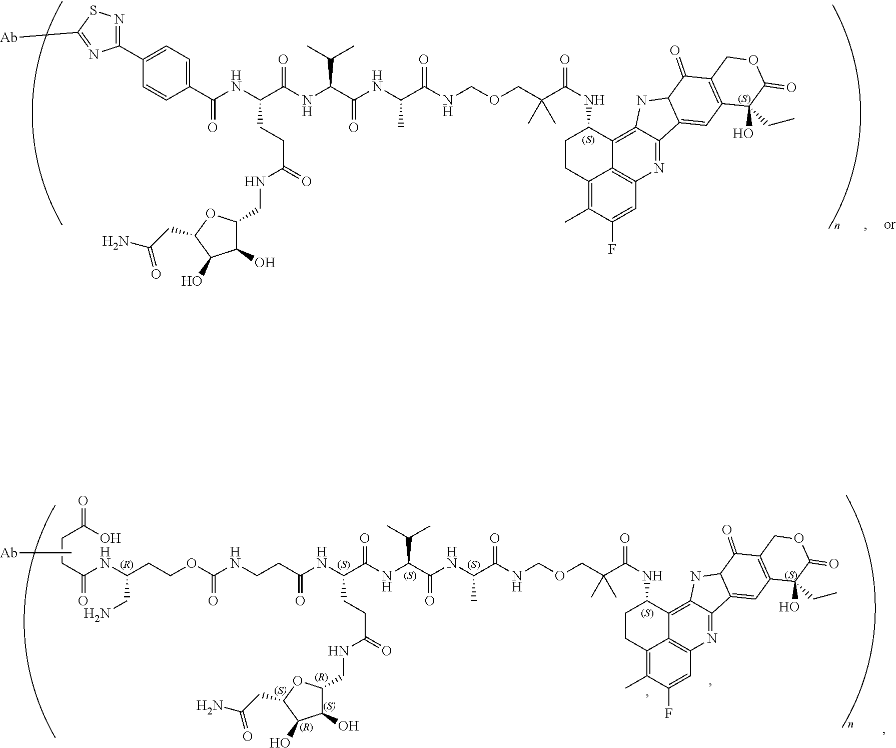

or a tautomer, pharmaceutically acceptable salt, solvate, or hydrate thereof;
where n is from 4 to 10, e.g., 4, 5, 6, 7, 8, 9, or 10;
Ab is an antibody or antigen-binding fragment thereof that binds CEA; and
the antibody or antigen-binding fragment comprises:
(i)
   three heavy chain CDRs:
   HCDR1 comprising an amino acid sequence as set forth in SEQ ID NO:24
   HCDR2 comprising an amino acid sequence as set forth in SEQ ID NO:25,
   HCDR3 comprising an amino acid sequence as set forth in SEQ ID NO:26, and
   three light chain CDRs:
   LCDR1 comprising an amino acid sequence as set forth in SEQ ID NO:27,
   LCDR2 comprising an amino acid sequence as set forth in SEQ ID NO:28,
   LCDR3 comprising an amino acid sequence as set forth in SEQ ID NO:23; or
(ii)
   three heavy chain CDRs:
   HCDR1 comprising an amino acid sequence as set forth in SEQ ID NO:7
   HCDR2 comprising an amino acid sequence as set forth in SEQ ID NO:8,
   HCDR3 comprising an amino acid sequence as set forth in SEQ ID NO:9, and
   three light chain CDRs:
   LCDR1 comprising an amino acid sequence as set forth in SEQ ID NO:10,
   LCDR2 comprising an amino acid sequence as set forth in SEQ ID NO:11,
   LCDR3 comprising an amino acid sequence as set forth in SEQ ID NO:6; or
(iii)
   three heavy chain CDRs:
   HCDR1 comprising an amino acid sequence as set forth in SEQ ID NO:41
   HCDR2 comprising an amino acid sequence as set forth in SEQ ID NO:42,
   HCDR3 comprising an amino acid sequence as set forth in SEQ ID NO:43, and
   three light chain CDRs:
   LCDR1 comprising an amino acid sequence as set forth in SEQ ID NO:44,
   LCDR2 comprising an amino acid sequence as set forth in SEQ ID NO:45,
   LCDR3 comprising an amino acid sequence as set forth in SEQ ID NO:40; or
(iv) a heavy chain variable region comprising SEQ ID NO:31, and a light chain variable region comprising SEQ ID NO:32;
(v) a heavy chain variable region comprising SEQ ID NO:48, and a light chain variable region comprising SEQ ID NO:49; or
(vi) a heavy chain variable region comprising SEQ ID NO:14, and a light chain variable region comprising SEQ ID NO:15; or
(vii) a heavy chain variable region comprising a sequence having at least 85%, 90%, 95%, 96%, 97%, 98%, or 99% identity to SEQ ID NO:31, and a light chain variable region comprising a sequence having at least 85%, 90%, 95%, 96%, 97%, 98%, or 99% identity to SEQ ID NO:32; or
(viii) a heavy chain variable region comprising a sequence having at least 85%, 90%, 95%, 96%, 97%, 98%, or 99% identity to SEQ ID NO:48, and a light chain variable region comprising a sequence having at least 85%, 90%, 95%, 96%, 97%, 98%, or 99% identity to SEQ ID NO:49; or
(ix) a heavy chain variable region comprising a sequence having at least 85%, 90%, 95%, 96%, 97%, 98%, or 99% identity to SEQ ID NO:14, and a light chain variable region comprising a sequence having at least 85%, 90%, 95%, 96%, 97%, 98%, or 99% identity to SEQ ID NO:15.

In certain embodiments, n is about 8.

In certain embodiments, the present disclosure is directed to a pharmaceutical composition comprising the antibody drug conjugate as set forth above and herein and a pharmaceutically acceptable carrier.

In certain embodiments, the present disclosure is directed to a method of treating a subject (e.g., patient) having a CEA-related disease or disorder, for example, a CEA-expressing or accumulating cell, comprising administering to a subject (e.g., patient) in need thereof an effective amount of an antibody drug conjugate set forth herein, or a pharmaceutical composition comprising the same. In some embodiments, the CEA-expressing or accumulating cell is a cancerous cell.

In certain embodiments, the present disclosure is directed to an anti-CEA antibody conjugated to a Compound comprising the formula:

C-L-D

or a pharmaceutically acceptable salt, solvate, or hydrate thereof, wherein
C is a conjugator;
L is a linker; and
D is the cytotoxic agent.

In certain embodiments, C comprises the following formula (C-I′), (C-II′), (C-III′), or (C-IV′):

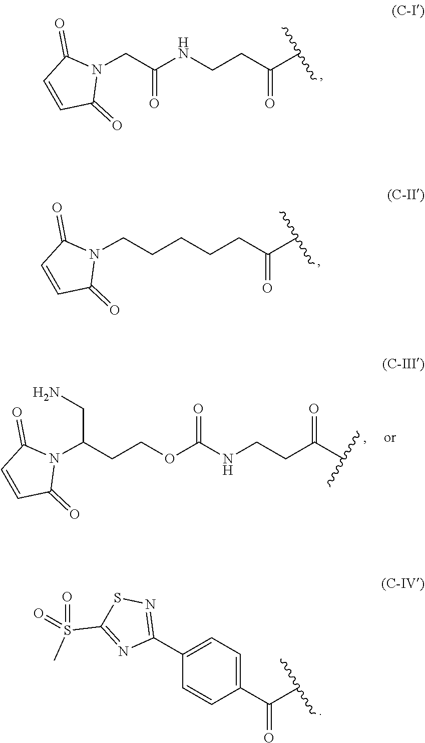 [SMILES: *C(=O)CCNC(=O)CN1C(=O)C=CC1=O ; *C(=O)CCCCCN1C(=O)C=CC1=O ; *C(=O)CCNC(=O)OCCC(CN)N1C(=O)C=CC1=O ; *C(=O)c1ccc(-c2nsc(S(C)(=O)=O)n2)cc1]

In certain embodiments, L comprises the following formula (L-I), (L-II), or (L-III):

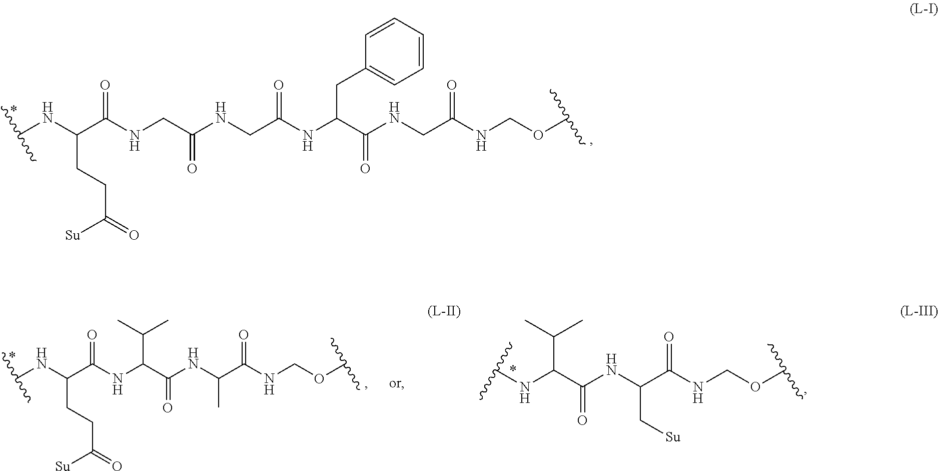

wherein Su is a hydrophilic residue; and
* marks the bond where the linker connects to the conjugator.

In certain embodiments, Su is

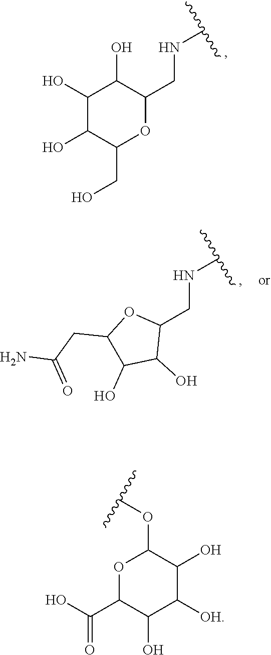 [SMILES: *NCC1OC(CO)C(O)C(O)C1O ; *NCC1OC(CC(N)=O)C(O)C1O ; *OC1OC(C(=O)O)C(O)C(O)C1O]

In certain embodiments, Su is

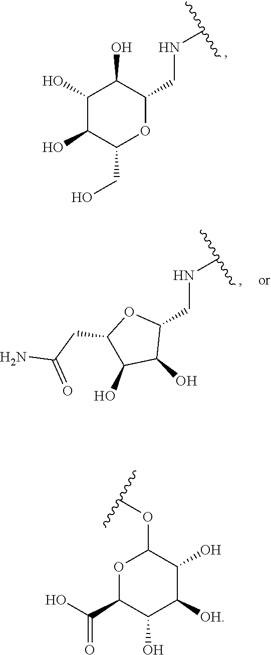 [SMILES: *NC[C@@H]1O[C@H](CO)[C@@H](O)[C@H](O)[C@H]1O ; *NC[C@H]1O[C@@H](CC(N)=O)[C@H](O)[C@@H]1O ; *OC1O[C@H](C(=O)O)[C@@H](O)[C@H](O)[C@H]1O]

In certain embodiments, the cytotoxic agent (D) is

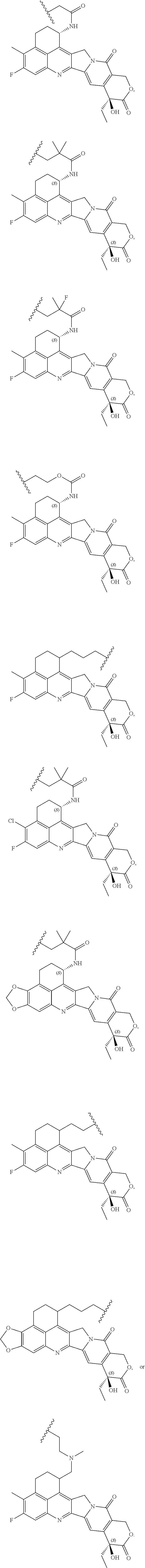

In certain embodiments, the cytotoxic agent (D) is

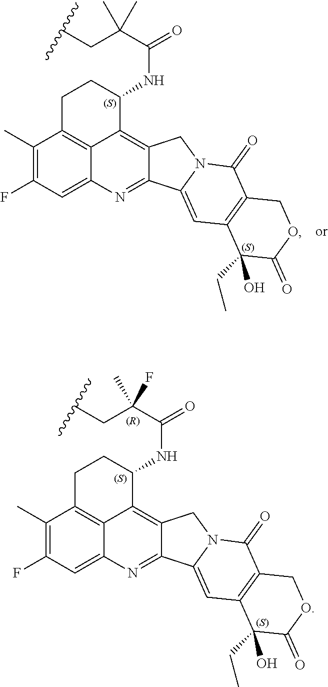

In certain embodiments, the Compound is

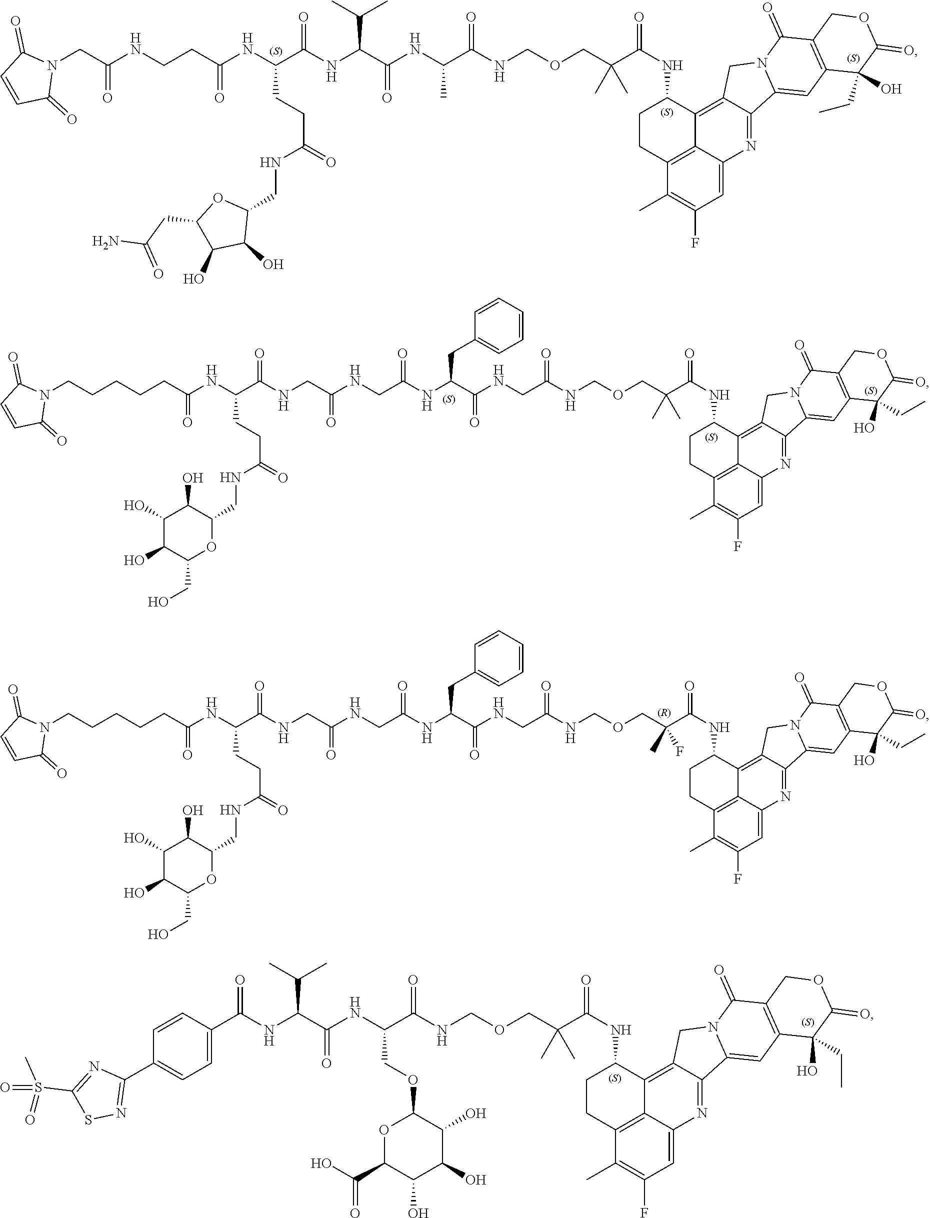

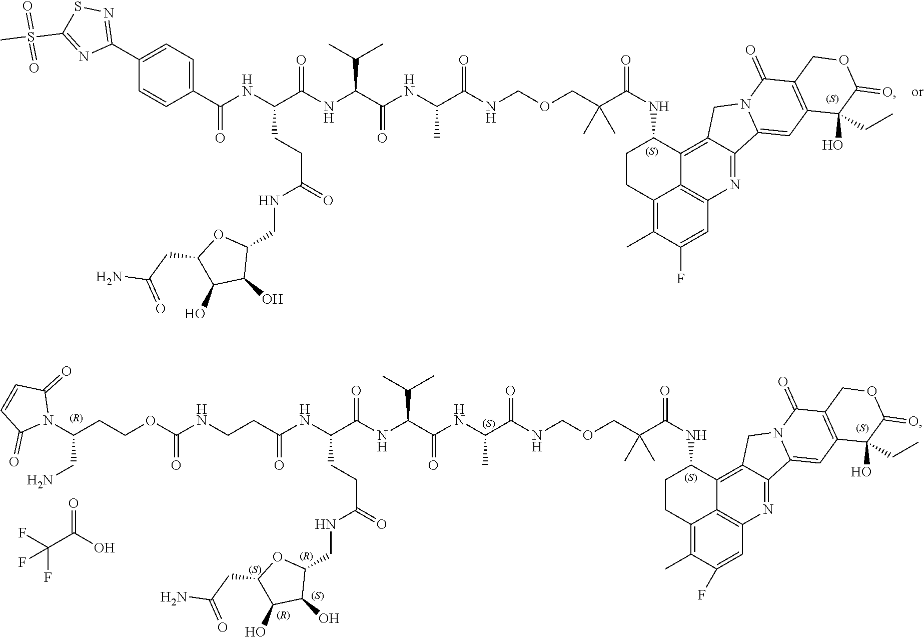

or a pharmaceutically acceptable salt, solvate, or hydrate thereof.

In certain embodiments, the present disclosure is directed to a method of producing an anti-CEA antibody drug conjugate as described above, comprising:
(i) culturing a host cell which has been transformed by an isolated nucleic acid comprising a sequence encoding an anti-CEA antibody or antigen-binding fragment thereof, wherein the antibody or fragment thereof comprises
   a) a heavy chain comprising an amino acid sequence of SEQ ID NO:99 and a light chain comprising an amino acid sequence of SEQ ID NO:100, or a sequence having at least 85%, 90%, 95%, 96%, 97%, 98%, or 99% identity to a heavy chain comprising an amino acid sequence of SEQ ID NO:99 and a light chain comprising an amino acid sequence of SEQ ID NO:100, wherein certain CDRs of the heavy and light chains shown as bold/underline in Table 20 are retained; or
   b) three heavy chain CDRs:
HCDR1 comprising an amino acid sequence as set forth in SEQ ID NO:24
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:23; or
   c) three heavy chain CDRs:
HCDR1 comprising an amino acid sequence as set forth in SEQ ID NO:7
HCDR2 comprising an amino acid sequence as set forth in SEQ ID NO:8,
HCDR3 comprising an amino acid sequence as set forth in SEQ ID NO:9, and
three light chain CDRs:
LCDR1 comprising an amino acid sequence as set forth in SEQ ID NO:10,
LCDR2 comprising an amino acid sequence as set forth in SEQ ID NO:11,
LCDR3 comprising an amino acid sequence as set forth in SEQ ID NO:6; or
   d) three heavy chain CDRs:
HCDR1 comprising an amino acid sequence as set forth in SEQ ID NO:41
HCDR2 comprising an amino acid sequence as set forth in SEQ ID NO:42,
HCDR3 comprising an amino acid sequence as set forth in SEQ ID NO:43, and
three light chain CDRs:
LCDR1 comprising an amino acid sequence as set forth in SEQ ID NO:44,
LCDR2 comprising an amino acid sequence as set forth in SEQ ID NO:45,
LCDR3 comprising an amino acid sequence as set forth in SEQ ID NO:40; or
   e) a heavy chain variable region comprising SEQ ID NO:31, and a light chain variable region comprising SEQ ID NO:32;
   f) a heavy chain variable region comprising SEQ ID NO:48, and a light chain variable region comprising SEQ ID NO:49; or
   g) a heavy chain variable region comprising SEQ ID NO:14, and a light chain variable region comprising SEQ ID NO:15; or
   h) a heavy chain variable region comprising a sequence having at least 85%, 90%, 95%, 96%, 97%, 98%, or 99% identity to SEQ ID NO:31, and a light chain variable region comprising a sequence having at least 85%, 90%, 95%, 96%, 97%, 98%, or 99% identity to SEQ ID NO:32; or
   i) a heavy chain variable region comprising a sequence having at least 85%, 90%, 95%, 96%, 97%, 98%, or 99% identity to SEQ ID NO:48, and a light chain variable region comprising a sequence having at least 85%, 90%, 95%, 96%, 97%, 98%, or 99% identity to SEQ ID NO:49; or
   j) a heavy chain variable region comprising a sequence having at least 85%, 90%, 95%, 96%, 97%, 98%, or 99% identity to SEQ ID NO:14, and a light chain variable region comprising a sequence having at least 85%, 90%, 95%, 96%, 97%, 98%, or 99% identity to SEQ ID NO:15; and
(ii) expressing said antibody or antigen-binding fragment thereof;
(iii) recovering the expressed antibody or antigen-binding fragment thereof; and
(iv) conjugating or linking at least one Compound to the antibody or fragment thereof optionally using a linker, such that an antibody drug conjugate is formed.

In certain embodiments, use of any of the antibody drug conjugates set forth herein (e.g., in the form a pharmaceutical composition) for a treatment set forth herein (e.g., treatment of a subject having a CEA-expressing and/or accumulating cell is provided.

In certain embodiments, provided are antibody drug conjugates set forth herein (e.g., in the form a pharmaceutical composition) for use as set forth herein (e.g., for treating a subject having a CEA-expressing and/or accumulating cell).

In certain embodiments, use of any of the antibody drug conjugates set forth herein (e.g., in the form a pharmaceutical composition) in the manufacture of a medicament for a treatment set forth herein (e.g., treatment of a subject having a CEA-expressing and/or accumulating cell) is provided.

In certain embodiments, provided is a kit comprising any one or more the antibody drug conjugates disclosed herein (e.g., in the form of a composition) and instructions for using the same. In some embodiments, the kit further comprises instructions for a detection assay, wherein the antibody drug conjugate forms a complex with CEA that is detected by an assay comprising an enzyme linked immunosorbent assay (ELISA), radioimmune assay (RIA), and/or Western blot.

In certain embodiments, the present disclosure is directed to a kit comprising an anti-CEA antibody drug conjugate, and instructions for using the same, the antibody drug conjugate comprising an antibody or antigen-binding fragment thereof, comprising:
(i)
   three heavy chain CDRs:
   HCDR1 comprising an amino acid sequence as set forth in SEQ ID NO:24
   HCDR2 comprising an amino acid sequence as set forth in SEQ ID NO:25,
   HCDR3 comprising an amino acid sequence as set forth in SEQ ID NO:26, and
   three light chain CDRs:
   LCDR1 comprising an amino acid sequence as set forth in SEQ ID NO:27,
   LCDR2 comprising an amino acid sequence as set forth in SEQ ID NO:28,
   LCDR3 comprising an amino acid sequence as set forth in SEQ ID NO:23; or
(ii)
   three heavy chain CDRs:
   HCDR1 comprising an amino acid sequence as set forth in SEQ ID NO:7
   HCDR2 comprising an amino acid sequence as set forth in SEQ ID NO:8,
   HCDR3 comprising an amino acid sequence as set forth in SEQ ID NO:9, and
   three light chain CDRs:
   LCDR1 comprising an amino acid sequence as set forth in SEQ ID NO:10,
   LCDR2 comprising an amino acid sequence as set forth in SEQ ID NO:11,
   LCDR3 comprising an amino acid sequence as set forth in SEQ ID NO:6; or
(iii)
   three heavy chain CDRs:
   HCDR1 comprising an amino acid sequence as set forth in SEQ ID NO:41
   HCDR2 comprising an amino acid sequence as set forth in SEQ ID NO:42,
   HCDR3 comprising an amino acid sequence as set forth in SEQ ID NO:43, and
   three light chain CDRs:
   LCDR1 comprising an amino acid sequence as set forth in SEQ ID NO:44,
   LCDR2 comprising an amino acid sequence as set forth in SEQ ID NO:45,
   LCDR3 comprising an amino acid sequence as set forth in SEQ ID NO:40; or
(iv) a heavy chain variable region comprising SEQ ID NO:31, and a light chain variable region comprising SEQ ID NO:32;
(v) a heavy chain variable region comprising SEQ ID NO:48, and a light chain variable region comprising SEQ ID NO:49; or
(vi) a heavy chain variable region comprising SEQ ID NO:14, and a light chain variable region comprising SEQ ID NO: 15; or
(vii) a heavy chain variable region comprising a sequence having at least 85%, 90%, 95%, 96%, 97%, 98%, or 99% identity to SEQ ID NO:31, and a light chain variable region comprising a sequence having at least 85%, 90%, 95%, 96%, 97%, 98%, or 99% identity to SEQ ID NO:32; or
(viii) a heavy chain variable region comprising a sequence having at least 85%, 90%, 95%, 96%, 97%, 98%, or 99% identity to SEQ ID NO:48, and a light chain variable region comprising a sequence having at least 85%, 90%, 95%, 96%, 97%, 98%, or 99% identity to SEQ ID NO:49; or
(ix) a heavy chain variable region comprising a sequence having at least 85%, 90%, 95%, 96%, 97%, 98%, or 99% identity to SEQ ID NO:14, and a light chain variable region comprising a sequence having at least 85%, 90%, 95%, 96%, 97%, 98%, or 99% identity to SEQ ID NO:15.

In certain embodiments, the present disclosure is directed to a kit comprising an anti-CEA antibody drug conjugate and instructions for using the same, wherein the antibody drug conjugate comprises an antibody comprising:
a. a VH sequence comprising the sequence set forth in SEQ ID NO:31, or a VH sequence comprising a sequence having at least 85%, 90%, 95%, 96%, 97%, 98%, or 99% identity to SEQ ID NO:31, and a VL sequence comprising the sequence set forth in SEQ ID NO:32, or a VL sequence comprising a sequence having at least 85%, 90%, 95%, 96%, 97%, 98%, or 99% identity to SEQ ID NO:32;
b. a VH sequence comprising the sequence set forth in SEQ ID NO:48 or a VH sequence comprising a sequence having at least 85%, 90%, 95%, 96%, 97%, 98%, or 99% identity to SEQ ID NO. 48, and a VL sequence comprising the sequence set forth in SEQ ID NO:49, or a VL sequence comprising a sequence having at least 85%, 90%, 95%, 96%, 97%, 98%, or 99% identity to SEQ ID NO:49; or
c. a VH sequence comprising the sequence set forth in SEQ ID NO:14 or a VH sequence comprising a sequence having at least 85%, 90%, 95%, 96%, 97%, 98%, or 99% identity to SEQ ID NO:14, and a VL sequence comprising the sequence set forth in SEQ ID NO:15, or a VL sequence comprising a sequence having at least 85%, 90%, 95%, 96%, 97%, 98%, or 99% identity to SEQ ID NO:15.

This Summary is neither intended as, nor should it be construed as, being representative of the full extent and scope of the present disclosure. Moreover, references made herein to “the present disclosure,” or aspects thereof, should be understood to mean certain embodiments of the present disclosure and should not be construed as limiting all embodiments to a particular description. The present disclosure is set forth in various levels of detail in this Summary as well as in the Detailed Description and accompanying drawings, and no limitation as to the scope of the present disclosure is intended by either the inclusion or non-inclusion of elements, components, etc. in this Summary. Features from any of the disclosed embodiments may be used in combination with one another, without limitation. In addition, other features and advantages of the present disclosure will become apparent to those of ordinary skill in the art through consideration of the following Detailed Description and the accompanying drawings.

### BRIEF DESCRIPTION OF THE DRAWINGS

FIG. 1 shows schematic diagrams of shed CEA (sCEA), chimeric CEA (CHIM), CEACAM6, and a CEA variant (CEA-v). In CEA, domains N, A1, B1, A2, B2, A3, B3, and GPI linker (GPI) are labeled; in CEACAM6, domains N′, A′, and B′ are labeled.

FIGS. 2A-B depict phylogenetic trees of anti-CEA domain B3 antibody VH (FIG. 2A) and VL (FIG. 2B) regions. The VH and VL sequences of candidate anti-CEA antibodies were aligned using DNASTAR's Megalign™ software. Sequence homology was displayed in phylogenetic trees.

FIG. 3A shows affinity determination of purified murine anti-CEA antibody BGA7592 on a chimeric construct (CHIM) by surface plasmon resonance (SPR). The various lines indicate binding capability of BGA7592 with CHIM at different concentrations, with the top line showing the binding capability of BGA7592 with CHIM at highest concentration, and sequential dilutions of CHIM forming the other lines. The rising curve of each line shows association rate and the declining curve shows dissociation rate.

FIG. 3B depicts binding profiles of BGA7592 by antigen ELISA.

FIGS. 4A-B show the effects of soluble CEA (sCEA) on CEA antibodies binding to patient-derived MKN45 gastric adenocarcinoma cells. FIG. 4A shows binding profiles of anti-domain B3 antibodies in the presence or absence of soluble CEA (sCEA); FIG. 4B shows the antibody binding profiles of FIG. 4A as histograms.

FIGS. 5A-B shows the randomization sites for generating an antibody library for affinity maturation of humanized BGA7592 antibody light chain CDR (LCDR) regions (FIG. 5A) (SEQ ID NOS: 82, 83, 84, respectively) and heavy chain CDR (HCDR) regions (FIG. 5B) (SEQ ID NOS: 80, 81, and 3, respectively).

FIG. 6 shows the amino acid changes of BGA7592 light chain CDR regions after four rounds of selection.

FIG. 7 shows the binding to LOVO cells of affinity matured, humanized BGA7592 variants by flow cytometry.

FIG. 8 shows the binding, as measured by flow cytometry, of anti-CEA antibodies to MKN45 cells.

FIGS. 9A and 9B are bar graphs showing off-target binding of antibody BGA5384 to various CEACAM family members by antigen ELISA (FIG. 9A) and flow cytometry (FIG. 9B).

FIG. 10 shows the effects of soluble CEA on BGA5384 binding to CEA-expressing MKN45 cells in the presence of various concentrations of soluble CEA.

FIG. 11 is a graph showing antibody dependent cellular cytotoxicity (ADCC) of antibody BGA6710 in vitro.

FIG. 12 shows the effect of BGA6710 antibody on tumor volume in a murine cancer model.

FIG. 13 shows kill curves for cells with varying CEA expression levels by a CEA antibody conjugated to the maytansine compound DM4.

FIG. 14 shows kill curves for cells with varying CEA expression levels by a CEA antibody conjugated to the auristatin MMAE.

FIG. 15 shows kill curves for cells with varying CEA expression levels by a CEA antibody conjugated to the topoisomerase inhibitor DXD.

FIG. 16 shows kills curves for MKN45 cells, which have a CEA specific antibody binding capacity (SABC) of ˜200K (CEA high) by various free cytotoxic agents.

FIG. 17 shows the cell killing effects of eight ADCs on MKN45 cells (CEA high).

FIG. 18 shows the cell killing effects of eight ADCs on H2122 patient-derived cells (lung adenocarcinoma) (CEA moderate).

FIG. 19 shows the cell killing effects of eight ADCs on LS174T patient-derived cells (colorectal adenocarcinoma) (CEA low).

FIG. 20 shows the cell killing effects of eight ADCs on MB-231 patient-derived cells (breast adenocarcinoma) (CEA negative).

FIG. 21 shows the anti-tumor efficacy of different concentrations of BGA-7650 and BGA-9962 versus controls in a cell-line derived xenograft (CDX) model using MKN-45 cells (CEA high).

FIG. 22 shows the anti-tumor efficacy of different concentrations of BGA-7650 and BGA-9962 versus controls in a CDX model using SW-1463 cells (CEA moderate) (rectal adenocarcinoma).

FIG. 23 shows the anti-tumor efficacy of different concentrations of BGA-7650 and BGA-9962 versus controls in a CDX model using H2122 cells (CEA low) (lung adenocarcinoma).

FIG. 24 shows the anti-tumor efficacy of different concentrations of BGA-9962 and BGA-7650 versus control in a patient-derived xenograft (PDX) model of gastric cancer (GC).

FIG. 25 is a graph of concentration versus time for various antibodies, ADCs, and free cytotoxic agents in Balb/c nude mice (non-tumor bearing).

FIG. 26 is a graph showing in vivo DAR over time in mouse for two ADCs (single dose, iv 3 mpk Balb/c nude mice, n=3 per group).

List of Abbreviations

| | |
|---|---|
| TEA | Triethyl amine |
| THF | Tetrahydrofuran |
| MeOH | Methanol |
| PhI(Oac)₂ | (Diacetoxyiodo)benzene |
| PyBOP | Benzotriazol-1-yl-oxytripyrrolidino- |
| | phosphonium hexafluorophosphate |
| HOBt | 1-Hydroxybenzotriazole |
| DIEA | N,N-Diisopropylethylamine |
| DMF | N,N-Dimethylformamide |
| DCM | Dichloromethane |
| NH₄Cl | Ammonium chloride |
| LiOH | Lithium hydroxide |
| HCl | Hydrochloric acid |
| NPC | Bis(4-nitrophenyl)carbonate |
| TFA | Trifluoroacetic acid |
| K₂CO₃ | Potassium carbonate |
| MeCN | Acetonitrile |
| MeI | Methyl iodide |
| Na₂SO₄ | Sodium sulfate |
| EtOAc | Ethyl acetate |
| MeONa | Sodium methoxide |
| Prep-HPLC | Preparative high performance |
| | liquid chromatography |
| TBSCl | t-Butyldimethylchlorosilane |
| Cu(Oac)₂ | Anhydrous cupric acetate |
| Pb(Oac)₄ | Lead tetraacetate |
| TSTU | 2-Succinimido-1,1,3,3-tetra- |
| | methyluronium tetrafluor |
| PE | Petroleum ether |
| Et₂NH | Diethylamine |
| r.t. | Room temperature |
| MS | Mass spectrometry |
| ESI | Electron spray ionization |
| FA | Formic acid |
| FCC | Flash column chromatography |
| NHS | N-hydroxysuccinimide |
| MTBE | Methyl tert-butyl ether |
| Fmoc | 9-fluorenylmethoxycarbonyl protecting group |
| DBU | 1,8-diazabicyclo[5.4.0]undec-7-ene |
| DCC | Dicyclohexylcarbodiimide |
| UPLC | Ultra performance liquid chromatography |
| Mc-Osu | Maleimidocaproyl N-hydroxysuccimidyl ester |
| DMSO | Dimethylsulfoxide |
| TCEP | Tris(2-chloroethyl)phosphate |

### Definitions

Unless specifically defined below or elsewhere in this document, all technical and scientific terms used herein have the meaning commonly understood by one of ordinary skill in the art.

As used herein, including in the appended claims, the singular forms of words such as “a,” “an,” and “the” include their corresponding plural forms unless the context clearly indicates otherwise.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±110%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The term “or” is used to mean, and is used interchangeably with, the term “and/or” unless the context clearly indicates otherwise.

The term “Carcinoembryonic antigen” or “CEA” refers to an approximately 70-100 kDa glycoprotein, also known as CEACAM5 or CD66e. The amino acid sequence of human CEA, SEQ ID NO:52, can also be found at accession number P06731 or NM_004363.2.

The terms “administration,” and “administering,” as used herein, when applied to an animal, human, subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient comprising, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The term “affinity” as used herein refers to the strength of interaction between antibody and antigen. Within the antigen, the variable regions of the antibody interact through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL or Vic) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997); Lefranc, M.-P., The Immunologist, 7, 132-136 (1999); Lefranc, M.-P. et al., Dev. Comp. Immunol., 27, 55-77 (2003)).

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA, and IgY), or subclass (e.g., IgG1, IgG2, IgG3, IgG4, IgA1, and IgA2).

In some embodiments, the anti-CEA antibodies comprise at least one antigen-binding site. In some embodiments, the anti-CEA antibodies comprise an antigen-binding fragment from a CEA antibody described herein. In some embodiments, the anti-CEA antibody is isolated or recombinant.

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies can be obtained by methods known to those skilled in the art. See, for example, Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: a LABORATORY MANUAL, Cold Spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); nanobodies and antibodies formed from antibody fragments; and bicyclic peptides (Hurov, K. et al., 2021. Journal for ImmunoTherapy of Cancer, 9(11)).

As used herein, an antibody or antigen-binding antibody fragment, “specifically binds” or “selectively binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a blood, serum, plasma, or tissue sample. Thus, under certain designated immunoassay conditions, the antibodies or antigen-binding fragments thereof specifically bind to a particular antigen at least two times greater when compared to the background level and do not specifically bind in a significant amount to other antigens present in the sample. In one aspect, under designated immunoassay conditions, the antibody or antigen-binding fragment thereof specifically bind to a particular antigen at least ten times greater when compared to the background level of binding and does not specifically bind in a significant amount to other antigens present in the sample.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification, or for other reasons.

The term “equilibrium dissociation constant” or “KD” or “M” refers to the dissociation rate constant (kd, time⁻¹) divided by the association rate constant (ka, time⁻¹, M⁻¹).

Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹, 10⁻¹² M, or 10⁻¹³ M.

The terms “cancer” or “tumor” used herein has the broadest meaning as understood in the art and refers to the physiological condition in mammals that is typically characterized by unregulated cell growth. In the context of the present disclosure, the cancer or tumor is not limited to a certain type or location.

In the context of the present disclosure, when reference is made to an amino acid sequence, the term “conservative substitution” means substitution of the original amino acid by a new amino acid that does not substantially alter the chemical, physical, and/or functional properties of the antibody or fragment, e.g., its binding affinity to CEA. Common conservative substations of amino acids are well known in the art.

The terms “improve,” “increase,” “inhibit,” and “reduce” indicate values that are relative to a baseline or other reference measurement. In some embodiments, an appropriate reference measurement may comprise a measurement in a certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in a comparable system known or expected to respond in a comparable way, in presence of the relevant agent or treatment.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces, or VH/VL interfaces of antibodies (see, e.g., US 2011/0287009, US2007/0178552, WO 96/027011, WO 98/050431, and Zhu et al., 1997, Protein Science 6:781-788). In some embodiments, knob-into-holes ensure the correct pairing of two different heavy chains together during the manufacture of antibodies. For example, antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also ensure correct pairing. Examples of algorithms that are suitable for determining percent sequence identity and sequence similarity are the BLAST algorithms, which are described in Altschul et al., Nuc. Acids Res. 25:3389-3402, 1977; and Altschul et al., J. Mol. Biol. 215:403-410, 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915), alignments (B) of 50, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12, and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-CEA antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal, or epidermal administration (e.g., by injection or infusion).

The term “therapeutically effective amount” or “effective amount” as herein used refers to the amount of an agent that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination components.

The term “combination therapy” refers to the administration of two or more therapeutic agents to treat a therapeutic condition or disorder. Such administration encompasses co-administration of these therapeutic agents in a substantially simultaneous manner. Such administration also encompasses co-administration in multiple or in separate containers or formulations (e.g., capsules, powders, and liquids) for each active ingredient. Powders and/or liquids can be reconstituted or diluted to a desired dose prior to administration. In addition, “combination therapy” encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-CEA ADC is administered to the subject at the same time as, just before, or just after administration of an additional therapeutic agent. In certain embodiments, an anti-CEA ADC is administered as a co-formulation with an additional therapeutic agent.

The term “toxin” or “payload” or “cytotoxic agent” is used herein to reference a molecule that inhibits or reduces the expression of molecules in cells, inhibits or reduces the function of cells, induces apoptosis of cells, and/or causes death of cells. The term includes radioactive isotopes, chemotherapeutic agents, and toxins such as small molecule toxins or enzymatically active toxins of bacterial, fungal, plant, or animal origin, including fragments and/or variants thereof. Examples of cytotoxic agents include, but are not limited to, auristatins (e.g., auristatin E, auristatin F, MMAE, and MMAF), auromycins, maytansinoids, pyrrolobenzodiazepine (PBD), ricin, ricin A-chain, combrestatin, duocarmycins, dolastatins, doxorubicin, daunorubicin, taxols, cisplatin, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, and calicheamicin, as well as radioisotopes such as At211, I131, I125, Y90, Re186, Re188, Sm153, Bi212 or 213, P32, and Lu177.

An “alkyl” group is a saturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and n-hexyl; while saturated branched alkyls include -isopropyl, -sec-butyl, -isobutyl, -tert-butyl, -isopentyl, 2-methylpentyl, 3methylpentyl, 4-methylpentyl, 2,3-dimethylbutyl and the like. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

A “cycloalkyl” group is a saturated, or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3Himidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is a non-aromatic cycloalkyl in which one to four of the ring carbon atoms are independently replaced with a heteroatom independently selected from the group consisting of O, S, and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include but are not limited to 4-ethyl-morpholinyl, 4-propylmorpholinyl, furan-2-yl methyl, furan-3-yl methyl, pyridine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

An “alkoxy” or “alkoxyl” group is —O(alkyl), wherein alkyl is defined above.

An “alkoxyalkyl” group is -(alkyl)O(alkyl), wherein each alkyl is independently as defined above.

An “amine” group is a radical of the formula: —NH₂.

A “hydroxyl amine” group is a radical of the formula: N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: N(R^{#})O-aryl or NHOaryl, wherein R^{#} is as defined above.

An “alkylamine” group is a radical of the formula: NHalkyl or N(alkyl)₂, wherein each alkyl is independently as defined above.

An “aminocarbonyl” group is a radical of the formula: —C(═O)N(R^{#})₂, —C(═O)NH(R^{#}), or C(═O)NH₂, wherein each R^{#} is as defined above.

An “acylamino” group is a radical of the formula: NHC(═O)(R^{#}) or N(alkyl)C(═O)R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O⁻.

A “carboxy” group is a radical of the formula: C(═O)OH.

A “ketone” group is a radical of the formula: C(═O)(R^{#}), wherein R^{#} is as defined above.

An “aldehyde” group is a radical of the formula: —CH(═O).

An “ester” group is a radical of the formula: C(═O)O(R^{#}) or OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula: —N═C(R^{#})₂ or -C(R^{#})═N(R^{#}), wherein each R” is independently as defined above.

An “imide” group is a radical of the formula: —C(═O)N(R#)C(═O)(R^{#}) or N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})=NH, or -NHC(R^{#})=NH, wherein each R^{#} is independently as defined above.

A “guanidine” group is a radical of the formula: -N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)2, —N═C(NH(R^{#}))₂, or —N═C(NH₂)₂, wherein each R” is independently as defined above.

An “enamine” group is a radical of the formula: -N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or -C(R^{#})═C(R^{)(NH}₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N-N(R^{#})₂)(R^{#})₂, —C(═NNH(R^{#}))(R^{#})₂, —C(═N-NH₂)(R^{#})₂, —N(R^{#})(N═C(R^{#})₂), or —NH(N═C(R^{#})₂), wherein each R^{#} is independently as defined above.

An “azide” group is a radical of the formula: -N₃.

An “isocyanate” group is a radical of the formula: N═C═O.

An “isothiocyanate” group is a radical of the formula: N═C═S.

A “cyanate” group is a radical of the formula: OCN.

A “thiocyanate” group is a radical of the formula: SCN.

A “thioether” group is a radical of the formula; -S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R′), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, or —S(═O)₂NH(R^{#}), or —S(═O)₂NH₂, wherein each R^{#} is independently as defined above.

A “phosphonate” group is a radical of the formula: —P(═OX(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula: —P(R^{#})₂, wherein each R^{#} is independently as defined above.

When the groups described herein, with the exception of alkyl group are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

All pharmaceutically acceptable salts, solvates, and/or hydrates of compounds depicted herein are within the scope of the present disclosure.

As used herein and unless otherwise indicated, the term “prodrug” means a compound derivative that can hydrolyze, oxidize, or otherwise react under biological conditions (in vitro or in vivo) to provide an active compound, particularly a compound.

Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers. Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

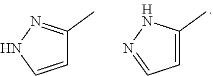

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²¹I), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂-, B—CH₂—CH₂—CH₂-, B—CH₂—CH₂—CH₂—CH₂—, B-CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O-, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C-I5 aryloxy, and 6 to 10 ring carbon atoms, i.e., C₆₋₁₀ aryloxy. Examples of aryloxy moieties include, but are not limited to phenoxy, naphthoxy, and anthroxy.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, and not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

As used herein, “inorganic acid residue” refers to the the ortho- and pyrophosphoric acid, phosphoric acid, and sulphuric acid residue.

As used herein, “organic acid residue” refers to the residue of alkanecarboxylic acid, amino acid, or oligopeptide. In one embodiment, the alkanecarboxylic acid is methanoic acid; ethanoic acid; propanoic acid; butanoic acid; pentanoic acid; hexanoic acid; heptanoic acid; octanoic acid; nonanoic acid; decanoic acid; undecanoic acid; dodecanoic acid; tridecanoic acid; tetradecanoic acid; pentadecanoic acid; hexadecanoic acid; heptadecanoic acid; octadecanoic acid; nonadecanoic acid; or icosanoic acid. In one embodiment, the alkanecarboxylic acid is methanoic acid; ethanoic acid; propanoic acid; or butanoic acid.

Certain groups, moieties, substituents, and atoms are depicted with a wiggly line that intersects a bond or bonds to indicate the atom through which the groups, moieties, substituents, atoms are bonded. For example, a phenyl group that is substituted with a propyl group depicted as:

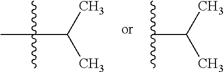

has the following structure:

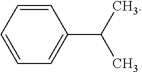

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains.

Illustrations showing substituents bonded to a non-cyclic group through a bond between two atoms are meant to indicate, unless specified otherwise, that the substituent may be bonded to either atom of the bond through which the substituent bond passes, according to techniques set forth herein or which are known in the field to which the instant disclosure pertains. Thus, for example,

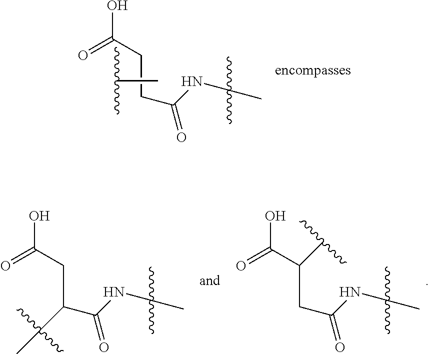

### DETAILED DESCRIPTION

The present disclosure provides for anti-CEA antibody drug conjugates (ADCs). The present disclosure also provides anti-CEA ADCs comprising antibodies that have desirable pharmacokinetic characteristics and other desirable attributes, and thus can be used for reducing the likelihood of or treating a subject having a CEA-expressing or accumulating cell, such as a cancer characterized by expression or accumulation of CEA. The present disclosure further provides pharmaceutical compositions comprising the anti-CEA ADC and methods of making and using the same or pharmaceutical compositions comprising the same.

In some embodiments, the anti-CEA ADCs are useful in the treatment of CEA-related diseases and disorders.

### Anti-CEA Antibodies

The present disclosure provides for anti-CEA ADCs comprising anti-CEA antibodies or antigen-binding fragments thereof that specifically bind to CEA. Antibodies or antigen-binding fragments of the present disclosure include, but are not limited to, the antibodies or antigen-binding fragments thereof produced as described below.

The present disclosure provides antibodies or antigen-binding fragments that specifically bind to CEA, wherein said antibodies or antigen-binding fragments comprise a VH domain having an amino acid sequence of SEQ ID NO:14, 31, or 48 (Table 1). The present disclosure also provides antibodies or antigen-binding fragments that specifically bind CEA, wherein said antibodies or antigen-binding fragments comprise a heavy chain CDR (HCDR) having an amino acid sequence of any one of the HCDRs listed in Table 1. In one aspect, the present disclosure provides antibodies or antigen-binding fragments that specifically bind to CEA, wherein said antibodies comprise (or alternatively, consist of) one, two, three, or more HCDRs having an amino acid sequence of any of the HCDRs listed in Table 1.

The present disclosure provides for antibodies or antigen-binding fragments that specifically bind to CEA, wherein said antibodies or antigen-binding fragments comprise a VH domain as described in Table 1, or any of the sets of HCDRs of Table 1 and a VL domain having an amino acid sequence of SEQ ID NO:15, 32, or 49 (Table 1). The present disclosure also provides antibodies or antigen-binding fragments that specifically bind to CEA, wherein said antibodies or antigen-binding fragments that comprise a light chain CDR (LCDR) having an amino acid sequence of any one of the LCDRs listed in Table 1. In particular, the disclosure provides for antibodies or antigen-binding fragments that specifically bind to CEA, said antibodies or antigen-binding fragments comprising (or alternatively, consisting of) one, two, three, or more LCDRs having an amino acid sequence of any of the LCDRs listed in Table 1.

The present disclosure provides antibodies or antigen-binding fragments that specifically bind to CEA, wherein said antibodies or antigen-binding fragments comprise
(i) a heavy chain variable region comprising SEQ ID NO:31, and a light chain variable region comprising SEQ ID NO:32; or
(ii) a heavy chain variable region comprising SEQ ID NO:48, and a light chain variable region comprising SEQ ID NO:49; or
(iii) a heavy chain variable region comprising SEQ ID NO:14, and a light chain variable region comprising SEQ ID NO:15; or
(iv) three heavy chain CDRs:
   HCDR1 comprising an amino acid sequence as set forth in SEQ ID NO:24,
   HCDR2 comprising an amino acid sequence as set forth in SEQ ID NO:25,
   HCDR3 comprising an amino acid sequence as set forth in SEQ ID NO:26, and
   three light chain CDRs:
   LCDR1 comprising an amino acid sequence as set forth in SEQ ID NO:27,
   LCDR2 comprising an amino acid sequence as set forth in SEQ ID NO:28,
   LCDR3 comprising an amino acid sequence as set forth in SEQ ID NO:23; or
(v) three heavy chain CDRs:
   HCDR1 comprising an amino acid sequence as set forth in SEQ ID NO:7,
   HCDR2 comprising an amino acid sequence as set forth in SEQ ID NO:8,
   HCDR3 comprising an amino acid sequence as set forth in SEQ ID NO:9, and three light chain CDRs:
   LCDR1 comprising an amino acid sequence as set forth in SEQ ID NO:10,
   LCDR2 comprising an amino acid sequence as set forth in SEQ ID NO:11,
   LCDR3 comprising an amino acid sequence as set forth in SEQ ID NO:6; or
(vi) three heavy chain CDRs:
   HCDR1 comprising an amino acid sequence as set forth in SEQ ID NO:41,
   HCDR2 comprising an amino acid sequence as set forth in SEQ ID NO:42,
   HCDR3 comprising an amino acid sequence as set forth in SEQ ID NO:43, and
   three light chain CDRs:
   LCDR1 comprising an amino acid sequence as set forth in SEQ ID NO:44,
   LCDR2 comprising an amino acid sequence as set forth in SEQ ID NO:45,
   LCDR3 comprising an amino acid sequence as set forth in SEQ ID NO:40.

The present disclosure provides ADCs comprising antibodies or antigen-binding fragments that specifically bind to CEA, wherein said antibodies or antigen-binding fragments comprise a VH domain having at least 85%, 90%, 95%, 96%, 97%, 98%, or 99% identity to a sequence as set forth in SEQ ID NO:14, 31, or 48 (Table 1).

The present disclosure provides ADCs comprising antibodies or antigen-binding fragments that specifically bind to CEA, wherein said antibodies or antigen-binding fragments comprise a VH domain having at least 85%, 90%, 95%, 96%, 97%, 98%, or 99% identity to the VH sequence of (i), (ii), or (iii); and a VL domain having at least 85%, 90%, 95%, 96%, 97%, 98%, or 99% identity to the VL sequence of (i), (ii), or (iii):
(i) a heavy chain variable region comprising SEQ ID NO:31, and a light chain variable region comprising SEQ ID NO:32;
(ii) a heavy chain variable region comprising SEQ ID NO:48, and a light chain variable region comprising SEQ ID NO:49; and
(iii) a heavy chain variable region comprising SEQ ID NO:14, and a light chain variable region comprising SEQ ID NO:15.

In some aspects, no more than 1, 2, 3, 4, or 5 amino acids have been changed (e.g., via insertion, deletion, or substitution) in the CDR regions when compared with the CDR regions depicted in the sequence described in Table 1.

Other antibodies of the present disclosure include those where the amino acids or nucleic acids encoding the amino acids have been changed, yet have at least 60%, 70%, 80%, 90%, 95%, or 99% percent identity to the variable region sequences described in Table 1. In some aspects, no more than 1, 2, 3, 4, or 5 amino acids have been changed (e.g., via insertion, deletion, or substitution) in the variable regions when compared with the variable regions depicted in the sequences described in Table 1, while retaining substantially the same therapeutic activity.

The present disclosure also provides nucleic acid sequences that encode VH, VL, the full length heavy chain, and the full length light chain of the antibodies that specifically bind to CEA. Such nucleic acid sequences can be optimized for expression in mammalian cells.

TABLE 1
Amino Acid and Nucleic Acid Sequences

| ANTIBODY | SEQ ID NO | | SEQUENCE |
|---|---|---|---|
| BGA5384 | SEQ ID NO: 1 | HCDR1 | GYIFTSYY |
| (IMGT) | | (IMGT) | |
| | SEQ ID NO: 2 | HCDR2 | INPQTGKT |
| | | (IMGT) | |
| | SEQ ID NO: 3 | HCDR3 | AREYGNYNYPLDY |
| | | (IMGT) | |
| | SEQ ID NO: 4 | LCDR1 | ENQYGY |
| | | (IMGT) | |
| | SEQ ID NO: 5 | LCDR2 | NY |
| | | (IMGT) | |
| | SEQ ID NO: 6 | LCDR3 | QHHLGTPYT |
| | | (IMGT) | |
| BGA5384 | SEQ ID NO: 7 | HCDR1 | SYYLH |
| (Kabat) | | (Kabat) | |
| | SEQ ID NO: 8 | HCDR2 | YINPQTGKTSYAQKFQG |
| | | (Kabat) | |
| | SEQ ID NO: 9 | HCDR3 | EYGNYNYPLDY |
| | | (Kabat) | |
| | SEQ ID NO: 10 | LCDR1 | RASENQYGYLA |
| | | (Kabat) | |
| | SEQ ID NO: 11 | LCDR2 | NYKNLVE |
| | | (Kabat) | |
| | SEQ ID NO: 6 | LCDR3 | QHHLGTPYT |
| | | (Kabat) | |
| BGA5384 | SEQ ID NO: 12 | HCDR1 | GYIFTSY |
| (Chothia) | | (Chothia) | |
| | SEQ ID NO: 13 | HCDR2 | NPQTGK |
| | | (Chothia) | |
| | SEQ ID NO: 9 | HCDR3 | EYGNYNYPLDY |
| | | (Chothia) | |
| | SEQ ID NO: 10 | LCDR1 | RASENQYGYLA |
| | | (Chothia) | |
| | SEQ ID NO: 11 | LCDR2 | NYKNLVE |
| | | (Chothia | |
| | SEQ ID NO: 6 | LCDR3 | QHHLGTPYT |
| | | (Chothia) | |
| BGA5384 | SEQ ID NO: 14 | VH | QVQLVQSGAEVKKPGASVKVSCKASGYIFTSY |
| | | | YLHWVRQAPGQGLEWIGYINPQTGKTSYAQK |
| | | | FQGRVTMTRDTSTSTVYMELSSLRSEDTAVYY |
| | | | CAREYGNYNYPLDYWGQGTLVTVSS |
| | SEQ ID | VL | DIQMTQSPSSLSASVGDRVTITCRASENQYGYL |
| | NO: 15 | | AWYQQKPGKVPKLLIYNYKNLVEGVPSRFSGS |
| | | | GSGTDFTLTISSLQPEDVATYYCQHHLGTPYTF |
| | | | GQGTKVEIK |
| | SEQ ID | VH DNA | CAGGTGCAGCTGGTGCAGAGCGGCGCGGAA |
| | NO: 16 | | GTGAAAAAACCGGGCGCGAGCGTGAAAGTG |
| | | | AGCTGCAAAGCGAGCGGCTATATTTTTACCA |
| | | | GCTATTACCTGCATTGGGTGCGCCAGGCGCC |
| | | | GGGCCAGGGCCTGGAATGGATTGGCTATATTA |
| | | | ACCCGCAGACCGGCAAGACCAGCTATGCCCA |
| | | | GAAATTTCAGGGCCGCGTGACCATGACCCGC |
| | | | GATACCAGCACCAGCACCGTGTATATGGAAC |
| | | | TGAGCAGCCTGCGCAGCGAAGATACCGCGGT |
| | | | GTATTATTGCGCGCGCGAATATGGCAACTATA |
| | | | ACTATCCGCTGGATTATTGGGGCCAGGGCAC |
| | | | CCTGGTGACCGTGAGCAGC |
| | SEQ ID | VL DNA | GATATTCAGATGACCCAGAGCCCGAGCAGCC |
| | NO: 17 | | TGAGCGCGAGCGTGGGCGATCGCGTGACCAT |
| | | | TACCTGCCGCGCGAGCGAAAACCAGTATGGC |
| | | | TATCTGGCGTGGTATCAGCAGAAACCGGGCA |
| | | | AAGTGCCGAAACTGCTGATTTATAACTATAAA |
| | | | AACCTGGTGGAAGGCGTGCCGAGCCGCTTTA |
| | | | GCGGCAGCGGCAGCGGCACCGATTTTACCCT |
| | | | GACCATTAGCAGCCTGCAGCCGGAAGATGTG |
| | | | GCGACCTATTATTGCCAGCATCATCTGGGCAC |
| | | | CCCGTATACCTTTGGCCAGGGCACCAAAGTG |
| | | | GAAATTAAA |
| BGA3715 (also | SEQ ID | HCDR1 | GFTFSSFG |
| known as | NO: 18 | (IMGT) | |
| BGA190) | SEQ ID | HCDR2 | ISIGSDII |
| (IMGT) | NO: 19 | (IMGT) | |
| | SEQ ID | HCDR3 | TRRSYRSYWYFDV |
| | NO: 20 | (IMGT) | |
| | SEQ ID | LCDRI | ENIYSY |
| | NO: 21 | (IMGT) | |
| | SEQ ID | LCDR2 | NA |
| | NO: 22 | (IMGT) | |
| | SEQ ID | LCDR3 | QHHFLSPWM |
| | NO: 23 | (IMGT) | |
| BGA3715 | SEQ ID | HCDR1 | SFGMH |
| (BGA190) | NO: 24 | (Kabat) | |
| (Kabat) | SEQ ID | HCDR2 | YISIGSDIIYYADTVKG |
| | NO: 25 | (Kabat) | |
| | SEQ ID | HCDR3 | RSYRSYWYFDV |
| | NO: 26 | (Kabat) | |
| | SEQ ID | LCDR1 | RTSENIYSYLA |
| | NO: 27 | (Kabat) | |
| | SEQ ID | LCDR2 | NAKTLAE |
| | NO: 28 | (Kabat) | |
| | SEQ ID | LCDR3 | QHHFLSPWM |
| | NO: 23 | (Kabat) | |
| BGA3715 | SEQ ID | HCDR1 | GFTFSSF |
| (BGA190) | NO: 29 | (Chothia) | |
| (Chothia) | SEQ ID | HCDR2 | SIGSDI |
| | NO: 30 | (Chothia) | |
| | SEQ ID | HCDR3 | RSYRSYWYFDV |
| | NO: 26 | (Chothia) | |
| | SEQ ID | LCDR1 | RTSENIYSYLA |
| | NO: 27 | (Chothia) | |
| | SEQ ID | LCDR2 | NAKTLAE |
| | NO: 28 | (Chothia) | |
| | SEQ ID | LCDR3 | QHHFLSPWM |
| | NO: 23 | (Chothia) | |
| BGA3715 | SEQ ID | VH | DVQLVESGGGLVQPGGSRELSCAASGFTFSSFG |
| (BGA190) | NO: 31 | | MHWVRQAPERGLEWVAYISIGSDIIYYADTVK |
| | | | GRFTISRDNPKNTLFLQMTSLRSEDTAVYYCTR |
| | | | RSYRSYWYFDVWGAGTTVTVSS |
| | SEQ ID | VL | DIQMTQSPASLSASVGETVTITCRTSENIYSYLA |
| | NO: 32 | | WYQQKQGKSPHLLVYNAKTLAEGVPSRFSGS |
| | | | GSGTQFSLKIISLQPEDFGTYYCQHHFLSPWMF |
| | | | GGGTKLEIK |
| | SEQ ID | VH DNA | GATGTGCAGCTGGTGGAGTCTGGGGGAGGCT |
| | NO: 33 | | TAGTGCAGCCTGGAGGGTCCCGGGAACTCTC |
| | | | CTGTGCAGCCTCTGGATTCACTTTCAGTAGCT |
| | | | TTGGAATGCACTGGGTTCGTCAGGCTCCAGA |
| | | | GAGGGGGCTGGAGTGGGTCGCATACATTAGT |
| | | | ATTGGCAGTGATATCATCTACTATGCAGACAC |
| | | | AGTGAAGGGCCGATTCACCATCTCCAGAGAC |
| | | | AATCCCAAGAACACCCTGTTCCTGCAAATGA |
| | | | CCAGTCTGAGGTCTGAGGACACGGCCGTGTA |
| | | | TTACTGTACAAGAAGGTCTTATAGGTCCTACT |
| | | | GGTACTTCGATGTCTGGGGCGCAGGGACCAC |
| | | | GGTCACCGTCTCCTCA |
| | SEQ ID | VL DNA | GACATCCAGATGACTCAGTCTCCAGCCTCCC |
| | NO: 34 | | TATCTGCATCTGTGGGAGAAACTGTCACCATC |
| | | | ACATGTCGAACAAGTGAGAATATTTACAGTTA |
| | | | TTTAGCATGGTATCAGCAGAAACAGGGAAAA |
| | | | TCTCCTCATCTCCTGGTCTATAATGCAAAAAC |
| | | | CTTAGCAGAGGGTGTGCCATCAAGGTTCAGT |
| | | | GGCAGTGGATCAGGCACACAGTTTTCTCTGA |
| | | | AGATCATCAGCCTGCAGCCTGAAGATTTTGG |
| | | | GACTTATTACTGTCAGCATCATTTTCTTAGTCC |
| | | | GTGGATGTTCGGTGGAGGCACCAAGCTGGA |
| | | | AATCAAA |
| BGA288 | SEQ ID | HCDR1 | GYTFTDYN |
| (IMGT) | NO: 35 | (IMGT) | |
| | SEQ ID | HCDR2 | IGPNYGGT |
| | NO: 36 | (IMGT) | |
| | SEQ ID | HCDR3 | ARRGSIPRAVDY |
| | NO: 37 | (IMGT) | |
| | SEQ ID | LCDR1 | QDIHNY |
| | NO: 38 | (IMGT) | |
| | SEQ ID | LCDR2 | RA |
| | NO: 39 | (IMGT) | |
| | SEQ ID | LCDR3 | LQYDEFPYT |
| | NO: 40 | (IMGT) | |
| BGA288 | SEQ ID | HCDR1 | DYNID |
| (Kabat) | NO: 41 | (Kabat) | |
| | SEQ ID | HCDR2 | DIGPNYGGTIYNQKFKG |
| | NO: 42 | (Kabat) | |
| | SEQ ID | HCDR3 | RGSIPRAVDY |
| | NO: 43 | (Kabat) | |
| | SEQ ID | LCDR1 | KASQDIHNYLS |
| | NO: 44 | (Kabat) | |
| | SEQ ID | LCDR2 | RANRLVD |
| | NO: 45 | (Kabat) | |
| | SEQ ID | LCDR3 | LQYDEFPYT |
| | NO: 40 | (Kabat) | |
| BGA288 | SEQ ID | HCDR1 | GYTFTDY |
| (Chothia) | NO: 46 | (Chothia) | |
| | SEQ ID | HCDR2 | GPNYGG |
| | NO: 47 | (Chothia) | |
| | SEQ ID | HCDR3 | RGSIPRAVDY |
| | NO: 43 | (Chothia) | |
| | SEQ ID | LCDR1 | KASQDIHNYLS |
| | NO: 44 | (Chothia) | |
| | SEQ ID | LCDR2 | RANRLVD |
| | NO: 45 | (Chothia) | |
| | SEQ ID | LCDR3 | LQYDEFPYT |
| | NO: 40 | (Chothia) | |
| BGA288 | SEQ ID | VH | EVLLQQSGPELVKPGASVKIFCKASGYTFTDY |
| | NO: 48 | | NIDWVQQSHGKSLEWIGDIGPNYGGTIYNQKF |
| | | | KGKATLTVAKSSSTAYMELRSLTSEDTAVYYCA |
| | | | RRGSIPRAVDYWGQGTSVTVSS |
| | SEQ ID | VL | DIKMTQSPSSMYASLGERVTITCKASQDIHNYL |
| | NO: 49 | | SWFQQKPGKSPKTLIYRANRLVDGVPSRFSGS |
| | | | GSGQDYSLTISSLEYEDMGIYYCLQYDEFPYTF |
| | | | GGGTKLEIK |
| | SEQ ID | VH DNA | GAGGTCCTGCTGCAACAGTCTGGACCTGAGC |
| | NO: 50 | | TGGTGAAGCCTGGGGCTTCAGTGAAGATTTT |
| | | | CTGTAAGGCTTCTGGATACACATTCACTGACT |
| | | | ACAACATAGACTGGGTGCAGCAGAGCCATGG |
| | | | AAAGAGCCTTGAGTGGATTGGAGATATTGGT |
| | | | CCTAATTATGGTGGTACTATCTACAACCAGAA |
| | | | GTTCAAGGGCAAGGCCACATTGACTGTAGCC |
| | | | AAGTCCTCCAGCACAGCCTACATGGAGCTCC |
| | | | GCAGCCTGACATCTGAGGACACTGCAGTCTA |
| | | | TTACTGTGCAAGACGCGGTAGCATCCCGAGG |
| | | | GCTGTGGACTACTGGGGTCAAGGAACCTCAG |
| | | | TCACCGTCTCCTCA |
| | SEQ ID | VL DNA | GACATCAAGATGACCCAGTCTCCATCTTCCAT |
| | NO: 51 | | GTATGCATCTCTAGGAGAGAGAGTCACTATC |
| | | | ACTTGCAAGGCGAGTCAGGACATTCATAACT |
| | | | ATTTAAGCTGGTTCCAGCAGAAACCAGGGAA |
| | | | ATCTCCTAAGACCCTGATCTATCGTGCAAACA |
| | | | GATTGGTAGATGGGGTCCCATCAAGGTTCAG |
| | | | TGGCAGTGGATCTGGGCAAGATTATTCTCTCA |
| | | | CCATCAGCAGCCTGGAGTATGAAGATATGGG |
| | | | AATTTATTATTGTCTACAGTATGATGAGTTTCC |
| | | | GTACACGTTCGGAGGGGGGACCAAGCTGGA |
| | | | AATAAAA |

The present disclosure provides ADCs comprising antibodies and antigen-binding fragments thereof that bind to an epitope of human CEA and any of the payloads described herein.

The present disclosure also provides for ADCs comprising antibodies and antigen-binding fragments thereof that bind to the same epitope as do the anti-CEA antibodies having one or more of the sequences disclosed in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of Table 1 to CEA demonstrates that the test antibody can compete with that antibody or antigen-binding fragment thereof of Table 1 for binding to CEA. Such an antibody can, without being bound to any one theory, bind to the same or a related (e.g., a structurally similar or spatially proximal) epitope on CEA as the antibody or antigen-binding fragment thereof with which it competes. In a certain aspect, the antibody that binds to the same epitope on CEA as the antibodies or antigen-binding fragments thereof of Table 1 is a human or humanized monoclonal antibody. Such human or humanized monoclonal antibodies can be prepared and isolated as described herein.

### Antibody Linkers

It is also understood that the domains and/or regions of the polypeptide chains of the antibodies disclosed herein can be separated by linker regions of various lengths. In some embodiments, the antigen binding domains are separated from each other, a CL, CH1, hinge, CH2, CH3, or the entire Fc region by a linker region. For example, VL1-CL-(linker)-VH2—CH1. Such linker region may comprise a random assortment of amino acids, or a restricted set of amino acids. Such linker regions can be flexible or rigid (see US2009/0155275).

In some embodiments, linkers can be used to conjugate compounds between a toxin or payload and the disclosed antibody. In some embodiments, the linker is cleavable under intracellular conditions, such that cleavage of the linker releases the toxin/payload from the antibody in the intracellular environment. In yet other embodiments, the linker unit is not cleavable and the toxin is released, for example, by antibody degradation. The linker can be without limitation, a cleavable linker, a non-cleavable linker, a hydrophilic linker, a procharged linker, or a dicarboxylic acid-based linker.

### Dimerization of Specific Amino Acids

In one embodiment, the antibodies disclosed herein comprise at least one dimerization-specific amino acid change. The dimerization-specific amino acid changes result in “knobs into holes” interactions, and increase the assembly of correct antibodies. The dimerization-specific amino acids can be within the CH1 domain or the CL domain or combinations thereof. Examples of dimerization-specific amino acids used to pair CH1 domains with other CH1 domains (CH1-CH1) and CL domains with other CL domains (CL-CL) can be found at least in the disclosures of WO2014082179, the WO2015181805 family, and WO2017059551. The dimerization-specific amino acids can also be within the Fc domain and can be in combination with dimerization-specific amino acids within the CH1 or CL domains. In one embodiment, the present disclosure provides an antibody comprising at least one dimerization-specific amino acid pair.

### Alteration of the Framework of Fc Region

In aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII, and FcRn have been mapped and variants with improved binding have been described (see Shields et al., J. Biol. Chem. 276:6591-6604, 2001).

In another aspect, the glycosylation of the antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for an antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures, which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G. L., et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of an antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M., et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S. M., et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev Immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate antibodies with low ADCC and CDC but with good stability, it is possible to modify the hinge and Fc region of human IgG4 and introduce a number of alterations. These modified IgG4 Fc molecules can be found in SEq ID NOs:83-88, U.S. Pat. No. 8,735,553 to Li et al.

### Antibody Production

Antibodies and antigen-binding fragments thereof for the disclosed ADCs can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The present disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide represented by SEQ ID NO:16, SEQ ID NO:33, or SEQ ID NO:50. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide chosen from SEQ ID NOs:17, 34, or 51.

The polynucleotides of the present disclosure can encode the variable region sequence of an anti-CEA antibody. They can also encode both a variable region and a constant region of the antibody. Some of the polynucleotide sequences encode a polypeptide that comprises variable regions of both the heavy chain and the light chain of the exemplified anti-CEA antibodies.

Also provided in the present disclosure are expression vectors and host cells for producing the anti-CEA antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-CEA antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be included for efficient expression of an anti-CEA antibody or antigen-binding fragment thereof. These elements may include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-CEA antibody vectors can be either prokaryotic or eukaryotic. E. coli is one prokaryotic host useful for cloning and expressing the polynucleotides of the present disclosure. Other microbial hosts suitable for use include bacilli, such as Bacillus subtilis, and other Enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters may be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like for initiating and completing transcription and translation. Other microbes, such as yeast, can also be employed to express anti-CEA antibodies. Insect cells in combination with baculovirus vectors can also be used.

In other aspects, mammalian host cells are used to express and produce the anti-CEA antibodies of the present disclosure. Examples include a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells, and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Antibody Drug Conjugates

The antibodies disclosed herein may be combined with a cytotoxic agent (“D” or “P” herein) to form an antibody drug conjugate. The cytotoxic agent may be any molecule that inhibits or reduces the expression of molecules in cells, inhibits or reduces the function of cells, induces apoptosis of cells, and/or causes death of cells. Examples of cytotoxic agents include those described herein. In embodiments, the cytotoxic agent is a topoisomerase inhibitor.

In embodiments, an antibody drug conjugate has the formula A:

Ab-(C-L-(D)ₘ)ₙ (A),

or a pharmaceutically acceptable salt, solvate, or hydrate thereof, wherein:
Ab is the antibody or antigen-binding fragment thereof;
C is a conjugator;
L is a linker;
D is the cytotoxic agent;
m is an integer from 1 to 8; and
n is from 1 to 10.

In particular embodiments, m is 1.

In embodiments, an antibody drug conjugate has the formula A-1:

Ab-(C-L-D)ₙ (A-1),

or a pharmaceutically acceptable salt, solvate, or hydrate thereof, wherein:
Ab is the antibody or antigen-binding fragment thereof;
C is a conjugator;
L is a linker;
D is the cytotoxic agent;
m is an integer from 1 to 8; and
n is from 1 to 10.

In embodiments, n is from 3 to 10, e.g., from 4 to 10, from 5 to 10, from 6 to 10, or from 7 to 9. In certain embodiments, n is about 8.

International Publication No. WO 2023/125530, the entire contents of which are incorporated herein by reference, discloses antibody drug conjugates, the linker payload portions of which are suitable for use in the context of the present disclosure, and linker payloads which are suitable for use in the context of the present disclosure. In some embodiments, a linker payload is a linker payload disclosed in WO 2023/125530.

In embodiments, an antibody drug conjugate has Formula (I):

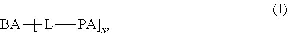

or a pharmaceutically acceptable salt, tautomer, solvate, stereoisomer, enantiomer, isotopologue, or prodrug thereof,
wherein BA is Ab, as that variable is described with respect to an antibody drug conjugate of the present disclosure (e.g., an antibody drug conjugate of Formula A or A-1); L is a covalent linker; PA is a payload residue (e.g., a cytotoxic agent (D), as that variable is described with respect to an antibody drug conjugate of the present disclosure (e.g., an antibody drug conjugate of Formula A or A-1); and subscript x is from 1 to 30 (e.g., n as that variable is described with respect to an antibody drug conjugate of the present disclosure (e.g., an antibody drug conjugate of Formula A or A-1). In some instances, x is from 1 to 4. In some instances, x is about 1. In some instances, x is about 2. In some instances, x is about 3. In some instances, x is about 4.

In further embodiments, the antibody drug conjugate has Formula (Ia):

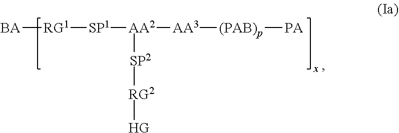

or a pharmaceutically acceptable salt tautomer, solvate, stereoisomer, enantiomer, isotopologue, or prodrug thereof,
wherein RG¹ is a reactive group residue; RG² is an optional reactive group residue; SP¹ and SP² are independently, in each instance, an optional spacer group residue; HG is a hydrophilic residue; PAB is an optional self-immolative unit; subscript p is 0 or 1; and subscript x is from 1 to 30. Values for the remaining variables (e.g., AA², AA³) and alternative values for the variables (e.g., x, p, PAB, HG, RG¹, RG², SP¹, SP², BA) are as described elsewhere herein.

In some embodiments, x is from 1 to 15. In some embodiments, x is from 2 to 10. In some embodiments, x is from 3 to 9. In one embodiment, x is about 3. In one embodiment, x is about 4. In one embodiment, x is about 5. In one embodiment, x is about 6. In one embodiment, x is about 7. In one embodiment, x is about 8. In one embodiment, x is about 9.

In some embodiments of a compound of Formula (Ia), AA² comprises formula (W):

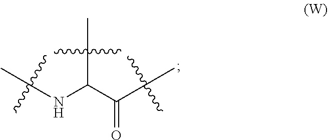 [SMILES: *NC(*)C(*)=O]

AA³ is a dipeptide residue of -valine-alanine-, -valine-citrulline-, or

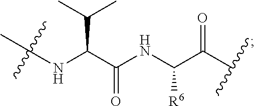

wherein R⁶ is —CH₃, or —(CH₂)₃—NHC(═O)NH₂.

In some embodiments,

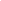

In some embodiments, AA³ is

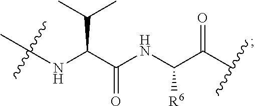

wherein R⁶ is —CH₃, or —(CH₂)₃—NHC(═O)NH₂. In further embodiments, R⁶ is —CH₃.

In some embodiments, PAB represents —NH—CH2-O—, formula (Y1):

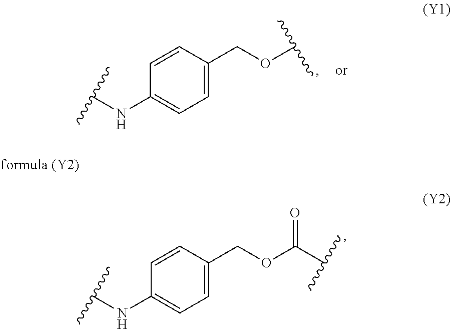 [SMILES: *Nc1ccc(CO*)cc1 ; *Nc1ccc(COC(*)=O)cc1]

wherein the

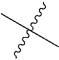

indicates the bond through which the PAB is bonded to the adjacent groups in the formula.

In some embodiments, PAB is —NH—CH₂—O—.

In some embodiments, RG¹ is

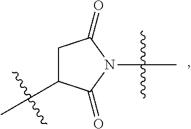 [SMILES: *C1CC(=O)N(*)C1=O]

-(succinimid-3-yl-N)-,

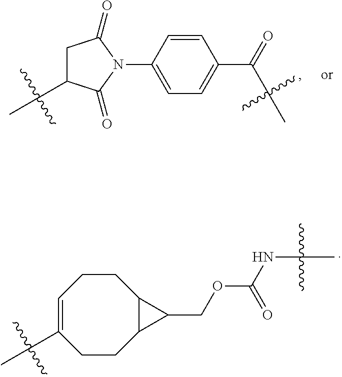 [SMILES: *C(=O)c1ccc(N2C(=O)CC(*)C2=O)cc1 ; *NC(=O)OCC1C2CC/C=C(/*)CCC21]

In some embodiments, RG¹ is

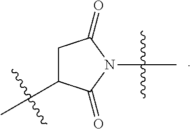 [SMILES: *C1CC(=O)N(*)C1=O]

In some embodiments, RG¹ is

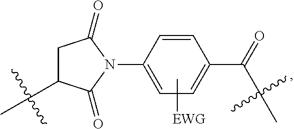

wherein EWG is an electrowithdrawing group, e.g., —CN, —NO₂, halogen, —CF₃, —C(═O)OR¹, or —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In some embodiments, RG¹ is

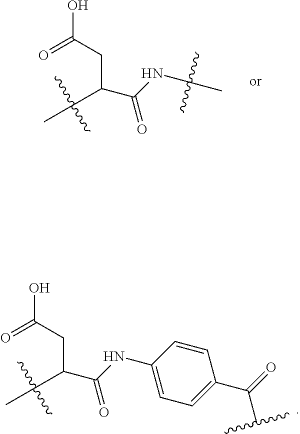 [SMILES: *NC(=O)C(*)CC(=O)O ; *C(=O)c1ccc(NC(=O)C(*)CC(=O)O)cc1]

In some embodiments, RG¹ is

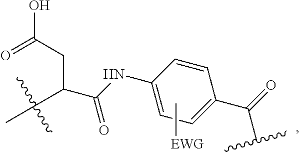

wherein EWG is an electrowithdrawing group, e.g., —CN, —NO₂, halogen, —CF₃, —C(═O)OR¹, or —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In some embodiments, RG¹ is an opened heterocyclic ring, such as the product resulting from conjugation of the maleimide ring of

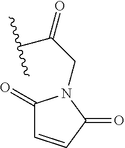 [SMILES: *C(=O)CN1C(=O)C=CC1=O]

with an antibody. It will be appreciated in this regard that conjugation of an antibody to a maleimide ring can occur at either one of the two carbons in the carbon-carbon double bond of the maleimide. Similarly, in the context of an opened ring, conjugation can occur at either one of the two carbon atoms in the double bond. In some embodiments, RG¹ is

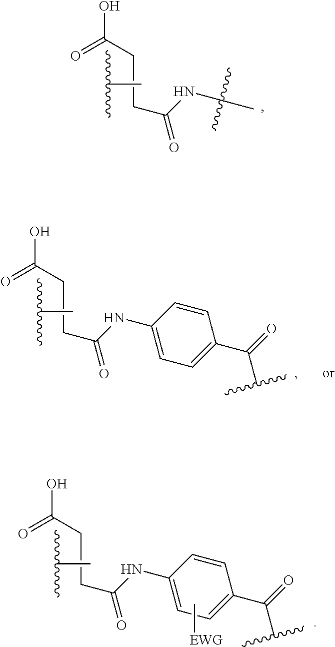

In some embodiments, RG¹ is

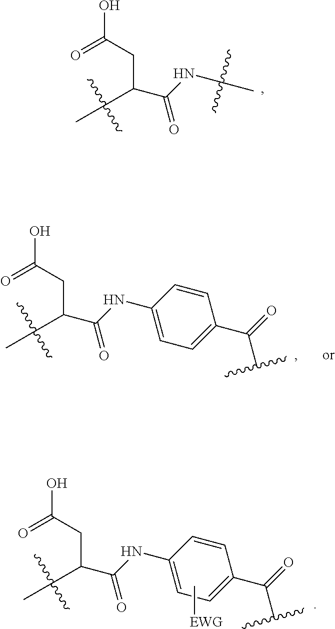

In some embodiments, RG¹ is

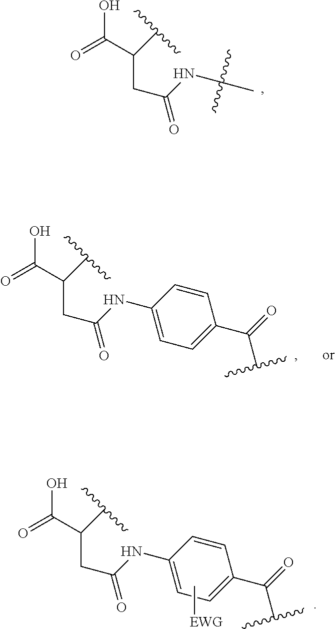

In some embodiments, RG¹ is

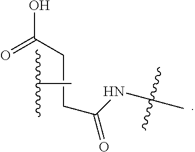

In some embodiments, RG¹ is

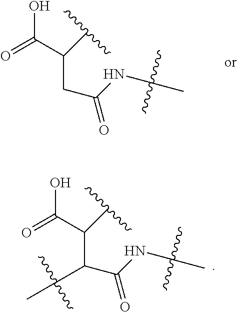 [SMILES: *NC(=O)CC(*)C(=O)O ; *NC(=O)C(*)C(*)C(=O)O]

some embodiments, RG² is a bond, —C(═O)—NH—, or —NHC(═O)—. In some embodiments, RG² is —C(═O)—NH—.

In some embodiments, SP¹ is —(CH₂)ₙ₁—C(═O)—, —(CH₂CH₂O)ₙ₂—CH₂CH₂—C(═O)—, —CH[—(CH₂)ₙ₃—COOH]—C(═O)—, —CH₂—C(═O)—NH—(CH₂)ₙ₄—C(═O)—, —CH₂—C(═O)—NH—(CH₂)ₙ₃—C(═O)—NH—(CH₂)ₙ₄—C(═O)—, or —C(═O)—*CH₂)ₙ₅—C(═O)—, wherein each of n1, n2, n3, n4, and n5 independently represents an integer of 1 to 8. In some embodiments, SP¹ is *—CH₂C(O)N(H)CH₂CH₂C(O)—, wherein asterisk marks the bond that connects to RG¹. In some embodiments, SP¹ is *—(CH₂)₅C(O)—, wherein asterisk marks the bond that connects to RG¹. In some embodiments, SP¹ is *—C(H)(CH₂NH₂)—(CH₂)₂OC(O)N(H)(CH₂)₂C(O)—, wherein asterisk marks the bond that connects to RG¹.

In some embodiments, SP² is —(CH₂)ₙ₆—; and n6 represents an integer of 1 to 8. In some embodiments, n6 is 2.

In some embodiments, HG is

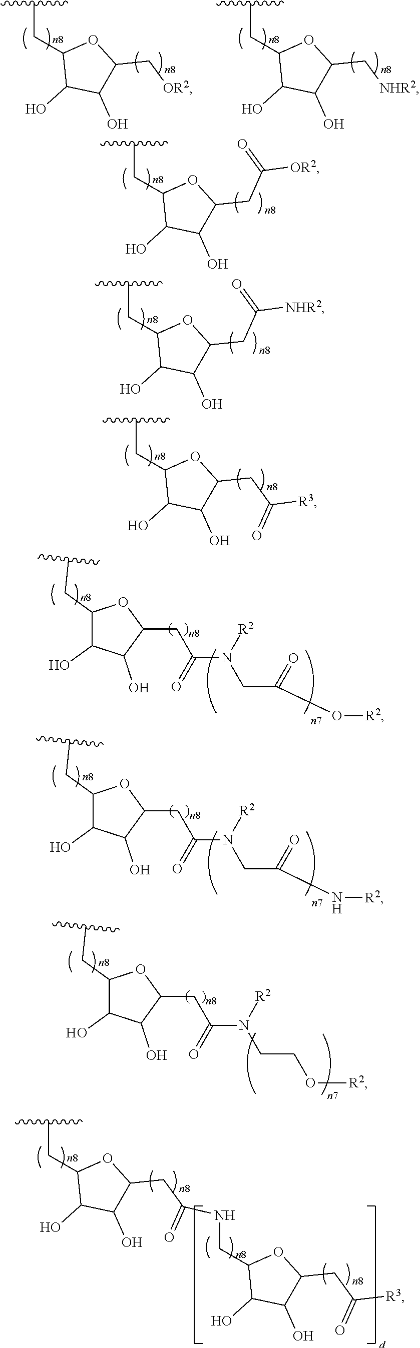

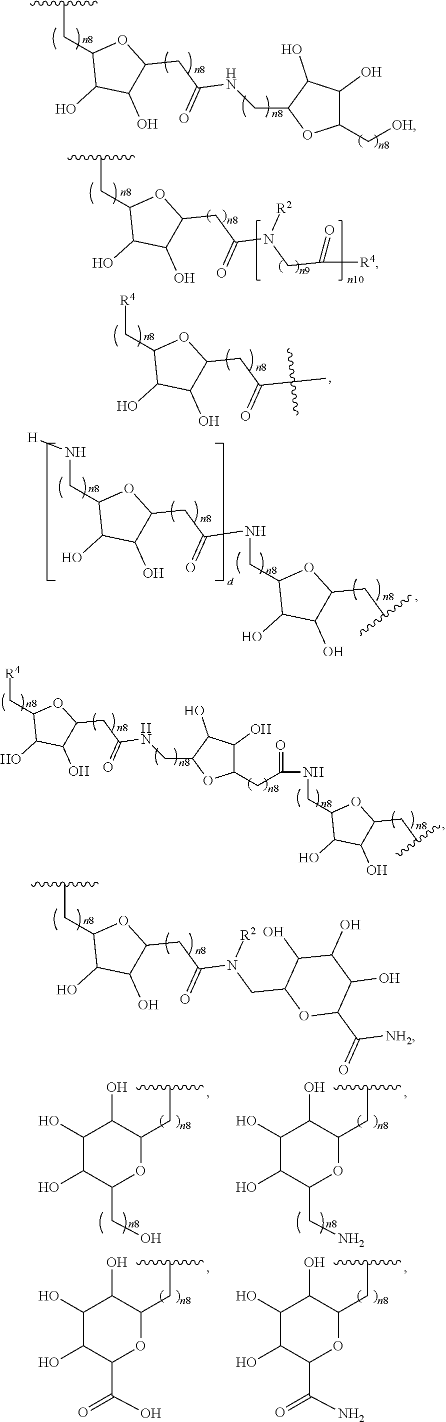

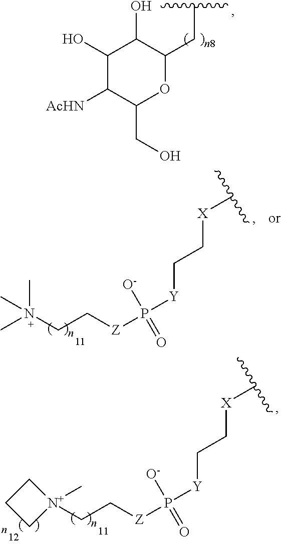

wherein each n7 is independently 1-15; each n8 is independently 0 or 1; each n9 is independently 1 or 2; each n10 is independently an integer of 4 to 16, such as 4, 8, or 12; each n11 is independently an integer of 0 to 5; n12 is an integer of 0 to 3; d is 0-3; R² is H or Me; R³ is —OH, —NH₂, —NHCH₂—CH₂-(PEG)ₓ-OH, or —NHCH₂—CH₂-(PEG)ₓ-OMe; R⁴ is OH or NH₂; and each of X, Y, and Z is independently —CH₂—, —NH—, —S— or —O—.

In some embodiments, HG is

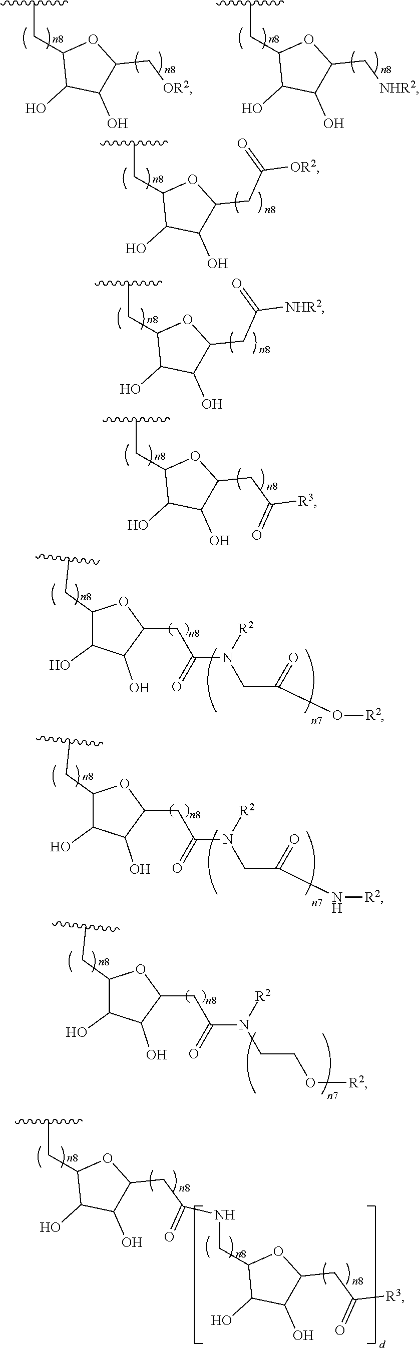

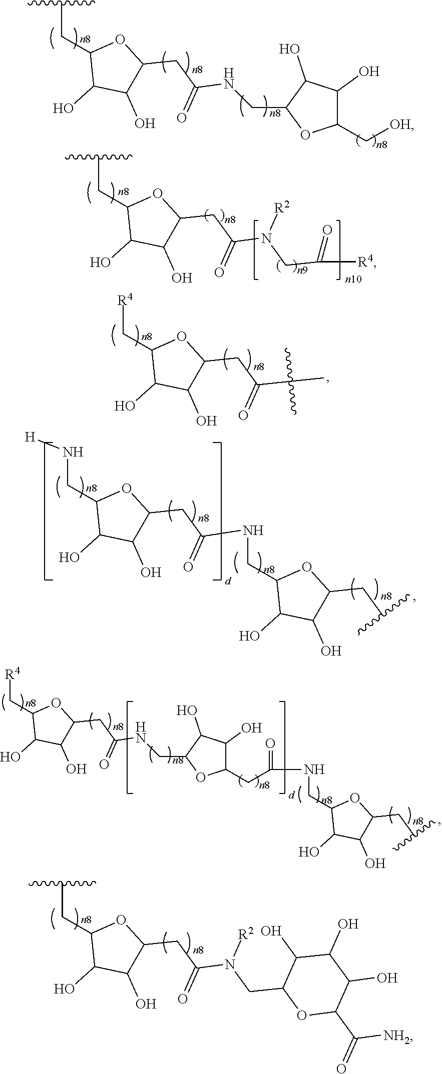

wherein each n7 is independently 1-15; each n8 is independently 0 or 1; each n9 is independently 1 or 2; each n10 is independently an integer of 4 to 16, such as 4, 8, or 12; d is 0-3; R² is H or Me; R³ is —OH, —NH₂, —NHCH₂—CH₂-(PEG)ₓ-OH, or —NHCH₂—CH₂-(PEG)ₓ-OMe; R⁴ is OH or NH₂.

In some embodiments, HG is

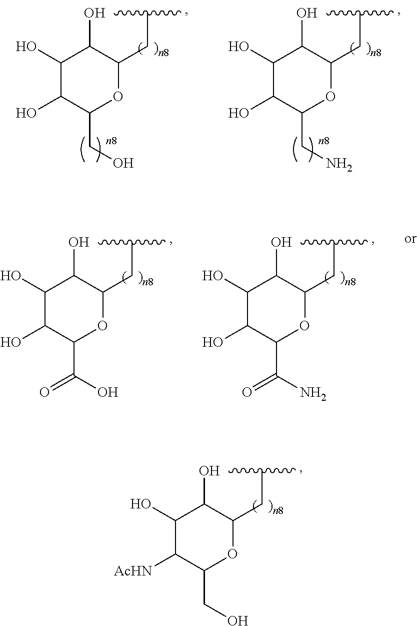

wherein each n8 is independently 0 or 1; and R¹ is H or Me.

In some embodiments, HG is

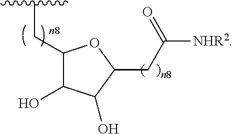

In some embodiments, HG is

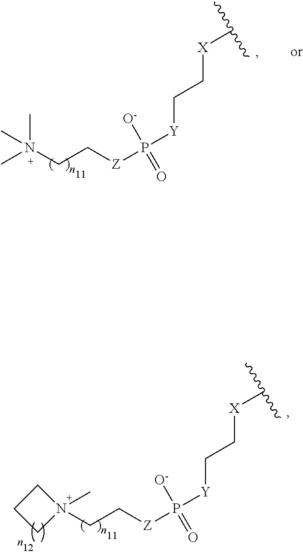

wherein each n11 is independently an integer of 0 to 5; n12 is an integer of 0 to 3; and each of X, Y, and Z is independently —CH₂—, —NH—, —S— or —O—.

In some embodiments, HG is —NHSO₂NH₂, —SO₃H, —SO₂NH₂, —PO₃H₂, and RG² is a bond.

In some embodiments, each PA independently represents a chromophore functional group.

In some embodiments, each chromophore functional group is independently a functional group selected from a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

In some embodiments, each PA is independently selected from the group consisting of Monomethyl auristatin E (MMAE), Monomethyl auristatin F (MMAF), Monomethyl auristatin D (MMAD), Mertansine (Maytansinoid DM1/DM4), Paclitaxel, Docetaxel, Epothilone B, Epothilone A, CYT997, Auristatin tyramine phosphate, Auristatin aminoquinoline, Halocombstatins, Calicheamicin theta, 7-Ethyl-10-hydroxy-camptothecin (SN-38), Pyrrolobenzodiazepine (PBD), Pancratistatin, Cyclophosphate, Cribrostatin-6, Kitastatin, Turbostatin 1-4, Halocombstatins, Eribulin, Hemiasterlin, PNU and Silstatins.

In some embodiments, each PA independently represents formula (D1):

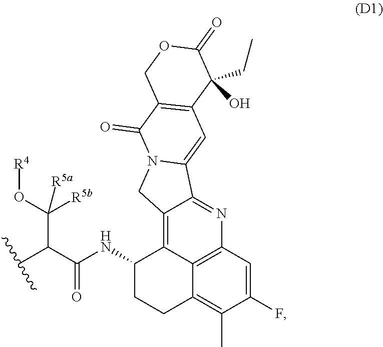

wherein each of R⁴, R^{5a}, and R^{5b} is independently hydrogen, sugar residue, substituted or unsubstituted inorganic or organic acid residue, substituted or unsubstituted C₁₋₈ alkyl, substituted or unsubstituted cycloalkyl, substituted or unsubstituted non-aromatic heterocyclyl, substituted or unsubstituted cycloalkylalkyl, or substituted or unsubstituted heterocyclylalkyl;
R^{5a} and R^{5b} together with the atoms to which they are attached, form a substituted or unsubstituted cycloalkyl, substituted or unsubstituted non-aromatic heterocyclyl.

In some embodiments, R⁴ is hydrogen,

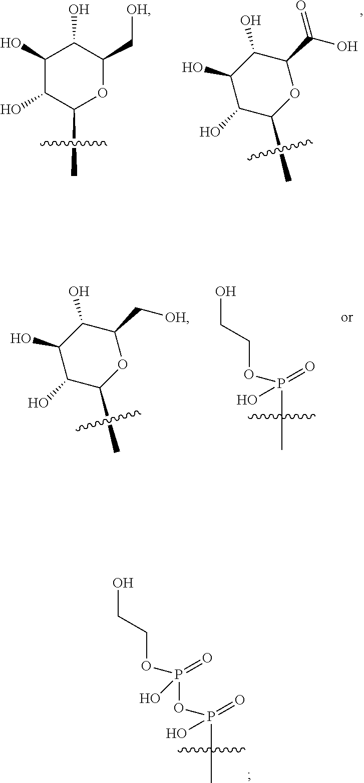

and
wherein each of R^{5a} and R^{5b} is independently H, CH₃, or CF₃; or
R^{5a} and R^{5b} together with the atoms to which they are attached, form a substituted or unsubstituted cycloalkyl, substituted or unsubstituted non-aromatic heterocyclyl.

In some embodiments, R⁴ is hydrogen,

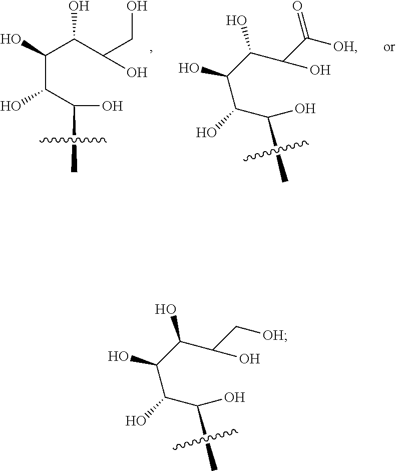

and
wherein each of R^{5a} and R^{5b} is independently H, CH₃, or CF₃; or
R^{5a} and R^{5b} together with the atoms to which they are attached, form a substituted or unsubstituted cycloalkyl, substituted or unsubstituted non-aromatic heterocyclyl.

In some embodiments, each PA independently represents

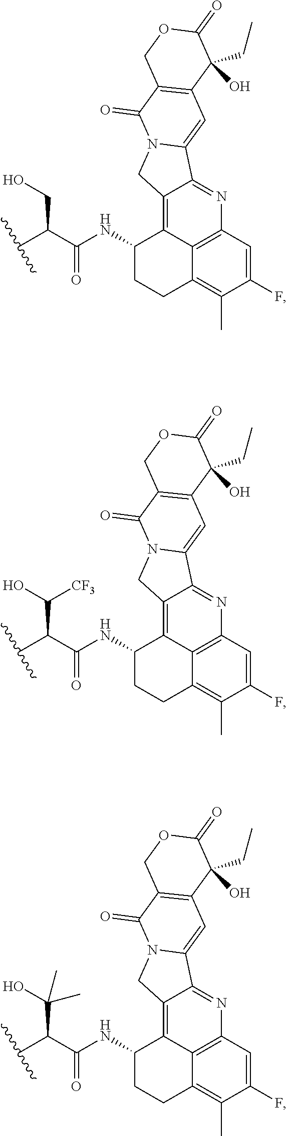 [SMILES: *[C@@H](CO)C(=O)N[C@H]1CCc2c(C)c(F)cc3nc4c(c1c23)Cn1c-4cc2c(c1=O)COC(=O)[C@]2(O)CC ; *[C@H](C(=O)N[C@H]1CCc2c(C)c(F)cc3nc4c(c1c23)Cn1c-4cc2c(c1=O)COC(=O)[C@]2(O)CC)C(O)C(F)(F)F ; *[C@H](C(=O)N[C@H]1CCc2c(C)c(F)cc3nc4c(c1c23)Cn1c-4cc2c(c1=O)COC(=O)[C@]2(O)CC)C(C)(C)O]

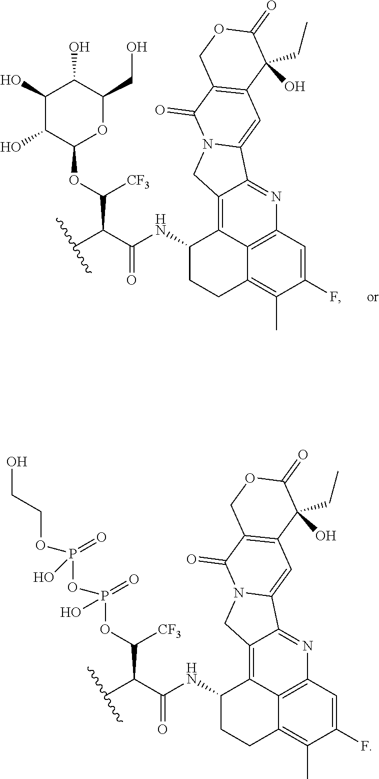 [SMILES: *[C@H](C(=O)N[C@H]1CCc2c(C)c(F)cc3nc4c(c1c23)Cn1c-4cc2c(c1=O)COC(=O)[C@]2(O)CC)C(O[C@@H]1O[C@H](CO)[C@@H](O)[C@H](O)[C@H]1O)C(F)(F)F ; *[C@H](C(=O)N[C@H]1CCc2c(C)c(F)cc3nc4c(c1c23)Cn1c-4cc2c(c1=O)COC(=O)[C@]2(O)CC)C(OP(=O)(O)OP(=O)(O)OCCO)C(F)(F)F]

In some embodiments, each PA independently represents formula (D2):

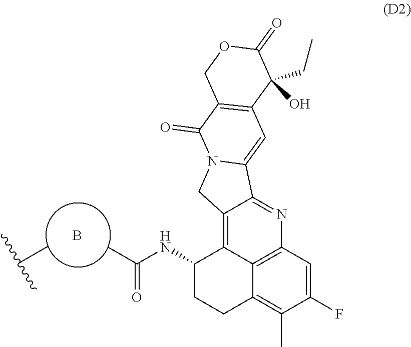

wherein ring B is a substituted or unsubstituted cycloalkyl, substituted or unsubstituted aryl, substituted or unsubstituted heterocyclyl, or substituted or unsubstituted heteroaryl.

In some embodiments, each PA independently represents

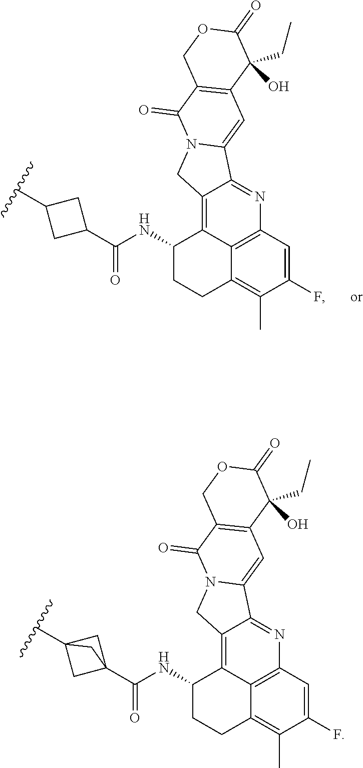 [SMILES: *C1CC(C(=O)N[C@H]2CCc3c(C)c(F)cc4nc5c(c2c34)Cn2c-5cc3c(c2=O)COC(=O)[C@]3(O)CC)C1 ; *C12CC(C(=O)N[C@H]3CCc4c(C)c(F)cc5nc6c(c3c45)Cn3c-6cc4c(c3=O)COC(=O)[C@]4(O)CC)(C1)C2]

In some embodiments, each PA independently represents formula (D3):

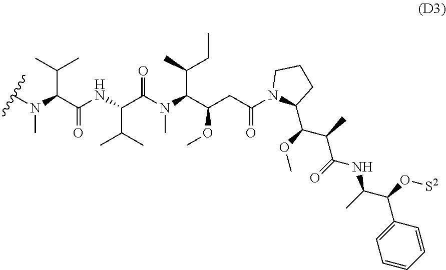

wherein S² is an enzyme hydrolyzable hydrophilic group.

In some embodiments, the S² group is hydrogen or represents one of the following formulas:

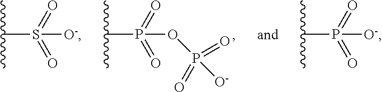

In some embodiments, each PA independently represents formula (E1):

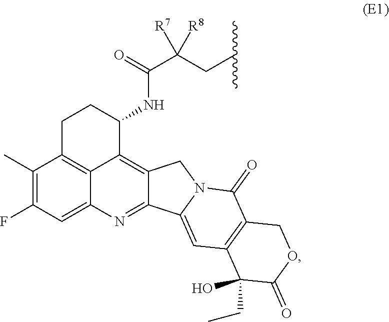

wherein each of R⁷ and R⁸ is, independently, hydrogen, halogen, or alkyl.

In some embodiments, R⁷ and R⁸ are hydrogen.

In some embodiments, R⁷ and R⁸ are methyl.

In some embodiments, R⁷ is methyl and R⁸ is F.

In some embodiments, the carbon to which R⁷ and R⁸ connect is in the S configuration.

In some embodiments, the carbon to which R⁷ and R⁸ connect is in the R configuration.

In some embodiments, each PA independently represents the following formula:

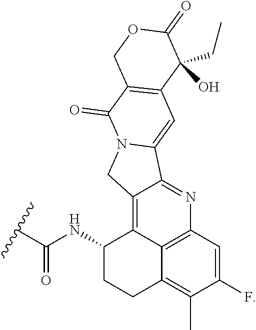 [SMILES: *C(=O)N[C@H]1CCc2c(C)c(F)cc3nc4c(c1c23)Cn1c-4cc2c(c1=O)COC(=O)[C@]2(O)CC]

In some embodiments, each PA is independently Dxd, or independently represents the following formula:

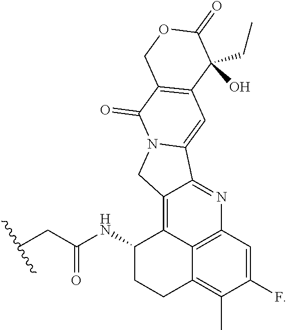 [SMILES: *CC(=O)N[C@H]1CCc2c(C)c(F)cc3nc4c(c1c23)Cn1c-4cc2c(c1=O)COC(=O)[C@]2(O)CC]

In some embodiments, each PA independently represents the following formula:

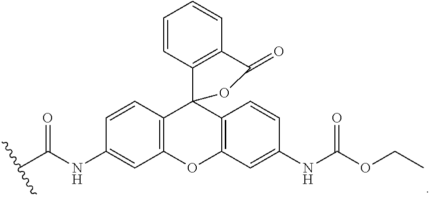 [SMILES: *C(=O)Nc1ccc2c(c1)Oc1cc(NC(=O)OCC)ccc1C21OC(=O)c2ccccc21]

In some embodiments,

AA² is an amino acid residue of glycine or

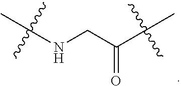 [SMILES: *NCC(*)=O]

In some embodiments of compounds of Formula (Ia) or a pharmaceutically acceptable salt tautomer, solvate, stereoisomer, enantiomer, isotopologue, or prodrug thereof, AA² comprises formula (W):

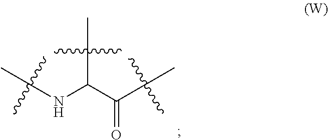 [SMILES: *NC(*)C(*)=O]

and
AA³ is a tetrapeptide residue of -glycine-glycine-phenylalanine-glycine- or

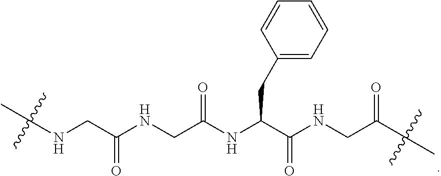 [SMILES: *NCC(=O)NCC(=O)N[C@@H](Cc1ccccc1)C(=O)NCC(*)=O]

In further embodiments, the antibody drug conjugate has Formula (Ib):

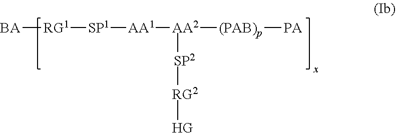

or a pharmaceutically acceptable salt tautomer, solvate, stereoisomer, enantiomer, isotopologue, or prodrug thereof,
wherein AA² comprises formula (W):

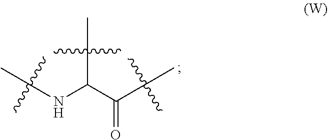 [SMILES: *NC(*)C(*)=O]

and
AA¹ is a dipeptide residue of -valine-alanine-, -valine-citrulline-, or

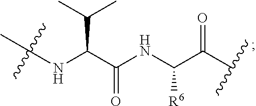

wherein R⁶ is —CH₃, or -(CH₂)₃—NHC(═O)NH₂. Values for the remaining variables (e.g., x, p, BA, HG, RG¹, RG², SP¹, SP², PAB, PA) and alternative values for the variables (e.g., AA¹, AA²) are as described elsewhere herein, for example, with respect to compounds of Formula Ia.

In some embodiments, AA² comprises formula (W):

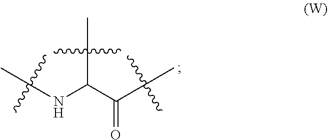 [SMILES: *NC(*)C(*)=O]

and
AA¹ is a tetrapeptide residue of -glycine-glycine-phenylalanine-glycine- or

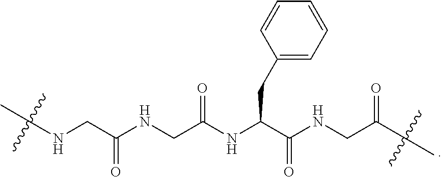 [SMILES: *NCC(=O)NCC(=O)N[C@@H](Cc1ccccc1)C(=O)NCC(*)=O]

In further embodiments, the antibody drug conjugate has Formula (Ic):

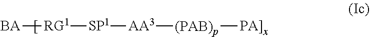

or a pharmaceutically acceptable salt tautomer, solvate, stereoisomer, enantiomer, isotopologue, or prodrug thereof,
wherein AA³ is a dipeptide residue of -valine-alanine-, -valine-citrulline-, or

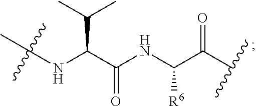

wherein R⁶ is —CH₃, or —(CH₂)₃—NHC(═O)NH₂. Values for the remaining variables (e.g., BA, RG¹, SP¹, PAB, p, PA, x) and alternative values for the variables (AA³, R⁴) are as described elsewhere herein, for example, with respect to a compound of formula Ia.

In some embodiments (e.g., of a compound of Formula (Ic)), AA³ is a tetrapeptide residue of -glycine-glycine-phenylalanine-glycine- or

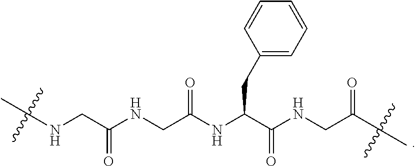 [SMILES: *NCC(=O)NCC(=O)N[C@@H](Cc1ccccc1)C(=O)NCC(*)=O]

In some embodiments, the antibody drug conjugate is selected from one of the following compounds, or a pharmaceutically acceptable salt tautomer, solvate, stereoisomer, enantiomer, isotopologue, or prodrug thereof:

| Example No. | Antibody drug conjugate |
|---|---|
| ADC1-2 | 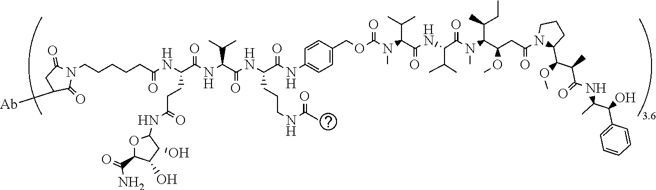 |
| ADC1-4 | 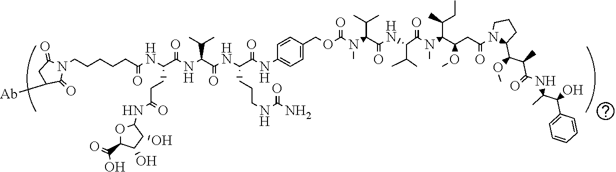 |
| ADC1-11 | 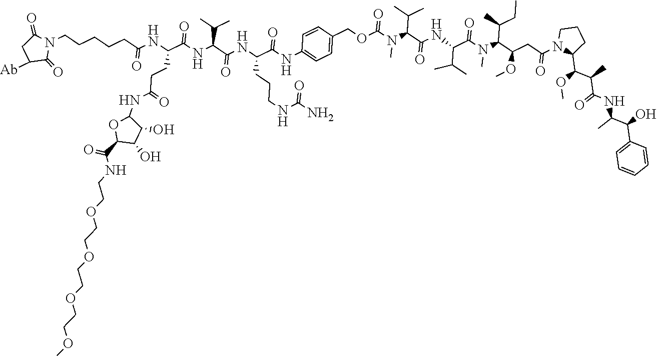 |
| ADC2-2 | 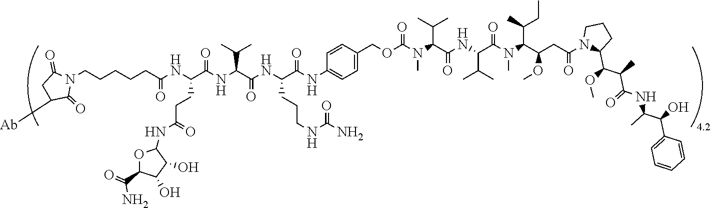 |
| ADC2-4 | 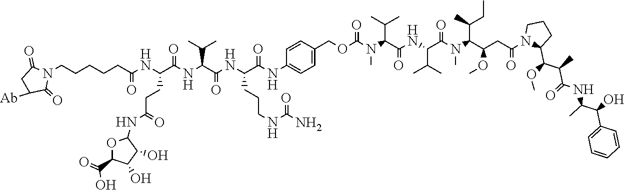 |
| ADC2-6-2 | 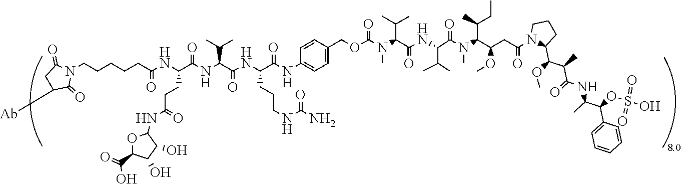 |
| ADC2-7-2 | 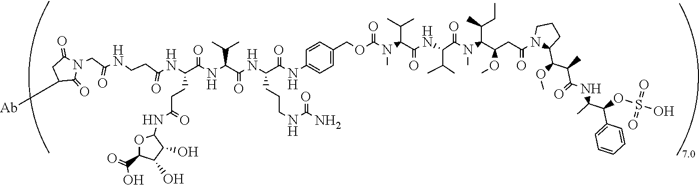 |
| ADC2-9-1 | 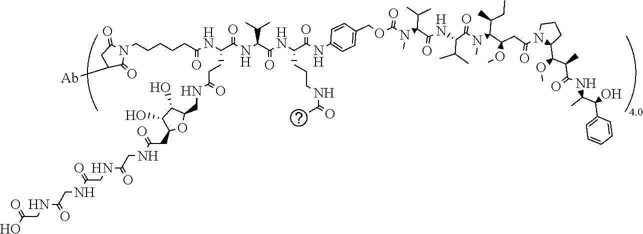 |
| ADC2-9-2 | 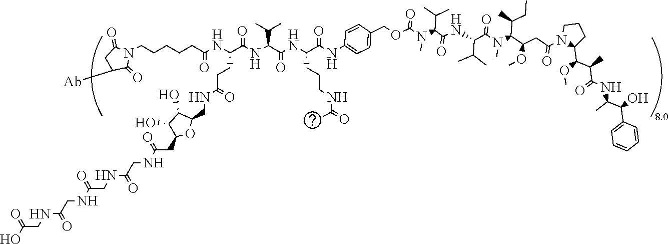 |
| ADC2-10 | 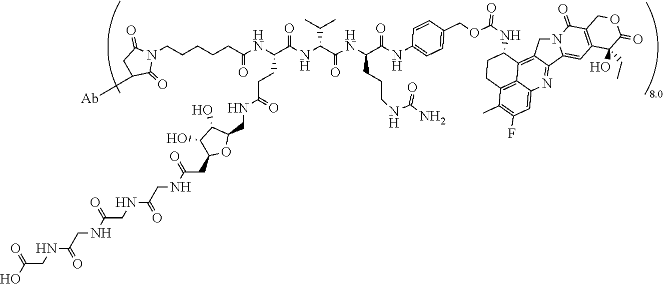 |
| ADC2-11 | 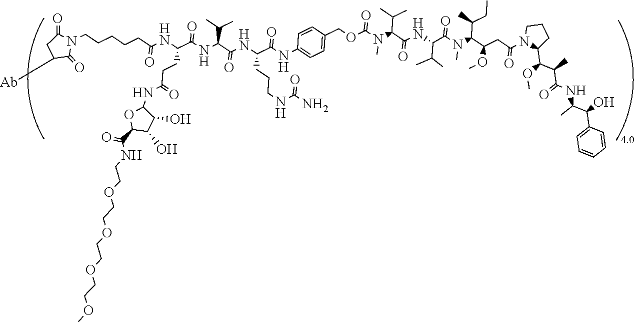 |
| ADC2-22 | 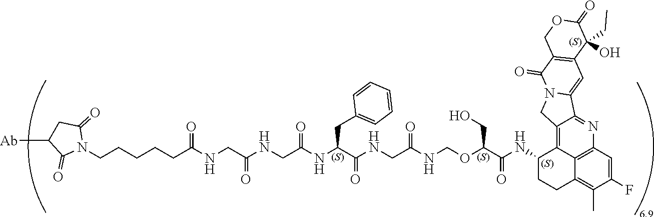 |
| ADC2-23 | 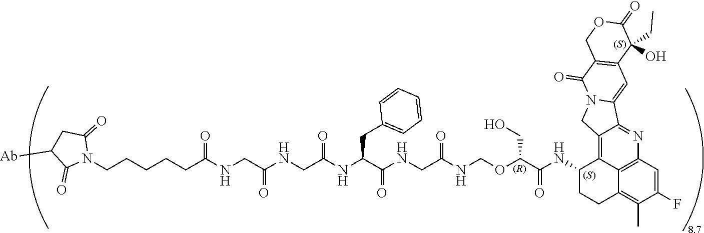 |
| ADC2-24 | 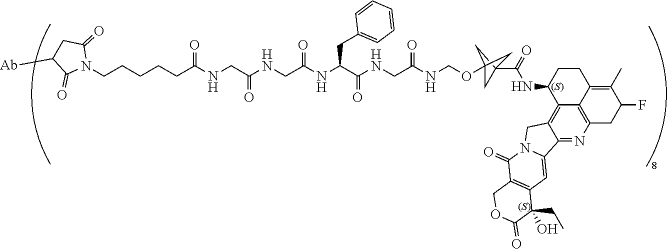 |
| ADC2-25 | 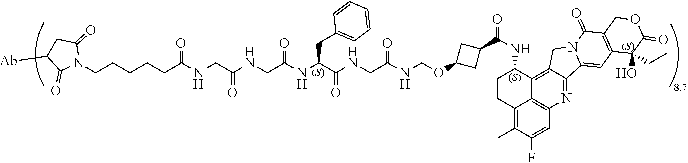 |
| ADC2-26 | 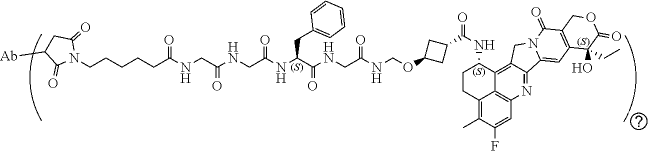 |
| ADC2-27 | 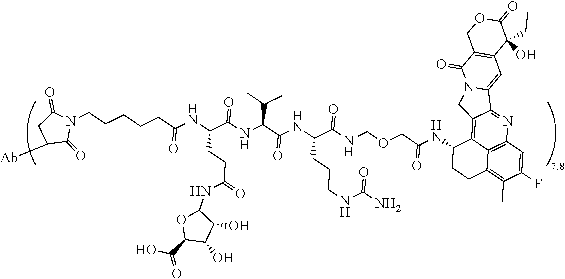 |
| ADC2-29 | 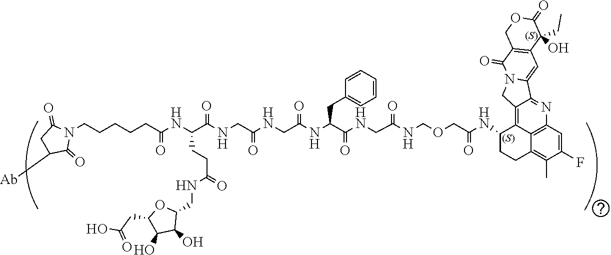 |
| ADC2-30 | 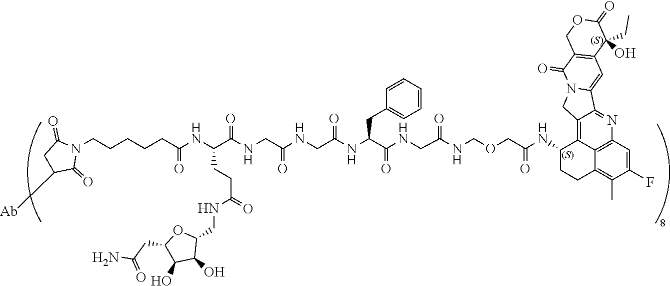 |
| ADC2-31 | 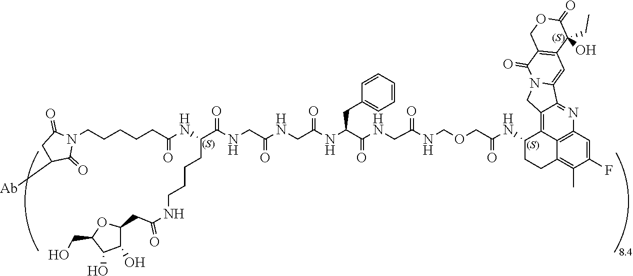 |
| ADC2-32 | 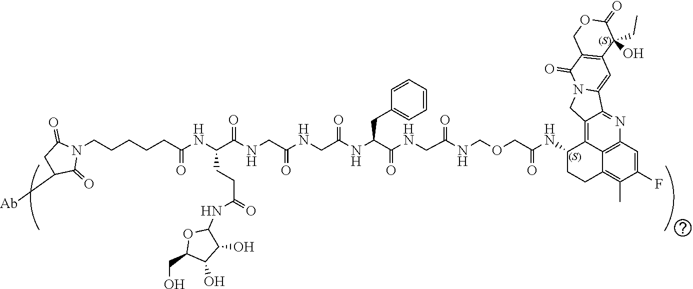 |
| ADC2-33 | 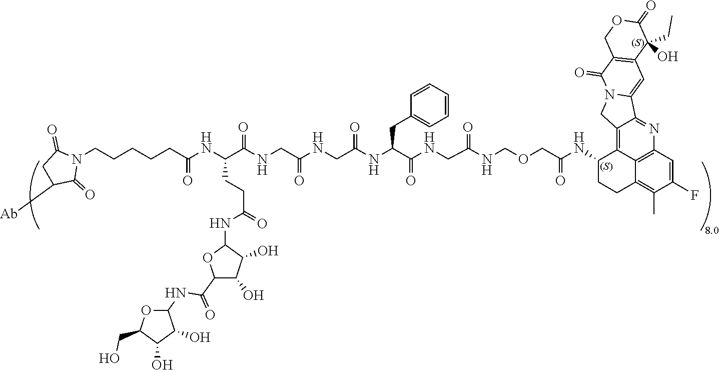 |
| ADC2-34 | 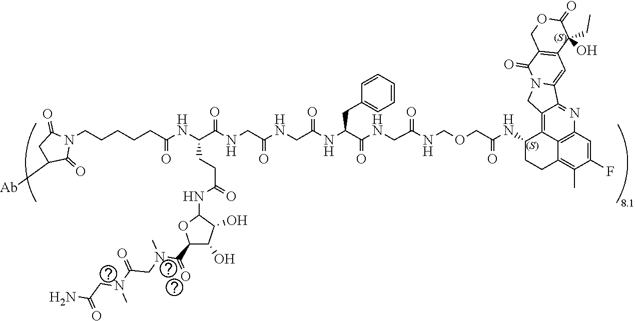 |
| ADC2-35 | 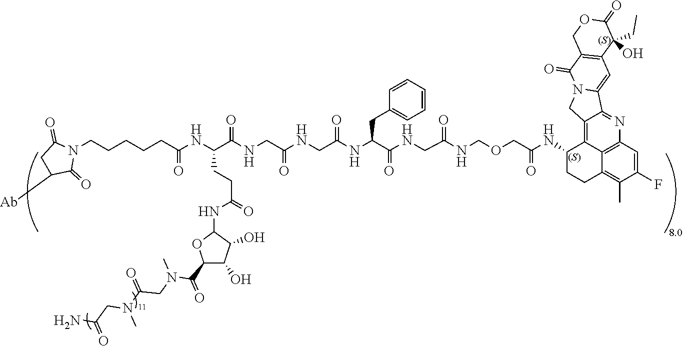 |
| ADC2-36-1 | 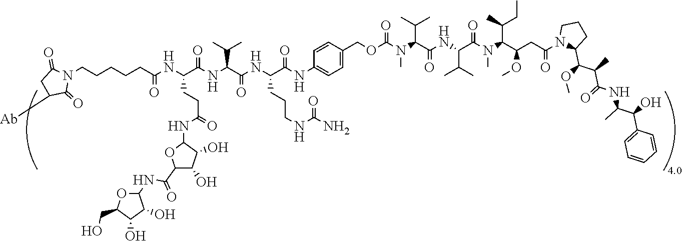 |
| ADC2-36-2 | 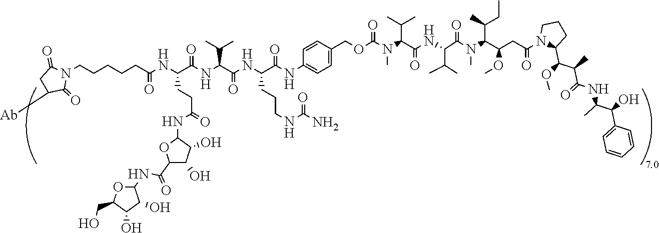 |
| ADC2-37 | 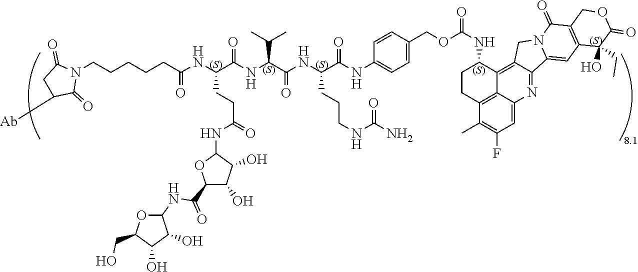 |
| ADC2-38 | 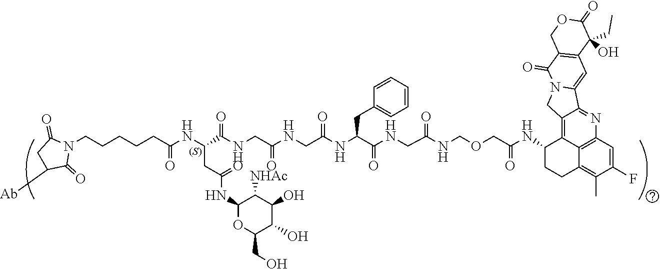 |
| ADC2-39 | 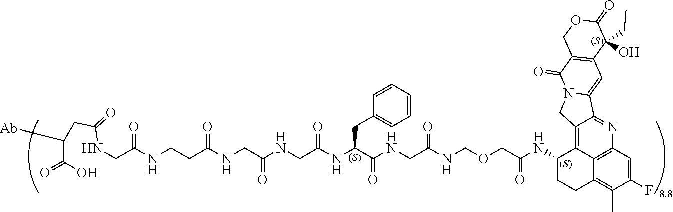 |
| ADC2-40 | 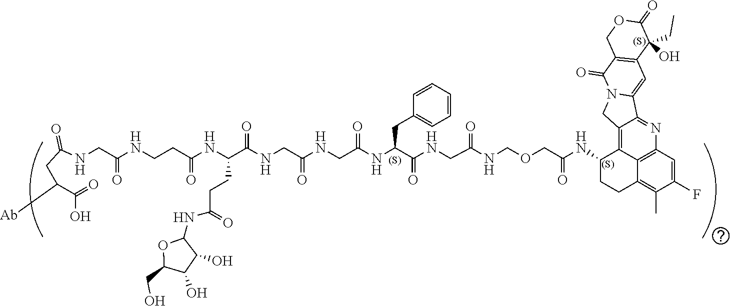 |
| ADC2-41 | 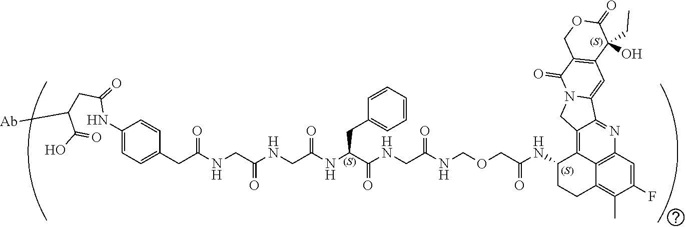 |
| ADC2-42 | 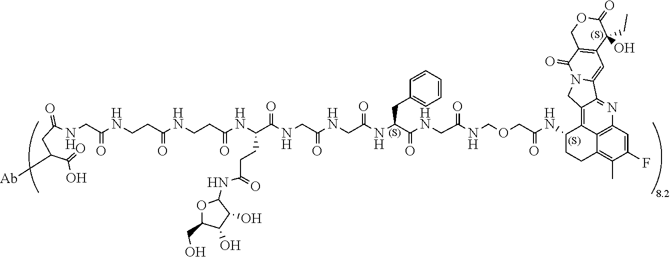 |
| ADC2-42-RO | 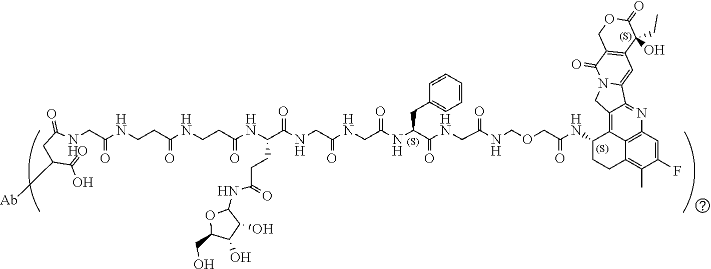 |
| ADC2-43 | 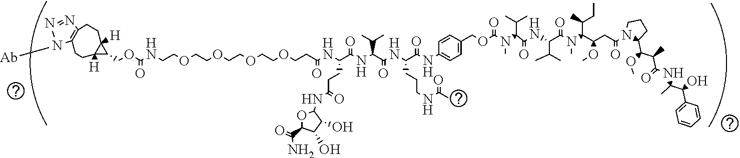 |

[symbol]

Connect conjugation

| | |
|---|---|
| ADC2-44 | 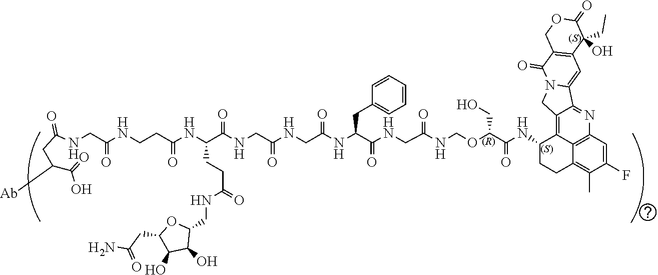 |
| ADC2-45 | 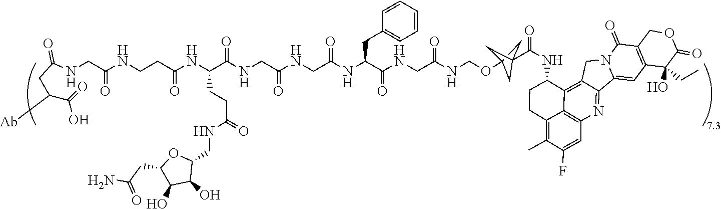 |
| ADC2-46 | 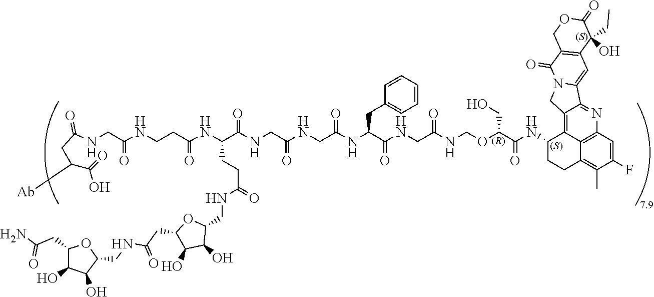 |
| ADC2-47 | 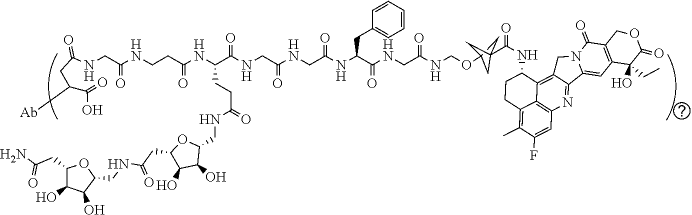 |
| ADC2-48 | 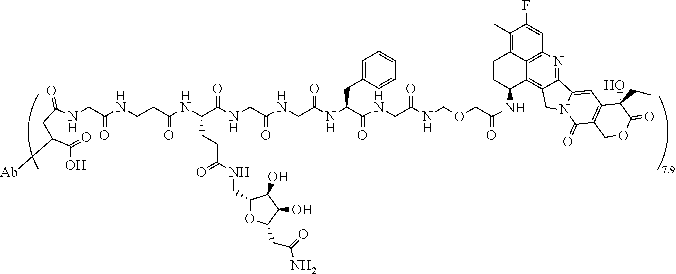 |
| ADC2-49 | 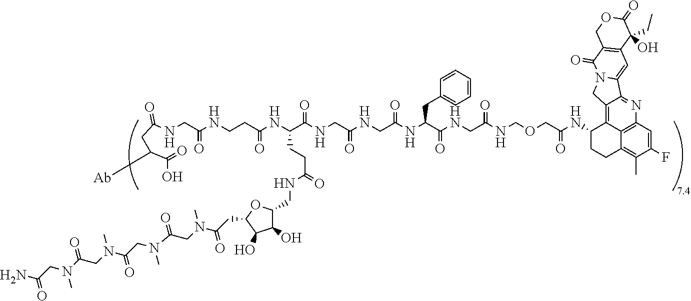 |
| ADC2-50 | 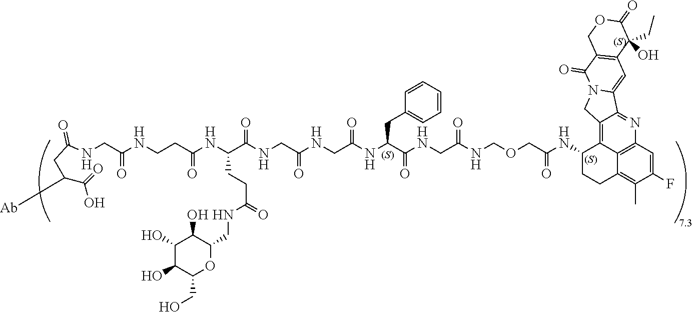 |
| ADC2-51 | 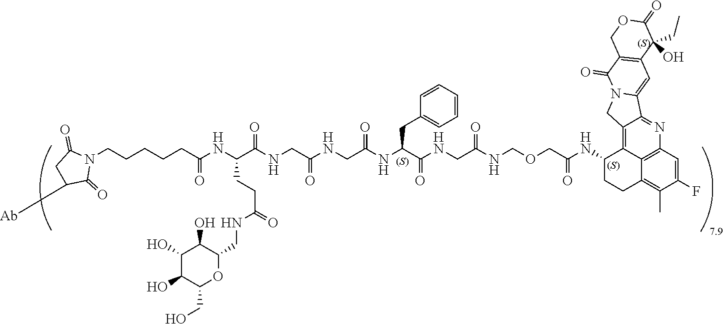 |
| ADC2-53 | 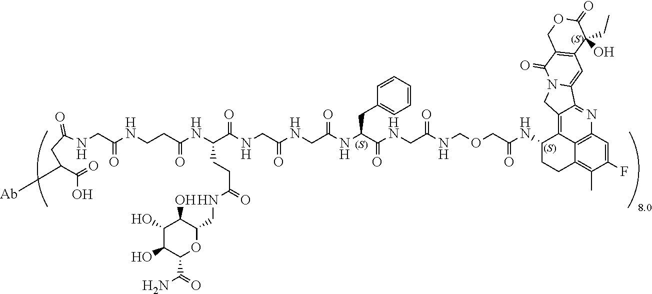 |
| ADC2-54 | 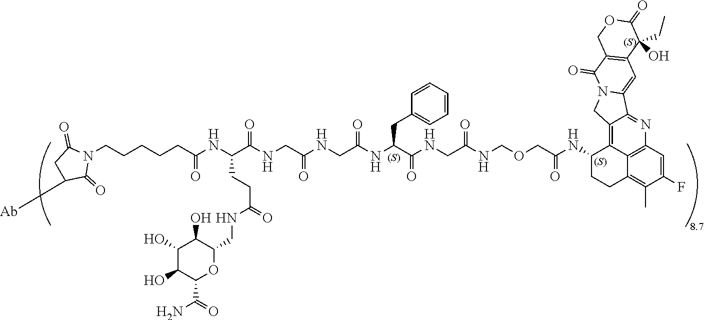 |
| ADC2-55 | 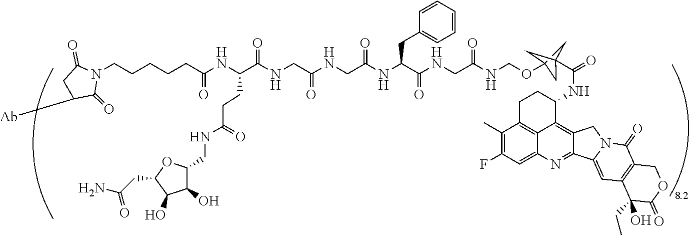 |
| ADC2-56 | 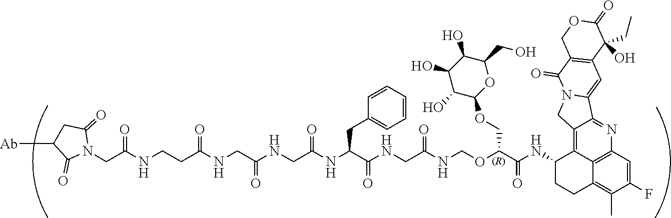 |
| ADC2-57 | 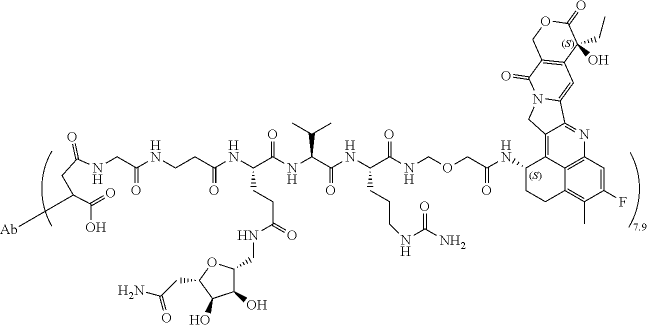 |
| ADC2-58 | 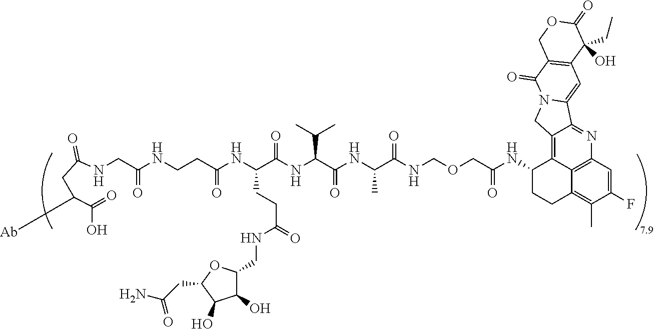 |
| ADC2-59 | 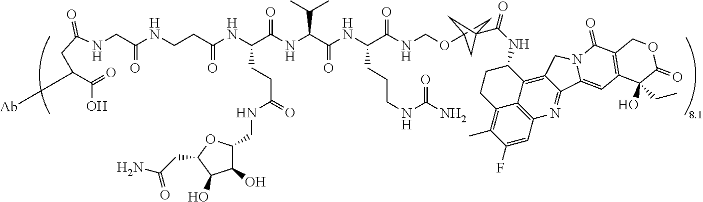 |
| ADC2-60 | 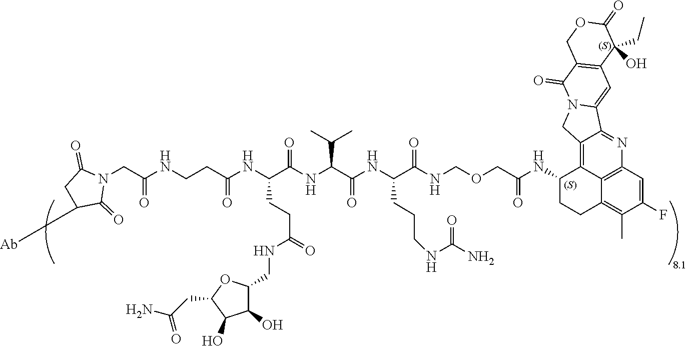 |
| ADC2-61 | 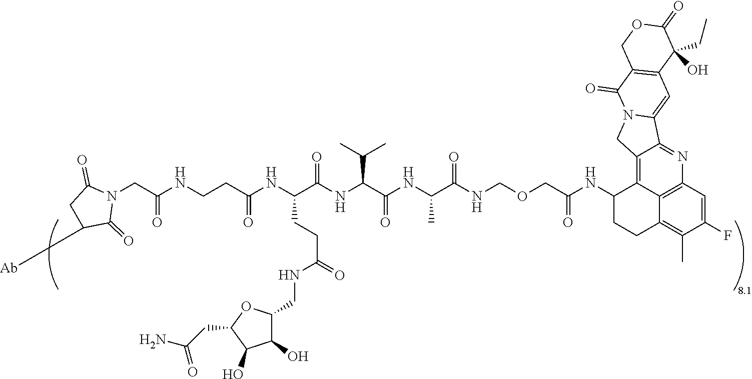 |
| ADC-2-62-1 | 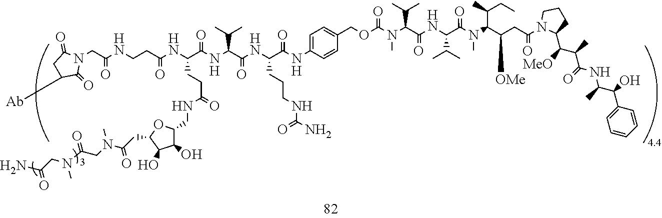 |
| ADC-2-62-2 | 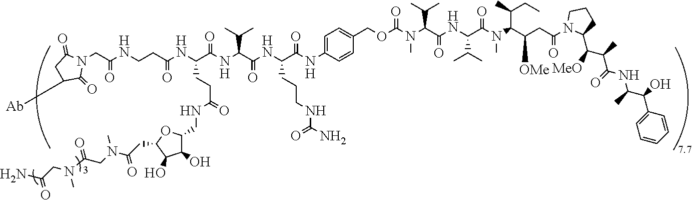 |
| ADC-2-63-1 | 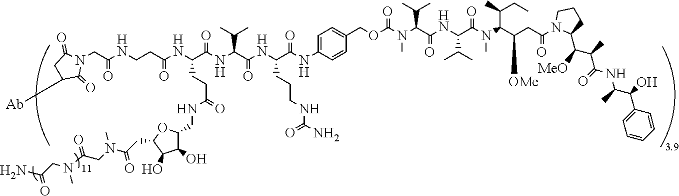 |
| ADC-2-63-2 | 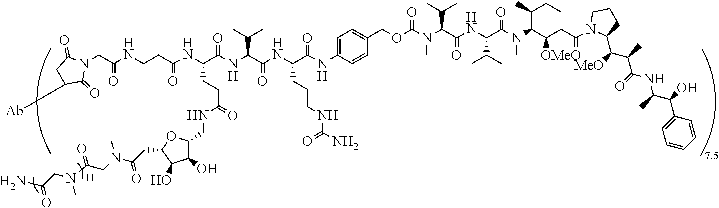 |
| ADC-2-64-1 | 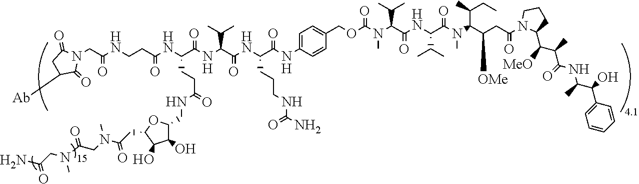 |
| ADC-2-64-2 | 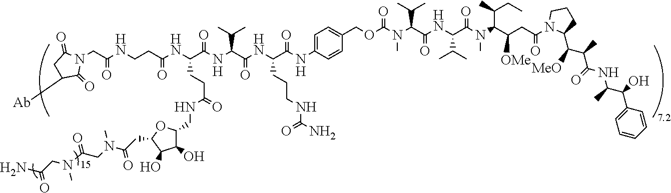 |
| ADC2-65-1 | 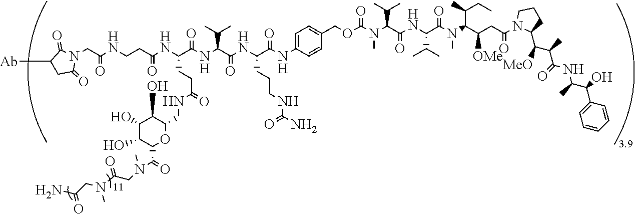 |
| ADC2-65-2 | 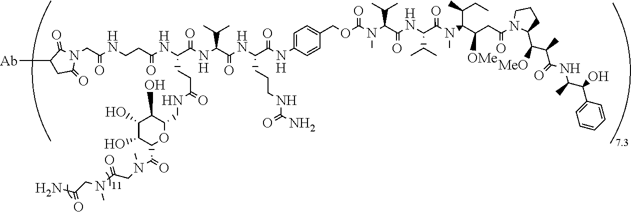 |
| ADC3-1 | 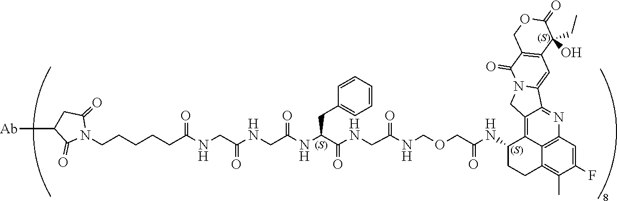 |
| ADC3-2 | 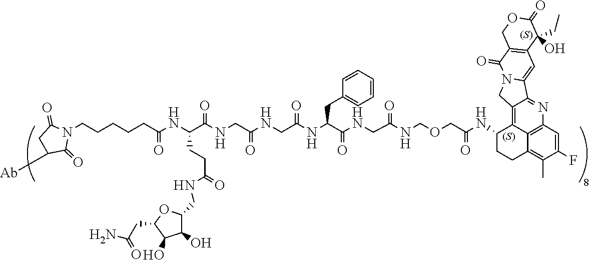 |
| ADC3-3 | 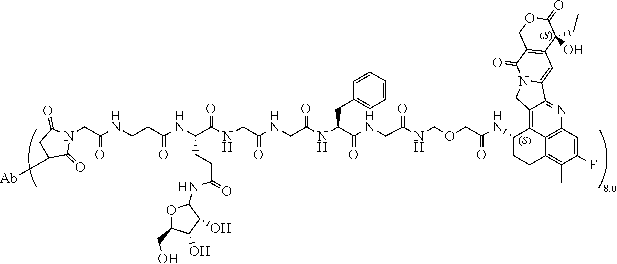 |
| ADC3-4 | 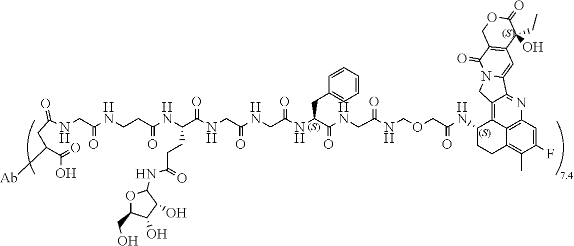 |
| ADC3-5 | 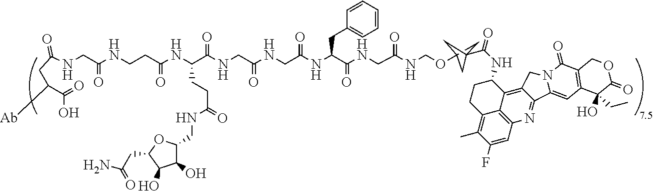 |
| ADC3-6 | 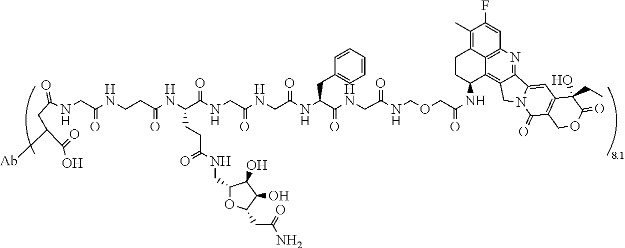 |
| ADC3-7 | 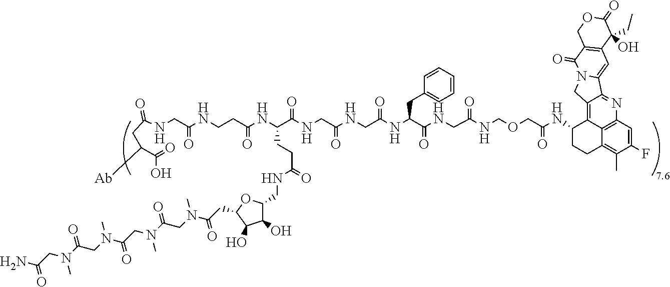 |
| ADC3-8 | 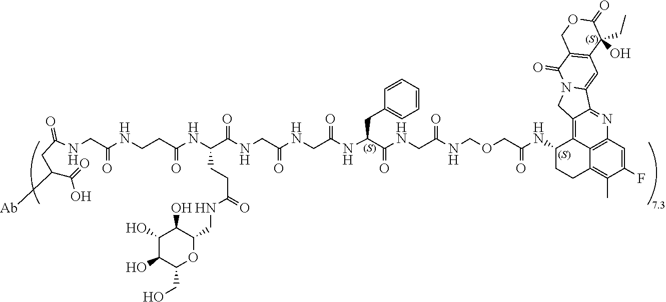 |
| ADC3-9 | 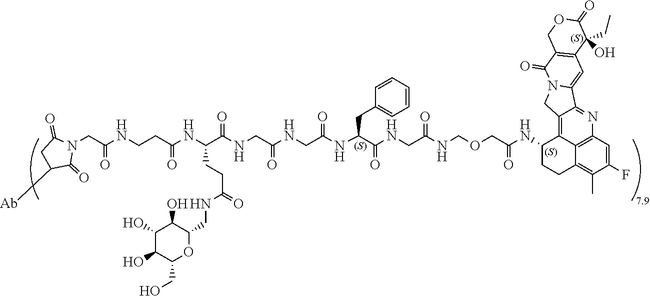 |
| ADC3-10 | 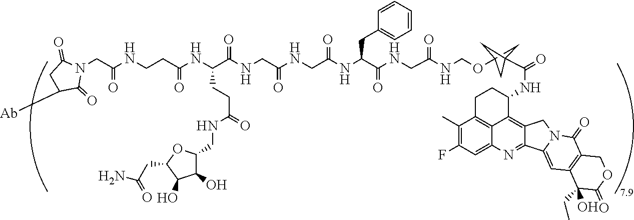 |
| ADC3-11 | 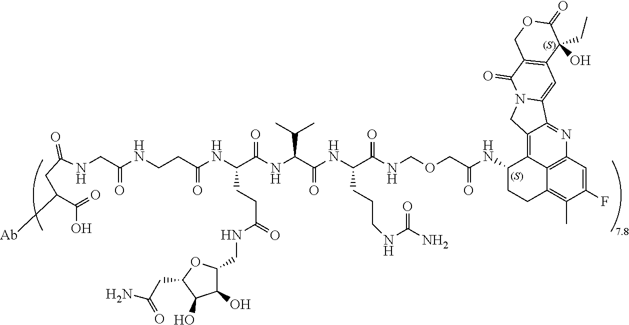 |
| ADC3-12 | 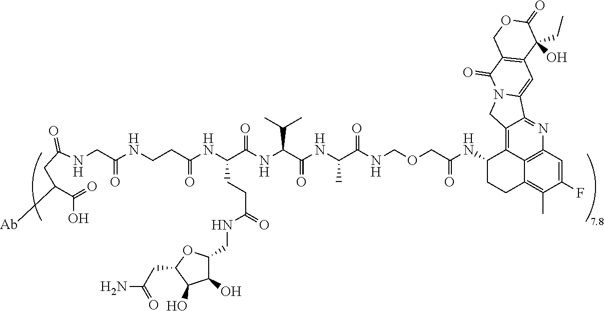 |
| ADC3-13 | 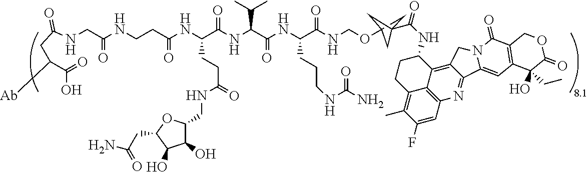 |
| ADC3-14 | 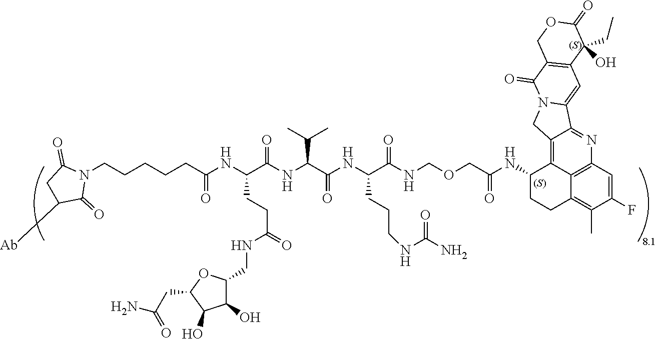 |
| ADC3-15 | 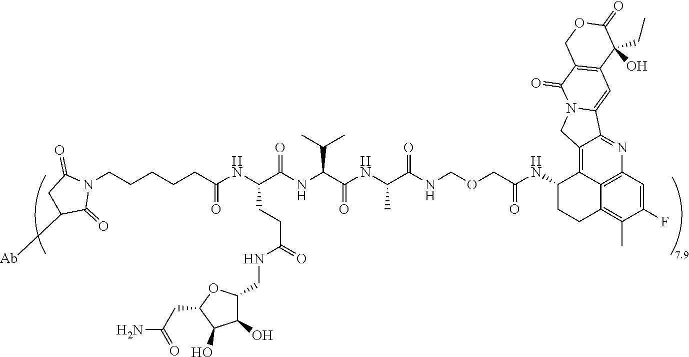 |
| ADC3-16 | 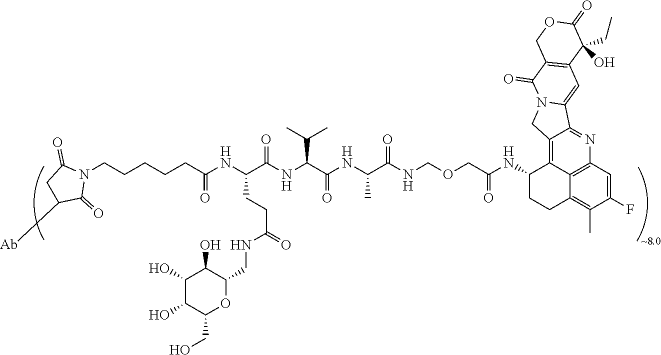 |
| ADC3-17 | 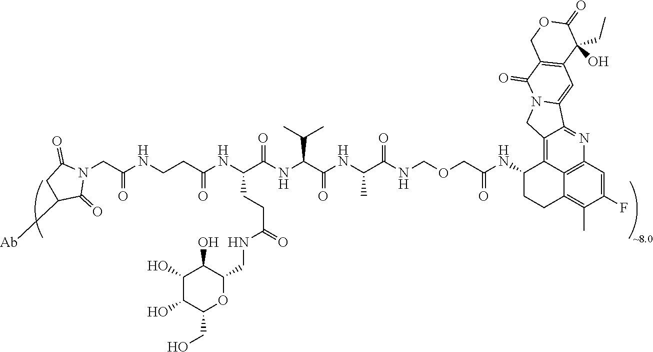 |
| ADC3-18 | 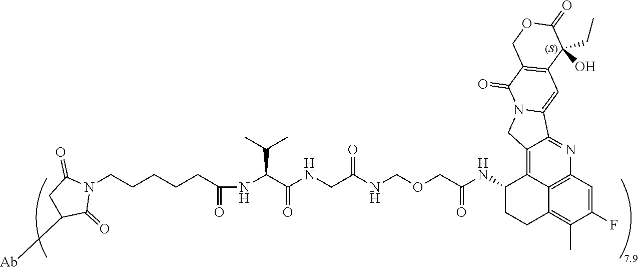 |
| ADC3-19 | 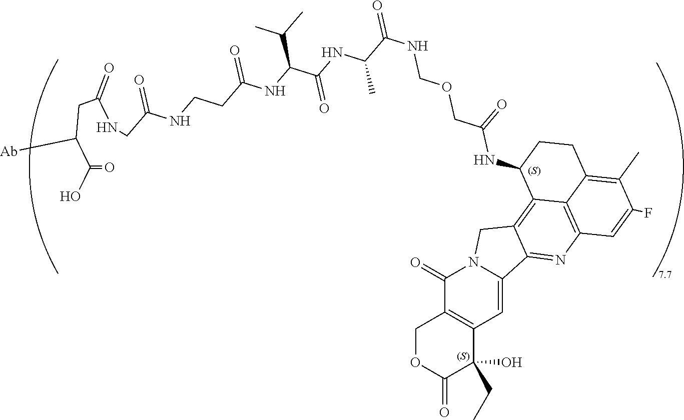 |
| ADC3-20 | 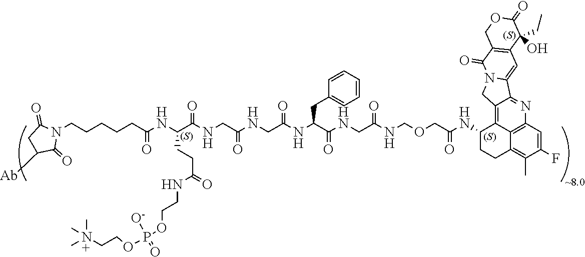 |
| ADC-C2 | 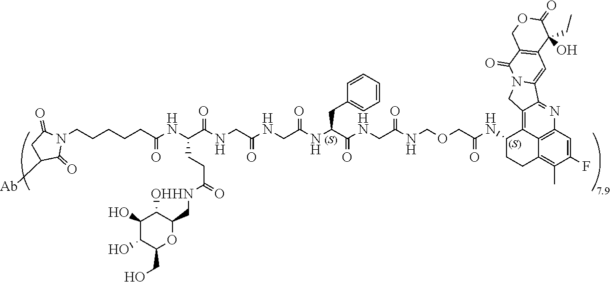 |
| ADC-C3 | 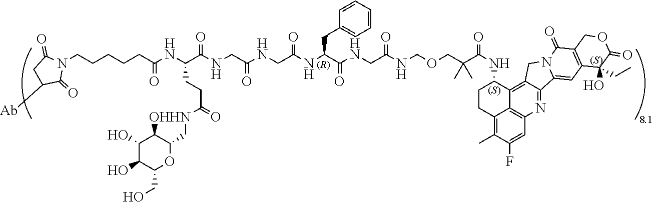 |
| ADC-C4 | 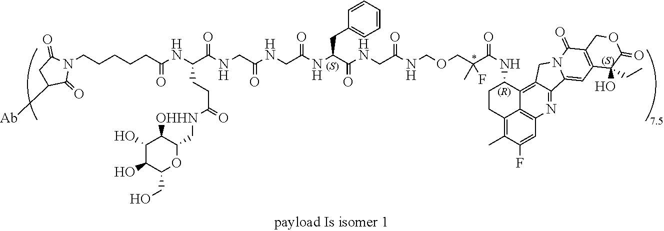 |
| ADC-C5 | 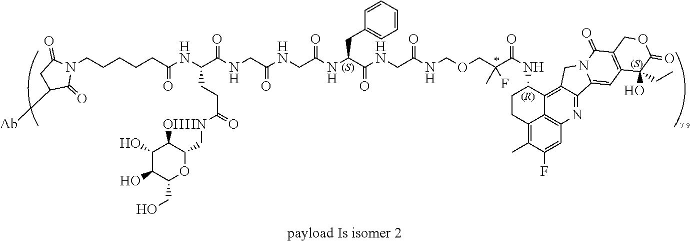 |

[symbol]

indicates data missing or illegible when filed

PCT Application No. PCT/CN2022/123665, the entire contents of which are incorporated herein by reference, discloses antibody drug conjugates, the linker payload portions of which are suitable for use in the context of the present disclosure, and linker payloads which are suitable for use in the context of the present disclosure. In some embodiments, a linker payload is a linker payload disclosed in PCT/CN2022/123665.

In some embodiments (e.g., of a compound of Formula I), PA is a residue of:

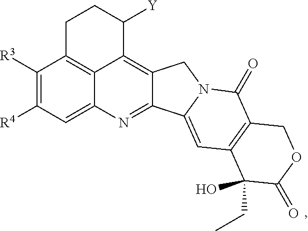

wherein

Y is -A-B-C′-D′-H;

A is a bond, CR¹R², or N-R¹;

B is a bond, —C(═O)—; or —C(═O)O—;

C′ is a bond, or a divalent group, wherein the divalent group is unsubstituted or substituted C₁₋₈alkyl, unsubstituted or substituted cycloalkyl, unsubstituted or substituted heterocyclyl, unsubstituted or substituted aryl, or unsubstituted or substituted heteroaryl;

D′ is a bond, NH, or O;

each of R¹, and R² is, independently, hydrogen, halogen, substituted or unsubstituted alkyl, or substituted or unsubstituted alkoxyl; or R¹, and R² together with the atom to which they are attached to, form unsubstituted or substituted cycloalkyl, unsubstituted or substituted heterocyclyl, unsubstituted or substituted aryl, or unsubstituted or substituted heteroaryl; and

each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl. In some embodiments, when R³ is methyl, and R⁴ is F, Y is not —NH—C(═O)—C—D-H.

In embodiments, in the residue of the payload depicted above, Y is -A-B-C′-D′-, the result of removal of -H from -A-B-C′-D′-H. It will be appreciated that while a payload residue can result from removal of hydrogen atom from a payload depicted herein, it also can result from removal of a hydroxy group, such as a hydroxy group formed when D′ is O in the payload depicted above (or a corresponding hydroxy group in any of the other payload structures depicted herein).

In some embodiments, the PA is a residue of:

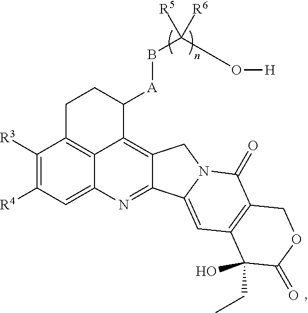

wherein

A is CR¹R², NH, or N-R¹;

B is a bond, —C(═O)—; or —C(═O)O—;

each of R¹, and R² is, independently, H, or C₁₋₄alkyl;
each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl;

each of R⁵, and R⁶ is, independently, hydrogen, halogen, substituted or unsubstituted alkyl, or substituted or unsubstituted alkoxyl; and

n is 1, 2, 3, 4, or 5.

In some embodiments, A is —CH₂—, and B is a bond.

In some embodiments, R⁵ and R⁶ are hydrogen, and n is 1, 2, or 3.

In some embodiments, R³ is methyl, and R⁴ is F.

In some embodiments, PA is a residue of:

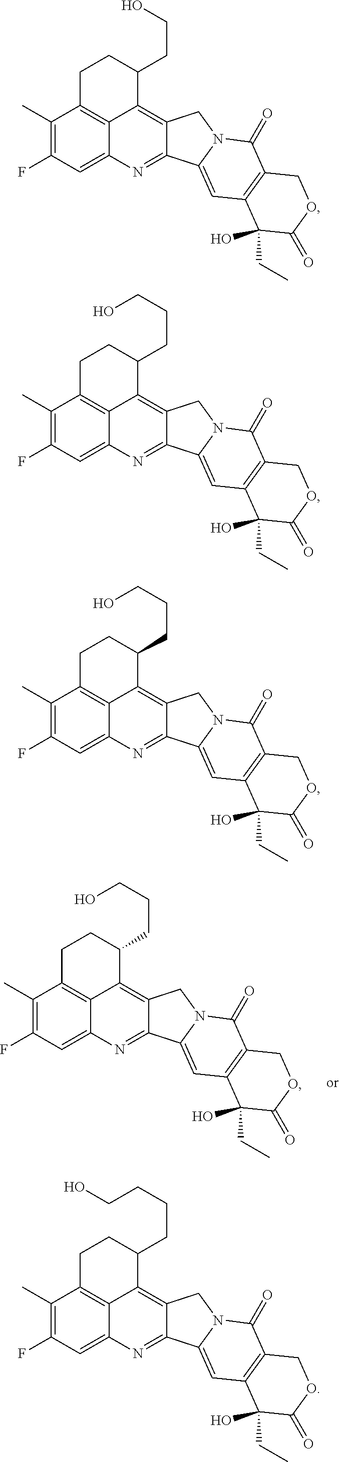 [SMILES: CC[C@@]1(O)C(=O)OCc2c1cc1n(c2=O)Cc2c-1nc1cc(F)c(C)c3c1c2C(CCO)CC3 ; CC[C@@]1(O)C(=O)OCc2c1cc1n(c2=O)Cc2c-1nc1cc(F)c(C)c3c1c2C(CCCO)CC3 ; CC[C@@]1(O)C(=O)OCc2c1cc1n(c2=O)Cc2c-1nc1cc(F)c(C)c3c1c2[C@H](CCCO)CC3 ; CC[C@@]1(O)C(=O)OCc2c1cc1n(c2=O)Cc2c-1nc1cc(F)c(C)c3c1c2[C@@H](CCCO)CC3 ; "or" ; CC[C@@]1(O)C(=O)OCc2c1cc1n(c2=O)Cc2c-1nc1cc(F)c(C)c3c1c2C(CCCCO)CC3]

In some embodiments, R³, and R⁴ together with the atoms to which they are attached, form an unsubstituted or substituted dioxole ring.

In some embodiments, PA is a residue of:

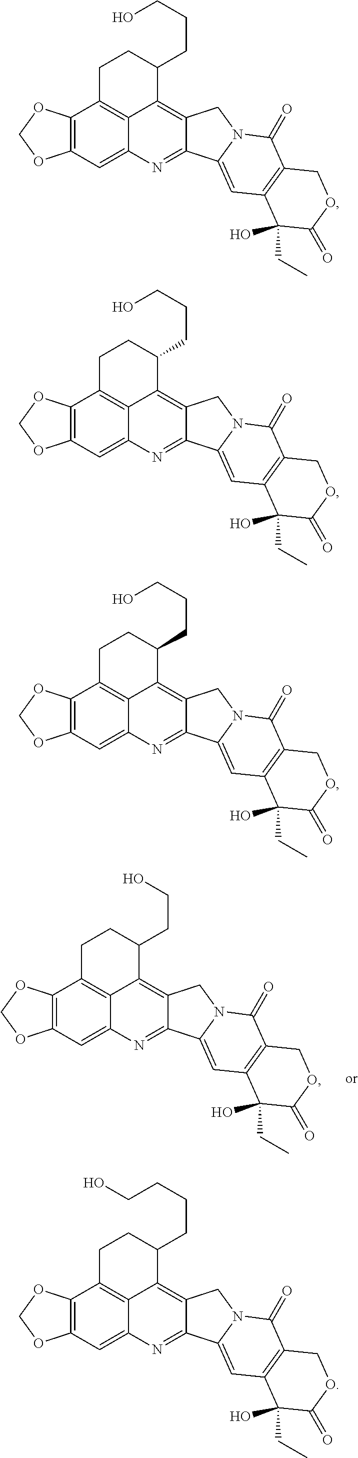 [SMILES: CC[C@@]1(O)C(=O)OCc2c1cc1n(c2=O)Cc2c-1nc1cc3c(c4c1c2C(CCCO)CC4)OCO3 ; CC[C@@]1(O)C(=O)OCc2c1cc1n(c2=O)Cc2c-1nc1cc3c(c4c1c2[C@@H](CCCO)CC4)OCO3 ; CC[C@@]1(O)C(=O)OCc2c1cc1n(c2=O)Cc2c-1nc1cc3c(c4c1c2[C@H](CCCO)CC4)OCO3 ; CC[C@@]1(O)C(=O)OCc2c1cc1n(c2=O)Cc2c-1nc1cc3c(c4c1c2C(CCO)CC4)OCO3 ; "or" ; CC[C@@]1(O)C(=O)OCc2c1cc1n(c2=O)Cc2c-1nc1cc3c(c4c1c2C(CCCCO)CC4)OCO3]

In some embodiments, A is —N(CH₃)—, and B is a bond.

In some embodiments, R⁵ and R⁶ are hydrogen, and n is 2.

In some embodiments, PA is a residue of:

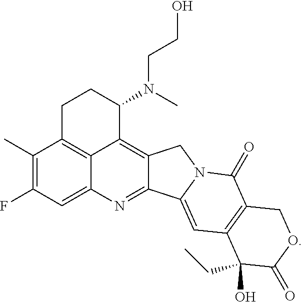 [SMILES: CC[C@@]1(O)C(=O)OCc2c1cc1n(c2=O)Cc2c-1nc1cc(F)c(C)c3c1c2[C@@H](N(C)CCO)CC3]

In some embodiments, A is —NH—, and B is —C(═O)O-. In further embodiments, R⁵ and R⁶ are hydrogen, and n is 2.

In some embodiments, PA is a residue of:

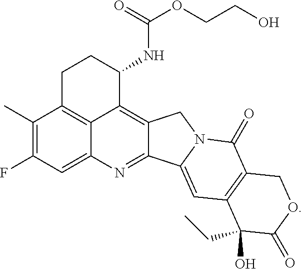 [SMILES: CC[C@@]1(O)C(=O)OCc2c1cc1n(c2=O)Cc2c-1nc1cc(F)c(C)c3c1c2[C@@H](NC(=O)OCCO)CC3]

In some embodiments, A is —NH—, and B is —C(═O)—. In further embodiments, R⁵ and R⁶ are hydrogen, and n is 2.

In some embodiments, PA is a residue of

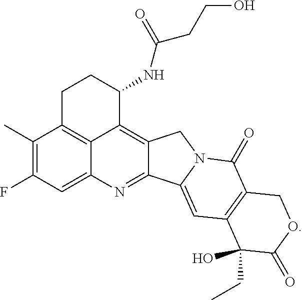 [SMILES: CC[C@@]1(O)C(=O)OCc2c1cc1n(c2=O)Cc2c-1nc1cc(F)c(C)c3c1c2[C@@H](NC(=O)CCO)CC3]

In some embodiments, R³ is Cl, R⁴ is F, and B is —C(═O)—.

In some embodiments, PA is a residue of

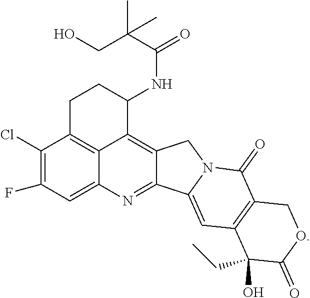 [SMILES: CC[C@@]1(O)C(=O)OCc2c1cc1n(c2=O)Cc2c-1nc1cc(F)c(Cl)c3c1c2C(NC(=O)C(C)(C)CO)CC3]

In some embodiments, R³ is methyl, R⁴ is Cl, and B is —C(═O)—.

In some embodiments, PA is a residue of

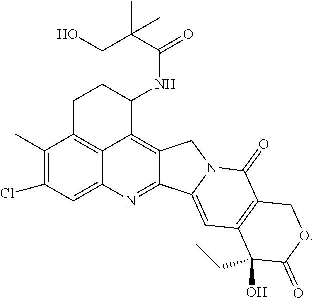 [SMILES: CC[C@@]1(O)C(=O)OCc2c1cc1n(c2=O)Cc2c-1nc1cc(Cl)c(C)c3c1c2C(NC(=O)C(C)(C)CO)CC3]

In some embodiments, R³, and R⁴ together with the atoms to which they are attached to, form unsubstituted or substituted heterocyclyl.

In some embodiments, R³, and R⁴ together with the atoms to which they are attached to, form an unsubstituted or substituted dioxole ring, and B is —C(═O)—.

In some embodiments, PA is a residue of:

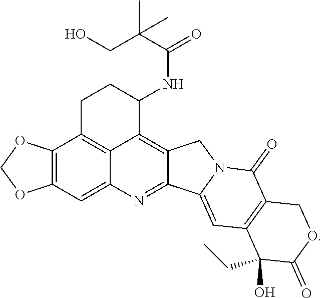 [SMILES: CC[C@@]1(O)C(=O)OCc2c1cc1n(c2=O)Cc2c-1nc1cc3c(c4c1c2C(NC(=O)C(C)(C)CO)CC4)OCO3]

In some embodiments, PA is a residue of:

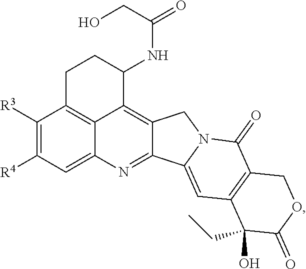

wherein values and alternative values for the variables (e.g., R³, R⁴) are as described elsewhere herein.

In some embodiments, R³ is methyl; and R⁴ is Cl.

In some embodiments, PA is a residue of:

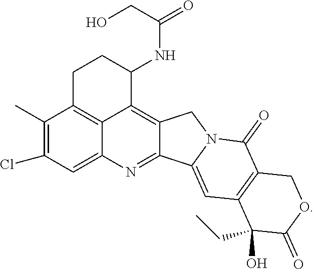 [SMILES: CC[C@@]1(O)C(=O)OCc2c1cc1n(c2=O)Cc2c-1nc1cc(Cl)c(C)c3c1c2C(NC(=O)CO)CC3]

In some embodiments, R³ is Cl; and R⁴ is F.

In some embodiments, PA is a residue of:

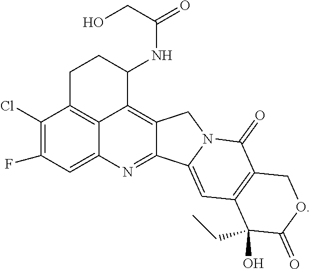 [SMILES: CC[C@@]1(O)C(=O)OCc2c1cc1n(c2=O)Cc2c-1nc1cc(F)c(Cl)c3c1c2C(NC(=O)CO)CC3]

In some embodiments, R³ is F; and R⁴ is F.

In some embodiments, PA is a residue of:

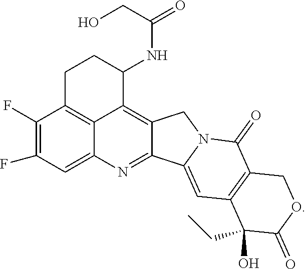 [SMILES: CC[C@@]1(O)C(=O)OCc2c1cc1n(c2=O)Cc2c-1nc1cc(F)c(F)c3c1c2C(NC(=O)CO)CC3]

In some embodiments, R³ is H; and R⁴ is F.

In some embodiments, PA is a residue of

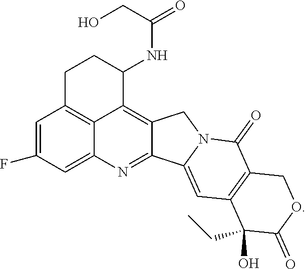 [SMILES: CC[C@@]1(O)C(=O)OCc2c1cc1n(c2=O)Cc2c-1nc1cc(F)cc3c1c2C(NC(=O)CO)CC3]

In some embodiments, R³ is H; and R⁴ is OH.

In some embodiments, PA is a residue of:

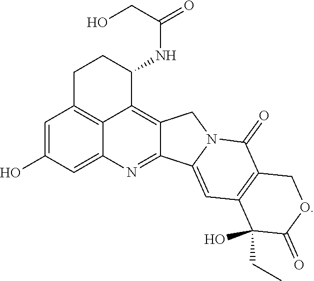 [SMILES: CC[C@@]1(O)C(=O)OCc2c1cc1n(c2=O)Cc2c-1nc1cc(O)cc3c1c2[C@@H](NC(=O)CO)CC3]

In some embodiments, R³ is methyl; and R⁴ is methyl.

In some embodiments, PA is a residue of:

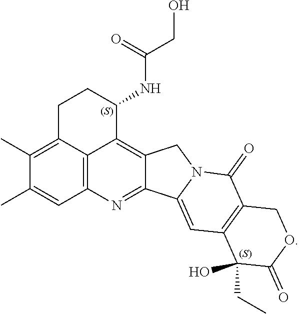

In some embodiments, R³ is methoxyl; and R⁴ is F.

In some embodiments, PA is a residue of:

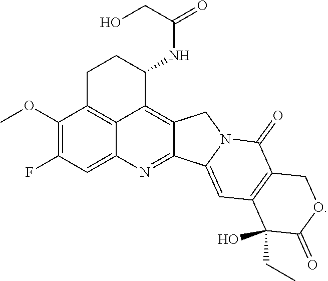 [SMILES: CC[C@@]1(O)C(=O)OCc2c1cc1n(c2=O)Cc2c-1nc1cc(F)c(OC)c3c1c2[C@@H](NC(=O)CO)CC3]

In some embodiments, R³ is H; and R⁴ is methoxyl.

In some embodiments, PA is a residue of

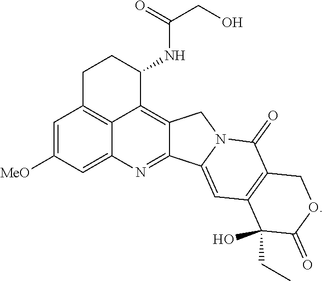 [SMILES: CC[C@@]1(O)C(=O)OCc2c1cc1n(c2=O)Cc2c-1nc1cc(OC)cc3c1c2[C@@H](NC(=O)CO)CC3]

In some embodiments, R³ is H; and R⁴ is Cl.

In some embodiments, PA is a residue of:

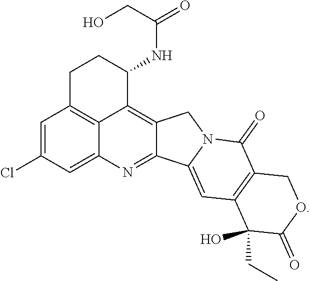 [SMILES: CC[C@@]1(O)C(=O)OCc2c1cc1n(c2=O)Cc2c-1nc1cc(Cl)cc3c1c2[C@@H](NC(=O)CO)CC3]

In some embodiments, R³ and R⁴ together with the atoms to which they are attached to, form unsubstituted or substituted heterocyclyl.

In some embodiments, R³, and R⁴ together with the atoms to which they are attached to, form an unsubstituted or substituted dioxole ring.

In some embodiments, PA is a residue of:

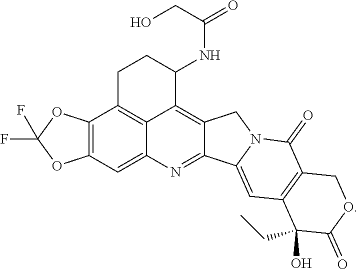 [SMILES: CC[C@@]1(O)C(=O)OCc2c1cc1n(c2=O)Cc2c-1nc1cc3c(c4c1c2C(NC(=O)CO)CC4)OC(F)(F)O3]

In some embodiments, PA is a residue of:

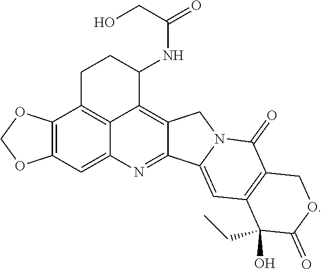 [SMILES: CC[C@@]1(O)C(=O)OCc2c1cc1n(c2=O)Cc2c-1nc1cc3c(c4c1c2C(NC(=O)CO)CC4)OCO3]

In some embodiments, PA is a residue of:

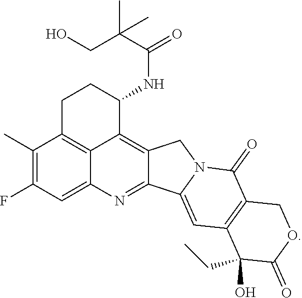 [SMILES: CC[C@@]1(O)C(=O)OCc2c1cc1n(c2=O)Cc2c-1nc1cc(F)c(C)c3c1c2[C@@H](NC(=O)C(C)(C)CO)CC3]

In some embodiments, PA is a residue of:

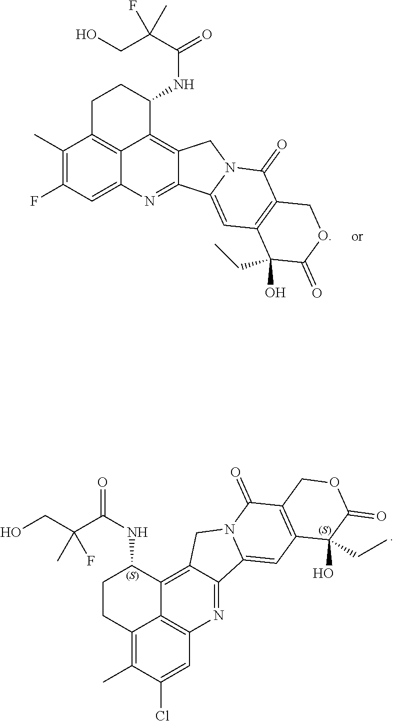

In some embodiments, PA is a residue of:

| Examples | Payload structure |
|---|---|
| 1-A | 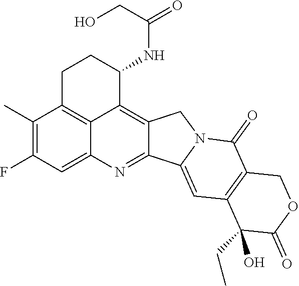 [SMILES: CC[C@@]1(O)C(=O)OCc2c1cc1n(c2=O)Cc2c-1nc1cc(F)c(C)c3c1c2[C@@H](NC(=O)CO)CC3] |
| 1-B | 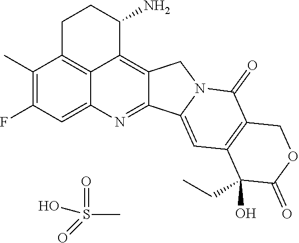 |
| 1-C | 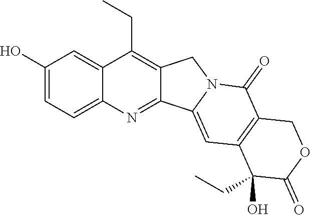 [SMILES: CCc1c2c(nc3ccc(O)cc13)-c1cc3c(c(=O)n1C2)COC(=O)[C@]3(O)CC] |
| 1-1 | 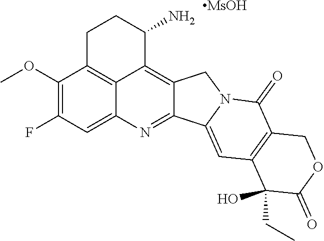 [SMILES: "·MsOH" ; CC[C@@]1(O)C(=O)OCc2c1cc1n(c2=O)Cc2c-1nc1cc(F)c(OC)c3c1c2[C@@H](N)CC3] |
| 1-2 | 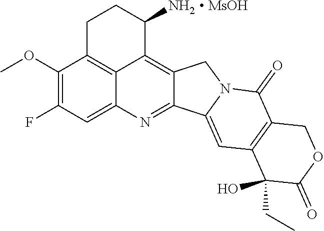 [SMILES: "MsOH" ; CC[C@@]1(O)C(=O)OCc2c1cc1n(c2=O)Cc2c-1nc1cc(F)c(OC)c3c1c2[C@H](N)CC3] |
| 1-3 | 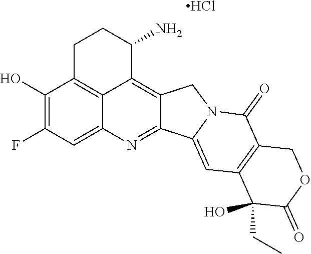 [SMILES: Cl ; CC[C@@]1(O)C(=O)OCc2c1cc1n(c2=O)Cc2c-1nc1cc(F)c(O)c3c1c2[C@@H](N)CC3] |
| 1-4 | 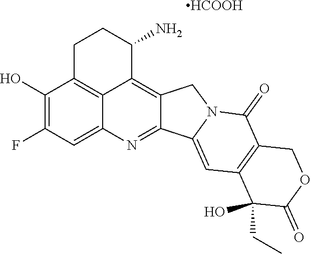 [SMILES: "·HCOOH" ; CC[C@@]1(O)C(=O)OCc2c1cc1n(c2=O)Cc2c-1nc1cc(F)c(O)c3c1c2[C@@H](N)CC3] |
| 1-5 | 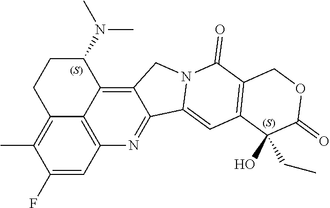 |
| 1-6 | 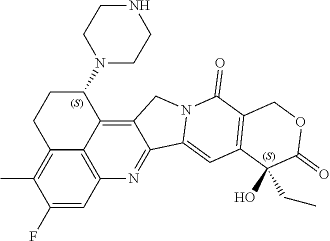 |
| 1-7 | 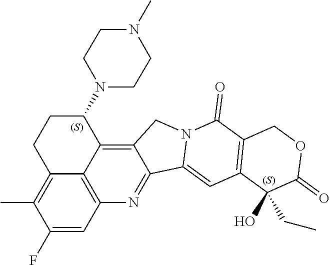 |
| 1-8 | 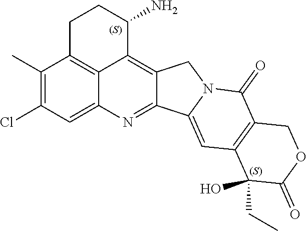 |
| 1-9 | 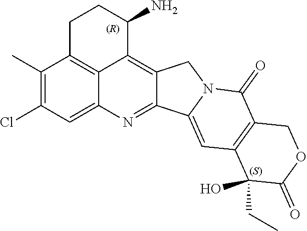 |
| 1-10 | 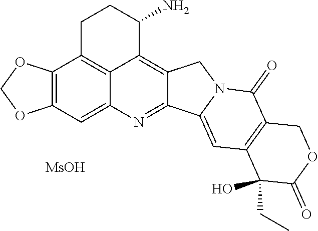 [SMILES: "MsOH" ; CC[C@@]1(O)C(=O)OCc2c1cc1n(c2=O)Cc2c-1nc1cc3c(c4c1c2[C@@H](N)CC4)OCO3] |
| 1-11 | 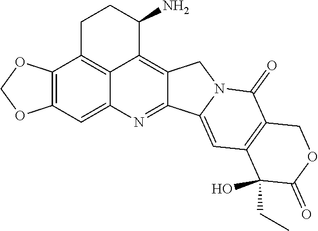 [SMILES: CC[C@@]1(O)C(=O)OCc2c1cc1n(c2=O)Cc2c-1nc1cc3c(c4c1c2[C@H](N)CC4)OCO3] |
| 1-12 | 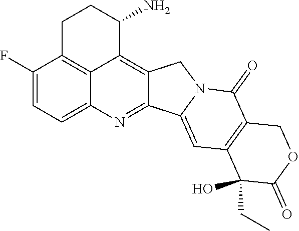 [SMILES: CC[C@@]1(O)C(=O)OCc2c1cc1n(c2=O)Cc2c-1nc1ccc(F)c3c1c2[C@@H](N)CC3] |
| 1-13 | 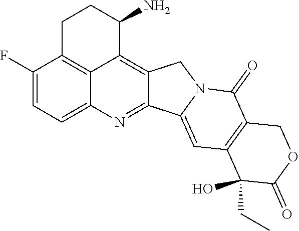 [SMILES: CC[C@@]1(O)C(=O)OCc2c1cc1n(c2=O)Cc2c-1nc1ccc(F)c3c1c2[C@H](N)CC3] |
| 1-14 | 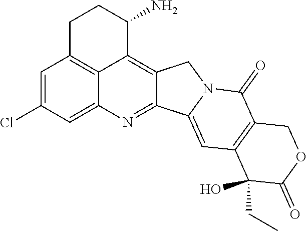 [SMILES: CC[C@@]1(O)C(=O)OCc2c1cc1n(c2=O)Cc2c-1nc1cc(Cl)cc3c1c2[C@@H](N)CC3] |
| 1-15 | 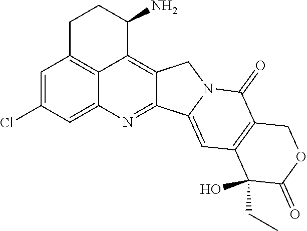 [SMILES: CC[C@@]1(O)C(=O)OCc2c1cc1n(c2=O)Cc2c-1nc1cc(Cl)cc3c1c2[C@H](N)CC3] |
| 1-16 | 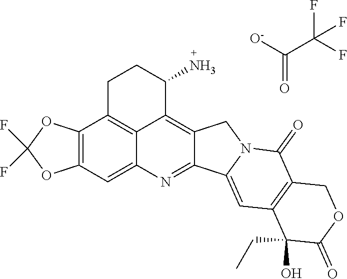 [SMILES: CC[C@@]1(O)C(=O)OCc2c1cc1n(c2=O)Cc2c-1nc1cc3c(c4c1c2[C@@H](N)CC4)OC(F)(F)O3 ; O=C([O-])C(F)(F)F] |
| 1-17 | 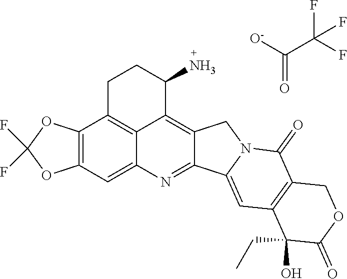 [SMILES: CC[C@@]1(O)C(=O)OCc2c1cc1n(c2=O)Cc2c-1nc1cc3c(c4c1c2[C@H](N)CC4)OC(F)(F)O3 ; O=C([O-])C(F)(F)F] |
| 1-18 | 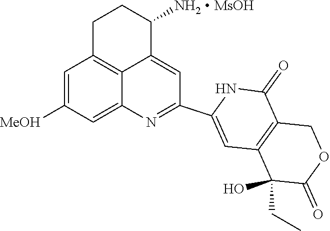 |
| 1-19 | 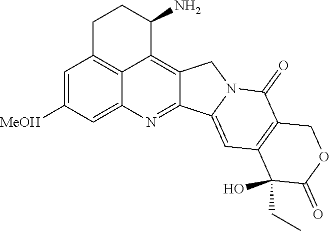 |
| 1-20 | 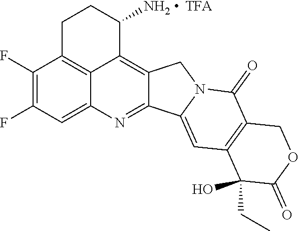 [SMILES: CC[C@@]1(O)C(=O)OCc2c1cc1n(c2=O)Cc2c-1nc1cc(F)c(F)c3c1c2[C@@H](N)CC3 ; "TFA"] |
| 1-21 | 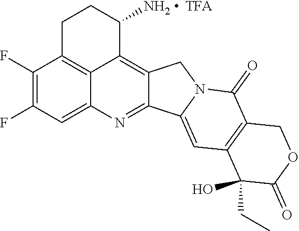 [SMILES: CC[C@@]1(O)C(=O)OCc2c1cc1n(c2=O)Cc2c-1nc1cc(F)c(F)c3c1c2[C@@H](N)CC3 ; "TFA"] |
| 1-22 | 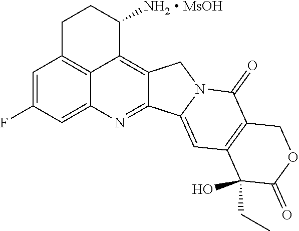 [SMILES: "MsOH" ; CC[C@@]1(O)C(=O)OCc2c1cc1n(c2=O)Cc2c-1nc1cc(F)cc3c1c2[C@@H](N)CC3] |
| 1-23 | 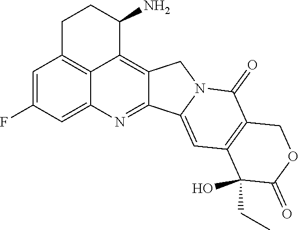 [SMILES: CC[C@@]1(O)C(=O)OCc2c1cc1n(c2=O)Cc2c-1nc1cc(F)cc3c1c2[C@H](N)CC3] |
| 1-24 | 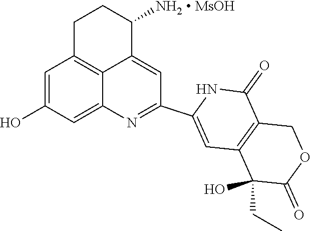 [SMILES: "MsOH" ; CC[C@@]1(O)C(=O)OCc2c1cc(-c1cc3c4c(cc(O)cc4n1)CC[C@@H]3N)[nH]c2=O] |
| 1-25 | 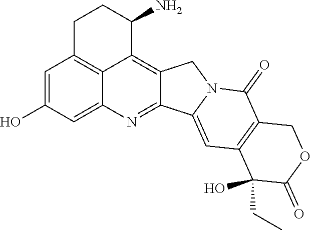 [SMILES: CC[C@@]1(O)C(=O)OCc2c1cc1n(c2=O)Cc2c-1nc1cc(O)cc3c1c2[C@H](N)CC3] |
| 1-26 | 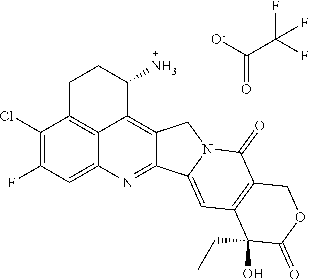 [SMILES: CC[C@@]1(O)C(=O)OCc2c1cc1n(c2=O)Cc2c-1nc1cc(F)c(Cl)c3c1c2[C@@H](N)CC3 ; O=C([O-])C(F)(F)F] |
| 1-27 | 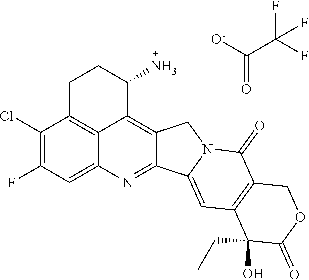 [SMILES: CC[C@@]1(O)C(=O)OCc2c1cc1n(c2=O)Cc2c-1nc1cc(F)c(Cl)c3c1c2[C@@H](N)CC3 ; O=C([O-])C(F)(F)F] |

In some embodiments, PA is a residue of:

| Compound | |
|---|---|
| number | Payload structure |
| 2-1 | 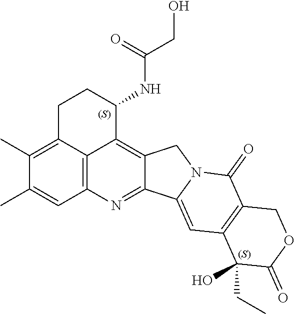 |
| 2-2 | 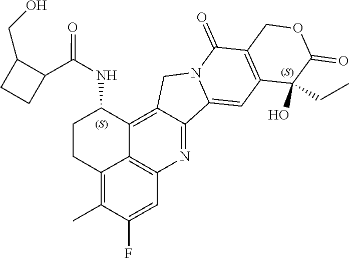 |
| 2-3 | 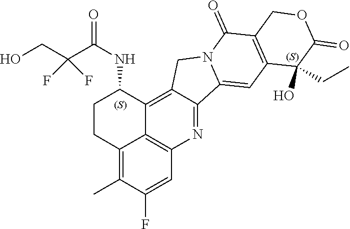 |
| 2-4 | 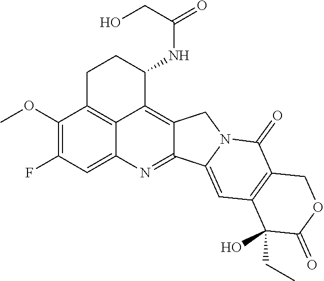 [SMILES: CC[C@@]1(O)C(=O)OCc2c1cc1n(c2=O)Cc2c-1nc1cc(F)c(OC)c3c1c2[C@@H](NC(=O)CO)CC3] |
| 2-5 | 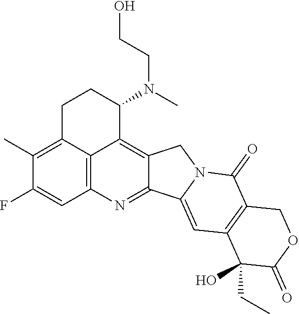 [SMILES: CC[C@@]1(O)C(=O)OCc2c1cc1n(c2=O)Cc2c-1nc1cc(F)c(C)c3c1c2[C@@H](N(C)CCO)CC3] |
| 2-6 | 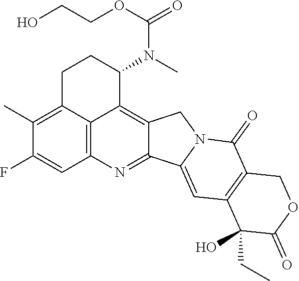 [SMILES: CC[C@@]1(O)C(=O)OCc2c1cc1n(c2=O)Cc2c-1nc1cc(F)c(C)c3c1c2[C@@H](N(C)C(=O)OCCO)CC3] |
| 2-7 | 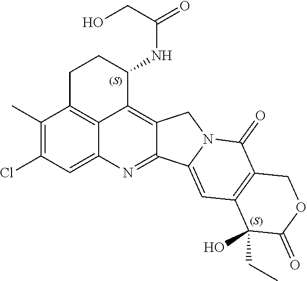 |
| 2-8 | 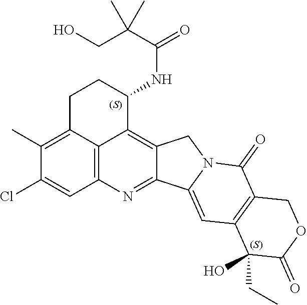 |
| 2-9 | 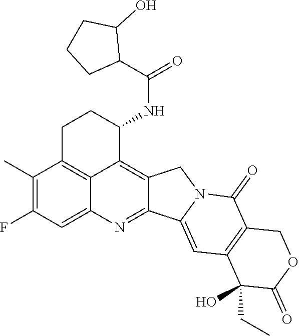 [SMILES: CC[C@@]1(O)C(=O)OCc2c1cc1n(c2=O)Cc2c-1nc1cc(F)c(C)c3c1c2[C@@H](NC(=O)C1CCCC1O)CC3] |
| 2-10 | 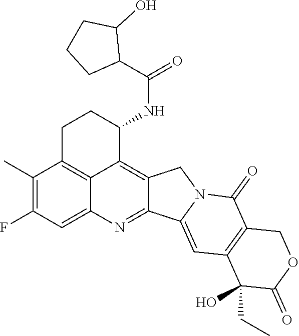 [SMILES: CC[C@@]1(O)C(=O)OCc2c1cc1n(c2=O)Cc2c-1nc1cc(F)c(C)c3c1c2[C@@H](NC(=O)C1CCCC1O)CC3] |
| 2-11 | 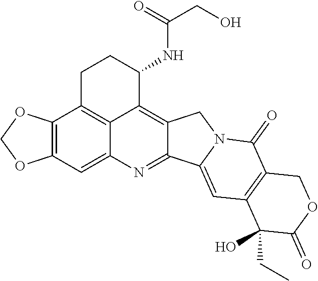 [SMILES: CC[C@@]1(O)C(=O)OCc2c1cc1n(c2=O)Cc2c-1nc1cc3c(c4c1c2[C@@H](NC(=O)CO)CC4)OCO3] |
| 2-12 | 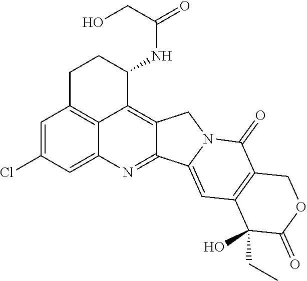 [SMILES: CC[C@@]1(O)C(=O)OCc2c1cc1n(c2=O)Cc2c-1nc1cc(Cl)cc3c1c2[C@@H](NC(=O)CO)CC3] |
| 2-13 | 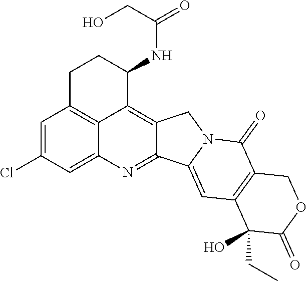 [SMILES: CC[C@@]1(O)C(=O)OCc2c1cc1n(c2=O)Cc2c-1nc1cc(Cl)cc3c1c2[C@H](NC(=O)CO)CC3] |
| 2-14 | 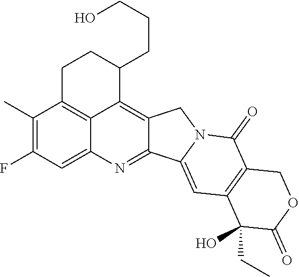 [SMILES: CC[C@@]1(O)C(=O)OCc2c1cc1n(c2=O)Cc2c-1nc1cc(F)c(C)c3c1c2C(CCCO)CC3] |
| 2-15 | 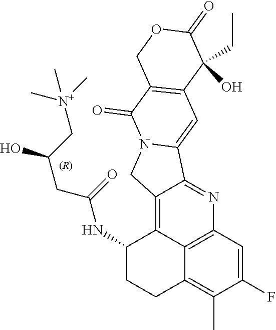 |
| 2-16 | 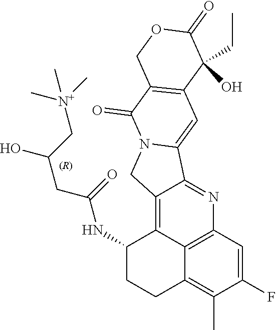 |
| 2-17 | 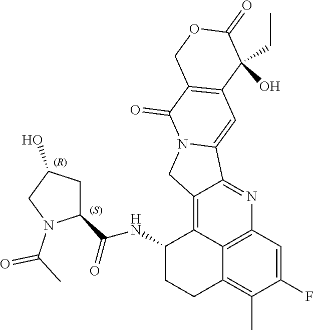 |
| 2-18 | 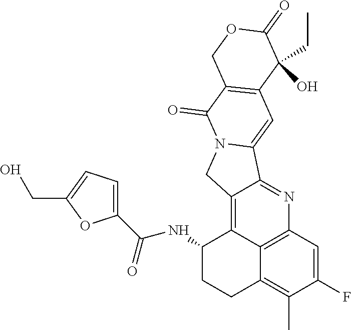 [SMILES: CC[C@@]1(O)C(=O)OCc2c1cc1n(c2=O)Cc2c-1nc1cc(F)c(C)c3c1c2[C@@H](NC(=O)c1ccc(CO)o1)CC3] |
| 2-19 | 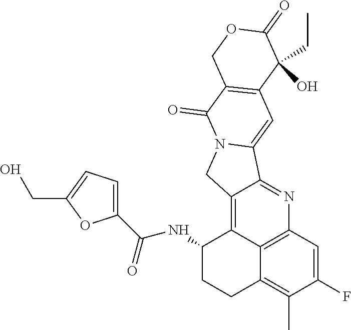 [SMILES: CC[C@@]1(O)C(=O)OCc2c1cc1n(c2=O)Cc2c-1nc1cc(F)c(C)c3c1c2[C@@H](NC(=O)c1ccc(CO)o1)CC3] |
| 2-20 | 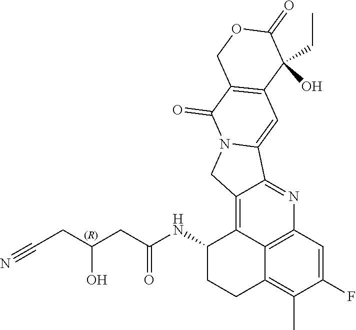 |
| 2-21 | 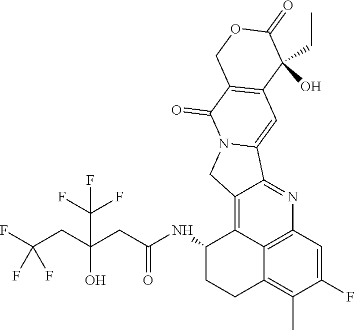 [SMILES: CC[C@@]1(O)C(=O)OCc2c1cc1n(c2=O)Cc2c-1nc1cc(F)c(C)c3c1c2[C@@H](NC(=O)CC(O)(CC(F)(F)F)C(F)(F)F)CC3] |
| 2-22 | 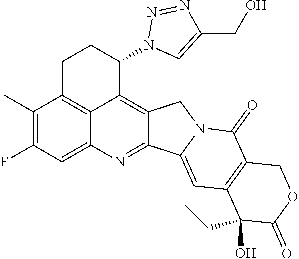 [SMILES: CC[C@@]1(O)C(=O)OCc2c1cc1n(c2=O)Cc2c-1nc1cc(F)c(C)c3c1c2[C@@H](n1cc(CO)nn1)CC3] |
| 2-23 | 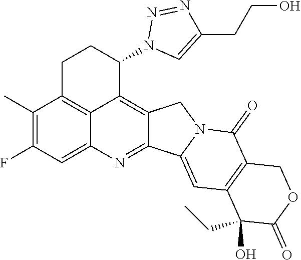 [SMILES: CC[C@@]1(O)C(=O)OCc2c1cc1n(c2=O)Cc2c-1nc1cc(F)c(C)c3c1c2[C@@H](n1cc(CCO)nn1)CC3] |
| 2-24 | 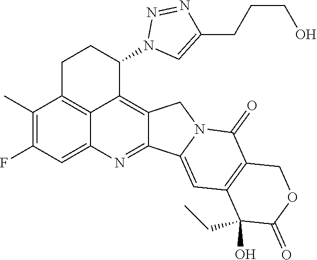 [SMILES: CC[C@@]1(O)C(=O)OCc2c1cc1n(c2=O)Cc2c-1nc1cc(F)c(C)c3c1c2[C@@H](n1cc(CCCO)nn1)CC3] |
| 2-25 | 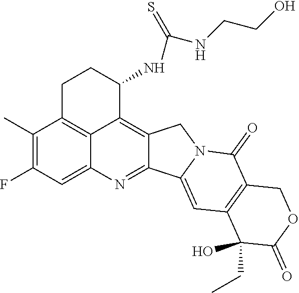 [SMILES: CC[C@@]1(O)C(=O)OCc2c1cc1n(c2=O)Cc2c-1nc1cc(F)c(C)c3c1c2[C@@H](NC(=S)NCCO)CC3] |
| 2-26 | 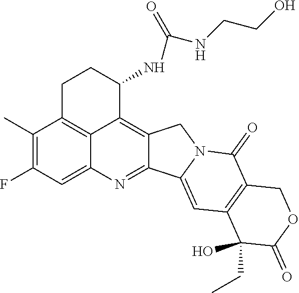 [SMILES: CC[C@@]1(O)C(=O)OCc2c1cc1n(c2=O)Cc2c-1nc1cc(F)c(C)c3c1c2[C@@H](NC(=O)NCCO)CC3] |
| 2-27 | 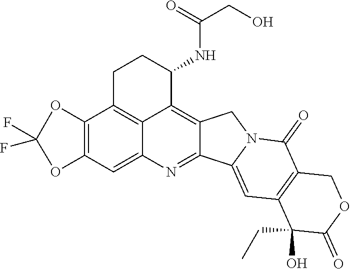 [SMILES: CC[C@@]1(O)C(=O)OCc2c1cc1n(c2=O)Cc2c-1nc1cc3c(c4c1c2[C@@H](NC(=O)CO)CC4)OC(F)(F)O3] |
| 2-28 | 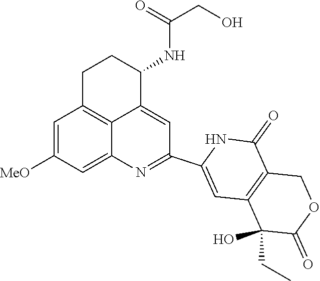 [SMILES: CC[C@@]1(O)C(=O)OCc2c1cc(-c1cc3c4c(cc(OC)cc4n1)CC[C@@H]3NC(=O)CO)[nH]c2=O] |
| 2-29 | 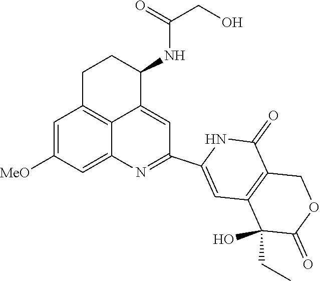 [SMILES: CC[C@@]1(O)C(=O)OCc2c1cc(-c1cc3c4c(cc(OC)cc4n1)CC[C@H]3NC(=O)CO)[nH]c2=O] |
| 2-30 | 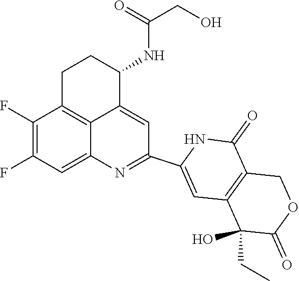 [SMILES: CC[C@@]1(O)C(=O)OCc2c1cc(-c1cc3c4c(c(F)c(F)cc4n1)CC[C@@H]3NC(=O)CO)[nH]c2=O] |
| 2-31 | 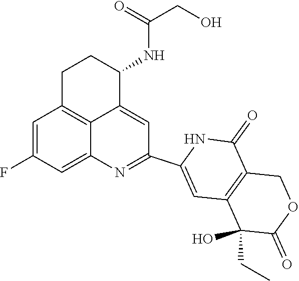 [SMILES: CC[C@@]1(O)C(=O)OCc2c1cc(-c1cc3c4c(cc(F)cc4n1)CC[C@@H]3NC(=O)CO)[nH]c2=O] |
| 2-32 | 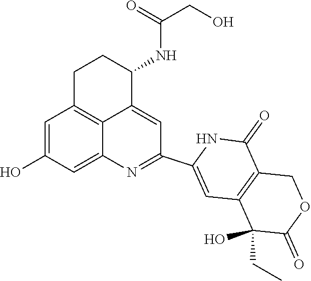 [SMILES: CC[C@@]1(O)C(=O)OCc2c1cc(-c1cc3c4c(cc(O)cc4n1)CC[C@@H]3NC(=O)CO)[nH]c2=O] |
| 2-34 | 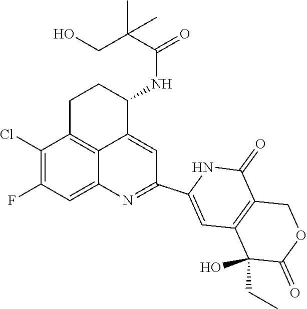 [SMILES: CC[C@@]1(O)C(=O)OCc2c1cc(-c1cc3c4c(c(Cl)c(F)cc4n1)CC[C@@H]3NC(=O)C(C)(C)CO)[nH]c2=O] |
| 2-35 | 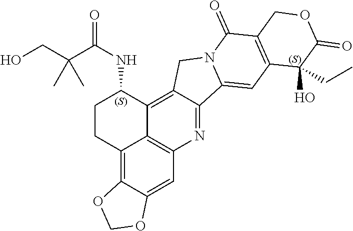 |
| 2-36 | 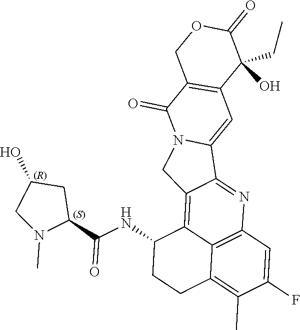 |
| 2-37 | 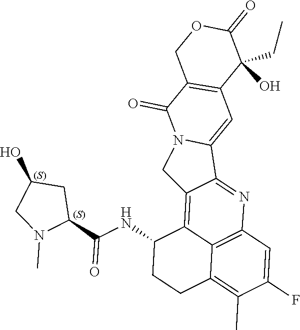 |
| 2-38 | 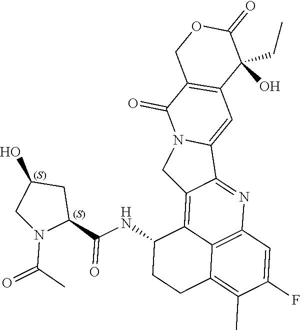 |
| 2-39 | 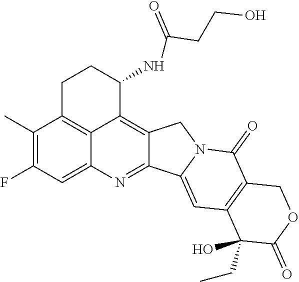 [SMILES: CC[C@@]1(O)C(=O)OCc2c1cc1n(c2=O)Cc2c-1nc1cc(F)c(C)c3c1c2[C@@H](NC(=O)CCO)CC3] |
| 2-40 | 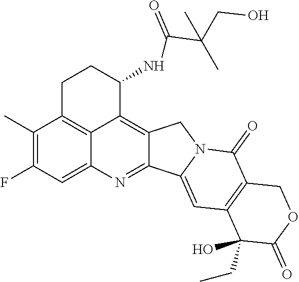 [SMILES: CC[C@@]1(O)C(=O)OCc2c1cc1n(c2=O)Cc2c-1nc1cc(F)c(C)c3c1c2[C@@H](NC(=O)C(C)(C)CO)CC3] |
| 2-41 | 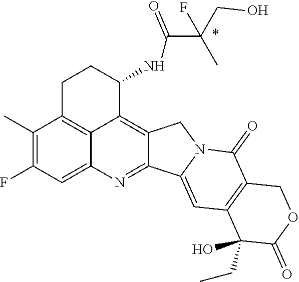 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] |
| 2-42 | 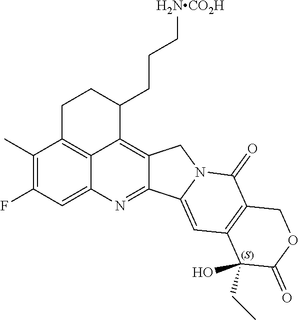 |
| 2-43 | 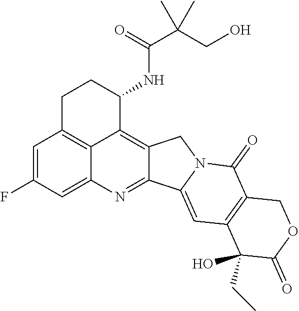 [SMILES: CC[C@@]1(O)C(=O)OCc2c1cc1n(c2=O)Cc2c-1nc1cc(F)cc3c1c2[C@@H](NC(=O)C(C)(C)CO)CC3] |
| 2-44 (isomer 1) 2-45 (isomer 2) | 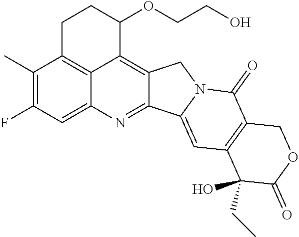 [SMILES: CC[C@@]1(O)C(=O)OCc2c1cc1n(c2=O)Cc2c-1nc1cc(F)c(C)c3c1c2C(OCCO)CC3] |
| 2-46 | 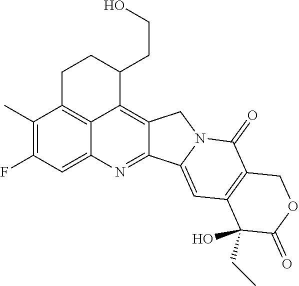 [SMILES: CC[C@@]1(O)C(=O)OCc2c1cc1n(c2=O)Cc2c-1nc1cc(F)c(C)c3c1c2C(CCO)CC3] |
| 2-47 | 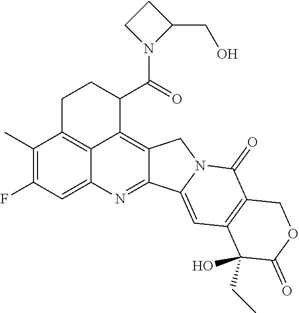 [SMILES: CC[C@@]1(O)C(=O)OCc2c1cc1n(c2=O)Cc2c-1nc1cc(F)c(C)c3c1c2C(C(=O)N1CCC1CO)CC3] |
| 2-48 | 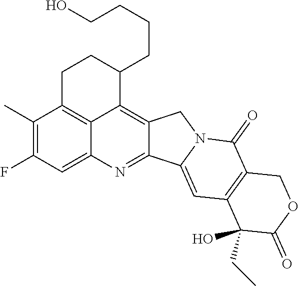 [SMILES: CC[C@@]1(O)C(=O)OCc2c1cc1n(c2=O)Cc2c-1nc1cc(F)c(C)c3c1c2C(CCCCO)CC3] |
| 2-49 (isomer 1) 2-50 (isomer 2) | 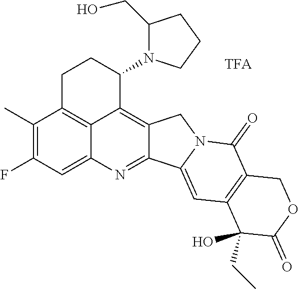 [SMILES: CC[C@@]1(O)C(=O)OCc2c1cc1n(c2=O)Cc2c-1nc1cc(F)c(C)c3c1c2[C@@H](N1CCCC1CO)CC3] |
| 2-51 (isomer 1) 2-52 (isomer 2) | 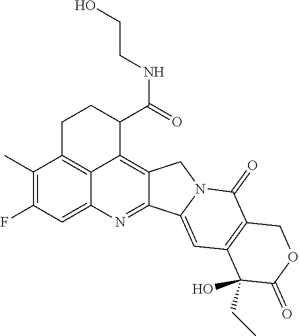 [SMILES: CC[C@@]1(O)C(=O)OCc2c1cc1n(c2=O)Cc2c-1nc1cc(F)c(C)c3c1c2C(C(=O)NCCO)CC3] |
| 2-53 | 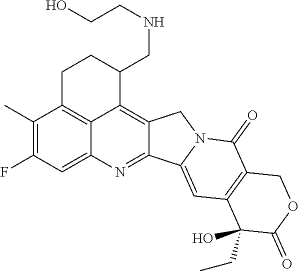 [SMILES: CC[C@@]1(O)C(=O)OCc2c1cc1n(c2=O)Cc2c-1nc1cc(F)c(C)c3c1c2C(CNCCO)CC3] |
| 2-54 | 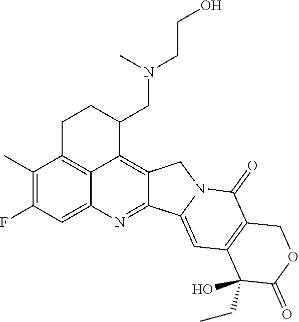 [SMILES: CC[C@@]1(O)C(=O)OCc2c1cc1n(c2=O)Cc2c-1nc1cc(F)c(C)c3c1c2C(CN(C)CCO)CC3] |
| 2-55 | 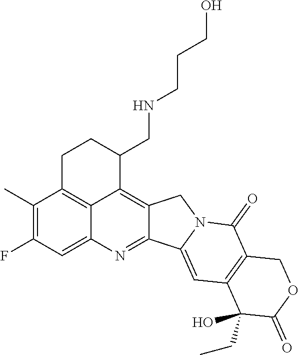 [SMILES: CC[C@@]1(O)C(=O)OCc2c1cc1n(c2=O)Cc2c-1nc1cc(F)c(C)c3c1c2C(CNCCCO)CC3] |
| 2-56 (isomer 1) 2-57 (isomer 2) | 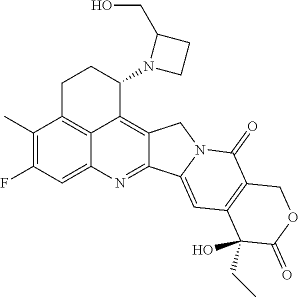 [SMILES: CC[C@@]1(O)C(=O)OCc2c1cc1n(c2=O)Cc2c-1nc1cc(F)c(C)c3c1c2[C@@H](N1CCC1CO)CC3] |
| 2-58 | 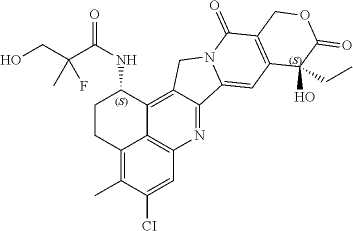 |
| 2-59 (isomer 1) 2-60 (isomer 2) | 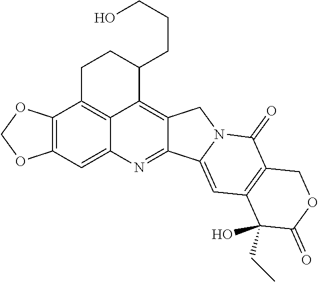 [SMILES: CC[C@@]1(O)C(=O)OCc2c1cc1n(c2=O)Cc2c-1nc1cc3c(c4c1c2C(CCCO)CC4)OCO3] |
| 2-61 | 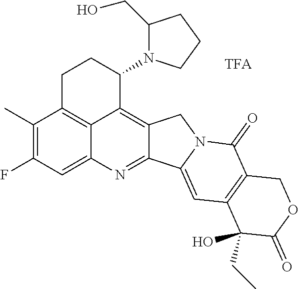 [SMILES: CC[C@@]1(O)C(=O)OCc2c1cc1n(c2=O)Cc2c-1nc1cc(F)c(C)c3c1c2[C@@H](N1CCCC1CO)CC3] |
| 2-62 | 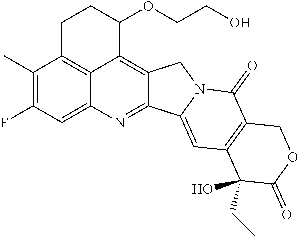 [SMILES: CC[C@@]1(O)C(=O)OCc2c1cc1n(c2=O)Cc2c-1nc1cc(F)c(C)c3c1c2C(OCCO)CC3] |
| 2-63 | 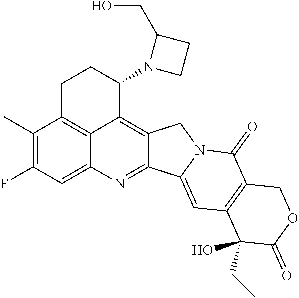 [SMILES: CC[C@@]1(O)C(=O)OCc2c1cc1n(c2=O)Cc2c-1nc1cc(F)c(C)c3c1c2[C@@H](N1CCC1CO)CC3] |
| 2-64 | 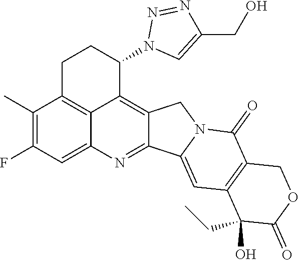 [SMILES: CC[C@@]1(O)C(=O)OCc2c1cc1n(c2=O)Cc2c-1nc1cc(F)c(C)c3c1c2[C@@H](n1cc(CO)nn1)CC3] |
| 2-65 | 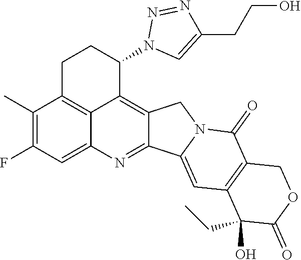 [SMILES: CC[C@@]1(O)C(=O)OCc2c1cc1n(c2=O)Cc2c-1nc1cc(F)c(C)c3c1c2[C@@H](n1cc(CCO)nn1)CC3] |

In some embodiments, PA is a residue of:

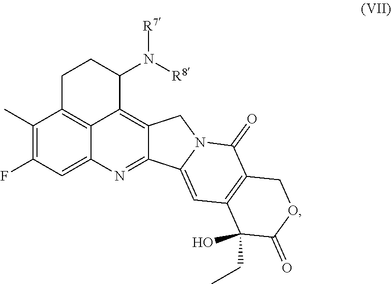

wherein

each of R^{7′}, and R^{8′} is, independently, hydrogen, or substituted or unsubstituted alkyl; or R^{7′}, and R^{8′} together with the nitrogen atom to which they are attached to, form unsubstituted or substituted heterocyclyl, or unsubstituted or substituted heteroaryl.

In some embodiments. PA is a residue of:

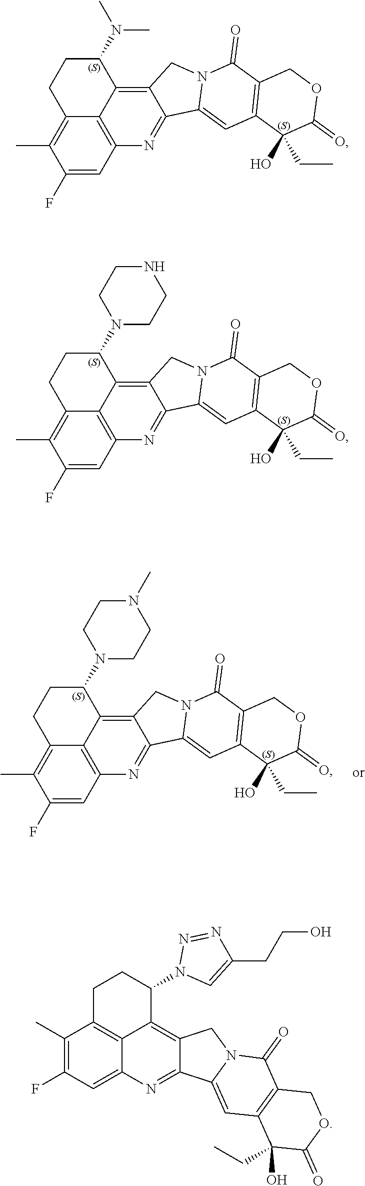

In some embodiments, an antibody drug conjugate has formula (V):

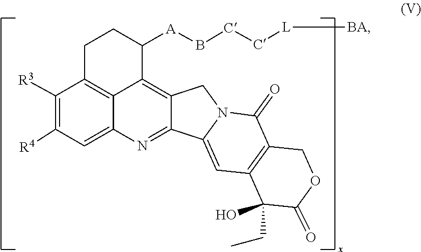

or a pharmaceutically acceptable salt tautomer, solvate, stereoisomer, enantiomer, isotopologue, or prodrug thereof, wherein values and alternative values for the variables (e.g., A, B, C′, D′, L, R³, R⁴, and x) are as described elsewhere herein.

In some embodiments, an antibody drug conjugate has a structure of formula (VIIIa), (VIIIb), or (VIIIc):

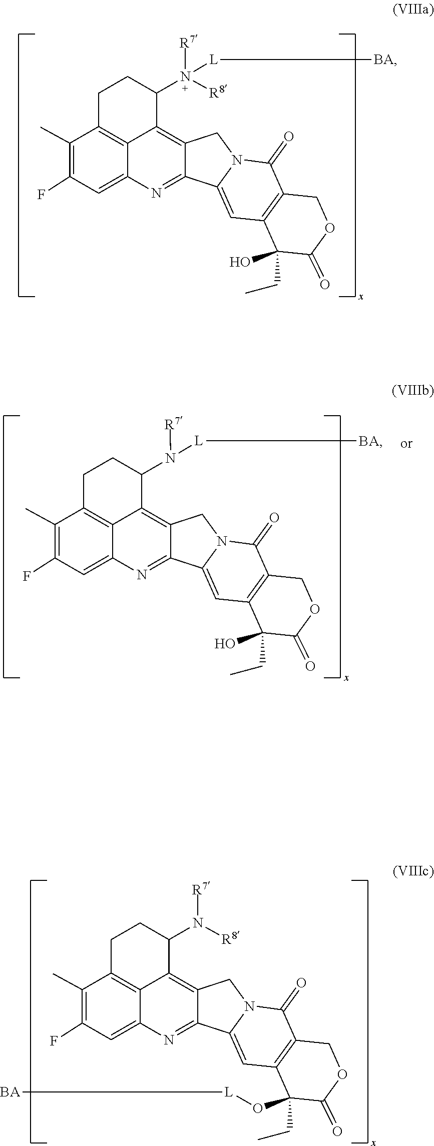

or a pharmaceutically acceptable salt tautomer, solvate, stereoisomer, enantiomer, isotopologue, or prodrug thereof, wherein values and alternative values for the variables (e.g., L, R⁷, R⁸, and x) are as described elsewhere herein.
In some embodiments, an antibody drug conjugate has a structure of any one of the following formulas:

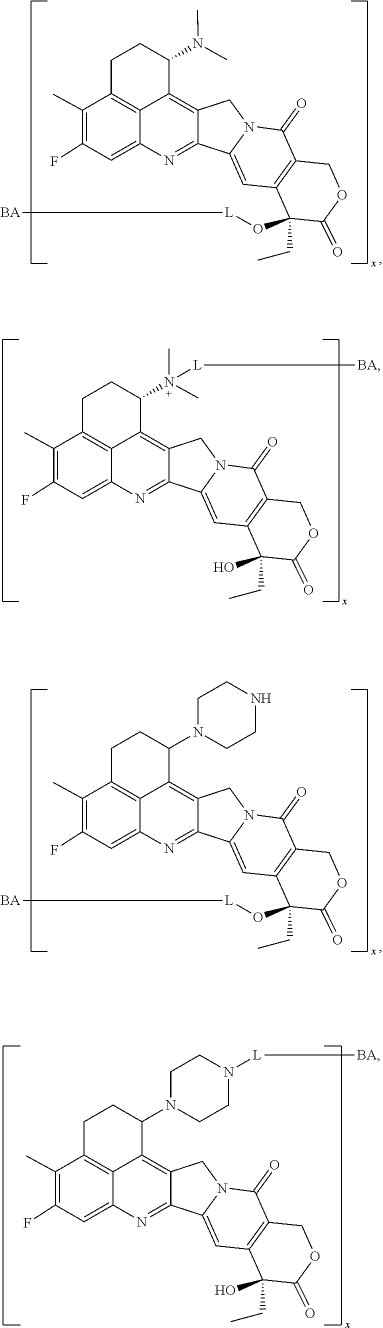

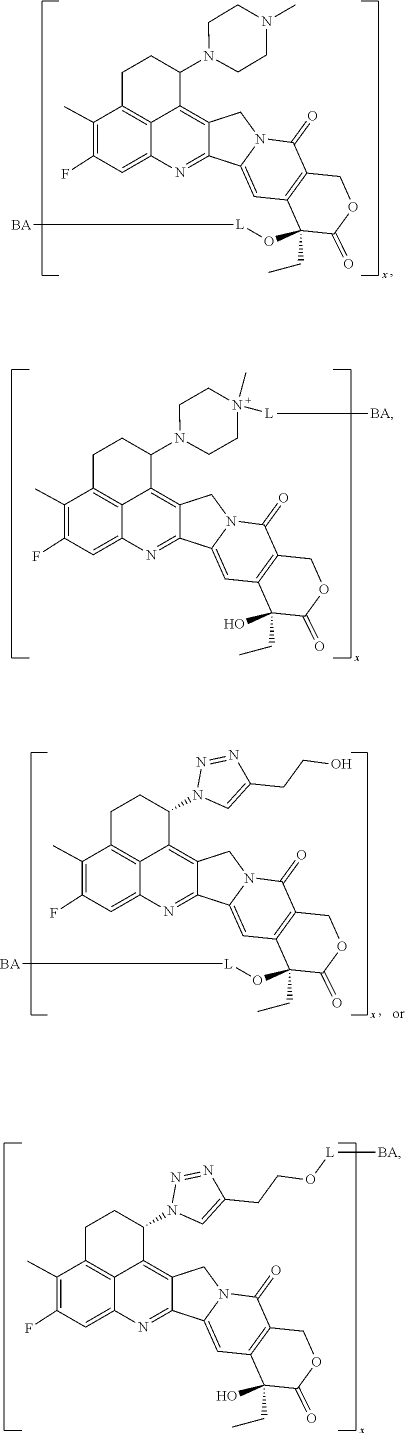

or a pharmaceutically acceptable salt tautomer, solvate, stereoisomer, enantiomer, isotopologue, or prodrug thereof, wherein values and alternative values for the variables (e.g., L and x) are as described elsewhere herein.

In some embodiments, L is

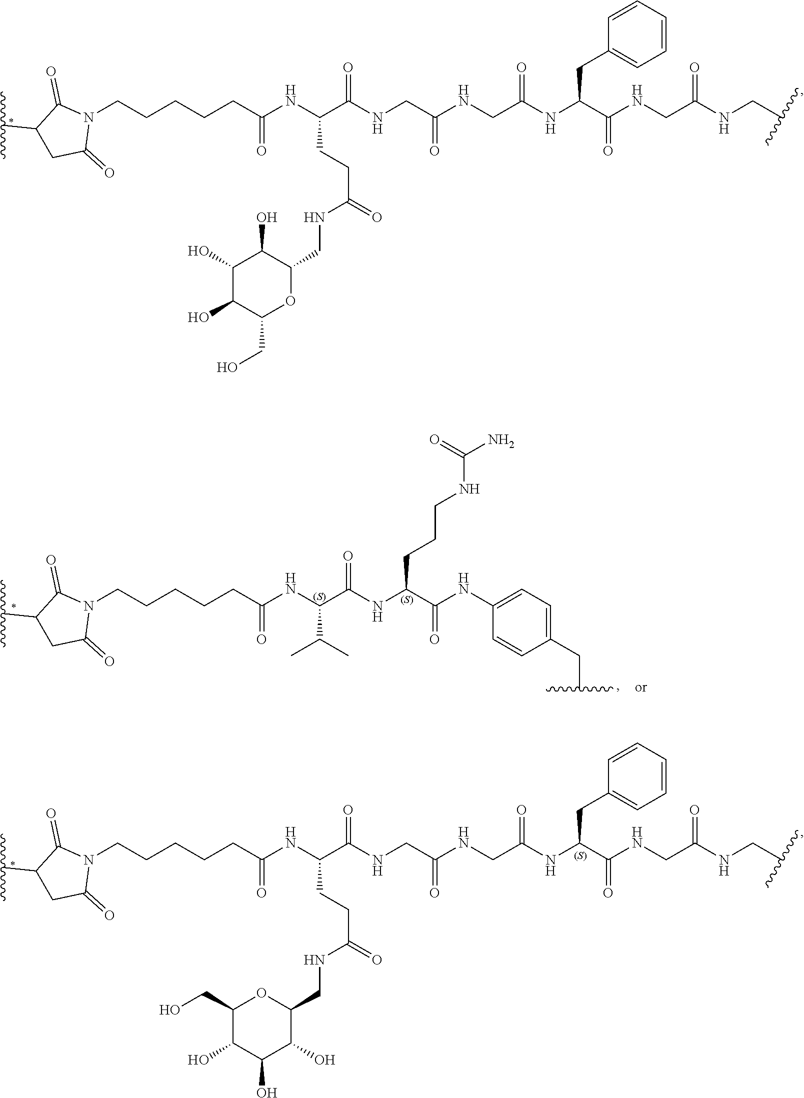

wherein the bond marked with asterisk is connected to BA.

In some embodiments, L is

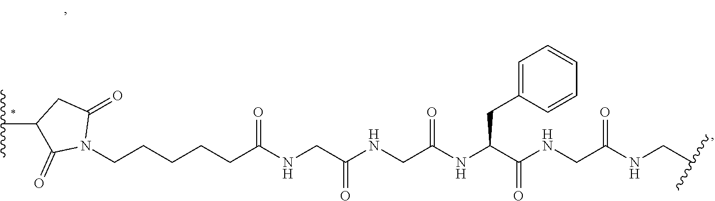 [SMILES: *CNC(=O)CNC(=O)[C@H](Cc1ccccc1)NC(=O)CNC(=O)CNC(=O)CCCCCN1C(=O)CC(*)C1=O]

wherein the bond marked with asterisk is connected to BA.

In some embodiments, L is:

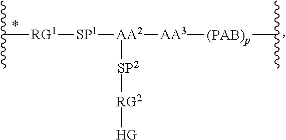

wherein values and alternative values for the variables (e.g., RG¹, SP¹, AA², AA³, PAB, p, SP²RG², and HG) are as described herein.

In some embodiments, L is:

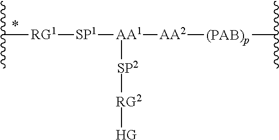

wherein values and alternative values for the variables (e.g., RG¹, SP¹, AA¹, AA², PAB, p, SP² RG², and HG) are as described elsewhere herein.

In some embodiments, L is:

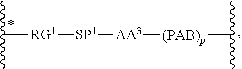

wherein values and alternative values for the variables (e.g., RG¹, SP¹, AA³, PAB, and p) are as described elsewhere herein.

In some embodiments, -AA²(SP²—RG²-HG)-AA³-(PAB)ₚ— is

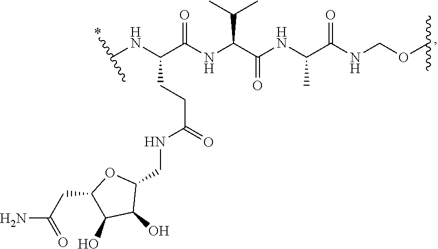

wherein *marks the bond that connects to SP¹.

In some embodiments, an antibody drug conjugate is selected from the following, or a pharmaceutically acceptable salt tautomer, solvate, stereoisomer, enantiomer, isotopologue, or prodrug thereof, wherein Ab is any of the anti-CEA antibodies disclosed herein:

| Examples | Structures |
|---|---|
| ADC-P1 BGB-C- 269 | 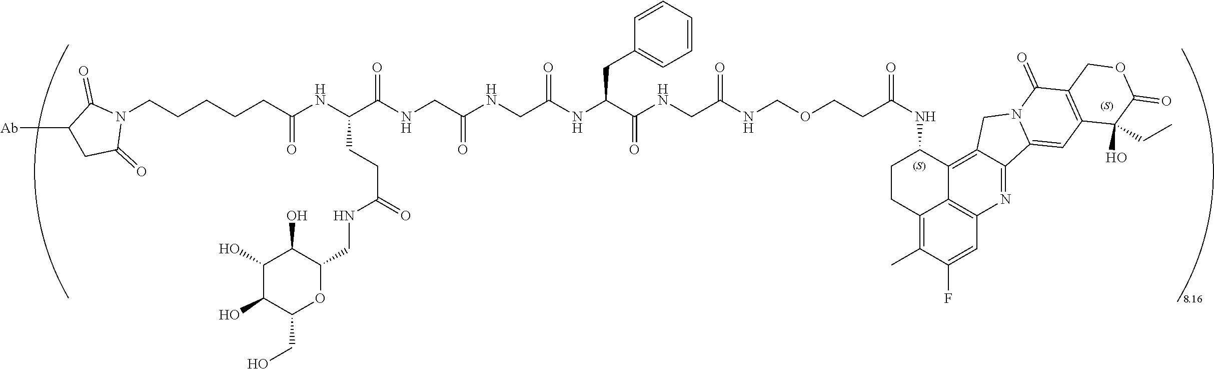 |
| ADC-P2 BGB-C- 276 | 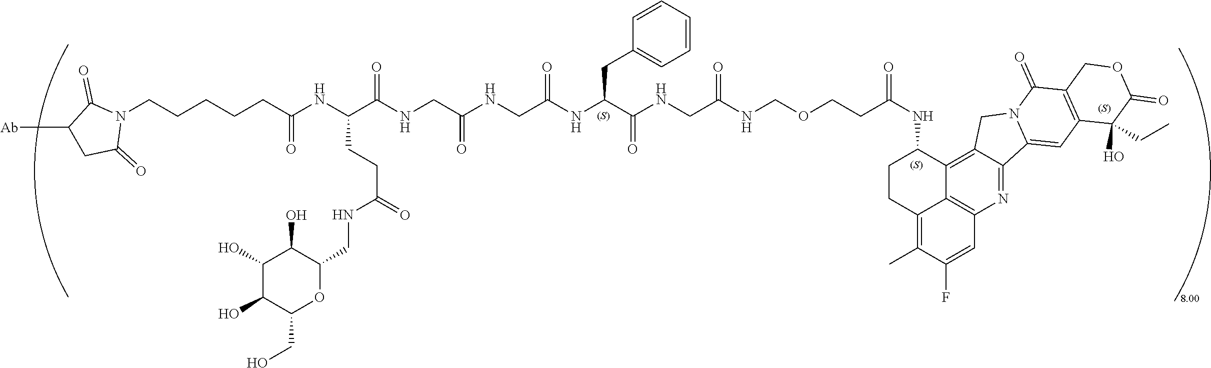 |
| ADC-P3 BGB-C- 290 | 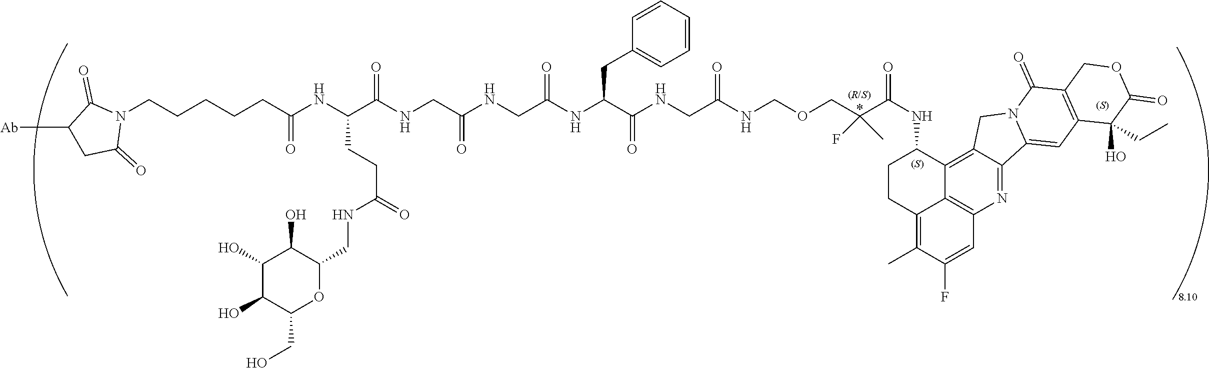 |
| ADC-1 BGB-C- 81 | 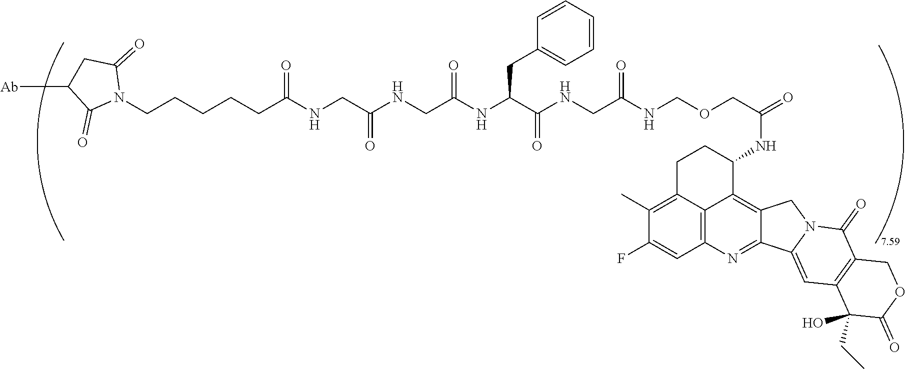 |
| ADC-2 BGB-C- 115 | 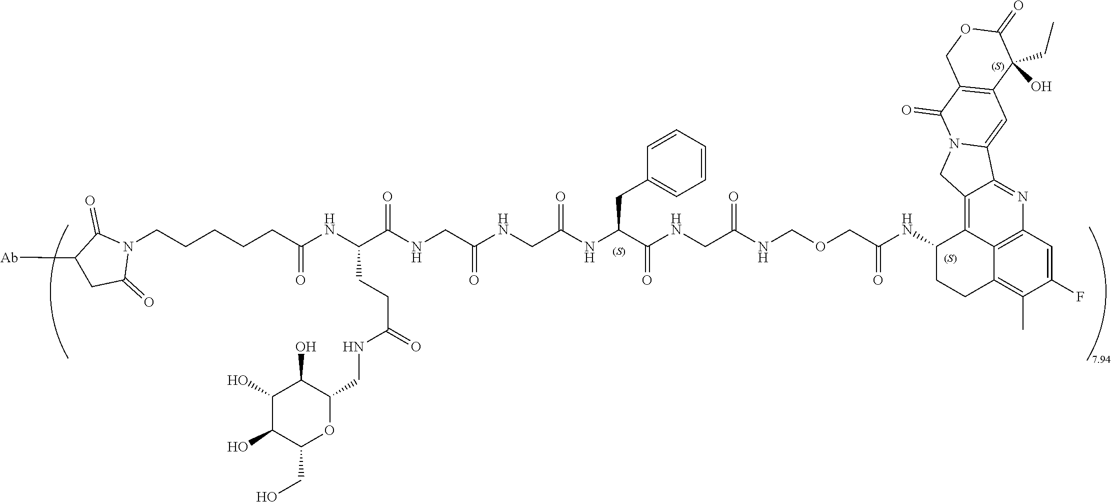 |
| ADC-3 BGB-C- 245 | 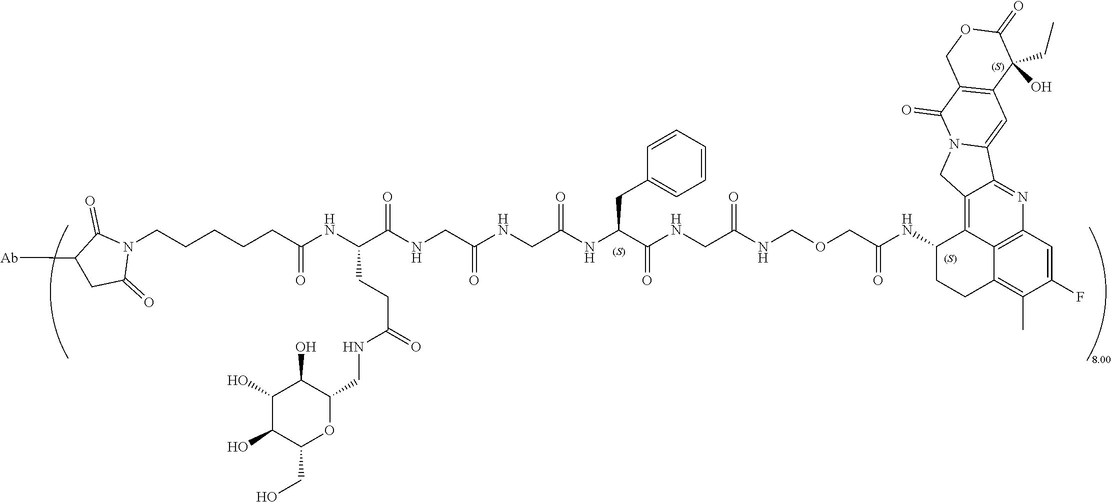 |
| ADC-4 BGB-C- 318 | 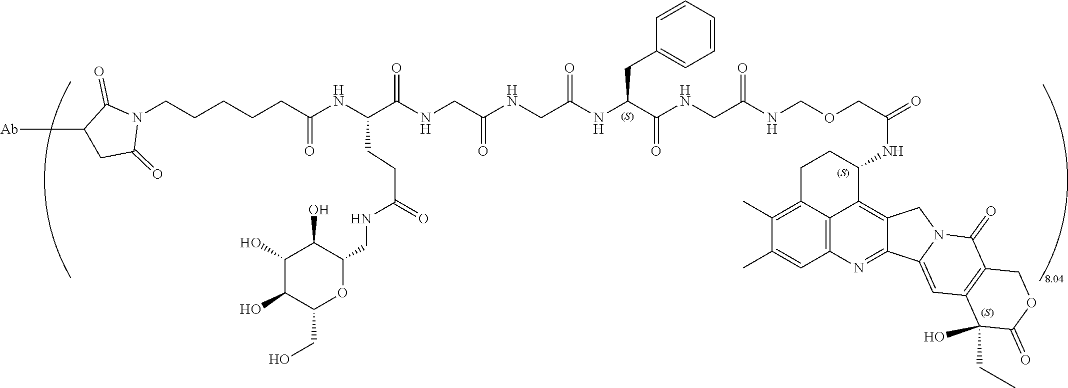 |
| ADC-5 BGB-C- 277 | 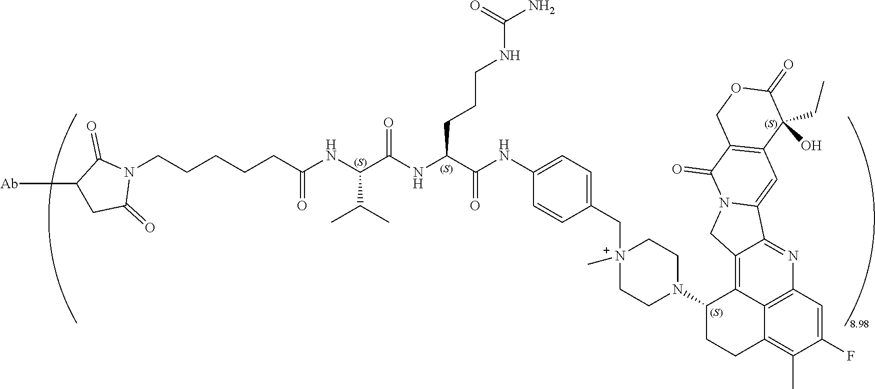 |
| ADC-6 BGB-C- 306 | 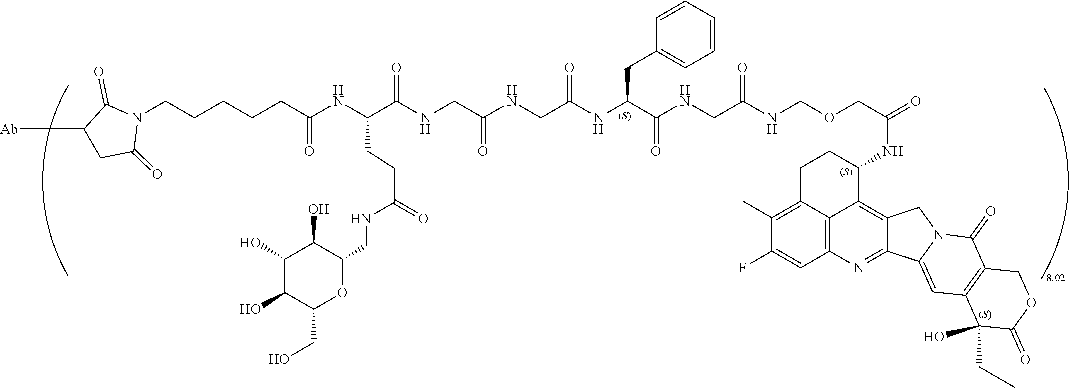 |
| ADC-7 BGB-C- 329 | 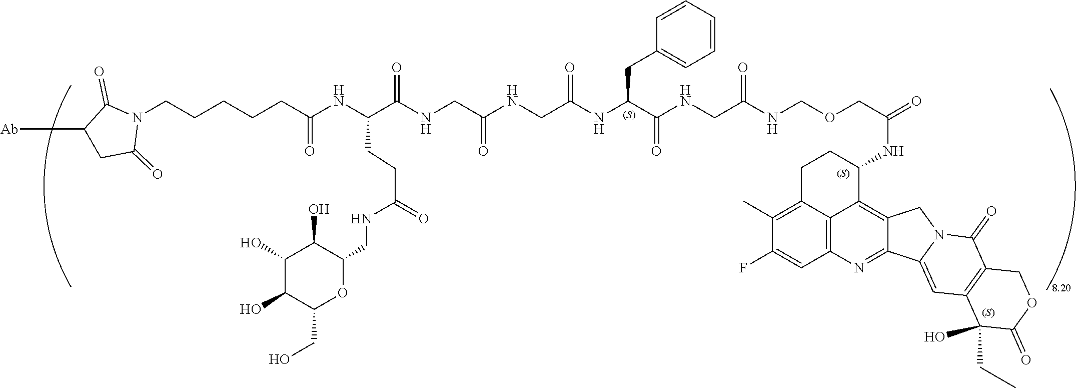 |
| ADC-8 BGB-C- 323 | 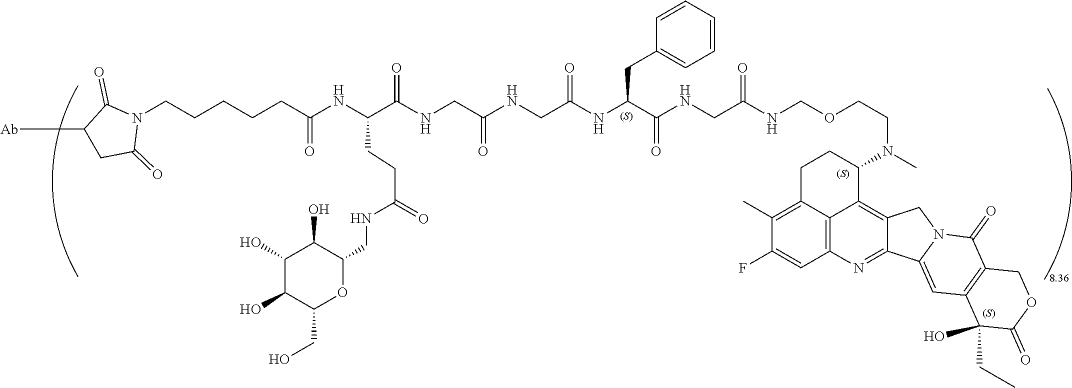 |
| ADC-9 BGB-C- 331 | 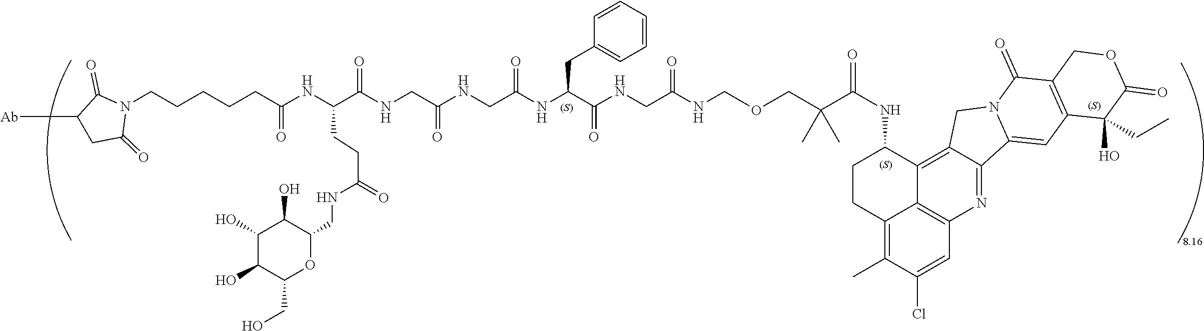 |
| ADC-10 BGB-C- 325 | 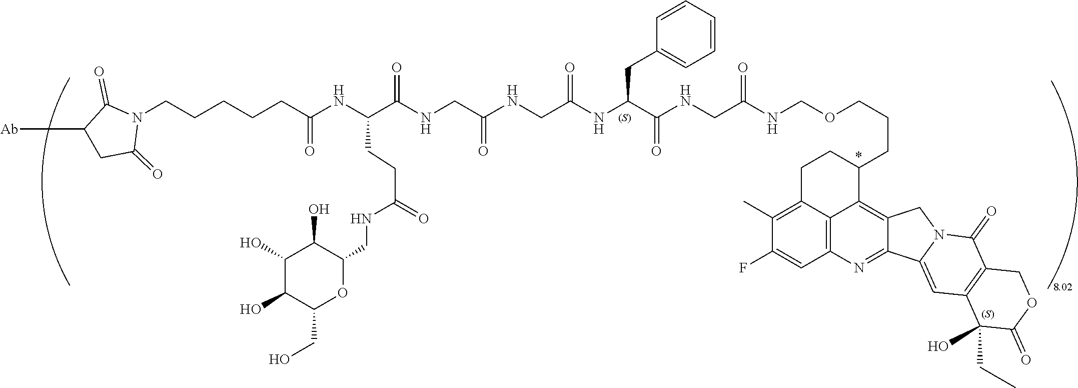 |
| ADC-11 BGB-C- 307 | 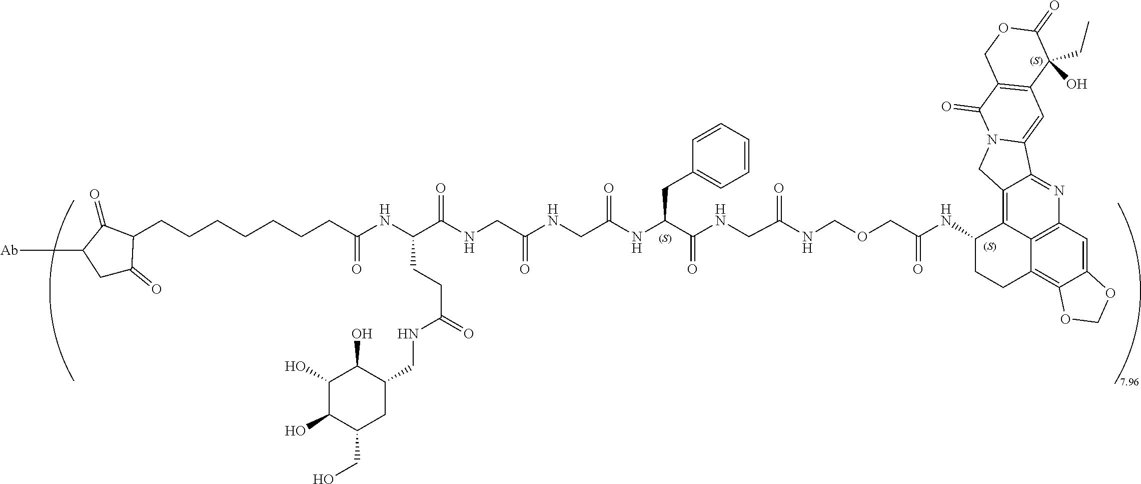 |
| ADC-12 BGB-C- 287 | 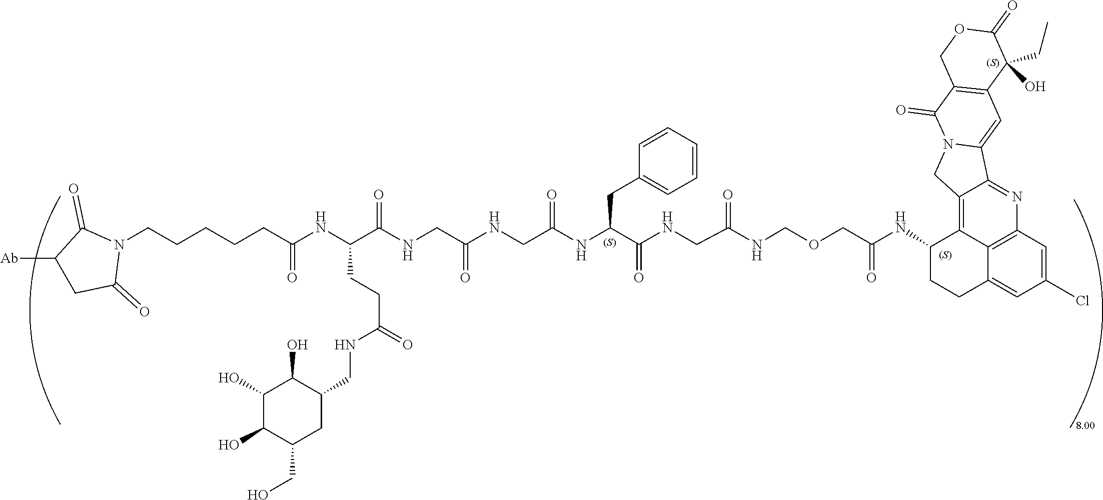 |
| ADC-13 BGB-C- 288 | 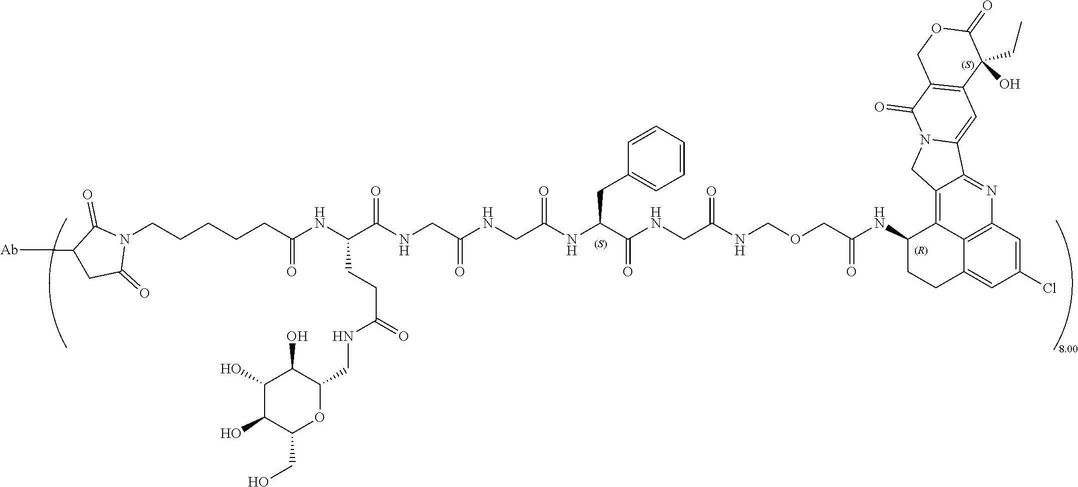 |
| ADC-14 BGB-C- 317 | 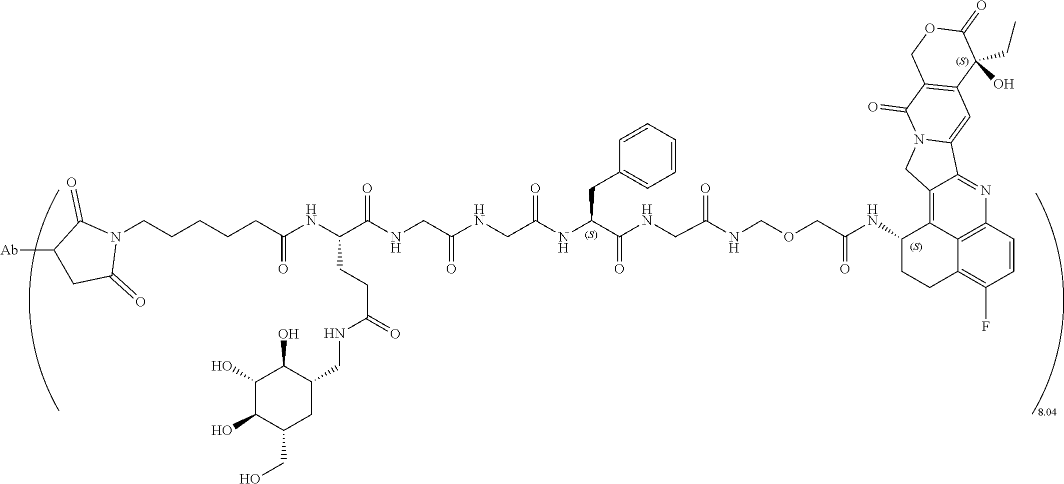 |
| ADC-15 BGB-C- 292 | 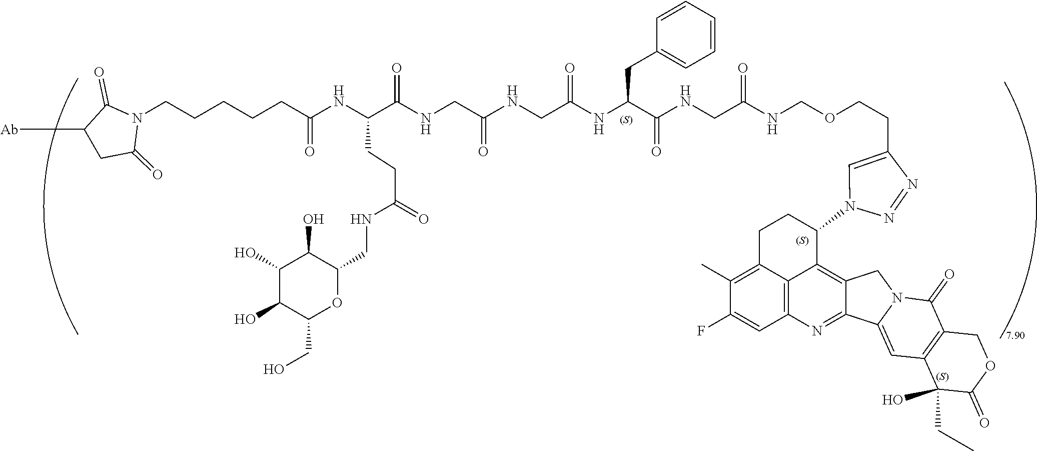 |
| ADC-16 BGB-C- 348 | 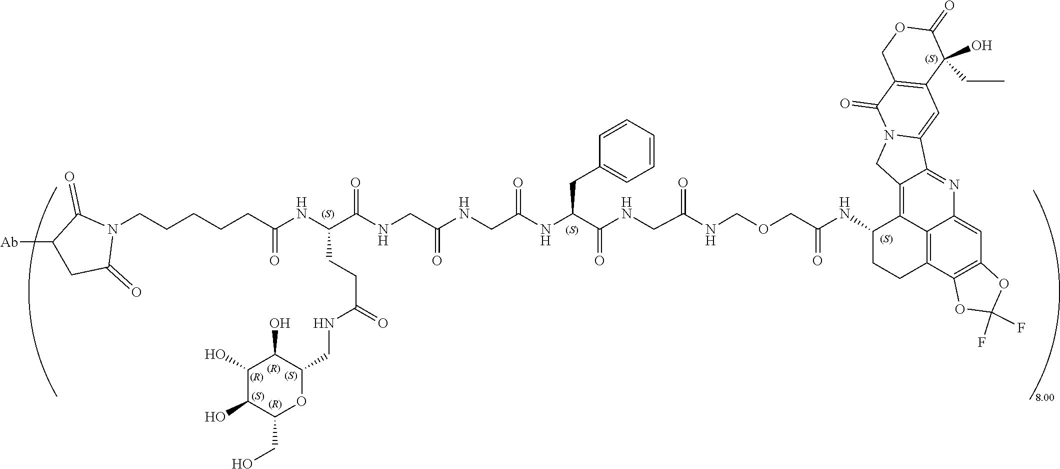 |
| ADC-17 BGB-C- 319 | 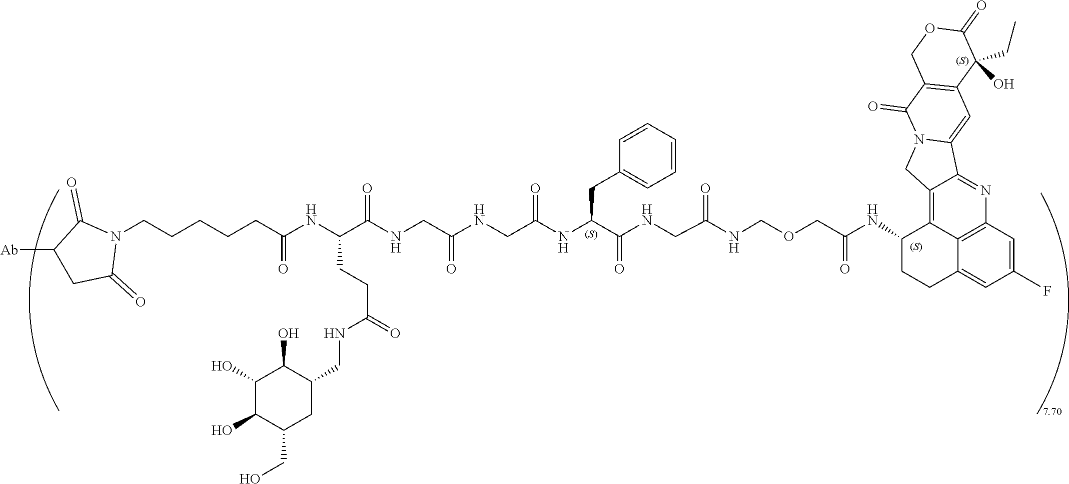 |
| ADC-18 BGB-C- 343 | 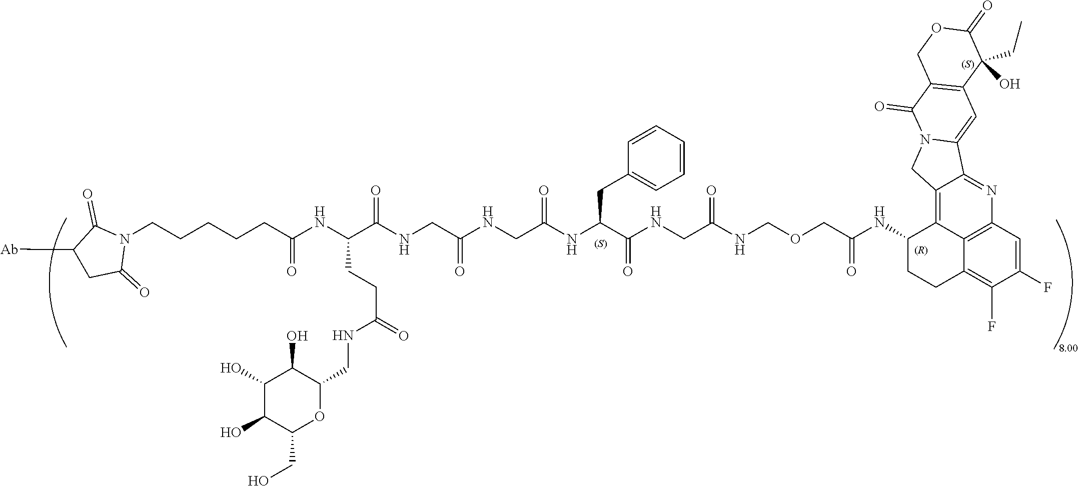 |
| ADC-19 BGB-C- 302 | 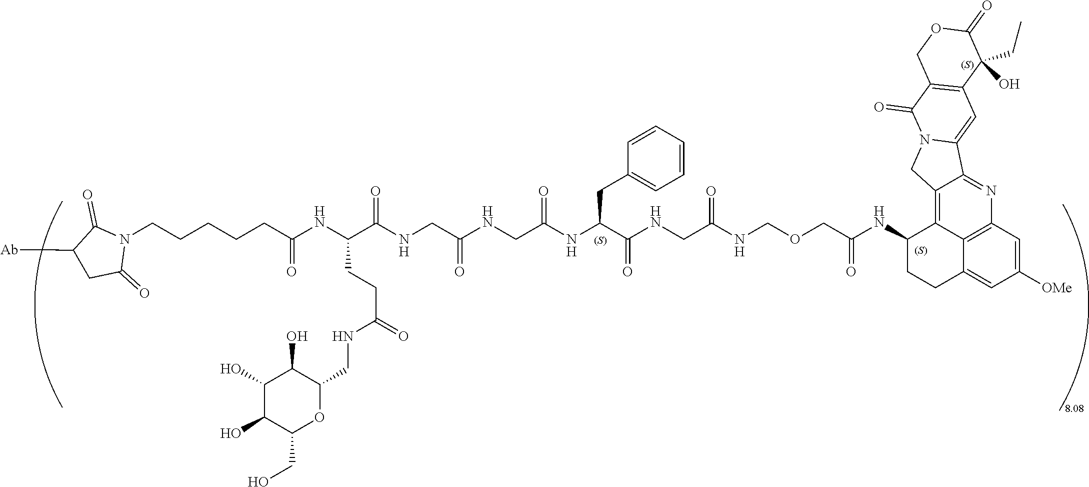 |
| ADC-20 BGB-C- 320 | 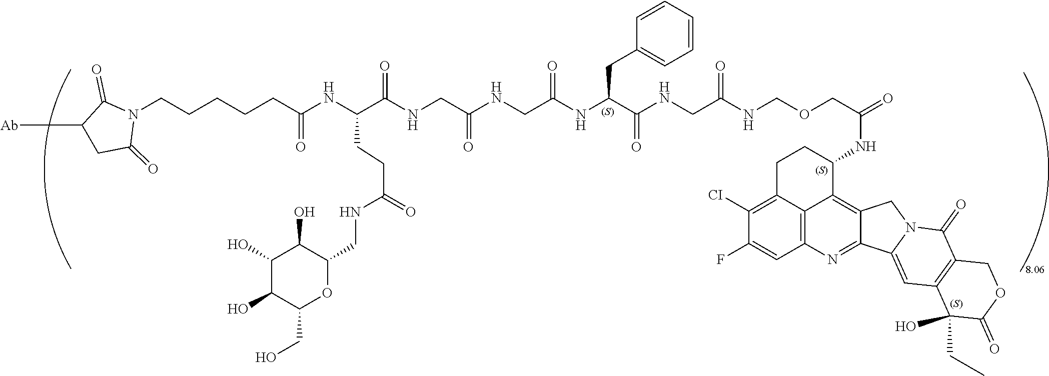 |
| ADC-21 BGB-C- 330 | 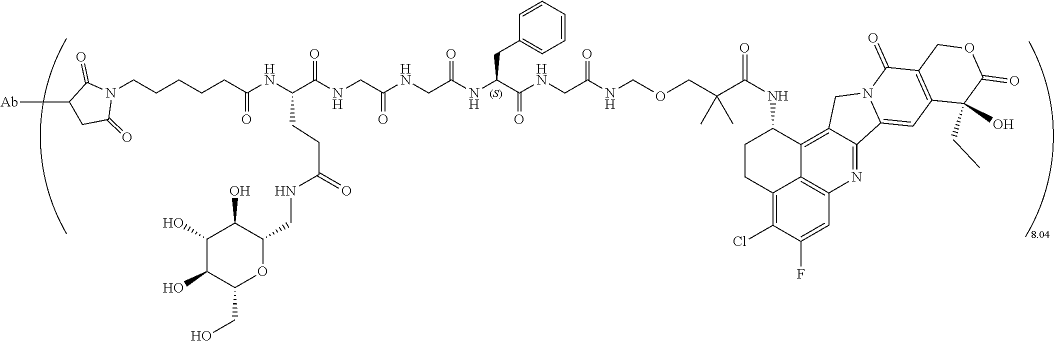 |
| ADC-22 BGB-C- 346 | 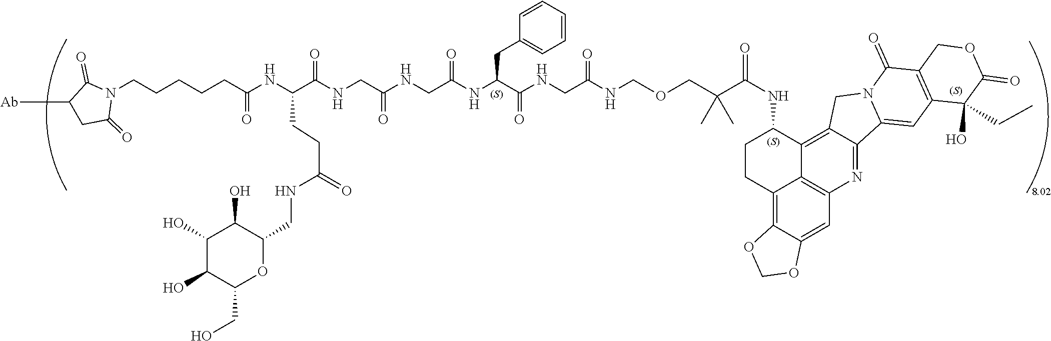 |
| ADC-23 BGB-C- 358 | 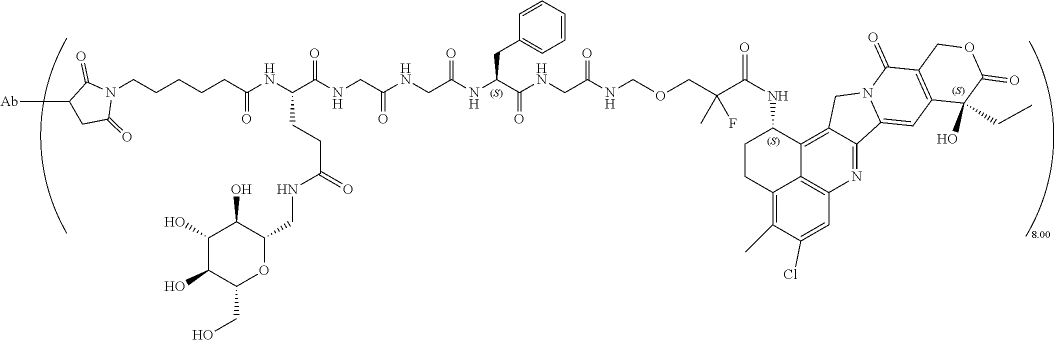 |
| ADC-24 BGB-C- 373 | 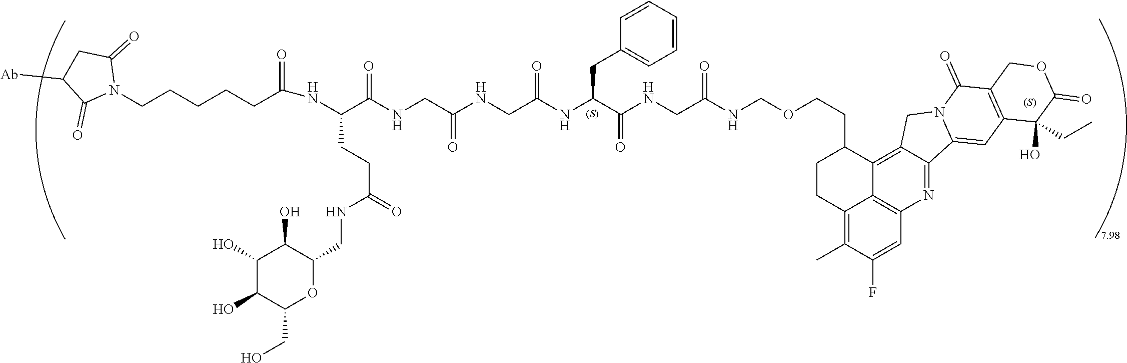 |
| ADC-25 BGB-C- 421 | 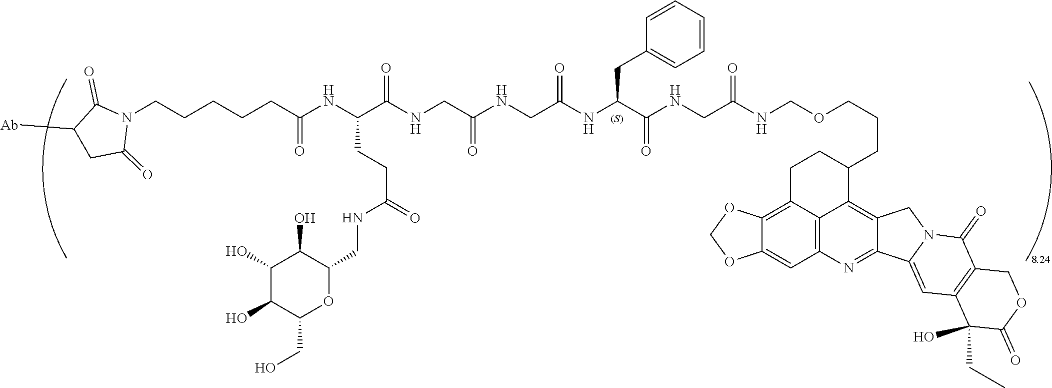 |
| ADC-26 BGB-C- 395 | 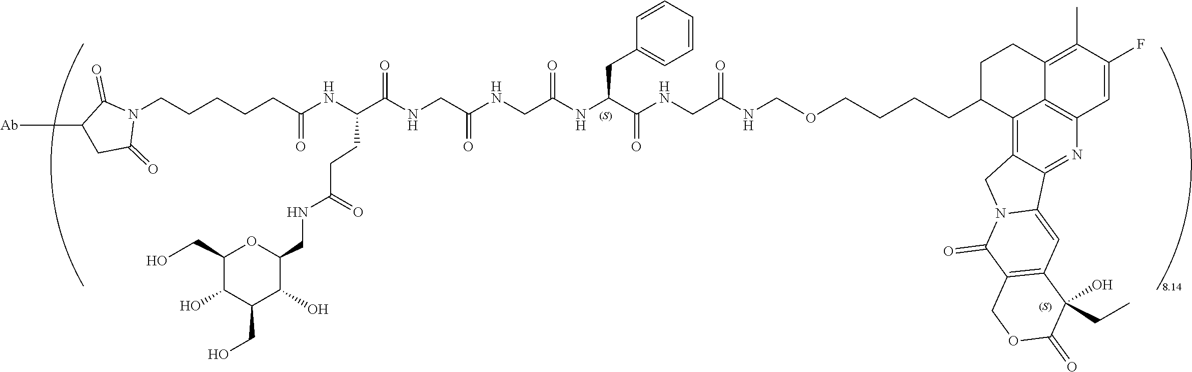 |

All possible combinations of linkers and payloads are contemplated herein. In this regard, it will be appreciated that L, used in the context of Formulas I-VIII herein, encompasses C-L of a compound of Formula A or A-1. In addition, it will be appreciated that C, used in the context of Formula A or A-1, corresponds to RG¹-SP¹.

In embodiments, D is:

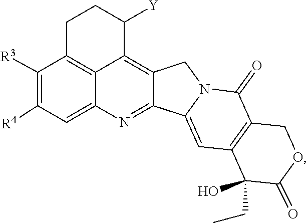

wherein

Y is -A-B-C-D-*, wherein * marks the bond where D connects to L;

A is a bond, CR¹R², or N-R¹;

B is a bond, —C(═O)—; or —C(═O)O—;

C is a bond, or a divalent group, wherein the divalent group is unsubstituted or substituted C₁₋₈alkyl, unsubstituted or substituted cycloalkyl, unsubstituted or substituted heterocyclyl, unsubstituted or substituted aryl, or unsubstituted or substituted heteroaryl;

D is a bond, NH, or O;

each of R¹, and R² is, independently, hydrogen, halogen, substituted or unsubstituted alkyl, or substituted or unsubstituted alkoxyl; or R¹, and R² together with the atom to which they are attached to, form unsubstituted or substituted cycloalkyl, unsubstituted or substituted heterocyclyl, unsubstituted or substituted aryl, or unsubstituted or substituted heteroaryl;

each of R³, and R⁴ is, independently, hydrogen, halogen, substituted or unsubstituted alkyl, or substituted or unsubstituted alkoxyl; or R³, and R⁴ together with the atoms to which they are attached to, form unsubstituted or substituted cycloalkyl, unsubstituted or substituted heterocyclyl, unsubstituted or substituted aryl, or unsubstituted or substituted heteroaryl.

In embodiments, D is

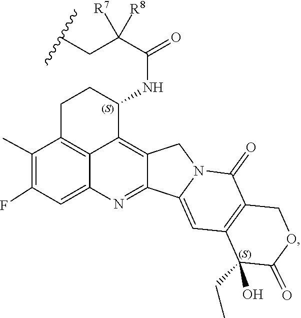

wherein R⁷ and R⁸ are each independently hydrogen, halogen, or alkyl.

In embodiments, the cytotoxic agent has the following formula:

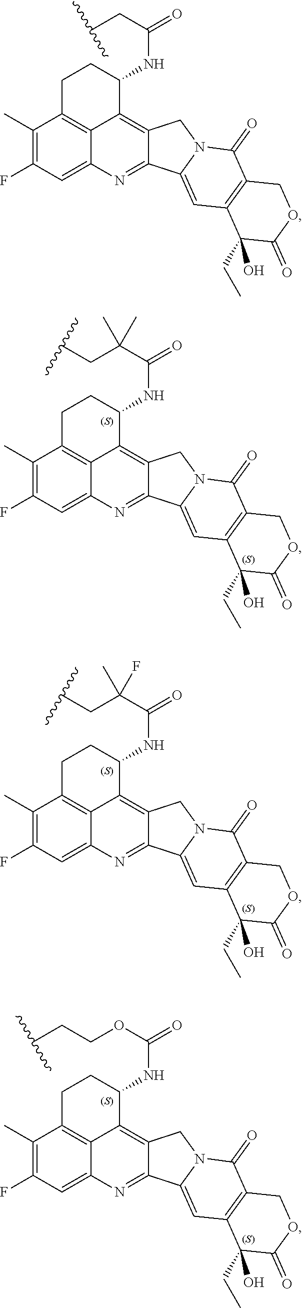

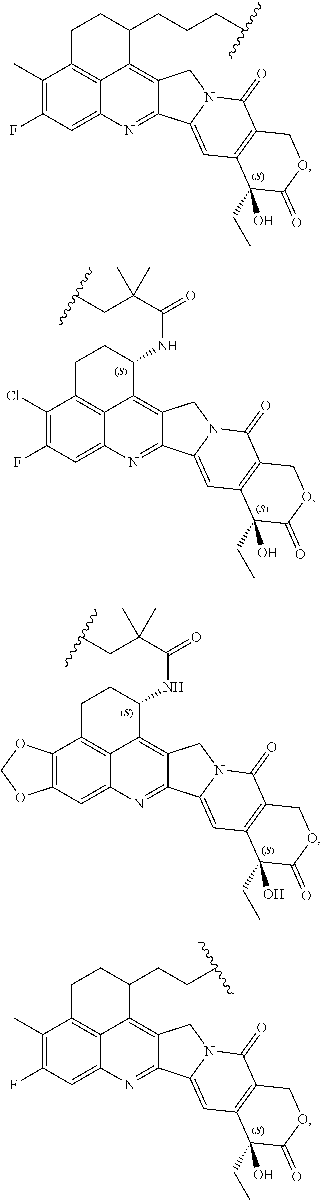

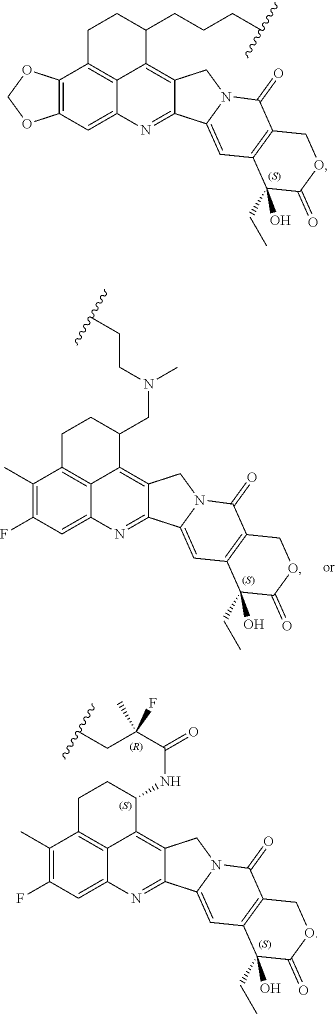

In certain embodiments, the cytotoxic agent (D) is

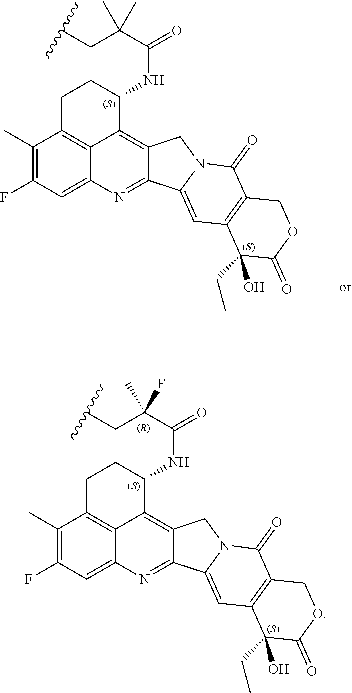

In certain embodiments, D is

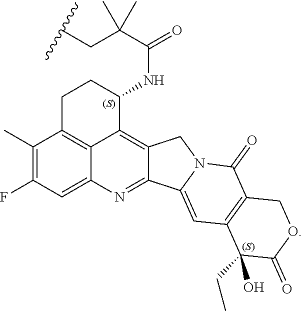

Each antibody drug conjugate may include one or more than one molecule of cytotoxic agent, such as one, two, three, four, five, six, seven, or eight molecules. The number of cytotoxic agent molecules conjugated to a single antibody or antibody fragment may be described as a drug to antibody ratio (DAR). In the formula Ab-(C-L-(D)ₘ)ₙ, DAR is the product of m and n.

The cytotoxic agent may be directly joined to an anti-CEA antibody, or indirectly joined to an anti-CEA antibody, via a linker (L). In embodiments, the linker is cleavable, such as by an enzyme, to release the cytotoxic agent. In embodiments, such as when the cytotoxic agent is hydrophobic, the linker is hydrophilic. In embodiments, the linker has the following formula, in which * marks the bond where L may be joined to a conjugator (C):

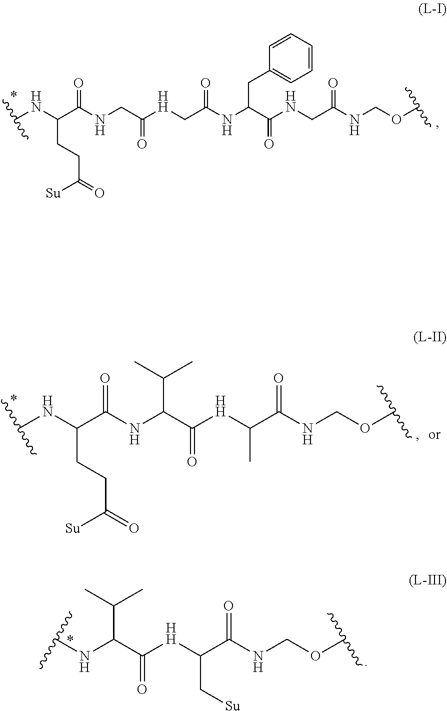

In the foregoing formulae, Su may be a sugar-like moiety. The moiety may be derived from a natural or non-natural sugar. The moiety may be hydrophilic. In embodiments, such as when the cytotoxic agent is hydrophobic, inclusion of a hydrophilic Su moiety may reduce the likelihood of antibody drug conjugate aggregation and thereby reduce clearance rate in vivo.

In embodiments, Su is a hydrophilic residue.

In embodiments, Su is

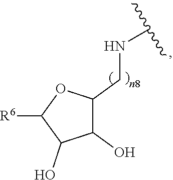

wherein n8 is 0 or 1; R⁶ is —OR², —N(H)R², —C(O)OR², —C(O)N(H)R², —CH₂—OR², —CH₂—N(H)R², —CH₂—C(O)OR², or —CH₂—C(O)N(H)R²; and R² is hydrogen or methyl. In certain embodiments, n8 is 1. In certain embodiments, n8 is 0. In certain embodiments, R² is hydrogen. In certain embodiments, R² is methyl. In certain embodiments, R⁶ is —OR², —N(H)R², —C(O)OR² or —C(O)N(H)R². In certain embodiments R⁶ is —CH₂—OR², —CH₂—N(H)R², —CH₂—C(O)OR², or —CH₂—C(O)N(H)R². In certain embodiments, R⁶ is —CH₂—C(O)N(H)R², e.g., —CH₂—C(O)NH₂.

In embodiments, Su is

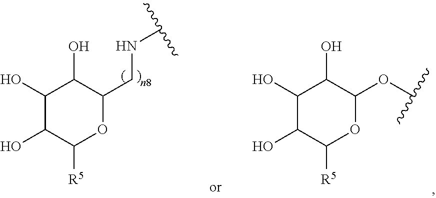

wherein n8 is 0 or 1; R⁵ is —OH, —NH₂, —C(O)OH, —C(O)NH₂, —CH₂—OH, or —CH₂—NH₂. In certain embodiments, Su is

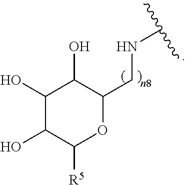

In certain embodiments, Su is

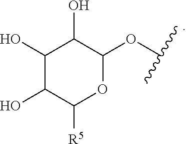

In certain embodiments, n8 is 0. In certain embodiments, n8 is 1. In certain embodiments, R⁵ is —CH₂OH. In certain embodiments, R⁵ is —C(O)OH.

In embodiments, Su is

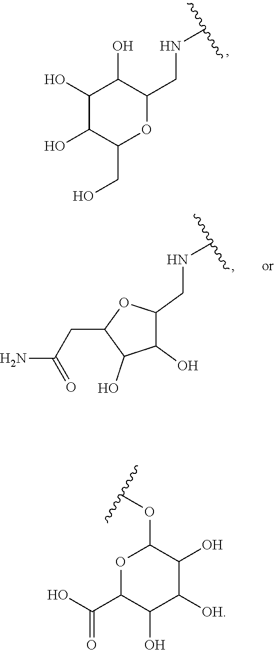 [SMILES: *NCC1OC(CO)C(O)C(O)C1O ; *NCC1OC(CC(N)=O)C(O)C1O ; *OC1OC(C(=O)O)C(O)C(O)C1O]

In certain embodiments, Su is

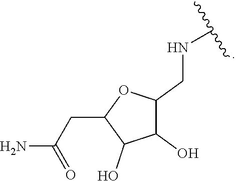 [SMILES: *NCC1OC(CC(N)=O)C(O)C1O]

In embodiments, Su is

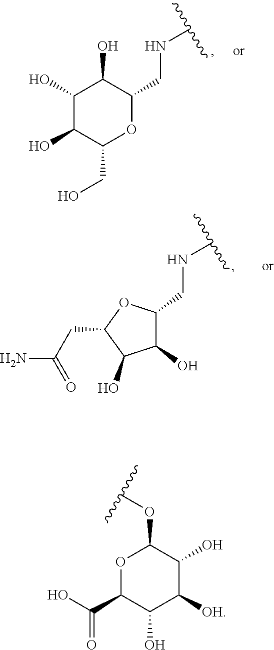 [SMILES: *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

In certain embodiments, Su

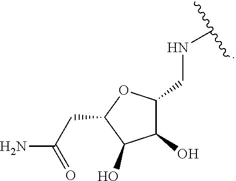 [SMILES: *NC[C@H]1O[C@@H](CC(N)=O)[C@H](O)[C@@H]1O]

In embodiments, Su is:

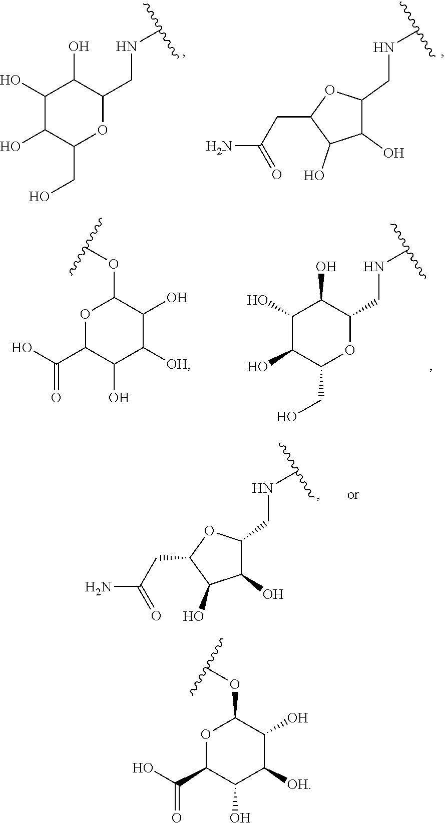 [SMILES: *NCC1OC(CO)C(O)C(O)C1O ; *NCC1OC(CC(N)=O)C(O)C1O ; *OC1OC(C(=O)O)C(O)C(O)C1O ; *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

In embodiments, L is

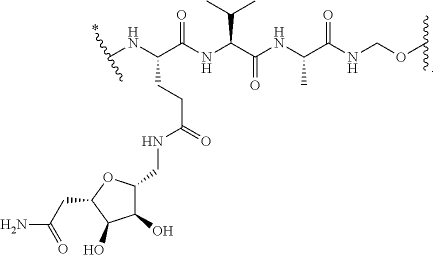 [SMILES: *N[C@@H](CCC(=O)NC[C@H]1O[C@@H](CC(N)=O)[C@H](O)[C@@H]1O)C(=O)N[C@H](C(=O)N[C@@H](C)C(=O)NCO*)C(C)C]

The antibody drug conjugates disclosed herein may include a conjugator (C). The conjugator may be indirectly joined to the cytotoxic agent via the linker. The conjugator may help avoid or reduce deconjugation of the cytotoxic agent in vivo, which may help maintain a stable drug-to-antibody ratio (DAR). The conjugator, e.g., prior to conjugation with an antibody, may have the following formula:

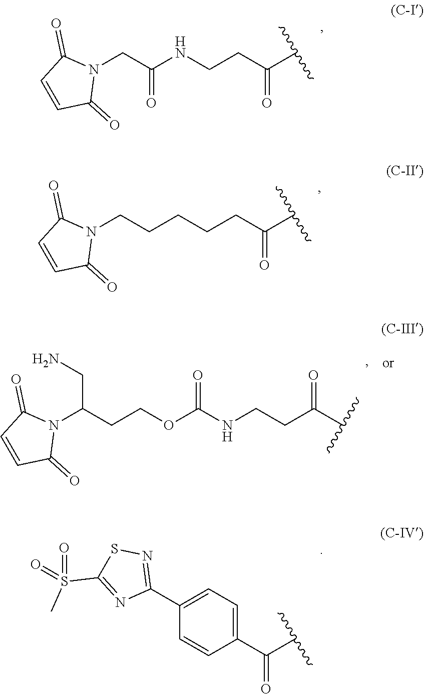 [SMILES: *C(=O)CCNC(=O)CN1C(=O)C=CC1=O ; *C(=O)CCCCCN1C(=O)C=CC1=O ; *C(=O)CCNC(=O)OCCC(CN)N1C(=O)C=CC1=O ; *C(=O)c1ccc(-c2nsc(S(C)(=O)=O)n2)cc1]

When the conjugator is joined directly to an anti-CEA antibody, the conjugator may have the following formula, in which * marks the bond where the conjugator connects to the antibody:

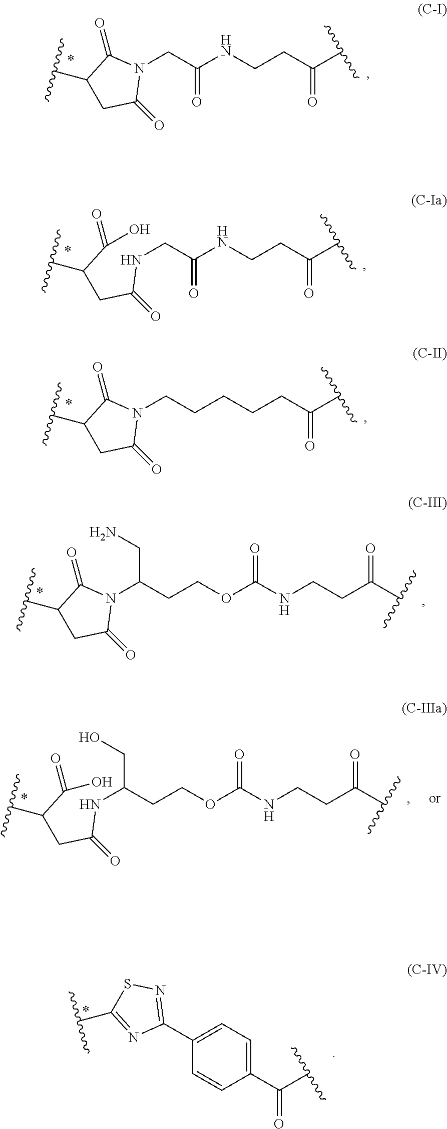 [SMILES: *C(=O)CCNC(=O)CN1C(=O)CC(*)C1=O ; *C(=O)CCNC(=O)CNC(=O)CC(*)C(=O)O ; *C(=O)CCCCCN1C(=O)CC(*)C1=O ; *C(=O)CCNC(=O)OCCC(CN)N1C(=O)CC(*)C1=O ; *C(=O)CCNC(=O)OCCC(CO)NC(=O)CC(*)C(=O)O ; *C(=O)c1ccc(-c2nsc(*)n2)cc1]

The conjugator, linker, sugar moiety, and cytotoxic agent, may be included in the presently disclosed antibody drug conjugates in any combination. Non-limiting examples of conjugator (pre-conjugation)-linker-cytotoxic agent combinations include the following:

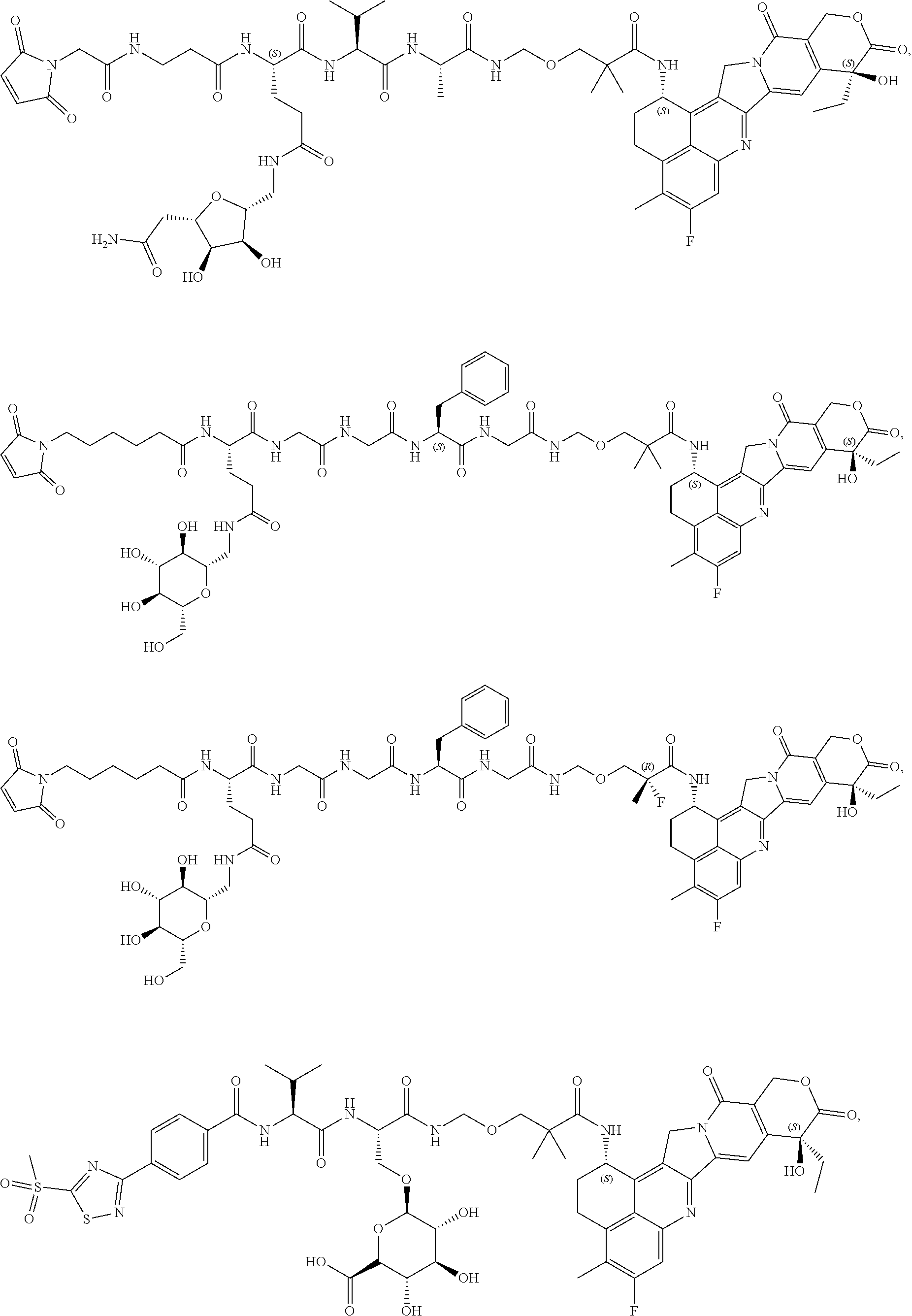

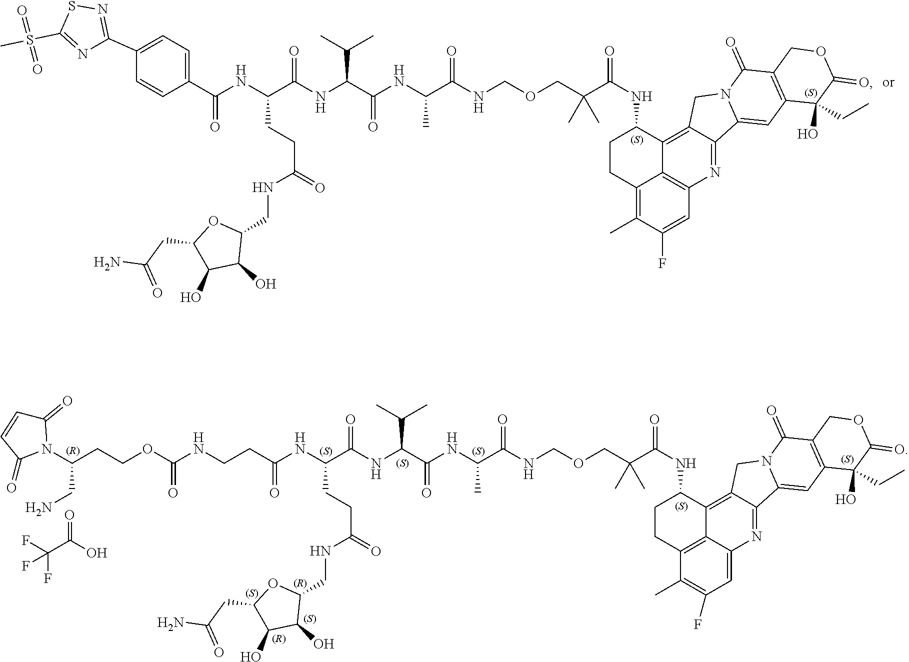

Each antibody drug conjugate may include more than one compound of conjugator-linker-cytotoxic agent (C-L-D), such as one, two, three, four, five, six, seven, eight, nine, or ten C-L-D. In embodiments, each antibody drug conjugate includes from 1 to 10, e.g., from 3 to 10, from 4 to 10, from 5 to 10, from 6 to 10, from 7 to 9, or about 8.

In certain embodiments, —C-L-(D)ₘ is:

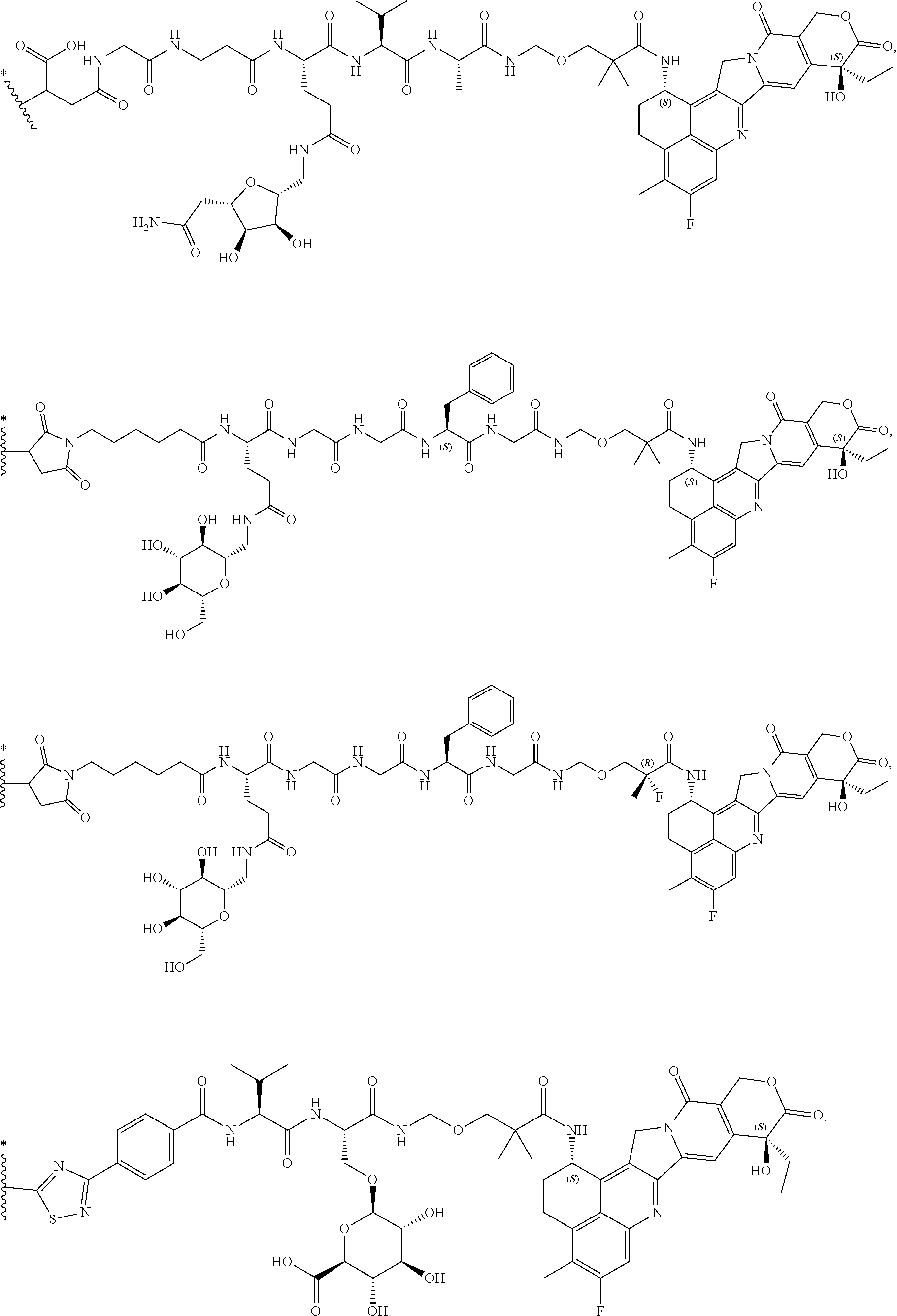

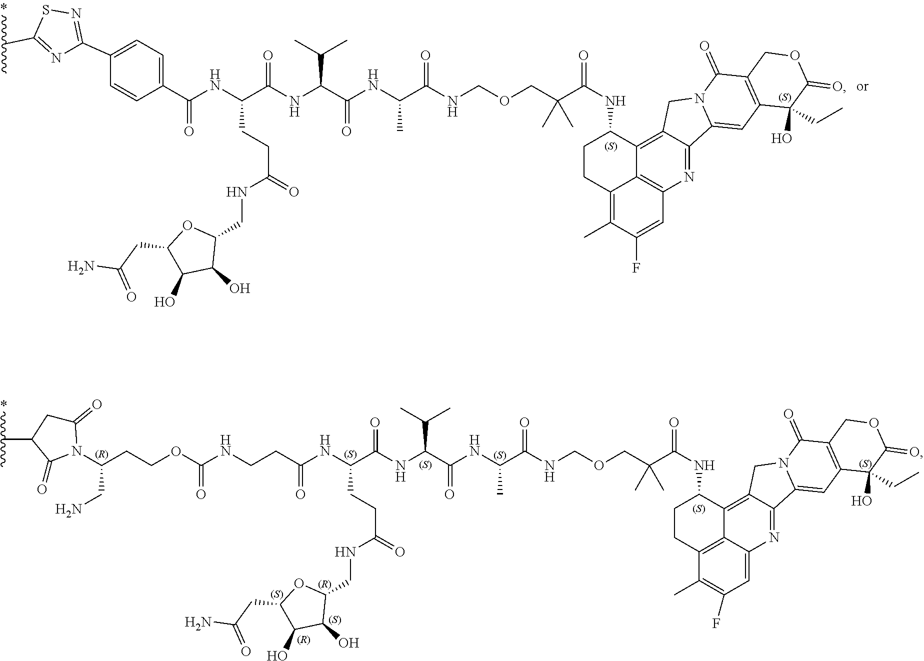

where * marks the bond where C connects to Ab. In certain embodiments, —C-L-(D)ₘ is:

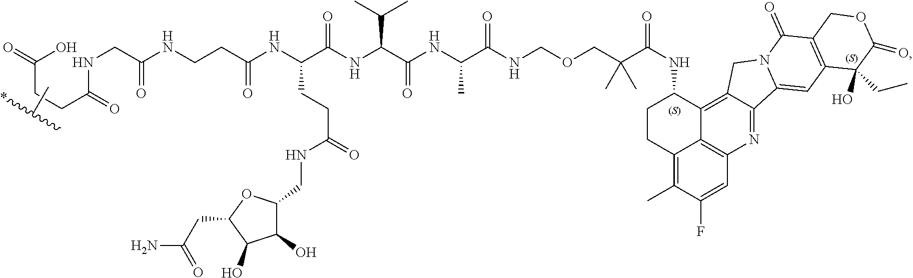

where * marks the bond where C connects to Ab. In certain embodiments, —C-L-(D)ₘ is:

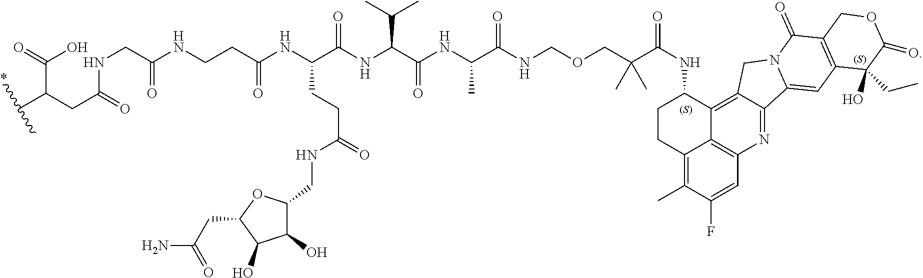

In certain embodiments, C-L-(D)ₘ is:

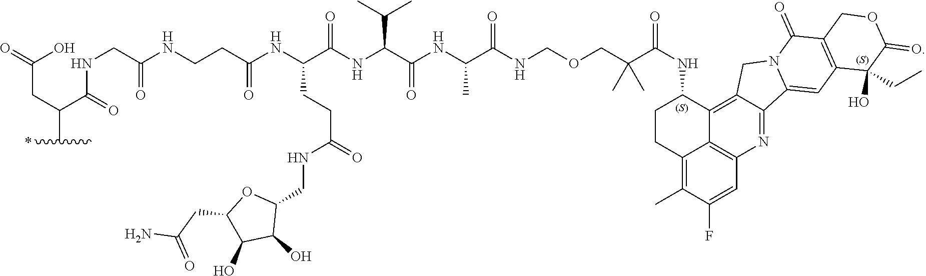

In embodiments, the antibody drug conjugate is

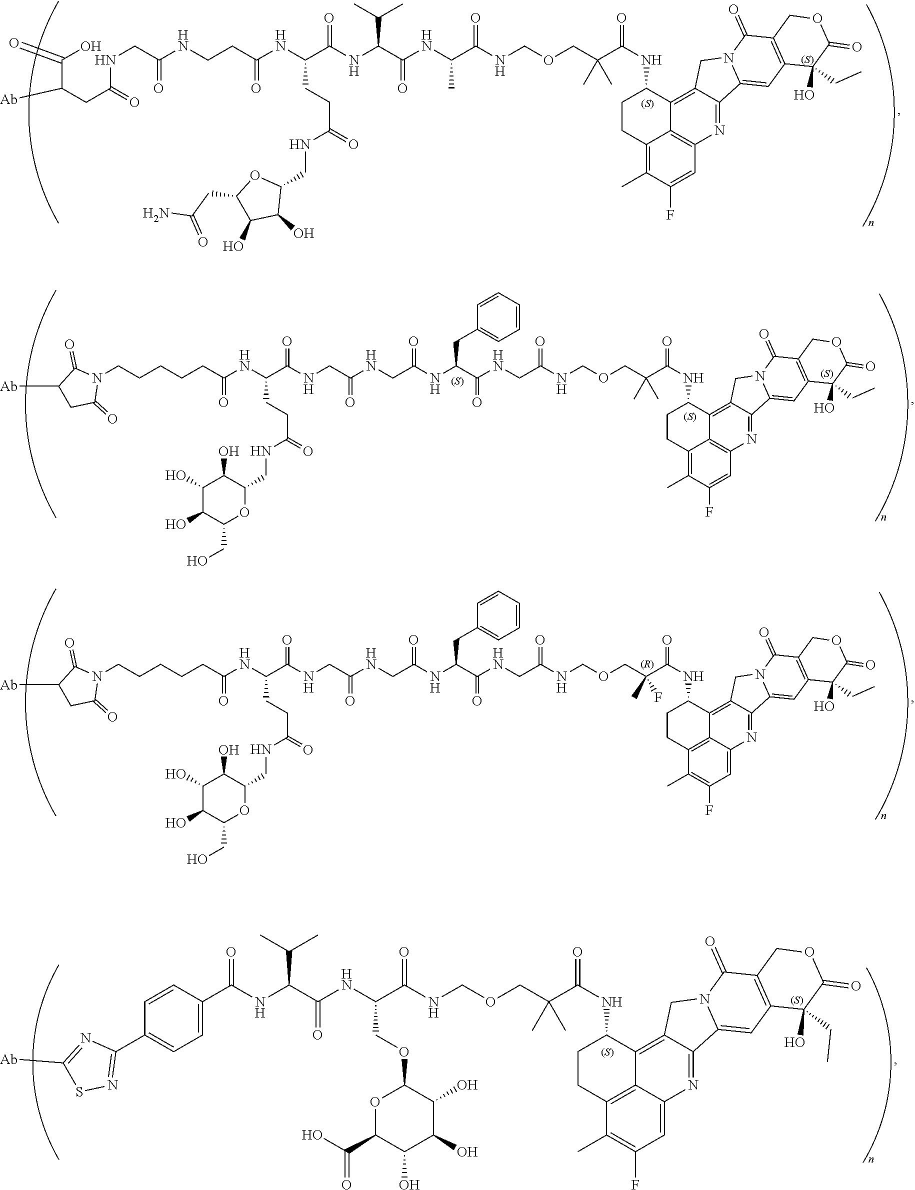

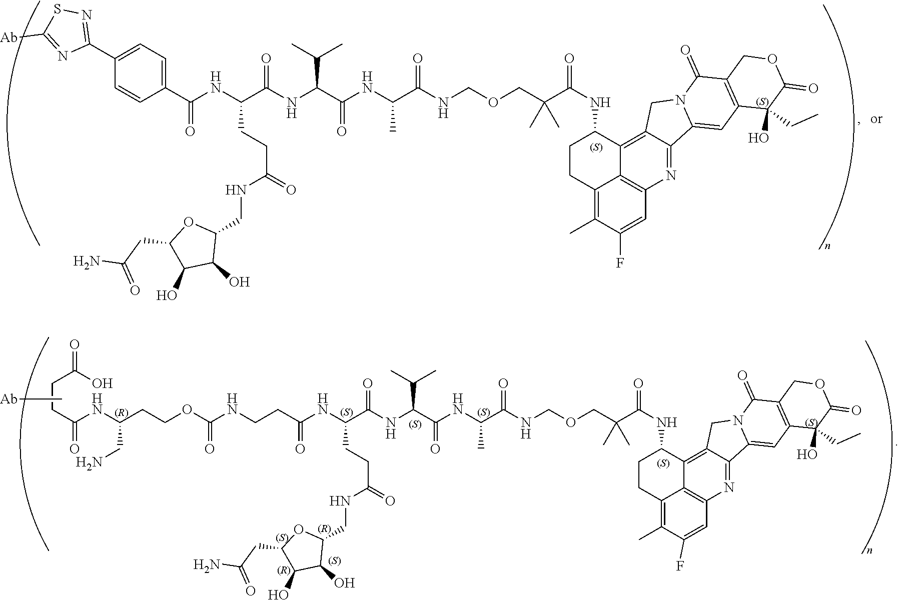

In certain embodiments, the antibody drug conjugate is of the following formula:

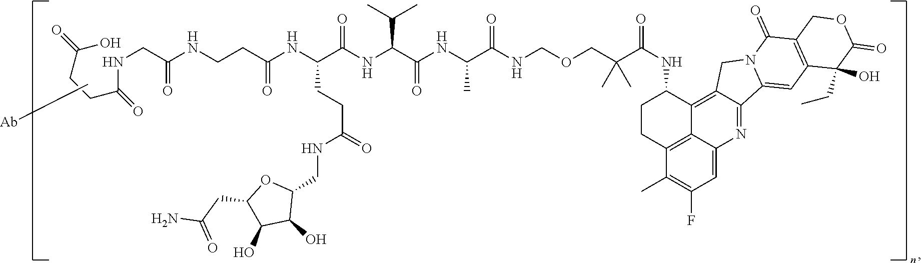

or a tautomer, pharmaceutically acceptable salt, solvate or hydrate thereof, wherein Ab and n are as described herein. In certain embodiments, the antibody drug conjugate is of the following formula:

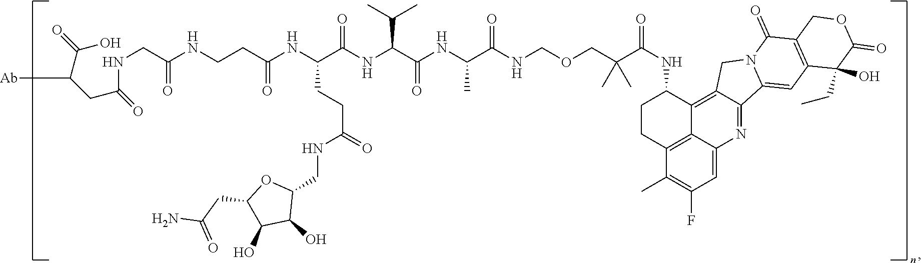

or a tautomer, pharmaceutically acceptable salt, solvate or hydrate thereof, wherein Ab and n are as described herein. In certain embodiments, the antibody drug conjugate is of the following formula:

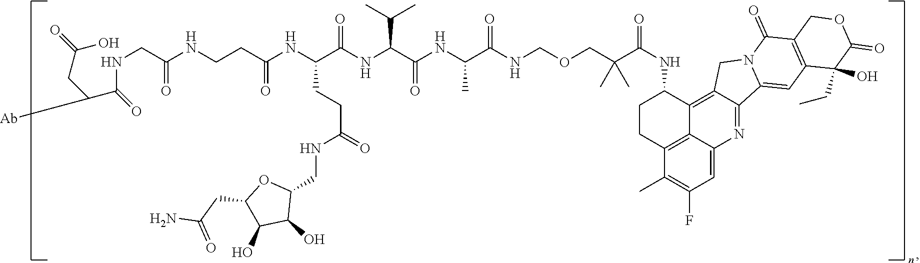

or a tautomer, pharmaceutically acceptable salt, solvate or hydrate thereof, wherein Ab and n are as described herein.

### Methods of Making Antibody Drug Conjugates

The antibody drug conjugates disclosed herein may be produced by any method known in the art. In one example, a host cell that has been transformed by an isolated nucleic acid comprising a sequence encoding an anti-CEA antibody or antigen-binding fragment thereof is cultured under suitable culturing conditions. The antibody or antigen-binding fragment thereof is thereby expressed and may be recovered from the cell culture.

The cytotoxic agent is conjugated to the antibody or antigen-binding fragment thereof using a linker disclosed herein to produce an antibody drug conjugate. In embodiments, a conjugator is also joined to the linker, such as between the antibody and linker.

### Methods of Treatment

The antibody drug conjugates of the present disclosure are useful in a variety of applications including, but not limited to, methods for the treatment of a CEA-associated disorder or disease. In one aspect, the CEA-associated disorder or disease is characterized by a CEA overexpressing or accumulating cell. In some embodiments, the cell is cancerous.

In certain aspects, the method comprises administering to a subject (e.g., patient) in need thereof an effective amount of an anti-CEA antibody drug conjugate. The subject can include, without limitation, a subject with a CEA-expressing cancer, CEA-accumulating cancer, CEA-responsive cancer, and gastric or rectal cancers and metastases thereof. In embodiments, the cancer is a lung cancer (e.g., non-small-cell lung cancer), gastrointestinal cancer (e.g., gastric cancer), or a colorectal cancer (e.g., rectal cancer).

The antibody drug conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments, or antibody drug conjugates, of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### Combination Therapy

In one aspect, anti-CEA antibody drug conjugates can be used in combination with other therapeutic agents. Other therapeutic agents that can be used with the anti-CEA antibody drug conjugates of the present disclosure include, but are not limited to, a chemotherapeutic agent (e.g., paclitaxel or a paclitaxel agent (e.g. Abraxane®), docetaxel, carboplatin, topotecan, cisplatin, irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor) (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, or Polo-like kinase 1 inhibitor (e.g., BI 672).

In another aspect, the anti-CEA antibody drug conjugates can be used in combination with an anti-PD1 antibody. Anti-PD1 antibodies can include, without limitation, tislelizumab, pembrolizumab, and nivolumab. Tislelizumab is disclosed in U.S. Pat. No. 8,735,553. Pembrolizumab (formerly MK-3475), as disclosed by Merck in U.S. Pat. Nos. 8,354,509 and 8,900,587, is a humanized IgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human IgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

### Pharmaceutical Compositions and Formulations

Also provided are compositions, including pharmaceutical formulations, comprising an anti-CEA antibody drug conjugate comprising an anti-CEA antibody or antigen-binding fragment thereof, or polynucleotides comprising sequences encoding an anti-CEA antibody or antigen-binding fragment, and a toxic drug conjugate. These compositions can further comprise suitable carriers, such as pharmaceutically acceptable excipients including buffers, which are well known in the art.

Pharmaceutical formulations of an anti-CEA antibody drug conjugate as described herein are prepared by mixing such antibody or antigen-binding fragment and antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody or antibody drug conjugate, which matrices are in the form of shaped articles, e.g. films, or microcapsules.

The formulations to be used for in vivo administration are generally sterile. Sterility can be readily accomplished, e.g., by filtration through sterile filtration membranes.

### EXAMPLES

### Example 1: Generation of Anti-CEA Monoclonal Antibody

### CEA Recombinant Proteins for Immunization and Binding Assays

Several recombinant proteins were designed and expressed for antibody screening (see Table 2). Antibodies against CEA that cross-react with human and Macaca mulatta CEA in the membrane peripheral region containing domain B3 (amino acids 596-674 of SEQ ID NO:52, see Beauchemin et al., “Isolation and characterization of full-length functional cDNA clones for human carcinoembryonic antigen.” Mol. Cell Biol., 1987, 7(9):3321-3330)). These antibodies lack off-target binding with other human CEACAM members.

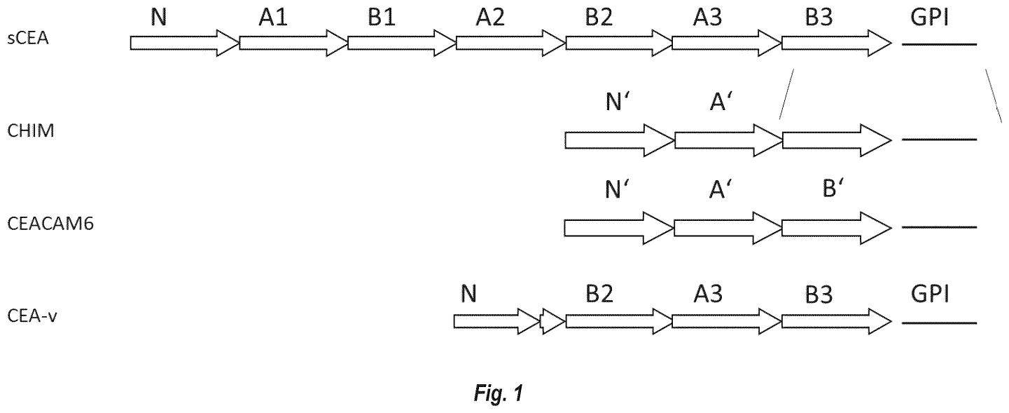

The cDNA coding regions for the full-length human CEA (SEQ ID NO:52), Macaca CEA (SEQ ID NO:53) and the full-length human CEACAM6 (SEQ ID NO:54) were ordered based on the GenBank sequence. For human CEA (Accession No:NM_004363.2), the gene is available from Sinobio, Cat. No. HG11077-UT. For Macaca CEA (Accession No:NM_001047125), the gene is available from GenScripts, Cat. No. OMb23865D. For human CEACAM6 (Accession No:NM_002483.4), the gene is available from Sinobio, Cat. No. HG10823-UT. The schematic presentation of CEA fusion proteins is shown in FIG. 1. It is reported a splice variant of human CEA is expressed concomitantly with full-length CEA on tumors (Peng et al., PloS one, 7, e36412-e36412 (2012)), and the variant (CEA-v) was prepared accordingly. To generate this construct, the coding region of extracellular domain (ECD) consisting of amino acid (AA) 1-687 of huCEA (SEQ ID NO:55), the region of amino acid (AA) 1-690 of monkey CEA (SEQ ID NO:56) and the region of amino acid (AA) 1-320 of CEACAM6 (SEQ ID NO:57) were PCR-amplified. The regions of CEA amino acid (AA) 1-78 (SEQ ID NO:58) and amino acids 398-687 of CEA (SEQ ID NO:59) were PCR-amplified, and then conjugated by overlap-PCR to make a CEA variant (CEA-v) (SEQ ID NO:60). Alternatively, the regions of CEACAM6 amino acid (AA) 1-273 (SEQ ID NO:61), and the membrane-peripheral region containing domain B3 of CEA amino acid (AA) 596-687 of (SEQ ID NO:62) were PCR-amplified, and then conjugated by overlap-PCR to make a chimeric construct (CHIM) (SEQ ID NO:63). All constructs were then cloned into a pcDNA3.1-based expression vector (Invitrogen, Carlsbad, CA, USA) with C-terminus fused with 6×His tags individually, which resulted in five recombinant fusion protein expression plasmids, CEA, monkey CEA, CEACAM6, CEA-v and CHIM. For recombinant fusion protein production, CEA, monkey CEA, CEACAM6, CEA-v and CHIM plasmids were transiently transfected into a HEK293-based mammalian cell expression system (generated in house) and cultured for 5-7 days in a CO₂ incubator equipped with a rotating shaker. The supernatants containing the recombinant proteins were collected and cleared by centrifugation. Recombinant proteins were purified using a Ni-NTA agarose (Cat. No. R90115, Invitrogen). All recombinant proteins were dialyzed against phosphate buffered saline (PBS) and stored in −80° C. freezer in small aliquots.

### Stable Expression in Cell Lines

To establish stable cell lines that express full-length human CEA (Accession No:NM_004363.2, the cDNA expressing CEA was cloned into a retroviral vector pFB-Neo (Cat. No. 217561, Agilent, USA). Dual-tropic retroviral vectors were generated according to a previous protocol (Zhang et al., Blood. 2005 106(5):1544-51). Viral vectors containing human CEA were transduced into L929 (ATCC, Manassas, VA, USA) and CT26 cells (ATCC, Manassas, VA, USA), in order to generate human CEA expressing cell lines. The high expression cell lines were selected by culture in complete RPMI1640 medium containing 10% FBS with G418, and then verified via FACS binding assay.

### Immunization, Hybridoma Fusion and Cloning

Eight to twelve week-old Balb/c mice (HFK BIOSCIENCE CO., LTD, Beijing, China) were immunized intraperitoneally (i.p.) with 500 μL of 1×10⁷ L929/huCEA cells with or without a water-soluble adjuvant (Cat. No. KX0210041, KangBiQuan, Beijing, China). The procedure was repeated two weeks later in order to boost antibody production. Two weeks after the third immunization, mouse sera were evaluated for soluble CEA (sCEA) binding by ELISA and FACS. Splenocytes were isolated and fused to the murine myeloma cell line, SP2/0 cells (ATCC, Manassas, VA, USA), using the standard techniques (Colligan J E, et al., CURRENT PROTOCOLS IN IMMUNOLOGY, 1993).

### Assessment of CEA Binding Activity of Antibodies by ELISA and FACS

To screen for antibodies that bound human CEA, but did not bind CEACAM6 or sCEA, antibodies which bound to CHIM but not to sCEA, CEACAM6 and CEA-v, and antibodies which bind to CHIM, sCEA and CEA-v, but not to CEACAM6 were screened and counter-screened. The supernatants of hybridoma clones were initially screened by ELISA as described in (Methods in Molecular Biology (2007) 378:33-52) with some modifications. Briefly, sCEA, CHIM, CEACAM6 or CEA-v were coated in 96-well plates at a low concentration of 3 μg/ml, individually. The HRP-linked anti-mouse IgG antibody (Cat. No. 7076S, Cell Signaling Technology, USA) and substrate (Cat. No. 00-4201-56, eBioscience, USA) were used for development, and absorbance signal at the wavelength of 450 nm was measured using a plate reader (SpectraMax Paradigm™, Molecular Devices, USA). The ELISA-positive clones were further verified by FACS using either the L929/huCEA and/or MKN45 cells (ATCC). MKN45 cells are of human gastric cancer origin. CEA-expressing cells (105 cells/well) were incubated with ELISA-positive hybridoma supernatants, followed by binding with Alexa Fluro-647-labeled goat anti-mouse IgG antibody (Cat. No. A0473, Beyotime Biotechnology, China). Cell fluorescence was quantified using a flow cytometer (Guava easyCyte™ 8HT, Merck-Millipore, USA).

The conditioned media from the hybridomas that showed positive signals in FACS screening, and binding to CHIM but not CEACAM6 and sCEA were subjected to functional assays to evaluate the presence of sCEA on the binding of CEA antibodies to CEA expressing cells (see the Examples below). The antibodies with the desired binding specificity and functional activities were further sub-cloned and characterized.

### Subcloning and Adaptation of Hybridomas to Serum-Free or Low Serum Medium

After screening primarily by ELISA, FACS and functional assays, the positive hybridoma clones were sub-cloned by limiting dilution. The top antibody subclones verified through functional assays were adapted for growth in the CDM4MAb medium (Cat. No. SH30801.02, Hyclone, USA) with 3% FBS.

### Expression and Purification of Monoclonal Antibodies

Hybridoma cells were cultured in CDM4MAb medium (Cat. No. SH30801.02, Hyclone), and incubated in a CO₂ incubator for 5 to 7 days at 37° C. The conditioned medium was collected through centrifugation and filtration by passing through a 0.22 μm membrane before purification. Murine antibody-containing supernatants were applied and bound to a Protein A column (Cat. No. 17127901, GE Life Sciences) following the protocol in the manufacturer's guide. The procedure usually yielded antibodies at purity above 90%. The Protein A-affinity purified antibodies were either dialyzed against PBS or further purified using a HiLoad 16/60 Superdex™ 200 column (Cat. No. 17531801, GE Life Sciences) to remove aggregates. Protein concentrations were determined by measuring absorbance at 280 nm. The final antibody preparations were stored in aliquots in −80° C. freezer.

TABLE 2
Amino acid and nucleic acid sequences

| SEQ ID | | |
|---|---|---|
| NO: | Construct | SEQUENCE |
| SEQ ID | full-length | MESPSAPPHRWCIPWQRLLLTASLLTFWNPPTTAKLTIESTPFNV |
| NO: 52 | human CEA | AEGKEVLLLVHNLPQHLFGYSWYKGERVDGNRQIIGYVIGTQ |
| | | QATPGPAYSGREIIYPNASLLIQNIIQNDTGFYTLHVIKSDLVNE |
| | | EATGQFRVYPELPKPSISSNNSKPVEDKDAVAFTCEPETQDATY |
| | | LWWVNNQSLPVSPRLQLSNGNRTLTLFNVTRNDTASYKCETQ |
| | | NPVSARRSDSVILNVLYGPDAPTISPLNTSYRSGENLNLSCHAA |
| | | SNPPAQYSWFVNGTFQQSTQELFIPNITVNNSGSYTCQAHNSD |
| | | TGLNRTTVTTITVYAEPPKPFITSNNSNPVEDEDAVALTCEPEIQ |
| | | NTTYLWWVNNQSLPVSPRLQLSNDNRTLTLLSVTRNDVGPYE |
| | | CGIQNELSVDHSDPVILNVLYGPDDPTISPSYTYYRPGVNLSLS |
| | | CHAASNPPAQYSWLIDGNIQQHTQELFISNITEKNSGLYTCQA |
| | | NNSASGHSRTTVKTITVSAELPKPSISSNNSKPVEDKDAVAFTC |
| | | EPEAQNTTYLWWVNGQSLPVSPRLQLSNGNRTLTLFNVTRND |
| | | ARAYVCGIQNSVSANRSDPVTLDVLYGPDTPIISPPDSSYLSGA |
| | | NLNLSCHSASNPSPQYSWRINGIPQQHTQVLFIAKITPNNNGTY |
| | | ACFVSNLATGRNNSIVKSITVSASGTSPGLSAGATVGIMIGVLV |
| | | GVALI |
| SEQ ID | Macaca | MGSPSAPLHRWCIPWQTLLLTASLLTFWNPPTTAQLTIESRPEN |
| NO: 53 | CEA | VAEGKEVLLLAHNVSQNLFGYIWYKGERVDASRRIGSCVIRTQ |
| | | QITPGPAHSGRETIDFNASLLIHNVTQSDTGSYTIQVIKEDLVNE |
| | | EATGQFRVYPELPKPYISSNNSNPVEDKDAVALTCEPETQDTTY |
| | | LWWVNNQSLPVSPRLELSSDNRTLTVFNIPRNDTTSYKCETQN |
| | | PVSVRRSDPVTLNVLYGPDAPTISPLNTPYRAGENLNLTCHAA |
| | | SNPTAQYFWFVNGTFQQSTQELFIPNITVNNSGSYMCQAHNSA |
| | | TGLNRTTVTAITVYAELPKPYITSNNSNPIEDKDAVTLTCEPETQ |
| | | DTTYLWWVNNQSLSVSSRLELSNDNRTLTVFNIPRNDTTFYEC |
| | | ETQNPVSVRRSDPVTLNVLYGPDAPTISPLNTPYRAGENLNLS |
| | | CHAASNPAAQYSWFVNGTFQQSTQELFIPNITVNNSGSYMCQ |
| | | AHNSATGLNRTTVTAITVYVELPKPYISSNNSNPIEDKDAVTLT |
| | | CEPVAENTTYLWWVNNQSLSVSPRLQLSNGNRILTLLSVTRND |
| | | TGPYECGIQNSESAKRSDPVTLNVTYGPDTPIISPPDLSYRSGA |
| | | NLNLSCHSDSNPSPQYSWLINGTLRQHTQVLFISKITSNNSGAY |
| | | ACFVSNLATGRNNSIVKNISVSSGDSAPGSSGLSARATVGIIIGM |
| | | LVGVALM |
| SEQ ID | full-length | MGPPSAPPCRLHVPWKEVLLTASLLTFWNPPTTAKLTIESTPFN |
| NO: 54 | human | VAEGKEVLLLAHNLPQNRIGYSWYKGERVDGNSLIVGYVIGT |
| | CEACAM6 | QQATPGPAYSGRETIYPNASLLIQNVTQNDTGFYTLQVIKSDLV |
| | | NEEATGQFHVYPELPKPSISSNNSNPVEDKDAVAFTCEPEVQNT |
| | | TYLWWVNGQSLPVSPRLQLSNGNMTLTLLSVKRNDAGSYEC |
| | | EIQNPASANRSDPVTLNVLYGPDVPTISPSKANYRPGENLNLSC |
| | | HAASNPPAQYSWFINGTFQQSTQELFIPNITVNNSGSYMCQAH |
| | | NSATGLNRTTVTMITVSGSAPVLSAVATVGITIGVLARVALI |
| SEQ ID | 1-687 of | MESPSAPPHRWCIPWQRLLLTASLLTFWNPPTTAKLTIESTPFNV |
| NO: 55 | huCEA | AEGKEVLLLVHNLPQHLFGYSWYKGERVDGNRQIIGYVIGTQ |
| | | QATPGPAYSGREIIYPNASLLIQNIIQNDTGFYTLHVIKSDLVNE |
| | | EATGQFRVYPELPKPSISSNNSKPVEDKDAVAFTCEPETQDATY |
| | | LWWVNNQSLPVSPRLQLSNGNRTLTLFNVTRNDTASYKCETQ |
| | | NPVSARRSDSVILNVLYGPDAPTISPLNTSYRSGENLNLSCHAA |
| | | SNPPAQYSWFVNGTFQQSTQELFIPNITVNNSGSYTCQAHNSD |
| | | TGLNRTTVTTITVYAEPPKPFITSNNSNPVEDEDAVALTCEPEIQ |
| | | NTTYLWWVNNQSLPVSPRLQLSNDNRTLTLLSVTRNDVGPYE |
| | | CGIQNELSVDHSDPVILNVLYGPDDPTISPSYTYYRPGVNLSLS |
| | | CHAASNPPAQYSWLIDGNIQQHTQELFISNITEKNSGLYTCQA |
| | | NNSASGHSRTTVKTITVSAELPKPSISSNNSKPVEDKDAVAFTC |
| | | EPEAQNTTYLWWVNGQSLPVSPRLQLSNGNRTLTLFNVTRND |
| | | ARAYVCGIQNSVSANRSDPVTLDVLYGPDTPIISPPDSSYLSGA |
| | | NLNLSCHSASNPSPQYSWRINGIPQQHTQVLFIAKITPNNNGTY |
| | | ACFVSNLATGRNNSIVKSITVSASGTSPGLSAGAHHHHHH |
| SEQ ID | 1-690 of | MGSPSAPLHRWCIPWQTLLLTASLLTFWNPPTTAQLTIESRPFN |
| NO: 56 | monkey | VAEGKEVLLLAHNVSQNLFGYIWYKGERVDASRRIGSCVIRTQ |
| | CEA | QITPGPAHSGRETIDFNASLLIHNVTQSDTGSYTIQVIKEDLVNE |
| | | EATGQFRVYPELPKPYISSNNSNPVEDKDAVALTCEPETQDTTY |
| | | LWWVNNQSLPVSPRLELSSDNRTLTVFNIPRNDTTSYKCETQN |
| | | PVSVRRSDPVTLNVLYGPDAPTISPLNTPYRAGENLNLTCHAA |
| | | SNPTAQYFWFVNGTFQQSTQELFIPNITVNNSGSYMCQAHNSA |
| | | TGLNRTTVTAITVYAELPKPYITSNNSNPIEDKDAVTLTCEPETQ |
| | | DTTYLWWVNNQSLSVSSRLELSNDNRTLTVFNIPRNDTTFYEC |
| | | ETQNPVSVRRSDPVTLNVLYGPDAPTISPLNTPYRAGENLNLS |
| | | CHAASNPAAQYSWFVNGTFQQSTQELFIPNITVNNSGSYMCQ |
| | | AHNSATGLNRTTVTAITVYVELPKPYISSNNSNPIEDKDAVTLT |
| | | CEPVAENTTYLWWVNNQSLSVSPRLQLSNGNRILTLLSVTRND |
| | | TGPYECGIQNSESAKRSDPVTLNVTYGPDTPIISPPDLSYRSGA |
| | | NLNLSCHSDSNPSPQYSWLINGTLRQHTQVLFISKITSNNSGAY |
| | | ACFVSNLATGRNNSIVKNISVSSGDSAPGSSGLSARAHHHHHH |
| SEQ ID | 1-320 of | MGPPSAPPCRLHVPWKEVLLTASLLTFWNPPTTAKLTIESTPEN |
| NO: 57 | CEACAM6 | VAEGKEVLLLAHNLPQNRIGYSWYKGERVDGNSLIVGYVIGT |
| | | QQATPGPAYSGRETIYPNASLLIQNVTQNDTGFYTLQVIKSDLV |
| | | NEEATGQFHVYPELPKPSISSNNSNPVEDKDAVAFTCEPEVQNT |
| | | TYLWWVNGQSLPVSPRLQLSNGNMTLTLLSVKRNDAGSYEC |
| | | EIQNPASANRSDPVTLNVLYGPDVPTISPSKANYRPGENLNLSC |
| | | HAASNPPAQYSWFINGTFQQSTQELFIPNITVNNSGSYMCQAH |
| | | NSATGLNRTTVTMITVSGHHHHHH |
| SEQ ID | 1-78 of | MESPSAPPHRWCIPWQRLLLTASLLTFWNPPTTAKLTIESTPFNV |
| NO: 58 | huCEA | AEGKEVLLLVHNLPQHLFGYSWYKGERVDGNRQIIGYVIGTQ |
| | | QATPGPAYSGREITYPNASLLIQNIIQN |
| SEQ ID | 398-687 of | ELSVDHSDPVILNVLYGPDDPTISPSYTYYRPGVNLSLSCHAAS |
| NO: 59 | huCEA | NPPAQYSWLIDGNIQQHTQELFISNITEKNSGLYTCQANNSASG |
| | | HSRTTVKTITVSAELPKPSISSNNSKPVEDKDAVAFTCEPEAQN |
| | | TTYLWWVNGQSLPVSPRLQLSNGNRTLTLFNVTRNDARAYVC |
| | | GIQNSVSANRSDPVTLDVLYGPDTPIISPPDSSYLSGANLNLSC |
| | | HSASNPSPQYSWRINGIPQQHTQVLFIAKITPNNNGTYACFVSN |
| | | LATGRNNSIVKSITVSASGTSPGLSAGA |
| SEQ ID | CEA-v | MESPSAPPHRWCIPWQRLLLTASLLTFWNPPTTAKLTIESTPFNV |
| NO: 60 | | AEGKEVLLLVHNLPQHLFGYSWYKGERVDGNRQIIGYVIGTQ |
| | | QATPGPAYSGREITYPNASLLIQNIIQNELSVDHSDPVILNVLYGP |
| | | DDPTISPSYTYYRPGVNLSLSCHAASNPPAQYSWLIDGNIQQH |
| | | TQELFISNITEKNSGLYTCQANNSASGHSRTTVKTITVSAELPK |
| | | PSISSNNSKPVEDKDAVAFTCEPEAQNTTYLWWVNGQSLPVSP |
| | | RLQLSNGNRTLTLFNVTRNDARAYVCGIQNSVSANRSDPVTL |
| | | DVLYGPDTPIISPPDSSYLSGANLNLSCHSASNPSPQYSWRINGI |
| | | PQQHTQVLFIAKITPNNNGTYACFVSNLATGRNNSIVKSITVSA |
| | | SGTSPGLSAGAHHHHHH |
| SEQ ID | 1-273 of | MGPPSAPPCRLHVPWKEVLLTASLLTFWNPPTTAKLTIESTPFN |
| NO: 61 | CEACAM6 | VAEGKEVLLLAHNLPQNRIGYSWYKGERVDGNSLIVGYVIGT |
| | | QQATPGPAYSGRETIYPNASLLIQNVTQNDTGFYTLQVIKSDLV |
| | | NEEATGQFHVYPELPKPSISSNNSNPVEDKDAVAFTCEPEVQNT |
| | | TYLWWVNGQSLPVSPRLQLSNGNMTLTLLSVKRNDAGSYEC |
| | | EIQNPASANRSDPVTLNVLYGPDVPTIS |
| SEQ ID | 596-687 of | PIISPPDSSYLSGANLNLSCHSASNPSPQYSWRINGIPQQHTQVL |
| NO: 62 | huCEA | FIAKITPNNNGTYACFVSNLATGRNNSIVKSITVSASGTSPGLSA |
| | | GA |
| SEQ ID | CHIM | MGPPSAPPCRLHVPWKEVLLTASLLTFWNPPTTAKLTIESTPFN |
| NO: 63 | | VAEGKEVLLLAHNLPQNRIGYSWYKGERVDGNSLIVGYVIGT |
| | | QQATPGPAYSGRETIYPNASLLIQNVTQNDTGFYTLQVIKSDLV |
| | | NEEATGQFHVYPELPKPSISSNNSNPVEDKDAVAFTCEPEVQNT |
| | | TYLWWVNGQSLPVSPRLQLSNGNMTLTLLSVKRNDAGSYEC |
| | | EIQNPASANRSDPVTLNVLYGPDVPTISPIISPPDSSYLSGANLN |
| | | LSCHSASNPSPQYSWRINGIPQQHTQVLFIAKITPNNNGTYACF |
| | | VSNLATGRNNSIVKSITVSASGTSPGLSAGAHHHHHH |
| SEQ ID | CEACAM1 | MGHLSAPLHRVRVPWQGLLLTASLLTFWNPPTTAQLTTESMPF |
| NO: 64 | | NVAEGKEVLLLVHNLPQQLFGYSWYKGERVDGNRQIVGYAIG |
| | | TQQATPGPANSGRETIYPNASLLIQNVTQNDTGFYTLQVIKSDL |
| | | VNEEATGQFHVYPELPKPSISSNNSNPVEDKDAVAFTCEPETQD |
| | | TTYLWWINNQSLPVSPRLQLSNGNRTLTLLSVTRNDTGPYECE |
| | | IQNPVSANRSDPVTLNVTYGPDTPTISPSDTYYRPGANLSLSCY |
| | | AASNPPAQYSWLINGTFQQSTQELFIPNITVNNSGSYTCHANN |
| | | SVTGCNRTTVKTIIVTELSPVVAKPQIKASKTTVTGDKDSVNLT |
| | | CSTNDTGISIRWFFKNQSLPSSERMKLSQGNTTLSINPVKREDA |
| | | GTYWCEVFNPISKNQSDPIMLNVNYNALPQENGLSPGAIAGIV |
| | | IGVVALVALIAVALACFLHFGKTGSSGPLQ |
| SEQ ID | CEACAM3 | MGPPSASPHRECIPWQGLLLTASLLNFWNPPTTAKLTIESMPLS |
| NO: 65 | | VAEGKEVLLLVHNLPQHLFGYSWYKGERVDGNSLIVGYVIGT |
| | | QQATPGAAYSGRETIYTNASLLIQNVTQNDIGFYTLQVIKSDLV |
| | | NEEATGQFHVYQENAPGLPVGAVAGIVTGVLVGVALVAALVCF |
| | | LLLAKTGRTSIQRDLKEQQPQALAPGRGPSHSSAFSMSPLSTA |
| | | QAPLPNPRTAASIYEELLKHDTNIYCRMDHKAEVAS |
| SEQ ID | CEACAM7 | MGSPSACPYRVCIPWQGLLLTASLLTFWNLPNSAQTNIDVVPF |
| NO: 66 | | NVAEGKEVLLVVHNESQNLYGYNWYKGERVHANYRIIGYVK |
| | | NISQENAPGPAHNGRETIYPNGTLLIQNVTHNDAGIYTLHVIKE |
| | | NLVNEEVTRQFYVFSEPPKPSITSNNFNPVENKDIVVLTCQPET |
| | | QNTTYLWWVNNQSLLVSPRLLLSTDNRTLVLLSATKNDIGPYE |
| | | CEIQNPVGASRSDPVTLNVRYESVQASSPDLSAGTAVSIMIGVL |
| | | AGMALI |
| SEQ ID | CEACAM8 | MGPISAPSCRWRIPWQGLLLTASLFTFWNPPTTAQLTIEAVPSN |
| NO: 67 | | AAEGKEVLLLVHNLPQDPRGYNWYKGETVDANRRIIGYVISN |
| | | QQITPGPAYSNRETIYPNASLLMRNVTRNDTGSYTLQVIKLNL |
| | | MSEEVTGQFSVHPETPKPSISSNNSNPVEDKDAVAFTCEPETQN |
| | | TTYLWWVNGQSLPVSPRLQLSNGNRTLTLLSVTRNDVGPYEC |
| | | EIQNPASANFSDPVTLNVLYGPDAPTISPSDTYYHAGVNLNLSC |
| | | HAASNPPSQYSWSVNGTFQQYTQKLFIPNITTKNSGSYACHTT |
| | | NSATGRNRTTVRMITVSDALVQGSSPGLSARATVSIMIGVLARV |
| | | ALI |
| SEQ ID | full-length | ATGGAGTCTCCCTCGGCCCCTCCCCACAGATGGTGCATCCCC |
| NO: 68 | human CEA | TGGCAGAGGCTCCTGCTCACAGCCTCACTTCTAACCTTCTG |
| | DNA | GAACCCGCCCACCACTGCCAAGCTCACTATTGAATCCACGC |
| | | CGTTCAATGTCGCAGAGGGGAAGGAGGTGCTTCTACTTGTC |
| | | CACAATCTGCCCCAGCATCTTTTTGGCTACAGCTGGTACAAA |
| | | GGTGAAAGAGTGGATGGCAACCGTCAAATTATAGGATATGT |
| | | AATAGGAACTCAACAAGCTACCCCAGGGCCCGCATACAGTG |
| | | GTCGAGAGATAATATACCCCAATGCATCCCTGCTGATCCAGA |
| | | ACATCATCCAGAATGACACAGGATTCTACACCCTACACGTCA |
| | | TAAAGTCAGATCTTGTGAATGAAGAAGCAACTGGCCAGTTC |
| | | CGGGTATACCCGGAGCTGCCCAAGCCCTCCATCTCCAGCAA |
| | | CAACTCCAAACCCGTGGAGGACAAGGATGCTGTGGCCTTCA |
| | | CCTGTGAACCTGAGACTCAGGACGCAACCTACCTGTGGTGG |
| | | GTAAACAATCAGAGCCTCCCGGTCAGTCCCAGGCTGCAGCT |
| | | GTCCAATGGCAACAGGACCCTCACTCTATTCAATGTCACAA |
| | | GAAATGACACAGCAAGCTACAAATGTGAAACCCAGAACCC |
| | | AGTGAGTGCCAGGCGCAGTGATTCAGTCATCCTGAATGTCC |
| | | TCTATGGCCCGGATGCCCCCACCATTTCCCCTCTAAACACAT |
| | | CTTACAGATCAGGGGAAAATCTGAACCTCTCCTGCCACGCA |
| | | GCCTCTAACCCACCTGCACAGTACTCTTGGTTTGTCAATGGG |
| | | ACTTTCCAGCAATCCACCCAAGAGCTCTTTATCCCCAACATC |
| | | ACTGTGAATAATAGTGGATCCTATACGTGCCAAGCCCATAAC |
| | | TCAGACACTGGCCTCAATAGGACCACAGTCACGACGATCAC |
| | | AGTCTATGCAGAGCCACCCAAACCCTTCATCACCAGCAACA |
| | | ACTCCAACCCCGTGGAGGATGAGGATGCTGTAGCCTTAACC |
| | | TGTGAACCTGAGATTCAGAACACAACCTACCTGTGGTGGGT |
| | | AAATAATCAGAGCCTCCCGGTCAGTCCCAGGCTGCAGCTGT |
| | | CCAATGACAACAGGACCCTCACTCTACTCAGTGTCACAAGG |
| | | AATGATGTAGGACCCTATGAGTGTGGAATCCAGAACGAATT |
| | | AAGTGTTGACCACAGCGACCCAGTCATCCTGAATGTCCTCT |
| | | ATGGCCCAGACGACCCCACCATTTCCCCCTCATACACCTATT |
| | | ACCGTCCAGGGGTGAACCTCAGCCTCTCCTGCCATGCAGCC |
| | | TCTAACCCACCTGCACAGTATTCTTGGCTGATTGATGGGAAC |
| | | ATCCAGCAACACACACAAGAGCTCTTTATCTCCAACATCAC |
| | | TGAGAAGAACAGCGGACTCTATACCTGCCAGGCCAATAACT |
| | | CAGCCAGTGGCCACAGCAGGACTACAGTCAAGACAATCAC |
| | | AGTCTCTGCGGAGCTGCCCAAGCCCTCCATCTCCAGCAACA |
| | | ACTCCAAACCCGTGGAGGACAAGGATGCTGTGGCCTTCACC |
| | | TGTGAACCTGAGGCTCAGAACACAACCTACCTGTGGTGGGT |
| | | AAATGGTCAGAGCCTCCCAGTCAGTCCCAGGCTGCAGCTGT |
| | | CCAATGGCAACAGGACCCTCACTCTATTCAATGTCACAAGA |
| | | AATGACGCAAGAGCCTATGTATGTGGAATCCAGAACTCAGT |
| | | GAGTGCAAACCGCAGTGACCCAGTCACCCTGGATGTCCTCT |
| | | ATGGGCCGGACACCCCCATCATTTCCCCCCCAGACTCGTCTT |
| | | ACCTTTCGGGAGCGAACCTCAACCTCTCCTGCCACTCGGCC |
| | | TCTAACCCATCCCCGCAGTATTCTTGGCGTATCAATGGGATA |
| | | CCGCAGCAACACACACAAGTTCTCTTTATCGCCAAAATCAC |
| | | GCCAAATAATAACGGGACCTATGCCTGTTTTGTCTCTAACTT |
| | | GGCTACTGGCCGCAATAATTCCATAGTCAAGAGCATCACAGT |
| | | CTCTGCATCTGGAACTTCTCCTGGTCTCTCAGCTGGGGCCAC |
| | | TGTCGGCATCATGATTGGAGTGCTGGTTGGGGTTGCTCTGAT |
| | | ATAG |
| SEQ ID | monkey | ATGGGGTCTCCCTCAGCCCCTCTTCACAGATGGTGCATCCCC |
| NO: 69 | CEA DNA | TGGCAGACGCTCCTGCTCACAGCCTCACTTCTAACCTTCTG |
| | | GAACCCGCCCACCACTGCCCAGCTCACTATTGAATCCAGGC |
| | | CGTTCAATGTTGCAGAGGGGAAGGAGGTTCTTCTACTTGCC |
| | | CACAATGTGTCCCAGAATCTTTTTGGCTACATTTGGTACAAG |
| | | GGAGAAAGAGTGGATGCCAGCCGTCGAATTGGATCATGTGT |
| | | AATAAGAACTCAACAAATTACCCCAGGGCCCGCACACAGCG |
| | | GTCGAGAGACAATAGACTTCAATGCATCCCTGCTGATCCAC |
| | | AATGTCACCCAGAGTGACACAGGATCCTACACCATACAAGT |
| | | CATAAAGGAAGATCTTGTGAATGAAGAAGCAACTGGCCAGT |
| | | TCCGGGTATACCCGGAGCTGCCCAAGCCCTACATCTCCAGC |
| | | AACAACTCCAACCCCGTGGAGGACAAGGATGCTGTGGCCTT |
| | | AACCTGTGAACCTGAGACTCAGGACACAACCTACCTGTGGT |
| | | GGGTAAACAATCAGAGCCTCCCGGTCAGTCCCAGGCTGGA |
| | | GCTGTCCAGTGACAACAGGACCCTCACTGTATTCAATATTCC |
| | | AAGAAATGACACAACATCCTACAAATGTGAAACCCAGAACC |
| | | CAGTGAGTGTCAGACGCAGCGACCCAGTCACCCTGAACGT |
| | | CCTCTATGGCCCGGATGCGCCCACCATTTCCCCTCTAAACAC |
| | | ACCTTACAGAGCAGGGGAAAATCTGAACCTCACCTGCCACG |
| | | CAGCCTCTAACCCAACTGCACAGTACTTTTGGTTTGTCAATG |
| | | GGACGTTCCAGCAATCCACACAAGAGCTCTTTATACCCAAC |
| | | ATCACCGTGAATAATAGCGGATCCTATATGTGCCAAGCCCAT |
| | | AACTCAGCCACTGGCCTCAATAGGACCACAGTCACGGCGAT |
| | | CACAGTCTACGCGGAGCTGCCCAAGCCCTACATCACCAGCA |
| | | ACAACTCCAACCCCATAGAGGACAAGGATGCTGTGACCTTA |
| | | ACCTGTGAACCTGAGACTCAGGACACAACCTACCTGTGGTG |
| | | GGTAAACAATCAGAGCCTCTCGGTCAGTTCCAGGCTGGAGC |
| | | TGTCCAATGACAACAGGACCCTCACTGTATTCAATATTCCAA |
| | | GAAACGACACAACGTTCTACGAATGTGAGACCCAGAACCC |
| | | AGTGAGTGTCAGACGCAGCGACCCAGTCACCCTGAATGTCC |
| | | TCTATGGCCCGGATGCGCCCACCATTTCCCCTCTAAACACAC |
| | | CTTACAGAGCAGGGGAAAATCTGAACCTCTCCTGCCACGCA |
| | | GCCTCTAACCCAGCTGCACAGTACTCTTGGTTTGTCAATGGG |
| | | ACGTTCCAGCAATCCACACAAGAGCTCTTTATACCCAACATC |
| | | ACCGTGAATAATAGCGGATCCTATATGTGCCAAGCCCATAAC |
| | | TCAGCCACTGGCCTCAATAGGACCACAGTCACGGCGATCAC |
| | | AGTCTATGTGGAGCTGCCCAAGCCCTACATCTCCAGCAACA |
| | | ACTCCAACCCCATAGAGGACAAGGATGCTGTGACCTTAACC |
| | | TGTGAACCTGTGGCTGAGAACACAACCTACCTGTGGTGGGT |
| | | AAACAATCAGAGCCTCTCGGTCAGTCCCAGGCTGCAGCTCT |
| | | CCAATGGCAACAGGATCCTCACTCTACTCAGTGTCACACGG |
| | | AATGACACAGGACCCTATGAATGTGGAATCCAGAACTCAGA |
| | | GAGTGCAAAACGCAGTGACCCAGTCACCCTGAATGTCACCT |
| | | ATGGCCCAGACACCCCCATCATATCCCCCCCAGACTTGTCTT |
| | | ACCGTTCGGGAGCAAACCTCAACCTCTCCTGCCACTCGGAC |
| | | TCTAACCCATCCCCGCAGTATTCTTGGCTTATCAATGGGACA |
| | | CTGCGGCAACACACACAAGTTCTCTTTATCTCCAAAATCACA |
| | | TCAAACAATAGCGGGGCCTATGCCTGTTTTGTCTCTAACTTG |
| | | GCTACCGGTCGCAATAACTCCATAGTCAAGAACATCTCAGTC |
| | | TCCTCTGGCGATTCAGCACCTGGAAGTTCTGGTCTCTCAGCT |
| | | AGGGCTACTGTCGGCATCATAATTGGAATGCTGGTTGGGGTT |
| | | GCTCTGATGTAG |
| SEQ ID | full-length | ATGGGACCCCCCTCAGCCCCTCCCTGCAGATTGCATGTCCCC |
| NO: 70 | human | TGGAAGGAGGTCCTGCTCACAGCCTCACTTCTAACCTTCTG |
| | CEACAM6 | GAACCCACCCACCACTGCCAAGCTCACTATTGAATCCACGC |
| | DNA | CGTTCAATGTCGCAGAGGGGAAGGAGGTTCTTCTACTCGCC |
| | | CACAACCTGCCCCAGAATCGTATTGGTTACAGCTGGTACAA |
| | | AGGCGAAAGAGTGGATGGCAACAGTCTAATTGTAGGATATG |
| | | TAATAGGAACTCAACAAGCTACCCCAGGGCCCGCATACAGT |
| | | GGTCGAGAGACAATATACCCCAATGCATCCCTGCTGATCCAG |
| | | AACGTCACCCAGAATGACACAGGATTCTATACCCTACAAGT |
| | | CATAAAGTCAGATCTTGTGAATGAAGAAGCAACCGGACAGT |
| | | TCCATGTATACCCGGAGCTGCCCAAGCCCTCCATCTCCAGCA |
| | | ACAACTCCAACCCCGTGGAGGACAAGGATGCTGTGGCCTTC |
| | | ACCTGTGAACCTGAGGTTCAGAACACAACCTACCTGTGGTG |
| | | GGTAAATGGTCAGAGCCTCCCGGTCAGTCCCAGGCTGCAGC |
| | | TGTCCAATGGCAACATGACCCTCACTCTACTCAGCGTCAAA |
| | | AGGAACGATGCAGGATCCTATGAATGTGAAATACAGAACCC |
| | | AGCGAGTGCCAACCGCAGTGACCCAGTCACCCTGAATGTCC |
| | | TCTATGGCCCAGATGTCCCCACCATTTCCCCCTCAAAGGCCA |
| | | ATTACCGTCCAGGGGAAAATCTGAACCTCTCCTGCCACGCA |
| | | GCCTCTAACCCACCTGCACAGTACTCTTGGTTTATCAATGGG |
| | | ACGTTCCAGCAATCCACACAAGAGCTCTTTATCCCCAACATC |
| | | ACTGTGAATAATAGCGGATCCTATATGTGCCAAGCCCATAAC |
| | | TCAGCCACTGGCCTCAATAGGACCACAGTCACGATGATCAC |
| | | AGTCTCTGGAAGTGCTCCTGTCCTCTCAGCTGTGGCCACCG |
| | | TCGGCATCACGATTGGAGTGCTGGCCAGGGTGGCTCTGATAT |
| | | AG |
| SEQ ID | 1-687 of | ATGGAGTCTCCCTCGGCCCCTCCCCACAGATGGTGCATCCCC |
| NO: 71 | huCEA | TGGCAGAGGCTCCTGCTCACAGCCTCACTTCTAACCTTCTG |
| | DNA | GAACCCGCCCACCACTGCCAAGCTCACTATTGAATCCACGC |
| | | CGTTCAATGTCGCAGAGGGGAAGGAGGTGCTTCTACTTGTC |
| | | CACAATCTGCCCCAGCATCTTTTTGGCTACAGCTGGTACAAA |
| | | GGTGAAAGAGTGGATGGCAACCGTCAAATTATAGGATATGT |
| | | AATAGGAACTCAACAAGCTACCCCAGGGCCCGCATACAGTG |
| | | GTCGAGAGATAATATACCCCAATGCATCCCTGCTGATCCAGA |
| | | ACATCATCCAGAATGACACAGGATTCTACACCCTACACGTCA |
| | | TAAAGTCAGATCTTGTGAATGAAGAAGCAACTGGCCAGTTC |
| | | CGGGTATACCCGGAGCTGCCCAAGCCCTCCATCTCCAGCAA |
| | | CAACTCCAAACCCGTGGAGGACAAGGATGCTGTGGCCTTCA |
| | | CCTGTGAACCTGAGACTCAGGACGCAACCTACCTGTGGTGG |
| | | GTAAACAATCAGAGCCTCCCGGTCAGTCCCAGGCTGCAGCT |
| | | GTCCAATGGCAACAGGACCCTCACTCTATTCAATGTCACAA |
| | | GAAATGACACAGCAAGCTACAAATGTGAAACCCAGAACCC |
| | | AGTGAGTGCCAGGCGCAGTGATTCAGTCATCCTGAATGTCC |
| | | TCTATGGCCCGGATGCCCCCACCATTTCCCCTCTAAACACAT |
| | | CTTACAGATCAGGGGAAAATCTGAACCTCTCCTGCCACGCA |
| | | GCCTCTAACCCACCTGCACAGTACTCTTGGTTTGTCAATGGG |
| | | ACTTTCCAGCAATCCACCCAAGAGCTCTTTATCCCCAACATC |
| | | ACTGTGAATAATAGTGGATCCTATACGTGCCAAGCCCATAAC |
| | | TCAGACACTGGCCTCAATAGGACCACAGTCACGACGATCAC |
| | | AGTCTATGCAGAGCCACCCAAACCCTTCATCACCAGCAACA |
| | | ACTCCAACCCCGTGGAGGATGAGGATGCTGTAGCCTTAACC |
| | | TGTGAACCTGAGATTCAGAACACAACCTACCTGTGGTGGGT |
| | | AAATAATCAGAGCCTCCCGGTCAGTCCCAGGCTGCAGCTGT |
| | | CCAATGACAACAGGACCCTCACTCTACTCAGTGTCACAAGG |
| | | AATGATGTAGGACCCTATGAGTGTGGAATCCAGAACGAATT |
| | | AAGTGTTGACCACAGCGACCCAGTCATCCTGAATGTCCTCT |
| | | ATGGCCCAGACGACCCCACCATTTCCCCCTCATACACCTATT |
| | | ACCGTCCAGGGGTGAACCTCAGCCTCTCCTGCCATGCAGCC |
| | | TCTAACCCACCTGCACAGTATTCTTGGCTGATTGATGGGAAC |
| | | ATCCAGCAACACACACAAGAGCTCTTTATCTCCAACATCAC |
| | | TGAGAAGAACAGCGGACTCTATACCTGCCAGGCCAATAACT |
| | | CAGCCAGTGGCCACAGCAGGACTACAGTCAAGACAATCAC |
| | | AGTCTCTGCGGAGCTGCCCAAGCCCTCCATCTCCAGCAACA |
| | | ACTCCAAACCCGTGGAGGACAAGGATGCTGTGGCCTTCACC |
| | | TGTGAACCTGAGGCTCAGAACACAACCTACCTGTGGTGGGT |
| | | AAATGGTCAGAGCCTCCCAGTCAGTCCCAGGCTGCAGCTGT |
| | | CCAATGGCAACAGGACCCTCACTCTATTCAATGTCACAAGA |
| | | AATGACGCAAGAGCCTATGTATGTGGAATCCAGAACTCAGT |
| | | GAGTGCAAACCGCAGTGACCCAGTCACCCTGGATGTCCTCT |
| | | ATGGGCCGGACACCCCCATCATTTCCCCCCCAGACTCGTCTT |
| | | ACCTTTCGGGAGCGAACCTCAACCTCTCCTGCCACTCGGCC |
| | | TCTAACCCATCCCCGCAGTATTCTTGGCGTATCAATGGGATA |
| | | CCGCAGCAACACACACAAGTTCTCTTTATCGCCAAAATCAC |
| | | GCCAAATAATAACGGGACCTATGCCTGTTTTGTCTCTAACTT |
| | | GGCTACTGGCCGCAATAATTCCATAGTCAAGAGCATCACAGT |
| | | CTCTGCATCTGGAACTTCTCCTGGTCTCTCAGCTGGGGCCCA |
| | | TCACCATCACCATCAC |
| SEQ ID | 1-690 of | ATGGGGTCTCCCTCAGCCCCTCTTCACAGATGGTGCATCCCC |
| NO: 72 | monkey | TGGCAGACGCTCCTGCTCACAGCCTCACTTCTAACCTTCTG |
| | CEA DNA | GAACCCGCCCACCACTGCCCAGCTCACTATTGAATCCAGGC |
| | | CGTTCAATGTTGCAGAGGGGAAGGAGGTTCTTCTACTTGCC |
| | | CACAATGTGTCCCAGAATCTTTTTGGCTACATTTGGTACAAG |
| | | GGAGAAAGAGTGGATGCCAGCCGTCGAATTGGATCATGTGT |
| | | AATAAGAACTCAACAAATTACCCCAGGGCCCGCACACAGCG |
| | | GTCGAGAGACAATAGACTTCAATGCATCCCTGCTGATCCAC |
| | | AATGTCACCCAGAGTGACACAGGATCCTACACCATACAAGT |
| | | CATAAAGGAAGATCTTGTGAATGAAGAAGCAACTGGCCAGT |
| | | TCCGGGTATACCCGGAGCTGCCCAAGCCCTACATCTCCAGC |
| | | AACAACTCCAACCCCGTGGAGGACAAGGATGCTGTGGCCTT |
| | | AACCTGTGAACCTGAGACTCAGGACACAACCTACCTGTGGT |
| | | GGGTAAACAATCAGAGCCTCCCGGTCAGTCCCAGGCTGGA |
| | | GCTGTCCAGTGACAACAGGACCCTCACTGTATTCAATATTCC |
| | | AAGAAATGACACAACATCCTACAAATGTGAAACCCAGAACC |
| | | CAGTGAGTGTCAGACGCAGCGACCCAGTCACCCTGAACGT |
| | | CCTCTATGGCCCGGATGCGCCCACCATTTCCCCTCTAAACAC |
| | | ACCTTACAGAGCAGGGGAAAATCTGAACCTCACCTGCCACG |
| | | CAGCCTCTAACCCAACTGCACAGTACTTTTGGTTTGTCAATG |
| | | GGACGTTCCAGCAATCCACACAAGAGCTCTTTATACCCAAC |
| | | ATCACCGTGAATAATAGCGGATCCTATATGTGCCAAGCCCAT |
| | | AACTCAGCCACTGGCCTCAATAGGACCACAGTCACGGCGAT |
| | | CACAGTCTACGCGGAGCTGCCCAAGCCCTACATCACCAGCA |
| | | ACAACTCCAACCCCATAGAGGACAAGGATGCTGTGACCTTA |
| | | ACCTGTGAACCTGAGACTCAGGACACAACCTACCTGTGGTG |
| | | GGTAAACAATCAGAGCCTCTCGGTCAGTTCCAGGCTGGAGC |
| | | TGTCCAATGACAACAGGACCCTCACTGTATTCAATATTCCAA |
| | | GAAACGACACAACGTTCTACGAATGTGAGACCCAGAACCC |
| | | AGTGAGTGTCAGACGCAGCGACCCAGTCACCCTGAATGTCC |
| | | TCTATGGCCCGGATGCGCCCACCATTTCCCCTCTAAACACAC |
| | | CTTACAGAGCAGGGGAAAATCTGAACCTCTCCTGCCACGCA |
| | | GCCTCTAACCCAGCTGCACAGTACTCTTGGTTTGTCAATGGG |
| | | ACGTTCCAGCAATCCACACAAGAGCTCTTTATACCCAACATC |
| | | ACCGTGAATAATAGCGGATCCTATATGTGCCAAGCCCATAAC |
| | | TCAGCCACTGGCCTCAATAGGACCACAGTCACGGCGATCAC |
| | | AGTCTATGTGGAGCTGCCCAAGCCCTACATCTCCAGCAACA |
| | | ACTCCAACCCCATAGAGGACAAGGATGCTGTGACCTTAACC |
| | | TGTGAACCTGTGGCTGAGAACACAACCTACCTGTGGTGGGT |
| | | AAACAATCAGAGCCTCTCGGTCAGTCCCAGGCTGCAGCTCT |
| | | CCAATGGCAACAGGATCCTCACTCTACTCAGTGTCACACGG |
| | | AATGACACAGGACCCTATGAATGTGGAATCCAGAACTCAGA |
| | | GAGTGCAAAACGCAGTGACCCAGTCACCCTGAATGTCACCT |
| | | ATGGCCCAGACACCCCCATCATATCCCCCCCAGACTTGTCTT |
| | | ACCGTTCGGGAGCAAACCTCAACCTCTCCTGCCACTCGGAC |
| | | TCTAACCCATCCCCGCAGTATTCTTGGCTTATCAATGGGACA |
| | | CTGCGGCAACACACACAAGTTCTCTTTATCTCCAAAATCACA |
| | | TCAAACAATAGCGGGGCCTATGCCTGTTTTGTCTCTAACTTG |
| | | GCTACCGGTCGCAATAACTCCATAGTCAAGAACATCTCAGTC |
| | | TCCTCTGGCGATTCAGCACCTGGAAGTTCTGGTCTCTCAGCT |
| | | AGGGCTCATCACCATCACCATCAC |
| SEQ ID | 1-320 of | ATGGGACCCCCCTCAGCCCCTCCCTGCAGATTGCATGTCCCC |
| NO: 73 | CEACAM6 | TGGAAGGAGGTCCTGCTCACAGCCTCACTTCTAACCTTCTG |
| | DNA | GAACCCACCCACCACTGCCAAGCTCACTATTGAATCCACGC |
| | | CGTTCAATGTCGCAGAGGGGAAGGAGGTTCTTCTACTCGCC |
| | | CACAACCTGCCCCAGAATCGTATTGGTTACAGCTGGTACAA |
| | | AGGCGAAAGAGTGGATGGCAACAGTCTAATTGTAGGATATG |
| | | TAATAGGAACTCAACAAGCTACCCCAGGGCCCGCATACAGT |
| | | GGTCGAGAGACAATATACCCCAATGCATCCCTGCTGATCCAG |
| | | AACGTCACCCAGAATGACACAGGATTCTATACCCTACAAGT |
| | | CATAAAGTCAGATCTTGTGAATGAAGAAGCAACCGGACAGT |
| | | TCCATGTATACCCGGAGCTGCCCAAGCCCTCCATCTCCAGCA |
| | | ACAACTCCAACCCCGTGGAGGACAAGGATGCTGTGGCCTTC |
| | | ACCTGTGAACCTGAGGTTCAGAACACAACCTACCTGTGGTG |
| | | GGTAAATGGTCAGAGCCTCCCGGTCAGTCCCAGGCTGCAGC |
| | | TGTCCAATGGCAACATGACCCTCACTCTACTCAGCGTCAAA |
| | | AGGAACGATGCAGGATCCTATGAATGTGAAATACAGAACCC |
| | | AGCGAGTGCCAACCGCAGTGACCCAGTCACCCTGAATGTCC |
| | | TCTATGGCCCAGATGTCCCCACCATTTCCCCCTCAAAGGCCA |
| | | ATTACCGTCCAGGGGAAAATCTGAACCTCTCCTGCCACGCA |
| | | GCCTCTAACCCACCTGCACAGTACTCTTGGTTTATCAATGGG |
| | | ACGTTCCAGCAATCCACACAAGAGCTCTTTATCCCCAACATC |
| | | ACTGTGAATAATAGCGGATCCTATATGTGCCAAGCCCATAAC |
| | | TCAGCCACTGGCCTCAATAGGACCACAGTCACGATGATCAC |
| | | AGTCTCTGGACATCACCATCACCATCAC |
| SEQ ID | 1-78 of | ATGGAGTCTCCCTCGGCCCCTCCCCACAGATGGTGCATCCCC |
| NO: 74 | huCEA | TGGCAGAGGCTCCTGCTCACAGCCTCACTTCTAACCTTCTG |
| | DNA | GAACCCGCCCACCACTGCCAAGCTCACTATTGAATCCACGC |
| | | CGTTCAATGTCGCAGAGGGGAAGGAGGTGCTTCTACTTGTC |
| | | CACAATCTGCCCCAGCATCTTTTTGGCTACAGCTGGTACAAA |
| | | GGTGAAAGAGTGGATGGCAACCGTCAAATTATAGGATATGT |
| | | AATAGGAACTCAACAAGCTACCCCAGGGCCCGCATACAGTG |
| | | GTCGAGAGATAATATACCCCAATGCATCCCTGCTGATCCAGA |
| | | ACATCATCCAGAAT |
| SEQ ID | 398-687 of | GAATTAAGTGTTGACCACAGCGACCCAGTCATCCTGAATGT |
| NO: 75 | huCEA | CCTCTATGGCCCAGACGACCCCACCATTTCCCCCTCATACAC |
| | DNA | CTATTACCGTCCAGGGGTGAACCTCAGCCTCTCCTGCCATGC |
| | | AGCCTCTAACCCACCTGCACAGTATTCTTGGCTGATTGATGG |
| | | GAACATCCAGCAACACACACAAGAGCTCTTTATCTCCAACA |
| | | TCACTGAGAAGAACAGCGGACTCTATACCTGCCAGGCCAAT |
| | | AACTCAGCCAGTGGCCACAGCAGGACTACAGTCAAGACAA |
| | | TCACAGTCTCTGCGGAGCTGCCCAAGCCCTCCATCTCCAGC |
| | | AACAACTCCAAACCCGTGGAGGACAAGGATGCTGTGGCCT |
| | | TCACCTGTGAACCTGAGGCTCAGAACACAACCTACCTGTGG |
| | | TGGGTAAATGGTCAGAGCCTCCCAGTCAGTCCCAGGCTGCA |
| | | GCTGTCCAATGGCAACAGGACCCTCACTCTATTCAATGTCAC |
| | | AAGAAATGACGCAAGAGCCTATGTATGTGGAATCCAGAACT |
| | | CAGTGAGTGCAAACCGCAGTGACCCAGTCACCCTGGATGTC |
| | | CTCTATGGGCCGGACACCCCCATCATTTCCCCCCCAGACTCG |
| | | TCTTACCTTTCGGGAGCGAACCTCAACCTCTCCTGCCACTC |
| | | GGCCTCTAACCCATCCCCGCAGTATTCTTGGCGTATCAATGG |
| | | GATACCGCAGCAACACACACAAGTTCTCTTTATCGCCAAAA |
| | | TCACGCCAAATAATAACGGGACCTATGCCTGTTTTGTCTCTA |
| | | ACTTGGCTACTGGCCGCAATAATTCCATAGTCAAGAGCATCA |
| | | CAGTCTCTGCATCTGGAACTTCTCCTGGTCTCTCAGCTGGGG |
| | | CC |
| SEQ ID | CEA-v | ATGGAGTCTCCCTCGGCCCCTCCCCACAGATGGTGCATCCCC |
| NO: 76 | DNA | TGGCAGAGGCTCCTGCTCACAGCCTCACTTCTAACCTTCTG |
| | | GAACCCGCCCACCACTGCCAAGCTCACTATTGAATCCACGC |
| | | CGTTCAATGTCGCAGAGGGGAAGGAGGTGCTTCTACTTGTC |
| | | CACAATCTGCCCCAGCATCTTTTTGGCTACAGCTGGTACAAA |
| | | GGTGAAAGAGTGGATGGCAACCGTCAAATTATAGGATATGT |
| | | AATAGGAACTCAACAAGCTACCCCAGGGCCCGCATACAGTG |
| | | GTCGAGAGATAATATACCCCAATGCATCCCTGCTGATCCAGA |
| | | ACATCATCCAGAATGAATTAAGTGTTGACCACAGCGACCCA |
| | | GTCATCCTGAATGTCCTCTATGGCCCAGACGACCCCACCATT |
| | | TCCCCCTCATACACCTATTACCGTCCAGGGGTGAACCTCAGC |
| | | CTCTCCTGCCATGCAGCCTCTAACCCACCTGCACAGTATTCT |
| | | TGGCTGATTGATGGGAACATCCAGCAACACACACAAGAGCT |
| | | CTTTATCTCCAACATCACTGAGAAGAACAGCGGACTCTATAC |
| | | CTGCCAGGCCAATAACTCAGCCAGTGGCCACAGCAGGACTA |
| | | CAGTCAAGACAATCACAGTCTCTGCGGAGCTGCCCAAGCCC |
| | | TCCATCTCCAGCAACAACTCCAAACCCGTGGAGGACAAGGA |
| | | TGCTGTGGCCTTCACCTGTGAACCTGAGGCTCAGAACACAA |
| | | CCTACCTGTGGTGGGTAAATGGTCAGAGCCTCCCAGTCAGT |
| | | CCCAGGCTGCAGCTGTCCAATGGCAACAGGACCCTCACTCT |
| | | ATTCAATGTCACAAGAAATGACGCAAGAGCCTATGTATGTG |
| | | GAATCCAGAACTCAGTGAGTGCAAACCGCAGTGACCCAGT |
| | | CACCCTGGATGTCCTCTATGGGCCGGACACCCCCATCATTTC |
| | | CCCCCCAGACTCGTCTTACCTTTCGGGAGCGAACCTCAACC |
| | | TCTCCTGCCACTCGGCCTCTAACCCATCCCCGCAGTATTCTT |
| | | GGCGTATCAATGGGATACCGCAGCAACACACACAAGTTCTC |
| | | TTTATCGCCAAAATCACGCCAAATAATAACGGGACCTATGCC |
| | | TGTTTTGTCTCTAACTTGGCTACTGGCCGCAATAATTCCATA |
| | | GTCAAGAGCATCACAGTCTCTGCATCTGGAACTTCTCCTGG |
| | | TCTCTCAGCTGGGGCCCATCACCATCACCATCAC |
| SEQ ID | 1-273 of | ATGGGACCCCCCTCAGCCCCTCCCTGCAGATTGCATGTCCCC |
| NO: 77 | CEACAM6 | TGGAAGGAGGTCCTGCTCACAGCCTCACTTCTAACCTTCTG |
| | DNA | GAACCCACCCACCACTGCCAAGCTCACTATTGAATCCACGC |
| | | CGTTCAATGTCGCAGAGGGGAAGGAGGTTCTTCTACTCGCC |
| | | CACAACCTGCCCCAGAATCGTATTGGTTACAGCTGGTACAA |
| | | AGGCGAAAGAGTGGATGGCAACAGTCTAATTGTAGGATATG |
| | | TAATAGGAACTCAACAAGCTACCCCAGGGCCCGCATACAGT |
| | | GGTCGAGAGACAATATACCCCAATGCATCCCTGCTGATCCAG |
| | | AACGTCACCCAGAATGACACAGGATTCTATACCCTACAAGT |
| | | CATAAAGTCAGATCTTGTGAATGAAGAAGCAACCGGACAGT |
| | | TCCATGTATACCCGGAGCTGCCCAAGCCCTCCATCTCCAGCA |
| | | ACAACTCCAACCCCGTGGAGGACAAGGATGCTGTGGCCTTC |
| | | ACCTGTGAACCTGAGGTTCAGAACACAACCTACCTGTGGTG |
| | | GGTAAATGGTCAGAGCCTCCCGGTCAGTCCCAGGCTGCAGC |
| | | TGTCCAATGGCAACATGACCCTCACTCTACTCAGCGTCAAA |
| | | AGGAACGATGCAGGATCCTATGAATGTGAAATACAGAACCC |
| | | AGCGAGTGCCAACCGCAGTGACCCAGTCACCCTGAATGTCC |
| | | TCTATGGCCCAGATGTCCCCACCATTTCC |
| SEQ ID | 596-687 of | CCCATCATTTCCCCCCCAGACTCGTCTTACCTTTCGGGAGCG |
| NO: 78 | huCEA | AACCTCAACCTCTCCTGCCACTCGGCCTCTAACCCATCCCCG |
| | DNA | CAGTATTCTTGGCGTATCAATGGGATACCGCAGCAACACACA |
| | | CAAGTTCTCTTTATCGCCAAAATCACGCCAAATAATAACGGG |
| | | ACCTATGCCTGTTTTGTCTCTAACTTGGCTACTGGCCGCAAT |
| | | AATTCCATAGTCAAGAGCATCACAGTCTCTGCATCTGGAACT |
| | | TCTCCTGGTCTCTCAGCTGGGGCC |
| SEQ ID | CHIM DNA | ATGGGACCCCCCTCAGCCCCTCCCTGCAGATTGCATGTCCCC |
| NO: 79 | | TGGAAGGAGGTCCTGCTCACAGCCTCACTTCTAACCTTCTG |
| | | GAACCCACCCACCACTGCCAAGCTCACTATTGAATCCACGC |
| | | CGTTCAATGTCGCAGAGGGGAAGGAGGTTCTTCTACTCGCC |
| | | CACAACCTGCCCCAGAATCGTATTGGTTACAGCTGGTACAA |
| | | AGGCGAAAGAGTGGATGGCAACAGTCTAATTGTAGGATATG |
| | | TAATAGGAACTCAACAAGCTACCCCAGGGCCCGCATACAGT |
| | | GGTCGAGAGACAATATACCCCAATGCATCCCTGCTGATCCAG |
| | | AACGTCACCCAGAATGACACAGGATTCTATACCCTACAAGT |
| | | CATAAAGTCAGATCTTGTGAATGAAGAAGCAACCGGACAGT |
| | | TCCATGTATACCCGGAGCTGCCCAAGCCCTCCATCTCCAGCA |
| | | ACAACTCCAACCCCGTGGAGGACAAGGATGCTGTGGCCTTC |
| | | ACCTGTGAACCTGAGGTTCAGAACACAACCTACCTGTGGTG |
| | | GGTAAATGGTCAGAGCCTCCCGGTCAGTCCCAGGCTGCAGC |
| | | TGTCCAATGGCAACATGACCCTCACTCTACTCAGCGTCAAA |
| | | AGGAACGATGCAGGATCCTATGAATGTGAAATACAGAACCC |
| | | AGCGAGTGCCAACCGCAGTGACCCAGTCACCCTGAATGTCC |
| | | TCTATGGCCCAGATGTCCCCACCATTTCCCCCATCATTTCCCC |
| | | CCCAGACTCGTCTTACCTTTCGGGAGCGAACCTCAACCTCT |
| | | CCTGCCACTCGGCCTCTAACCCATCCCCGCAGTATTCTTGGC |
| | | GTATCAATGGGATACCGCAGCAACACACACAAGTTCTCTTTA |
| | | TCGCCAAAATCACGCCAAATAATAACGGGACCTATGCCTGTT |
| | | TTGTCTCTAACTTGGCTACTGGCCGCAATAATTCCATAGTCA |
| | | AGAGCATCACAGTCTCTGCATCTGGAACTTCTCCTGGTCTCT |
| | | CAGCTGGGGCCCATCACCATCACCATCAC |

### Example 2. Cloning and Sequence Analysis of CEA Antibodies

Murine hybridoma cells were harvested to prepare total RNAs using an Ultrapure RNA kit (Cat. No. 74104, QIAGEN, Germany) based on the manufacturer's protocol. The 1^{st} strand cDNAs were synthesized using a cDNA synthesis kit from Invitrogen (Cat. No. 18080-051) and PCR amplification of VH and VL genes of murine monoclonal antibodies was performed using a PCR kit (Cat. No. CW0686, CWBio, Beijing, China). The oligo primers used for antibody cDNAs cloning of heavy chain variable region (VH) and kappa light chain variable region (VL) were synthesized based on the sequences reported previously (Brocks et al., Mol Med. 2001 7(7):461-9.). PCR products were then subcloned into the pEASY-Blunt cloning vector (Cat. No. CB101-02, TransGen, China) and sequenced. The amino acid sequences of VH and VL regions were determined from the DNA sequencing results.

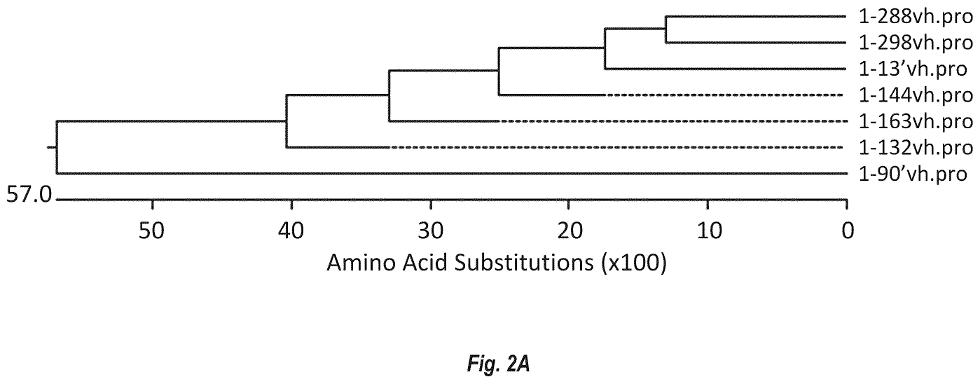

The monoclonal antibodies were analyzed by comparing sequence homology and grouped based on sequence similarity (FIG. 2). Complementary determinant regions (CDRs) were defined based on the IMGT (Lefranc et al., 1999 Nucleic Acids Research 27:209-212) system by sequence annotation. The amino acid sequences of a representative clone BGA7592 are listed in Table 3.

TABLE 3
Amino acid sequences

| | | | |
|---|---|---|---|
| BGA7592 | SEQ ID | HCDR1 | GYIFTSYW |
| (IMGT) | NO: 80 | (IMGT) | |
| | SEQ ID | HCDR2 | INPNTGYT |
| | NO: 81 | (IMGT) | |
| | SEQ ID | HCDR3 | AREYGNYNYPLDY |
| | NO: 3 | (IMGT) | |
| | SEQ ID | LCDR1 | ENIYGY |
| | NO: 82 | (IMGT) | |
| | SEQ ID | LCDR2 | NA |
| | NO: 83 | (IMGT) | |
| | SEQ ID | LCDR3 | QHHYGTPYT |
| | NO: 84 | (IMGT) | |
| BGA7592 | SEQ ID | VH | QVQLQQSGAELAKSGASVKMSCKASGYIFT |
| | NO: 85 | | SYWLHWVKQRPGQGLEWIGYINPNTGYTNY |
| | | | SQKFKDKATLTADKSSSTAYMQLSSLTSEDSA |
| | | | VYYCAREYGNYNYPLDYWGQGTSVTVSS |
| | SEQ ID | VL | DIQMTQSPASLSASVGETVTITCRASENIYGY |
| | NO: 86 | | LAWYQQKQGKSPQLLVFNAKNLVEGVPSRF |
| | | | SGSGSGTQFSLKINSLQPEDFGSYYCQHHYG |
| | | | TPYTFGGGTKLEIK |

### Example 3. Binding Profiles Determination of Purified Murine Anti-CEA Antibodies

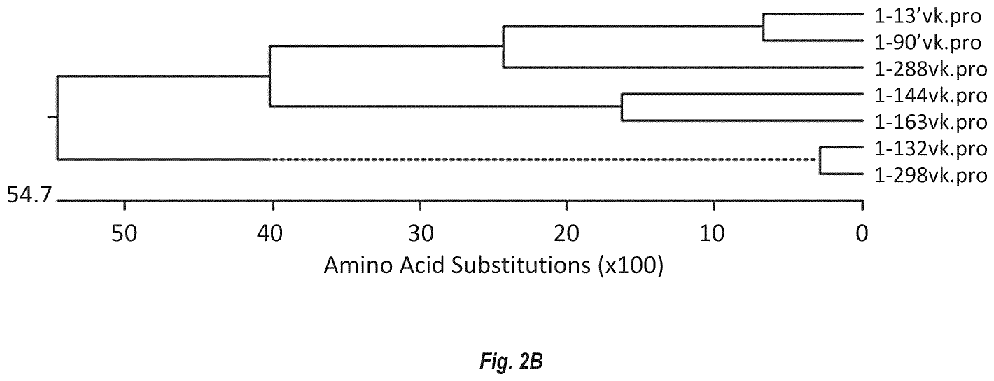

The CEA antibodies with specific binding for CEA as shown by ELISA and FACS, as well as without sCEA interference were characterized for their binding kinetics by surface plasmon resonance (SPR) assays using BIAcore™ T-200 (GE Life Sciences) (FIG. 3A). Briefly, anti-murine IgG antibody was immobilized on an activated CM5 biosensor chip (Cat. No. BR100530, GE Life Sciences). Purified murine antibodies were flowed over the chip surface and captured by anti-murine IgG antibody. Then a serial dilution (6.0 nM to 2150 nM) of purified CHIM, CEA-v, CEA, or monkey CEA recombinant proteins were flowed over the chip surface, and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of BGA7592 are shown below in Table 4.

TABLE 4
Comparison of BGA7592 binding affinities by SPR

| Antigen | K_{D} (M) |
|---|---|
| sCEA | ND |
| sCEA-v | 1.50E−07 |
| CHIM | 1.20E−07 |
| Monkey CEA | ND (not detectable due to weak binding |

The binding profiles of BGA7592 were checked via antigen ELISA. The binding of purified BGA7592 to huCEA and monkey CEA were observed, and indicated BGA7592 is a weak binder to soluble huCEA and monkey CEA, or that soluble CEA has a different conformation when immobilized (FIG. 3B). For this experiment, sCEA, CHIM, monkey CEA (“cynoCEA”), CEA-v, or bovine serum albumin (BSA) were coated in 96-well plates at a high concentration of 10 μg/ml overnight at 4° C. BGA7592 or control antibody ab4451 (Cat. No. ab4451, abcam, USA) were incubated for 1 hour at a concentration of 2 μg/ml. The HRP-linked anti-mouse IgG antibody (Cat. No. 7076S, Cell Signaling Technology, USA) and substrate (Cat. No. 00-4201-56, eBioscience, USA) were used for development, and absorbance signal at the wavelength of 450 nm was measured using a plate reader (SpectraMax Paradigm, Molecular Devices, USA).

### Example 4. Effects of Recombinant Soluble CEA on Binding of BGA7592 to CEA Expressing Cells

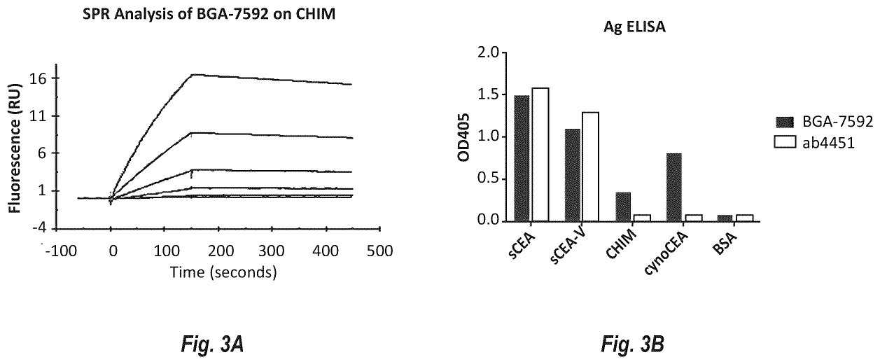

The presence of soluble CEA on the specific binding of various CEA antibodies to CEA expressing cells was evaluated via flow cytometry. In brief, human CEA-expressing cells (105 cells/well) were incubated with 2 μg/ml purified CEA murine monoclonal antibodies in the presence of 20 μg/ml extra recombinant soluble CEA proteins, followed by binding with Alexa Fluor-647-labeled goat anti-mouse IgG antibody (Cat. No. A0473, Beyotime Biotechnology, China). Cell fluorescence was quantified using a flow cytometer (Guava easyCyte™ 8HT, Merck-Millipore, USA). As shown in FIGS. 4A and 4B, the binding of BGA7592 to CEA expressing cells was not affected by the presence of soluble CEA.

### Example 5. Humanization of the Murine Anti-Human CEA Antibodies mAb Humanization and Engineering

For humanization of BGA7592, human germline IgG genes were searched for sequences that share high degrees of homology with the cDNA sequences of BGA7592 variable regions by sequence comparisons in the human immunoglobulin gene databases at IMGT and NCBI. The human IGVH and IGVL genes that are present in human antibody repertoires with high frequencies (Glanville et al., 2009 PNAS 106:20216-20221) and are highly homologous to BGA7592 were selected as the templates for humanization. Before humanization, BGA7592 heavy and light chain variable domains were fused to a wild type human IgG1 constant region designated as human IgG1 wt (SEQ ID NO:87) and a human kappa constant (CL) region (SEQ ID NO:88), respectively (Table 5).

TABLE 5
Amino acid sequences

| | | |
|---|---|---|
| SEQ ID | ASTKGPSVFPLAPSSKSTSGGTAALGCLVKDYFPEPVTVSWNSGALTS | IgG1wt |
| NO: 87 | GVHTFPAVLQSSGLYSLSSVVTVPSSSLGTQTYICNVNHKPSNTKVDK | |
| | KVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLT | |
| | VLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPGK | |
| SEQ ID | RTVAAPSVFIFPPSDEQLKSGTASVVCLLNNFYPREAKVQWKVDNAL | CL |
| NO: 88 | QSGNSQESVTEQDSKDSTYSLSSTLTLSKADYEKHKVYACEVTHQGL | |
| | SSPVTKSFNRGEC | |

Humanization was carried out by CDR-grafting (Methods in Molecular Biology, Vol 248: antibody Engineering, Methods and Protocols, Humana Press) and the BGA7592 antibody was engineered in the human IgG1 format. In the initial round of humanization, mutations from murine to human amino acid residues in framework regions were guided by the simulated 3D structure, and the murine framework residues of structural importance for maintaining the canonical structures of CDRs were retained in the 1′ version of humanized antibody BGA7592, BGA7592-1 (the amino acid sequences of the heavy chain and light chain are set forth in SEQ ID NOs:89 and 90) (Table 6).

TABLE 6
Amino acid sequences

| | | |
|---|---|---|
| SEQ ID | QVQLVQSGAEVKKPGASVKVSCKASGYIFTSYWLHWVRQAPGQGL | BGA7592-1 |
| NO: 89 | EWIGYINPNTGYTNYSQKFQGRVTMTRDTSTSTVYMELSSLRSEDTA | heavy |
| | VYYCAREYGNYNYPLDYWGQGTLVTVSSASTKGPSVFPLAPSSKSTS | chain |
| | GGTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKV | |
| | SNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQ | |
| | GNVFSCSVMHEALHNHYTQKSLSLSPGK | |
| SEQ ID | DIQMTQSPSSLSASVGDRVTITCRASENIYGYLAWYQQKPGKVPKLLI | BGA7592-1 |
| NO: 90 | YNAKNLVEGVPSRFSGSGSGTDFTLTISSLQPEDVATYYCQHHYGTPY | light |
| | TFGQGTKVEIKRTVAAPSVFIFPPSDEQLKSGTASVVCLLNNFYPREA | chain |
| | KVQWKVDNALQSGNSQESVTEQDSKDSTYSLSSTLTLSKADYEKHK | |
| | VYACEVTHQGLSSPVTKSFNRGEC | |

Specifically, CDRs of BGA7592-1 VL were grafted into the frameworks of human germline variable gene IGVK1-27 with 2 murine framework residues (N66 and V68) retained (the amino acid sequence of the light chain variable domain is set forth in SEQ ID NO:92). CDRs of BGA7592-1 VH were grafted into the frameworks of human germline variable gene IGVH1-46 with 5 murine framework (L39, I53, Y55, N66, S68) residues retained (the amino acid sequence of the heavy chain variable domain is set for in SEQ ID NO:91) (Table 7).

TABLE 7
Amino acid sequences

| | | |
|---|---|---|
| SEQ ID | QVQLVQSGAEVKKPGASVKVSCKASGYIFTSYWLHWVRQAPGQGL | BGA7592-1 |
| NO: 91 | EWIGYINPNTGYTNYSQKFQGRVTMTRDTSTSTVYMELSSLRSEDTA | VH |
| | VYYCAREYGNYNYPLDYWGQGTLVTVSS | |
| SEQ ID | DIQMTQSPSSLSASVGDRVTITCRASENIYGYLAWYQQKPGKVPKLLI | BGA7592-1 |
| NO: 92 | YNAKNLVEGVPSRFSGSGSGTDFTLTISSLQPEDVATYYCQHHYGTPY | VL |
| | TFGQGTKVEIK | |

BGA7592-1 was constructed as human full-length antibody format using in-house developed expression vectors that contain constant regions of a wild type human IgG1 with easy adapting subcloning sites. Expression and preparation of BGA7592-1 antibody was achieved by co-transfection of the above two constructs into 293G cells and by purification using a Protein A column (Cat. No. 17543802, GE Life Sciences). The purified antibodies were concentrated to 0.5-5 mg/mL in PBS and stored in aliquots in −80° C. freezer.

Using BGA7592-1, an additional number of single or multiple amino acid changes were made, converting the human residues in framework regions of VH and VL to corresponding murine germline residues, which include V68A, R72A and V79A in VH and V43S in VL, respectively. This resulted BGA7592-2 (V68A, R72A in VH), BGA7592-3 (V79A in VH), BGA7592-4 (V68A, R72A, V79A in VH), BGA7592-5 (V43S in VL), BGA7592-6 (V68A, R72A in VH, and V43S in VL), BGA7592-7 (V79A in VH, V43S in VL) and BGA7592-8 (V68A, R72A, V79A, in VH and V43S in VL). All antibodies which contained modifications had similar binding activities to BGA7592-1, and none of the changes abolished binding.

In order to remove post-translational modification (PTM) sites, further engineering was made by introducing mutations in CDRs and framework regions based on the BGA75921 sequence, which include N52T, N54Q, N59S, N102G, N104Q and S61A amino acid changes in the VH region. This resulted in BGA7592-1A (N52T (VH)), BGA7592-1B (N54Q (VH)), BGA7592-1C (N59S (VH)), BGA7592-1D (N102G (VH)), BGA7592-1E (N104Q (VH)) and BGA7592-1F (N54Q, N59S, S61 A (VH)) and all of the antibodies had similar binding specificity to BGA7592-1, with none of the changes abolishing binding. While maintaining specificity, amino acid compositions and expression levels were also considered. All humanization mutations were made using primers containing mutations at specific positions and a site directed mutagenesis kit (Cat. FM 111-02, TransGen, Beijing, China). The desired mutations were verified by sequence analysis. Comparing to BGA7592-1, BGA7592-1F had significantly reduced binding affinities with no glycosylation sites but had a high expression level (Table 8).

TABLE 8
Amino acid sequences

| | | |
|---|---|---|
| SEQ ID | DIQMTQSPSSLSASVGDRVTITCRASENIYGYLAWYQQKPGKVPKLLI | BGA7592-1 |
| NO: 93 | YNAKNLVEGVPSRFSGSGSGTDFTLTISSLQPEDVATYYCQHHYGTPY | and -1F VL |
| | TFGQGTKVEIK | |
| SEQ ID | QVQLVQSGAEVKKPGASVKVSCKASGYIFTSYWLHWVRQAPGQGL | BGA7592-1F |
| NO: 94 | EWIGYINPQTGYTSYAQKFQGRVTMTRDTSTSTVYMELSSLRSEDTA | VH |
| | VYYCAREYGNYNYPLDYWGQGTLVTVSS | |

### Example 6. Generation of Affinity Maturation Libraries

A phagemid vector pCANTAB 5E (GE Healthcare) was used by standard molecular biology techniques to construct a phagemid designed to display BGA7592-1F Fab fragments on the surface of M13 bacteriophage as a fusion with the N-terminus of a fragment of the gene-3 minor coat protein. There was an amber stop codon before the gene-3 sequence to allow expression of Fab fragments directly from phagemid clones. The phagemid was used as the template to construct phage-displayed libraries containing 10⁸ unique members.

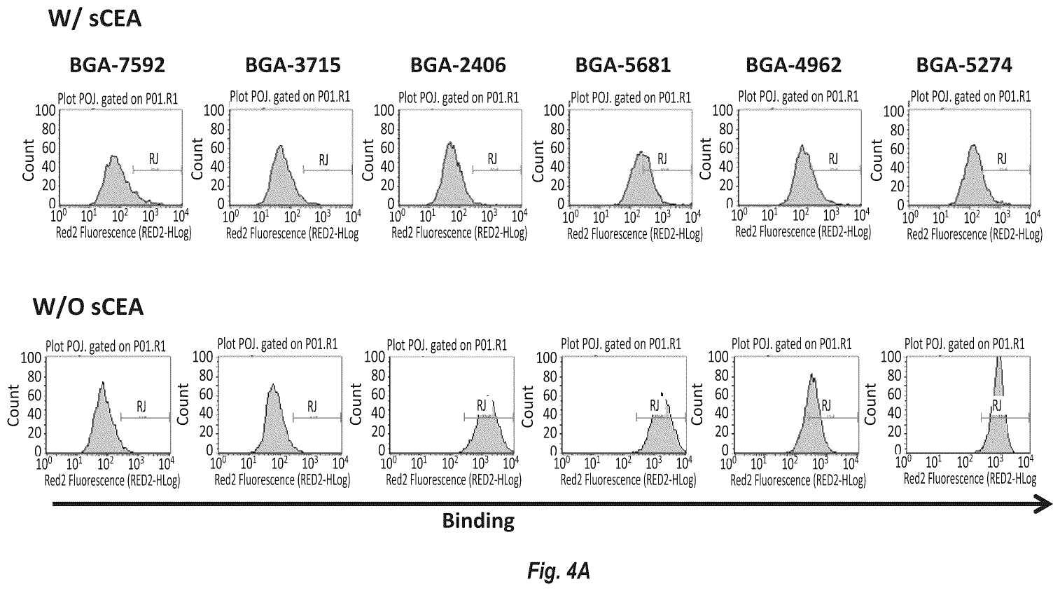

Two libraries (H-AM, L-AM) were constructed randomizing CDR positions in the heavy and light chains, respectively. All three CDRs were randomized in each library but each CDR had a maximum of one mutation in each clone except HCDR3, which could have two simultaneous mutations. Each position was randomized with an NNK codon (IUPAC code) encoding any amino acid or an amber stop codon. The combined heavy and light chain library designs had a potential diversity of 5.0×10⁶ unique full-length clones without stop or cysteine codons and an expected distribution of about 0.02%, 1.1%, 17% and 82% of clones with 0, 1, 2, and 3 mutations, respectively. A minor fraction of heavy chain clones was expected to have 4 mutations due to primer design in the HCDR3 region. As a first step, a DNA fragment was amplified using pCANTAB 5E as a template and primers which contains the randomized CDR3 positions (see FIGS. 5A and 5B). Then the PCR products were gel-purified and assembled with the primers which contain the randomized CDR2 positions. The procedure was repeated with the primers directed to random CDR1 positions. The resulting PCR products for heavy chain or light chain were then assembled with its corresponding CH fragment or CL fragment by overlapping PCR. The fragments were further assembled with the light chain or heavy chain with no mutations by overlapping PCR. The resulting fragments were then gel-purified and ligated with pCANTAB 5E after NcoI/NotI digestion. The purified ligations were transformed into TG1 bacteria by electroporation. Sequencing of 48 clones from each library confirmed the randomization of each position (data not shown), although not all amino acid mutations were observed in every position due to the limited sampling depth. About 52% and 55% of the light and heavy chain libraries had full-length randomized clones, enough to cover all the potential diversity of the design with the 10⁸ independent clones generated even with moderate incorporation biases in oligonucleotide synthesis and library construction.

### Example 7. Generation of Affinity Matured Humanized BGA7592 Variants

### Library Selection and Screening

Generation of affinity-matured humanized BGA7592 Fabs was carried out by phage display using standard protocols (Silacci et al., (2005) Proteomics, 5, 2340-50; Zhao et al., (2014) PLoS One, 9, e111339). For the first and second rounds of selections, competition selections were performed on immobilized CHIM in immune tubes (Cat. No. 470319, ThermoFisher). In brief, immunotubes were coated with 1 ml of CHIM (5 μg/ml in PBS) overnight at 4° C. All affinity maturation libraries were incubated with the coated immunotubes for 1 hour in the presence of various concentrations of BGA7592-1F IgG (round 1, 1 μg/ml; round 2, 5 μg/ml). For the third and fourth rounds of selections, cell panning was carried out using L929/huCEA cells (round 3) or LOVO cells (ATCC CCL-229) (round 4) with HEK293 cells as depletion cells. After four rounds of selections, individual clones were picked up and phage containing supernatants were prepared using standard protocols. ELISA-positive clones were sequenced, and mutation sites were analyzed.

### Analysis of Mutation Frequency in CDRs

The frequency of mutations in each CDR after four rounds of selection was relatively high, ranging from 17% in HCDR3 to 95% in LCDR2. Regarding the heavy chain, about half of clones identified in H-AM library were identical to the parental clone. The other clones contained one back-mutation at Q54N in HCDR2.

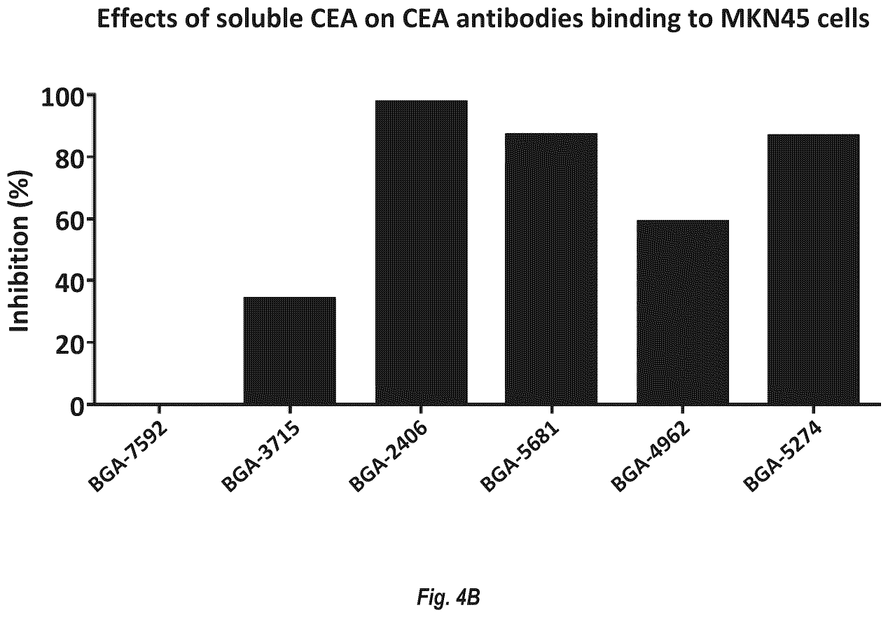

In analyzing the light chain, the mutations were much more diverse. Two sites had mutations occurring in almost all of clones in LCDR1, respectively. Light chain residues 29 and 31 were mutated from Ile to Gln and Gly to Gln in 47.09% and 35.29% of the clones, respectively. Position 29 not only had a high frequency of Gin mutation, but also had a subset of clones with a mutation to Tyrosine. Position 31 not only had a high frequency of Gln mutation, but also had about 12.5% chance to be mutated to Leu. Due to library design constraints, mutations in positions 29 and 31 were not found in combination with each other. However, mutations in each of these two sites were often combined with mutations in other CDRs. Regarding LCDR2, only A51 had mutations occurring in at least 64.71% clones, but with not any obvious pattern, which included large, hydrophobic and polar residues, such as Tyr, Phe, Thr and Asn. Regarding to LCDR3, two sites had mutations occurring in at least 50% clones. Light chain residues 90 and 92 were mutated form His to Leu and Tyr to Leu in 11.76% and 47.06% of the clones, respectively. FIG. 6 shows the sequence variance of CDR regions of light chain after four rounds of selections.

### Expression of Selected Humanized BGA 7592 Variants

Combinations of mutations were made. Light chain variable regions from selected phage clones were subcloned into a human kappa light chain expression mammalian expression vector. The light chain expression vectors were co-transfected into 293G cells with a mammalian expression vector expressing BGA7592-1F (also described herein as BGA5366) heavy chain at a 1:1 ratio. Versions of CEA antibodies were purified from culture supernatants by Protein A affinity chromatography (Cat. No. 17543802, GE Life Sciences). The purified antibodies were concentrated to 0.5-5 mg/mL in PBS and stored in aliquots in -80° C. freezer.

### Characterization of Affinity Matured Humanized BGA7592 Variants

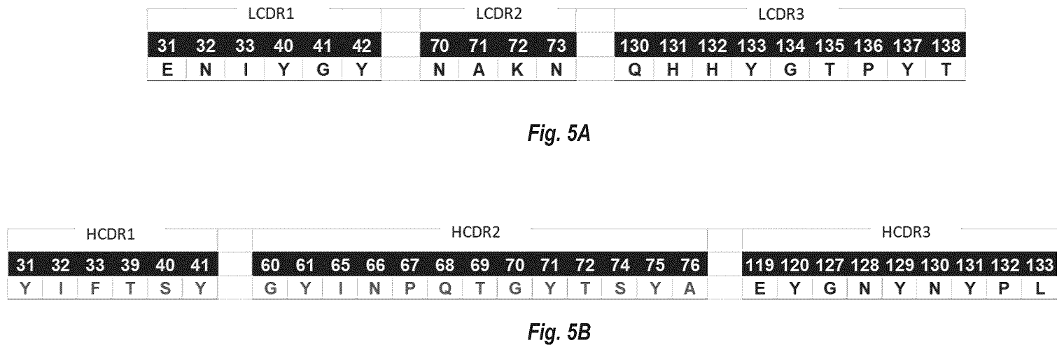

Affinity comparison of BGA7592-1F (BGA5366) and other affinity matured clones was made by SPR assay (Table 9) using BIAcore™ T-200 (GE Life Sciences) and flow cytometry (FIG. 7). For this experiment, anti-human IgG (Fc) antibody was immobilized on an activated CM5 biosensor chip (Cat. No. BR100839, GE Life Sciences). Anti-CEA antibodies were flowed over the chip surface and captured by anti-human Fab antibody. Then a serial dilution (1.37 nM to 333 nM) of CHIM was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). For flow cytometry, CEA-expressing cells (10′ cells/well) were incubated with various concentrations of purified affinity-matured antibodies, followed by binding with Alexa Fluro-647-labeled anti-hu IgG Fc antibody (Cat. No. 409320, BioLegend, USA). Cell fluorescence was quantified using a flow cytometer (Guava easyCyte™ 8HT, Merck-Millipore, USA). The equilibrium dissociation constant (KD) was calculated as the ratio k_{off}/kₒₙ. The BGA7592-1F-ph-L (BGA3676) (SEQ ID NO:95) and BGA7592-1F-ph-M (BGA2433) (SEQ ID NO:96) were shown with improved affinities to huCEA surface protein (Table 10).

TABLE 9
Comparison of binding affinities by SPR

| Sample ID | Ka | Kd | KD | Rmax |
|---|---|---|---|---|
| | (1/Ms) | (1/s) | (M) | (RU) |
| BGA7592-1F-ph-E (BGA7637) | 3.4E+3 | 3.5E−3 | 1.0E−6 | 284 |
| BGA7592-1F-ph-F (BGA9521) | 1.0E+2 | 1.2E−2 | 1.2E−4 | 4162 |
| BGA7592-1F-ph-G (BGA8232) | 6.4E+1 | 2.6E−3 | 4.1E−5 | 3487 |
| BGA7592-1F-ph-H (BGA5893) | 1.1E+2 | 2.8E−3 | 2.5E−5 | 1983 |
| BGA7592-1F-ph-I (BGA4429) | 2.0E+2 | 2.2E−3 | 1.1E−5 | 2248 |
| BGA7592-1F-ph-L (BGA3676) | 6.7E+3 | 3.0E−4 | 4.6E−8 | 184 |
| BGA7592-1F-ph-M (BGA2433) | 2.5E+3 | 2.5E−4 | 9.9E−8 | 224 |
| BGA7592-1F-ph-N (BGA1334) | 1.1E+3 | 1.0E−2 | 9.2E−6 | 1267 |
| BGA7592-1F (BGA5366) | 1.9E+3 | 1.5E−3 | 7.9E−7 | 367 |

TABLE 10
Amino acid sequences

| SEQ ID | DIQMTQSPSSLSASVGDRVTITCRASENQYGYLAWYQQKPGKVPK | BGA7592-1F- |
|---|---|---|
| NO: | LLIYNFKNLVEGVPSRFSGSGSGTDFTLTISSLQPEDVATYYCQHHL | ph-L |
| 95 | GTPYTFGQGTKVEIK | (BGA3676) VL |
| SEQ ID | DIQMTQSPSSLSASVGDRVTITCRASENQYGYLAWYQQKPGKVPK | BGA7592-1F- |
| NO: | LLIYNTKNLVEGVPSRFSGSGSGTDFTLTISSLQPEDVATYYCQHHL | ph-M |
| 96 | GTPYTFGQGTKVEIK | (BGA2433) VL |

### Example 8. Further Engineering of Affinity Matured Humanized BGA7592 Variants

Further engineering was made by introducing mutations in CDRs based on the BGA7592-1F-ph-M (BGA2433) template, which included W33Y, Q54N and S59N in the VH and T51Y in the VL. This resulted in BGA8179 (W33Y (VH)), BGA2107 (Q54N (VH)), BGA0089 (S59N (VH)), BGA1789 (T51Y (VL)) which all had improved binding activities compared to BGA7592-1F-ph-F (BGA9521), with the most improved antibody finally resulting in the BGA6710 antibody with the (W33Y (VH), T51Y (VL)) changes (Table 11), with the sequences shown in Table 12.

TABLE 11
Comparison of binding affinities to CHIM by SPR

| Sample ID | Ka(1/Ms) | Kd(1/s) | KD(M) | Rmax(RU) |
|---|---|---|---|---|
| BGA7592-1F-ph-M | 1.97E+04 | 2.48E−04 | 1.26E−08 | 97.2 |
| (BGA2433) | | | | |
| BGA8179 | 1.99E+04 | 2.82E−04 | 1.42E−08 | 129.1 |
| BGA2107 | 1.98E+04 | 3.80E−04 | 1.92E−08 | 71.1 |
| BGA0089 | 1.84E+04 | 3.94E−04 | 2.15E−08 | 59.4 |
| BGA1789 | 3.21E+04 | 4.63E−04 | 1.44E−08 | 92.9 |
| BGA6710 | 3.15E+04 | 4.75E−04 | 1.51E−08 | 112.4 |

TABLE 12
Amino acid and nucleic acid sequences of BGA6710

| | | | |
|---|---|---|---|
| BGA6710 | SEQ ID | VH | QVQLVQSGAEVKKPGASVKVSCKASGYIF |
| | NO: 97 | | TSYYLHWVRQAPGQGLEWIGYINPQTGY |
| | | | TSYAQKFQGRVTMTRDTSTSTVYMELSSL |
| | | | RSEDTAVYYCAREYGNYNYPLDYWGQGT |
| | | | LVTVSS |
| | SEQ ID | VL | DIQMTQSPSSLSASVGDRVTITCRASENQY |
| | NO: 15 | | GYLAWYQQKPGKVPKLLIYNYKNLVEGV |
| | | | PSRFSGSGSGTDFTLTISSLQPEDVATYYCQ |
| | | | HHLGTPYTFGQGTKVEIK |
| | SEQ ID | VH DNA | CAGGTGCAGCTGGTGCAGAGCGGCGCG |
| | NO: 98 | | GAAGTGAAAAAACCGGGCGCGAGCGTG |
| | | | AAAGTGAGCTGCAAAGCGAGCGGCTATA |
| | | | TTTTTACCAGCTATTACCTGCATTGGGTG |
| | | | CGCCAGGCGCCGGGCCAGGGCCTGGAAT |
| | | | GGATTGGCTATATTAACCCGCAGACCGGC |
| | | | TATACCAGCTATGCCCAGAAATTTCAGGG |
| | | | CCGCGTGACCATGACCCGCGATACCAGC |
| | | | ACCAGCACCGTGTATATGGAACTGAGCA |
| | | | GCCTGCGCAGCGAAGATACCGCGGTGTA |
| | | | TTATTGCGCGCGCGAATATGGCAACTATA |
| | | | ACTATCCGCTGGATTATTGGGGCCAGGGC |
| | | | ACCCTGGTGACCGTGAGCAGC |
| | SEQ ID | VL DNA | GATATTCAGATGACCCAGAGCCCGAGCA |
| | NO: 17 | | GCCTGAGCGCGAGCGTGGGCGATCGCGT |
| | | | GACCATTACCTGCCGCGCGAGCGAAAAC |
| | | | CAGTATGGCTATCTGGCGTGGTATCAGCA |
| | | | GAAACCGGGCAAAGTGCCGAAACTGCT |
| | | | GATTTATAACTATAAAAACCTGGTGGAAG |
| | | | GCGTGCCGAGCCGCTTTAGCGGCAGCGG |
| | | | CAGCGGCACCGATTTTACCCTGACCATTA |
| | | | GCAGCCTGCAGCCGGAAGATGTGGCGAC |
| | | | CTATTATTGCCAGCATCATCTGGGCACCC |
| | | | CGTATACCTTTGGCCAGGGCACCAAAGT |
| | | | GGAAATTAAA |

### Example 9. Optimization of BGA6710

To further improve the biochemical/biophysical properties, optimization of BGA6710 was made by introducing substitutions in CDR and framework regions (Table 13). The large, hydrophobic residues were chosen and changed to polar residues, except for K13 and Q53, which are selected based on observed differences among human VH germlines. The considerations include amino acid compositions, heat stability (Tm), surface hydrophobicity and isoelectronic points (pIs) while maintaining functional activities. The variants were expressed in Fab format by cloning into the vector pCANTAB-5E as described in Example 6. The Fab-containing supernatants were then screened by ELISA and SPR analysis for CEA binding. The variants without significant affinity reduction were selected and the residues which can tolerate substitutions were identified. It was demonstrated L92E in the light chain, K13E, Q54E, Y57D/E and Y57K in the heavy chain have minimal influence on the affinity.

Thus, the BGA6710 variants in IgG format with single identified mutation or combinations were expressed and purified as described in Example 8. SPR study and FACS analysis were performed and summarized in Table 14. It was confirmed that no changes in specificity and epitope occurred due to the introduced amino acid substitutions (data not shown). Taken together, the results demonstrated these single or combined mutations (K13E, Q54E, Y57D and Y57K in the heavy chain, L92E in the light chain) have minimal effects on the affinity, except for L92E, which slightly reduces the binding affinity to CEA. In summary, the Y57K change optimized the BGA6710 antibody for expression, CEA binding and affinity, which resulted in BGA5384 (Table 1).

TABLE 13
Summary of residues for substitution

| Residue | AA substitution |
|---|---|
| H:K13 | E |
| H:Y32 | H, N, Q, D, E, K |
| H:Y33 | H, N, Q, D, E, K |
| H:Q53 | A, D, G, N, S, T, Y, R, H, |
| H:Y57 | H, N, Q, D, E, K |
| H:Y100 | H, N, Q, D, E, K |
| H:Y105 | H, N, Q, D, E, K |
| L:V15 | T, P, L |
| L:Y30 | H, N, Q, D, E, K |
| L:Y32 | H, N, Q, D, E, K |
| L:Y49 | H, N, Q, D, E, K |
| L:P80 | S, T, A |
| L:L92 | H, N, Q, D, E, K |

TABLE 14
Summary of affinity measurement of BGA6710 variants by SPR

| | | | | Affinity | |
|---|---|---|---|---|---|
| | Kinetics (huCEA) | | | (MKN45 cells) | |
| BGA6710 | ka | kd | | | EC₅₀ |
| variants | (1/Ms) | (1/s) | KD (M) | Top | (μg/ml) |
| L92E, Y57K | 3.34E+04 | 1.77E−04 | 5.29E−09 | 969.9 | 1.09 |
| L92E | 4.17E+04 | 1.88E−04 | 4.50E−09 | 945.4 | 1.19 |
| Q54E, Y57K | 1.10E+05 | 2.60E−04 | 2.36E−09 | 901.9 | 0.58 |
| Y57K (BGA5384) | 1.02E+05 | 1.81E−04 | 1.77E−09 | 934.6 | 0.52 |
| K13E, Y57K | 1.28E+05 | 1.72E−04 | 1.34E−09 | 959.9 | 0.48 |
| Q54E, Y57E | 1.17E+05 | 2.50E−04 | 2.14E−09 | 926.9 | 0.65 |
| Y57D | 1.07E+05 | 1.95E−04 | 1.82E−09 | 949.3 | 0.56 |
| BGA6710 | 1.13E+05 | 1.69E−04 | 1.49E−09 | 919.3 | 0.49 |

### Example 10. Binding Profiles of Anti-CEA Antibody BGA5384

BGA5384 and a previously disclosed CEA antibody, designated as antibody 2F1 in US Patent Appln. Publication No. 2012/0251529, were generated in human IgG1 format and characterized for their binding kinetics by SPR assays using BIAcore™ T-200 (GE Life Sciences).

To obtain these data, anti-human IgG (Fc) antibody was immobilized on an activated CM5 biosensor chip (Cat. No. BR100839, GE Life Sciences). The BGA5384 antibody was flowed over the chip surface and captured by anti-human Fab antibody. Then a serial dilution (1.37 nM to 2150 nM) of soluble huCEA or cynoCEA (Cat.:CE5-C52H5, Acrobiosystem) were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (KD) was calculated as the ratio k_{off}/kₒₙ BGA5384 and and the 2F1 control antibody displayed different binding affinities. BGA5384 has very high affinity for human CEA and also a comparable affinity for cynoCEA, as shown in Table 15.

TABLE 15
Comparison of binding affinities of anti-CEA antibodies by SPR

| | BGA5384 | | | 2F1 Antibody | | |
|---|---|---|---|---|---|---|
| | Ka (1/Ms) | Kd (1/s) | KD (M) | Ka (1/Ms) | Kd (1/s) | KD (M) |
| huCEA | 1.02E+05 | 1.81E−04 | 1.77E−09 | 4.50E+4 | 3.00E−3 | 6.50E−8 |
| cynoCEA | 2.67E+04 | 5.34E−04 | 1.99E−08 | ND | ND | ND |

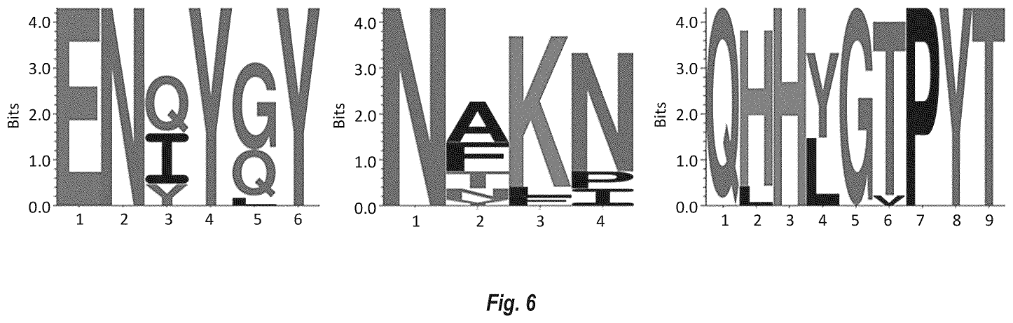

For flow cytometry, CEA-expressing MKN45 cells (105 cells/well) were incubated with various concentrations of purified affinity-matured antibodies, followed by binding with Alexa Fluor-647-labeled anti-hu IgG Fc antibody (Cat. No. 409320, BioLegend, USA). Cell fluorescence was quantified using a flow cytometer (Guava easyCyte™ 8HT, Merck-Millipore, USA). As shown in FIG. 8, BGA5384 demonstrated specific binding (as measured by mean fluorescent intensity, MFI) to native CEA on living cells in a dose-responsive manner with EC₅₀ of 2.92 pg/ml.

### Example 11. Evaluation of Off-Target Specificity

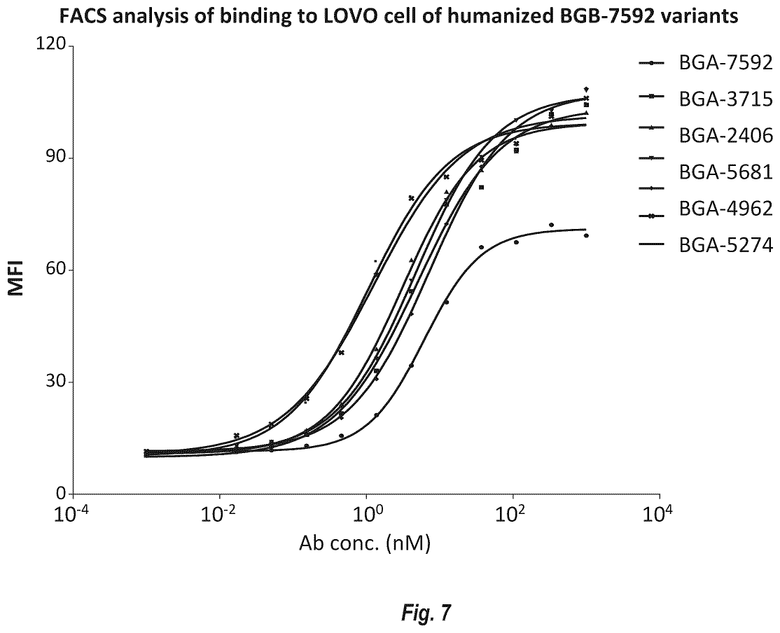

The off-target specificity of BGA5384 was evaluated via ELISA and flow cytometry. For flow cytometry CEACAM3 (SEQ ID NO:65), CEACAM7 (SEQ ID NO:66) or CEACAM8 (SEQ ID NO:67) were transiently transfected into HEK293 cells (105 cells/well) and then were incubated with 2 μg/ml purified BGA5384, followed by binding with Alexa Fluor-647-labeled anti-huIgG Fc antibody (Cat. No. 409320, BioLegend, USA). Cell fluorescence was quantified using a flow cytometer (Guava easyCyte™ 8HT, Merck-Millipore, USA). For antigen ELISA, CEACAM1(SEQ ID NO:64) (Cat. No. 10822-H08H, Sino Biological, China), CHIM (SEQ ID NO:63), CEA (SEQ ID NO:55) or CEACAM6 (SEQ ID NO:57) were coated in 96-well plates at a concentration of 10 μg/ml overnight at 4° C. The HRP-linked anti-human Fc (Fc specific) IgG antibody (Cat. No. A0170, Sigma, USA) and substrate (Cat. No. 00-4201-56, eBioscience, USA) were used for development, and absorbance signal at the wavelength of 450 nm was measured using a plate reader (SpectraMax Paradigm, Molecular Devices, USA). As shown in FIGS. 9A-9B, no cross-reactivity to other CEACAM family members was observed; BGA5384 demonstrated specificity only for CEA (CEACAM3 in FIGS. 9A-B).

### Example 12. Effects of Soluble huCEA on Binding of BGA5384 to CEA-Expressing Cells

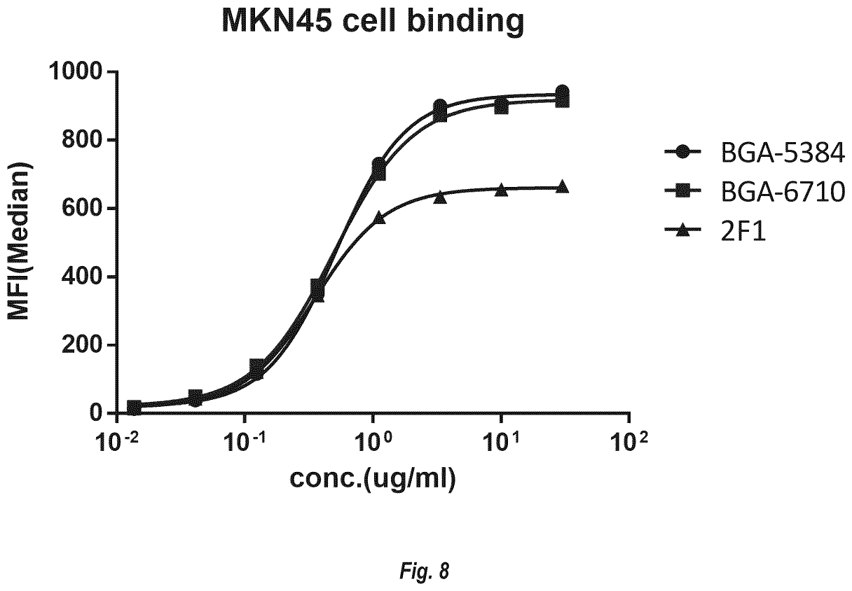

To determine if soluble CEA (sCEA) had any effect on the specific binding of BGA5384, various concentrations (0, 0.5, 1, or 2 μg/ml) of recombinant soluble CEA were premixed with (0.01-100 μg/ml) BGA5384 and incubated for 5 minutes. The mixtures were then incubated with 2×105 CEA-expressing cells, such as MKN45 cells, for 30 minutes at 4° C. The cells were stained with secondary antibody anti-huFc-APC (Cat. No. 409320, BioLegend, USA) and analyzed by flow cytometry. In the presence of 2 μg/ml of recombinant sCEA, the binding of BGA5384 to CEA-expressing cells was not affected. This result is shown for MKN45 cells (FIG. 10), and indicates BGA5384's specificity for the membrane-bound form of CEA.

### Example 13. BGA6710 Induces Potent ADCC Effects on CEA⁺ Tumor Cells

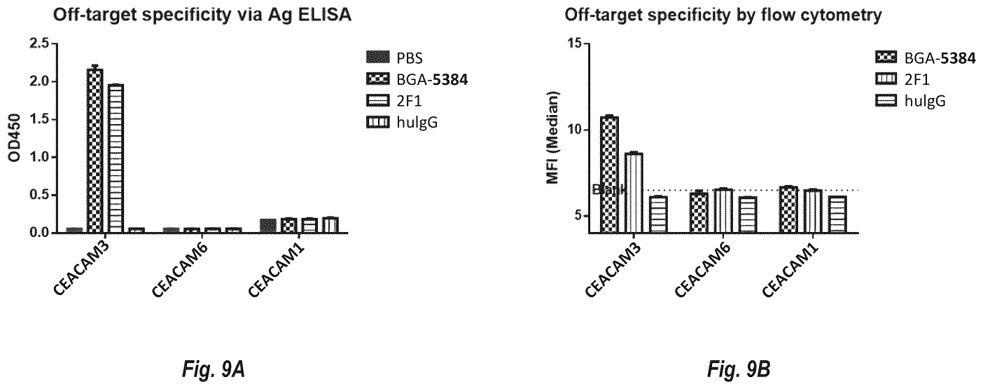

To determine whether BGA6710 in the wild-type IgG1 format can induce antibody dependent cytotoxicity (ADCC), CD16(V158)-expressing NK92MI cells (NK92MI/CD16V) were used as effector cells and were co-cultured with mouse colon cancer cells (CT26—ATCC CRL-2638) expressing CEA. The co-culture was performed at an E:T ratio of 1:1 for 5 hours in the presence of BGA6710 at indicated concentrations (0.00005-5 pg/ml), and cytotoxicity was determined by lactate dehydrogenase (LDH) release. The amounts of LDH in the supernatant were measured using the CytoTox™ 96 Non-Radioactive Cytotoxicity Assay kit (Promega, Madison, WI), and the amount of specific lysis was calculated according to the manufacturer's instruction. As shown in FIG. 11, BGA6710 could induce ADCC in vitro with an EC₅₀ around 6.7 ng/ml.

### Example 14. In Vivo Anti-Tumor Efficacy of BGA6710

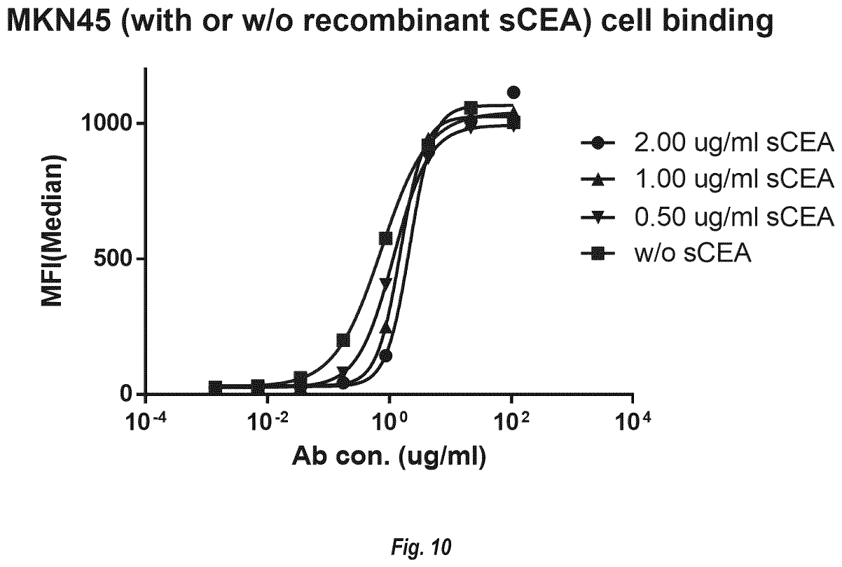

To determine the in vivo efficacy of BGA6710 against CEA⁺ tumor cells, NK92MI/CD16V cells (5×10⁶) were mixed with CT26/CEA cells (10⁶) and injected subcutaneously into NCG mice. BGA6710 (0.12, 0.62 or 3.1 mg/kg) or vehicle control was given twice per week starting on the day of tumor injection (7 mice per group). As compared to vehicle, BGA6710 at 3.1 mg/kg dosage showed a low amount of tumor inhibition, although the difference from vehicle control was not statistically significant (P>0.05) (FIG. 12).

### Example 15. Synthesis of Cytotoxic Agents (Payloads)

TABLE 16
Payload List

| Examples | Structures |
|---|---|
| P1 (DM4) | 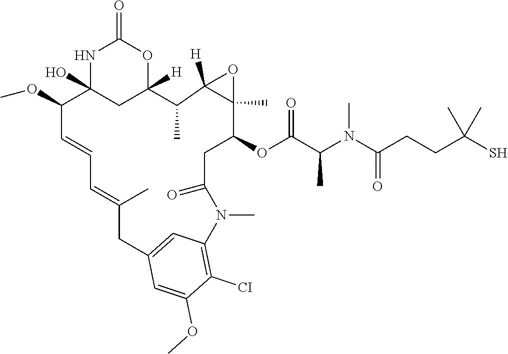 |
| P2 (DXD) | 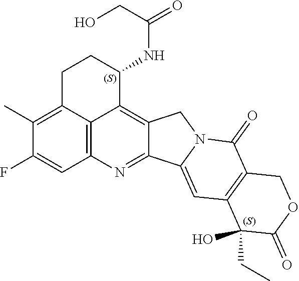 |
| P3 | 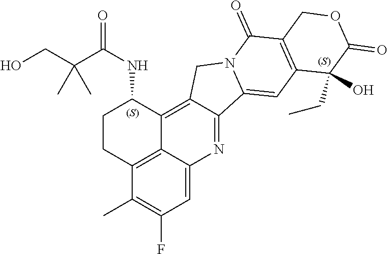 |
| P4 | 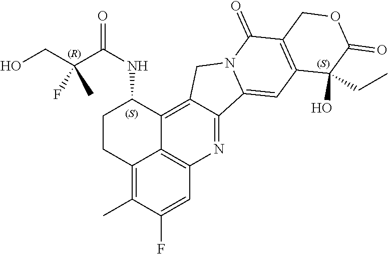 |

### UPLC Analysis Method:

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC®BEH C18 1.7 μm.

Method B: Mobile phase A: 0.1% FA in water, B: MeCN; Gradient: 10% B maintain 0.5 min, 10% -90°/B, 2.5 min, 90%1B maintain 0.2 min; Flow rate: 0.6 mL/min; Column: ACQUITY UPLC® BEH C18 1.7 μm.

Method C: Mobile phase A: 0.1% FA in water, B: MeCN; Gradient: 10% B maintain 0.2 min, 10% -90°/B, 1.3 min, 90% B maintain 0.3 min; Flow rate: 0.6 mL/min; Column: ACQUITY UPLC®BEH C18 1.7 μm.

P1 and P2 (Table 16) were commercially available and purchased from MedChemExpress CO. LTD (Shanghai).

### Synthetic Procedure of Payloads P3 and P4

### Payload P3

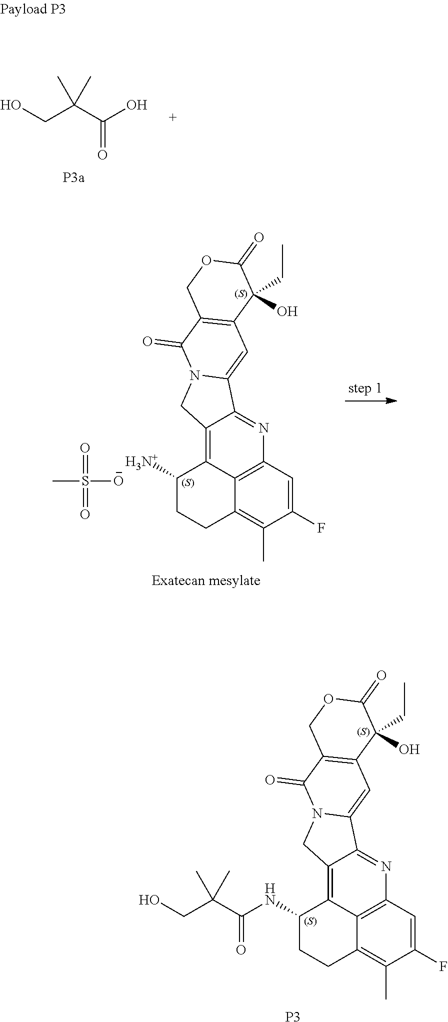

Step 1: N-((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)-3-hydroxy-2,2-dimethylpropanamide (P3). To a mixture of P3a (5.0 mg, 0.042 mmol) and HATU (16 mg, 0.042 mmol) in DMF (1 mL) were added DIEA (21 pL, 16 mg, 0.13 mmol) and Exatecan mesylate (23 mg, 0.043 mmol, Purchased from MedChemExpress CO. LTD). The resulting brown mixture was stirred at r.t. for 2 h. On completion of the reaction, the mixture was purified by prep-HPLC (TFA) (Method: column: XBridge Prep C18 OBD Sum 19*150 mm; Mobile phase: A-water (0.1% TFA): B-acetonitrile; Flow rate: 20 mL/min, the fraction was lyophilized to give P3 (15 mg, 65.5% yield) as a white powder. MS (ESI) m/z: 536.4 [M+H]⁺. ¹H NMR (400 MHz, DMSO-d₆) δ 8.00 (d, J=8.4 Hz, 1H), 7.79 (d, J=11.2 Hz, 1H), 7.31 (s, 1H), 6.52 (s, 1H), 5.59-5.54 (m, 1H), 5.42 (s, 2H), 5.18 (q, J=19.2 Hz, 2H), 4.87 (t, J=5.2 Hz, 1H), 3.45 (dd, J=10.2, 4.8 Hz, 1H), 3.41-3.28 (m, 1H), 3.15 (t, J=5.6 Hz, 2H), 2.40 (s, 3H), 2.24-2.07 (m, 2H), 1.92-1.80 (m, 2H), 1.11 (d, J=7.6 Hz, 6H), 0.87 (t, J=7.2 Hz, 3H).

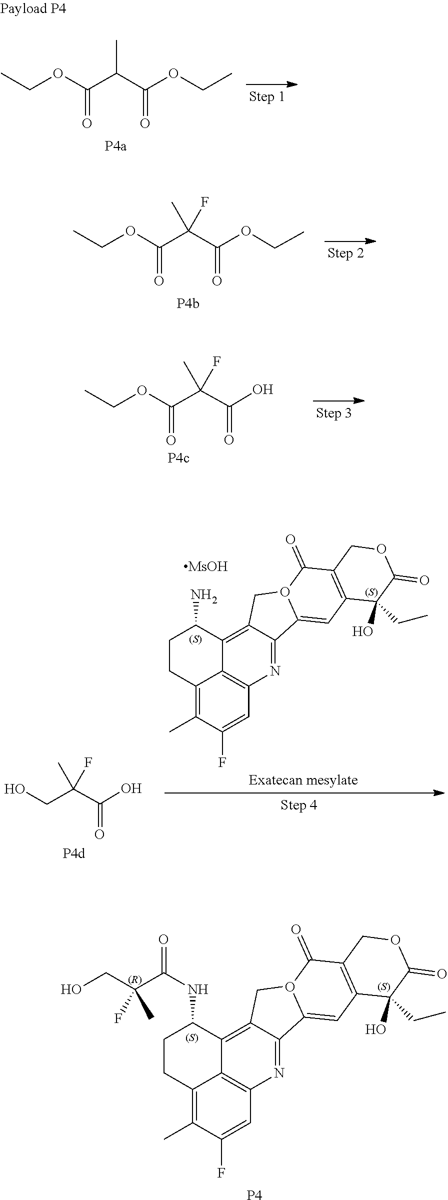

Step 1: Diethyl 2-fluoro-2-methylmalonate (P4b). A solution of compound a (10.00 g, 57.40 mmol) in THF (200 mL) was cooled to 0° C. 60% of NaH in oil (3.21 g, 80.37 mmol) was added into the mixture portion wise, stirred at 0° C. for 30 min. Then N-fluoro-N-(phenylsulfonyl)benzenesulfonamide (NSF1, 19.91 g, 63.20 mmol) was added into the mixture portion wise at 0° C., then warmed up to r.t. and stirred for 16 h. After the reaction was completed, the suspension was filtered and the filtrate was concentrated. PE (100 mL) was added into the residue, the precipitate was filtered and the filtrate was concentrated to give compound P4b (12.50 g, crude) as a light-yellow oil. ¹H NMR (400 MHz, CDCl₃) δ 4.30 (q, J=7.2 Hz, 4H), 1.79 (d, J=22.0 Hz, 3H), 1.31 (t, J=7.2 Hz, 6H). ¹⁹F NMR (376 MHz, CDCl₃) δ −157.50.

Step 2: 3-ethoxy-2-fluoro-2-methyl-3-oxopropanoic acid (P4c). To a solution of compound P4b (1.00 g, 5.20 mmol) in EtOH (5 mL) was added KOH solution (321 mg) in H₂O (50 μL) and EtOH (2 mL) dropwise at 0° C. The mixture was stirred at r.t. for 2 h. The mixture was diluted with 20 (mL), washed with DCM (20 mL*3). The aqueous solution was adjusted to pH=3 by 1 N HCl, then extracted by EtOAc (50 mL*3). The organic layer was dried combined and dried over anhydrous Na₂SO₄, filtered, and concentrated to give compound P4c (470 mg, 55.0% yield) as colorless oil. ¹H NMR (400 MHz, CDCl₃) δ 8.31 (br s, 1H), 4.32 (q, J=7.2 Hz, 2H), 1.83 (d, J=22.0 Hz, 3H), 1.33 (t, J=7.2 Hz, 3H). ¹⁹F NMR (376 MHz, CDCl₃) δ −157.59.

Step 3: 2-fluoro-3-hydroxy-2-methylpropanoic acid (P4d). To a solution of compound P4c (200 mg, 1.22 mmol) in isopropanol (4 mL) was added 2 M LiBH₄ (1.22 mL, 2.44 mmol) at 0° C. The mixture was stirred at r.t. for 2 h. The mixture was quenched by 2 N HCl (1.22 mL) dropwise at 0° C. The diluted with H₂O (10 mL), extracted with EtOAc (50 mL*3). The organic layer was combined and dried over anhydrous Na₂SO₄, filtered and concentrated to give compound P4d (92 mg, 61.7% yield) as colorless oil. ¹H NMR (400 MHz, CDCl₃) δ 4.01-3.81 (m, 2H), 1.58 (d, J=21.2 Hz, 3H). ¹⁹F NMR (376 MHz, CDCl₃) δ −163.98.

Step 4: N-((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)-2-fluoro-3-hydroxy-2-methylpropanamide (P4). To a solution of compound P4d (23 mg, 0.19 mmol) in DMF (2 mL) were added Exatecan mesylate (50 mg, 0.094 mmol), HATU (54 mg, 141 mmol) and DIEA (36 mg, 0.28 mmol). The mixture was stirred at r.t. for 1 h. The mixture was purified by prep-HPLC (FA) (Method: column: XBridge Prep C18 OBD Sum 19*150 mm; Mobile phase: A-water (0.1% formic acid): B-acetonitrile; Flow rate: 20 mL/min, the fraction was lyophilized to give two isomers:

Isomer 1: P4: white solid, (11 mg, 21.9% yield). UPLC-MS, RT=3.52 min. ¹H NMR (400 MHz, DMSO-d₆) δ 9.06 (dd, J=9.0, 2.8 Hz, 1H), 8.00 (d, J=10.9 Hz, 1H), 7.54 (s, 1H), 6.75 (s, 1H), 5.82 (d, J=8.0 Hz, 1H), 5.65 (s, 2H), 5.43 (dt, J=77.8, 12.4 Hz, 3H), 4.17-3.91 (m, 1H), 3.83 (ddd, J=18.0, 12.4, 5.6 Hz, 1H), 3.40-3.27 (m, 1H), 2.62 (s, 3H), 2.50-2.34 (m, 2H), 2.22-1.98 (m, 2H), 1.81 (d, J=21.4 Hz, 3H), 1.11 (t, 0.1=7.2 Hz, 3H); MS (ESI) m/z: 540.3 [M+H]⁺.

Isomer 2: P4-1: white solid, (8.4 mg, 16.6% yield). UPLC-MS, RT=3.86 min. ¹H NMR (400 MHz, DMSO-d₆) δ 8.72 (dd, J=8.4, 2.4 Hz, 1H), 7.78 (d, J=11.2 Hz, 1H), 7.31 (s, 1H), 6.52 (s, 1H), 5.58 (d, J=8.0 Hz, 1H), 5.42 (s, 2H), 5.32-5.05 (m, 3H), 3.83 (dd, J=26.8, 12.0 Hz, 1H), 3.61 (dd, J=21.6, 12.0 Hz, 1H), 3.22-3.07 (m, 2H), 2.46-2.30 (m, 3H), 2.28-2.05 (m, 2H), 2.02-1.74 (m, 2H), 1.45 (d, J=21.4 Hz, 3H), 0.87 (t, 0.1=7.2 Hz, 3H); MS (ESI) m/z: 540.3 [M+H]⁺.

### Example 16. Synthesis of Linker-Payloads

TABLE 17
Linker-payload List

| Linker-payload | Structure |
|---|---|
| LD2-1 | 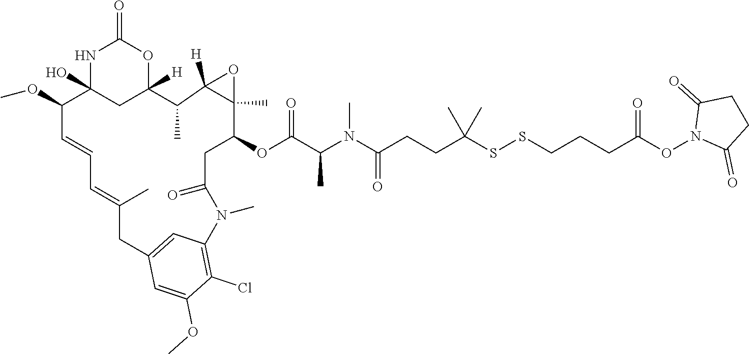 [SMILES: COc1cc2cc(c1Cl)N(C)C(=O)C[C@H](OC(=O)[C@H](C)N(C)C(=O)CCC(C)(C)SSCCCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2] |
| LD2-2 | 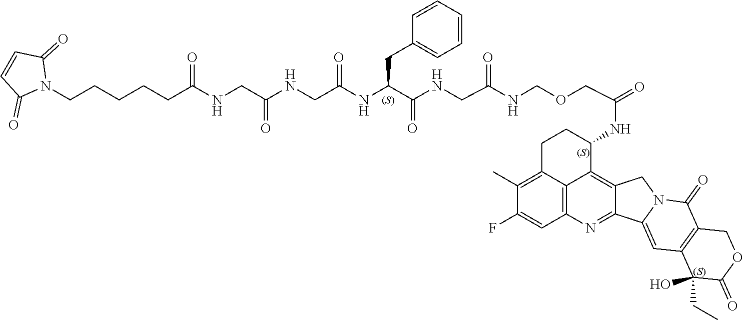 |
| LD2-3 | 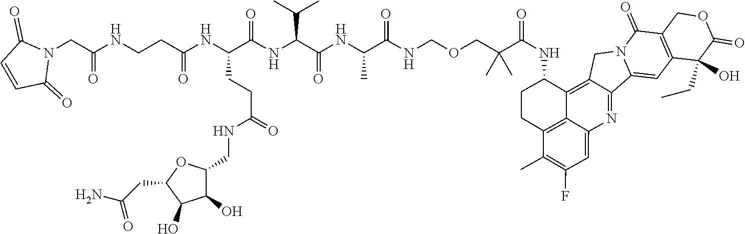 [SMILES: CC[C@@]1(O)C(=O)OCc2c1cc1n(c2=O)Cc2c-1nc1cc(F)c(C)c3c1c2[C@@H](NC(=O)C(C)(C)COCNC(=O)[C@H](C)NC(=O)[C@@H](NC(=O)[C@H](CCC(=O)NC[C@H]1O[C@@H](CC(N)=O)[C@H](O)[C@@H]1O)NC(=O)CCNC(=O)CN1C(=O)C=CC1=O)C(C)C)CC3] |
| LD2-4 | 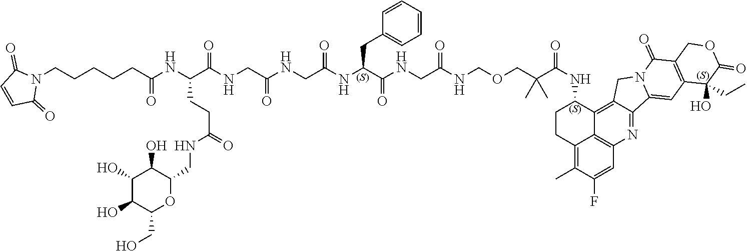 |
| LD2-5 | 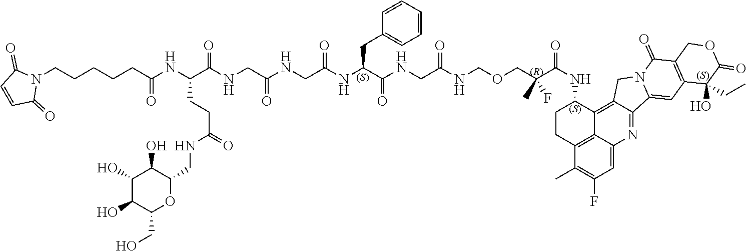 |
| LD2-6 | 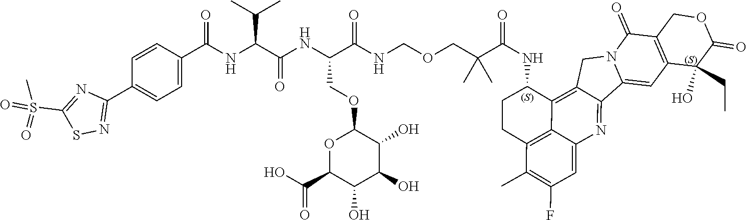 |
| LD2-7 | 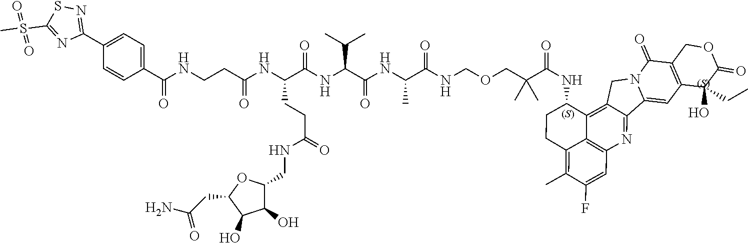 |
| LD2-8 | 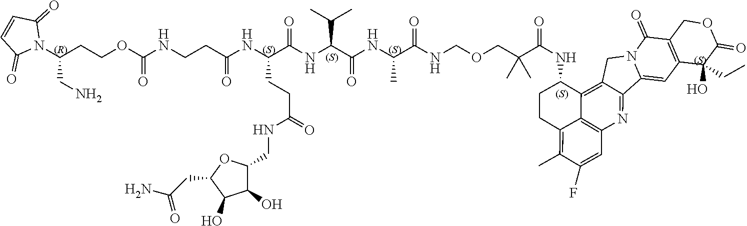 |

LD2-1 and LD2-2 (Table 17) were commercially available and purchased from MedChemExpress CO. LTD (Shanghai).

### Synthetic Procedure of Linker-Cytotoxic Agent LD2-3 to LD2-8

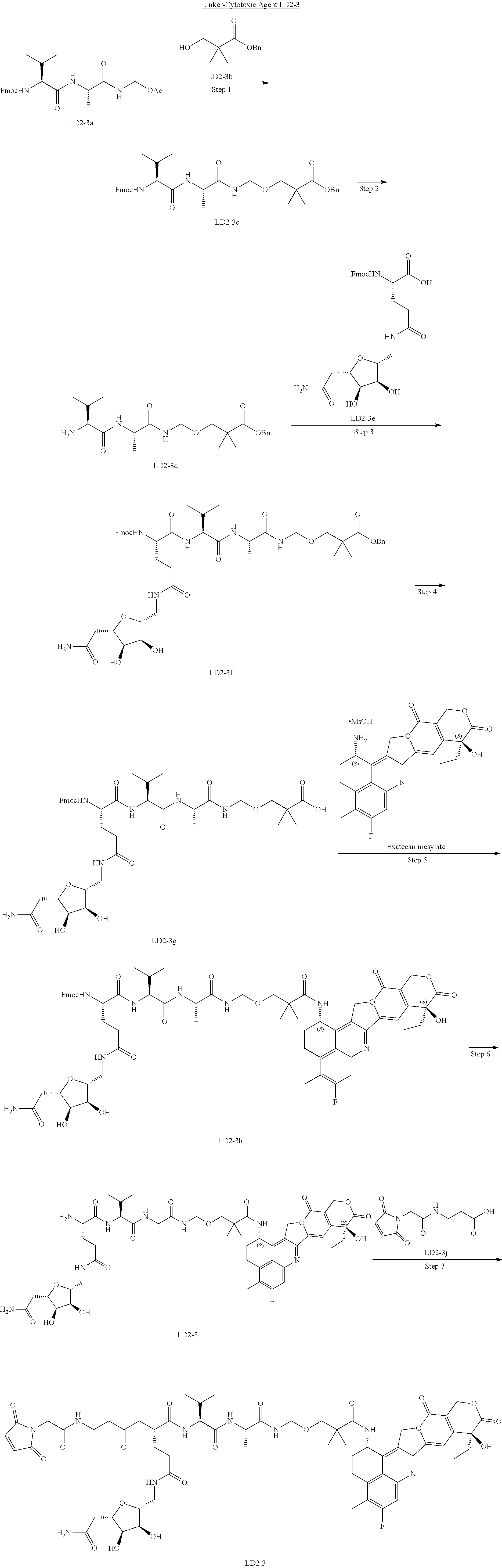

Step 1: benzyl (5S,8S)-1-({9H-fluoren-9-yl)-5-isopropyl-8,14,14-trimethyl-3,6,9-trioxo-2,12-dioxa-4,7,10-triazapentadecan-15-oate (LD2-3c). A white suspension mixture of LD2-3a (300 mg, 0.62 mmol, synthesized according to the reported procedures: ACS Med Chem. Lett. 2019, 10, 1386-1392 and U.S. Pat. No. 9,808,537B2), LD2-3b (260 mg, 1.25 mmol) and 4 Å molecular sieve in anhydrous THF (10 mL) was stirred at r.t. for 10 min. Sc(OTf)₃ (368 mg, 0.75 mmol) was added and the resulted yellow suspension was stirred at r.t. for 4 hr. The yellow suspension mixture was filtered through a pad of celite and washed with EtOAc (30 mL). Combined organic layers were washed with sat. NaH—CO₃ (30 mL) and brine (30 mL), dried over Na₂SO₄, filtered and the filtrate was concentrated under vacuum to give a residue. It was purified by silica gel column (MeOH/DCM=0%˜5%), fraction was concentrated under vacuum to give LD2-3c (274 mg, 69.8% yield) as a white solid. MS (ESI) m/z: 652.6 [M+Na]⁺.

Step 2: benzyl 3-(((S)-2-((S)-2-amino-3-methylbutanamido)propanamido)methoxy)-2,2-dimethylpropanoate (LD2-3d). To a solution of LD2-3c (274 mg, 0.44 mmol) in DMF (5 mL) was added Et₂NH (477 mg, 5.53 mmol). The mixture was stirred at r.t. for 20 min. The reaction mixture was concentrated under vacuum and co-evaporated with toluene two times to give LD2-3d (275 mg, crude) as a brown oil. MS (ESI) m/z: 430.4 [M+Na]⁺.

Step 3: benzyl (5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-11,17,17-trimethyl-3,6,9,12-tetraoxo-2,15-dioxa-4,7,10,13-tetraazaoctadecan-18-oate (LD2-3f). To a solution of LD2-3d (275 mg, crude) and LD2-3e (282 mg, 0.52 mmol, purchased from WuXi AppTec) in DMF (5 mL) were added HATU (198 mg, 0.52 mmol) and DIPEA (168 mg, 1.30 mmol). The mixture was stirred at r.t. for 10 min. The mixture was purified by reserved phase (C18, 60 g, 30%˜70%), the fraction was freeze-dried to give LD2-3f (370 mg, 91.5% yield) as a brown solid. MS (ESI) m/z: 953.8 [M+Na]⁺.

Step 4: (5S,8S,11S,17R)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-17-fluoro-8-isopropyl-11,17-dimethyl-3,6,9,12-tetraoxo-2,15-dioxa-4,7,10,13-tetraazaoctadecan-18-oic acid (LD2-3g). To a solution of compound LD2-3f (3.01 g, 3.21 mmol) in co-solvent DMF-MeOH (40 mL, 1:1, v:v) was added Pd/C (10%, 600 mg). The mixture was stirred at H₂ atmosphere (15 psi) for 7 h. The mixture was filtered through a pad of celite, concentrated to give compound LD2-3g (2.50 g, crude) as white solid. MS (ESI) m/z: 863.7 [M+Na]⁺.

Step 5: (9H-fluoren-9-yl)methyl ((6S,9S,12S)-1-((2R,3S,4R,5S)-5-(2-amino-2-oxoethyl)-3,4-dihydroxytetrahydrofuran-2-yl)-19-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-9-isopropyl-12,18,18-trimethyl-3,7,10,13,19-pentaoxo-16-oxa-2,8,11,14-tetraazanonadecan-6-yl)carbamate (LD2-3h). To a solution of compound Exatecan mesylate (1000 mg, 1.18 mmol, purchased from MedChemExpress CO. LTD) in DMF (20 mL) were added compound LD2-3g (692 mg, 1.30 mmol), HATU (675 mg, 1.78 mmol) and DIEA (459 mg, 3.55 mmol). The mixture was stirred at r.t. for 30 min. The mixture was concentrated and purified by a silica gel column chromatography (eluent: DCM/MeOH=0% to 20%) to give the title compound LD2-3h (1.32 g, 88.6% yield) as an off-white solid. MS (ESI) m/z: 1282.1 [M+Na]⁺.

Step 6: (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl)pentanediamide (LD2-3i). To a solution of compound LD2-3h (1000 mg, 0.79 mmol) in DMF (20 mL) was added Et₂NH (580 mg, 7.93 mmol). The mixture was stirred at r.t. for 30 min. The mixture was concentrated under high vacuum to give compound LD2-3i (825 mg, crude) as an off-white solid, which was used directly without further purification. MS (ESI) m/z: 1036.9 [M+H]⁺.

Step 7: (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl)pentanediamide (LD2-3). To the solution of LD2-3j (15 mg, 0.067 mmol) in dry DMF (1.0 mL) were added HATU (26 mg, 0.067 mmol) and DIEA (0.017 mL, 0.097 mmol), stirred at r.t. for 15 min. Then to the above mixture was added LD2-3i (50 mg, 0.048 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give LD2-3 (37 mg, 50.9% yield) as a yellow solid. MS (ESI) m/z: 1266.7 [M+Na]⁺.

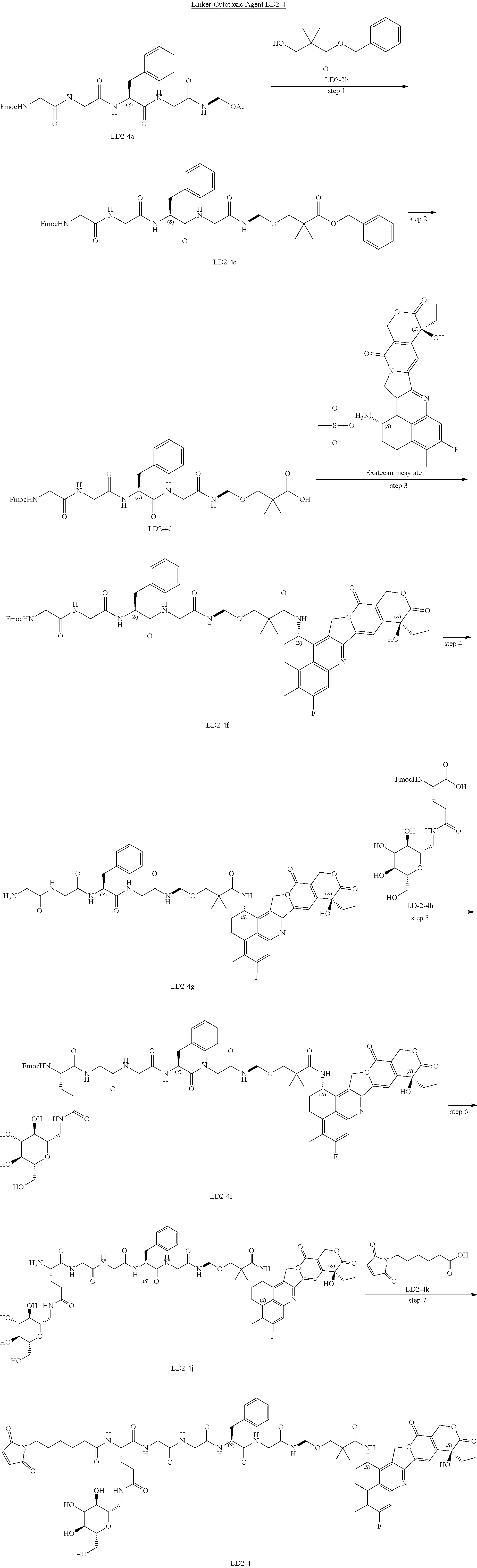

Step 1: Benzyl (S)-11-benzyl-1-(9H-fluoren-9-yl)-20,20-dimethyl-3 9,12,15-pentaoxo-2,18-dioxa-4,7,10,13,10-pentaazahenicosan-2l -oate (LD2-4c). To the solution of LD24a (250 mg, 0.40 mmol) and LD2-3b (83 mg, 0.40 mmol) in THF (5 mL) was added 4 Å molecular sieve. The mixture was stirred at r.t, for 10 min then Sc(OTf)₃ (195 mg, 0.40 mmol) was added and further reacted at r.t. for another 16 h. The suspension mixture was filtered through a pad of celite, and the cake was washed with THF (10 mL) then the filtrate was quenched by addition of Sat. NaHCO₃ (10 mL), extracted with EtOAc (30 mL*2). After separation, the combined organic layers were washed with brine (50 mL), dried over Na₂SO₄, filtered and the filtrate was concentrated under vacuum to give a residue which was further purified by silica gel column chromatography (A-DCM; B-MeOH, MeOH/DCM=0%˜5%) to provide LD2-4c (90 mg, 29.2% yield) as a white solid. MS (ESI) m/z: 800.5 [M+Na]⁺.

Step 2: (S)-11-benzyl-1-(9H-fluoren-9-yl)-20,20-dimethyl-3,6,9,12,15-pentaoxo-2,18-dioxa-4,7,10,13,16-pentaazahenicosan-21-oic acid (LD2-4d). To a solution of LD2-4c (80 mg, 0.10 mmol) in MeOH (3 mL) was added wet Pd/C (20 mg). The black suspension was purged with H₂ balloon for three times then reacted at r.t. for 2 h under H₂ balloon. After the reaction was completed, the black suspension was filtered off through a pad of celite and the cake wash with MeOH, the combined organic layers were concentrated under vacuum to provide LD2-4d (61 mg, 84.8% yield). MS (ESI) m/z: 710.4 [M+Na]⁺.

Step 3: (9H-fluoren-9-yl)methyl ((S)-7-benzyl-17-(((1S,9S)-9-ethyl-5-fluoro-9-hydroxy-4-methyl-10,13-dioxo-2,3,9,10,13,15-hexahydro-1H,12H-benz′[d]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)carbamate (LD2-4f). To a mixture of LD2-4d (60 mg, 0.087 mmol) and HATU (33 mg, 0.087 mmol) in DMF (2 mL) was added DIEA (43 μL, 34 mg, 0.26 mmol). The mixture was reacted at r.t. for 10 min. Exatecan mesylate (46 mg, 0.087 mmol) was added and reacted at the same temperature for another 1 hr. After the reaction was completed, the mixture was filtered and the filtrate was purified using prep-HPLC (Method; column: XBridge Prep C18 OBD 5 μm 19*150 mm; Mobile phase: A-water (0.1% formic acid): B-acetonitrile; Flow rate: 20 mL/min) to provide LD2-4f (85 mg, 88.2% yield). MS (ESI) m/z: 1105.5 [M+H]⁺.

Step 4: 3-(((S)-13-amino-7-benzyl-3,6,9,12-tetraoxo-2,5,8,11-tetraazatridecyl)oxy)-N-((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)-2,2-dimethylpropanamide (LD2-4g). To a solution of LD2-4f (85 mg, 0.062 mmol) in DMF (2 mL) was added Et₂NH (64 μL, 46 mg, 0.62 mmol). The mixture was stirred at r.t. for 0.5 h. On completion of the reaction. The mixture was concentrated under vacuum to give LD2-4g (86 mg, crude) as a yellow solid. MS (ESI) m/z: 883.5 [M+H]⁺.

Step 6: (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-24,24-dimethyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (LD2-4i). To a solution of LD2-4g (86 mg, crude) and LD2-4h (43 mg, 0.079 mmol, purchased from WuXi AppTec) in DMF (1.5 mL) was added DIEA (26 μL, 21 mg, 0.16 mmol). The mixture was stirred at r.t. for 1.5 h. On completion of the reaction. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 mL/min, the fraction was lyophilized to give LD2-4i (70 mg, 62.6% yield) as a white powder. MS (ESI) m/z: 1410.7 [M+H]⁺.

Step 7: (S)-2-amino-N1-((S)-7-benzyl-17-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (LD2-4j). To a solution of LD2-4i (70 mg, 0.050 mmol) in DMF (1 mL) was added Et₂NH (51 pL, 36 mg, 0.50 mmol).

The mixture was stirred at r.t. for 0.5 h. On completion of the reaction. The mixture was concentrated under vacuum to give LD2-4j (71 mg, crude) as a yellow solid. MS (ESI) m/z: 1188.2 [M+H]⁺. 10M1 Step 8: (S)—N′-((S)-7-benzyl-17-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2H-pyran-2-yl)methyl)pentanediamide (LD2-4). To a solution of LD2-4k (19 mg) in DMF (3 mL) were added HATU (34 mg, 0.088 mmol) and DIEA (10 μL, 7.6 mg, 0.059 mmol). The resulted yellow solution was stirred at r.t. for 5 min then LD2-4j (71 mg, crude) was added. The mixture was stirred at r.t. for 60 min. On completion of the reaction. The mixture was purified by prep-HPLC (FA) (Method: column: XBridge Prep C18 OBD Sum 19*150 mm; Mobile phase: A-water (0.1% formic acid): B-acetonitrile; Flow rate: 20 mL/min, the fraction was lyophilized to give LD2-4 (32 mg, 26.3% yield) as a white powder. MS (ESI) m/z: 1381.1 [M+H]⁺.

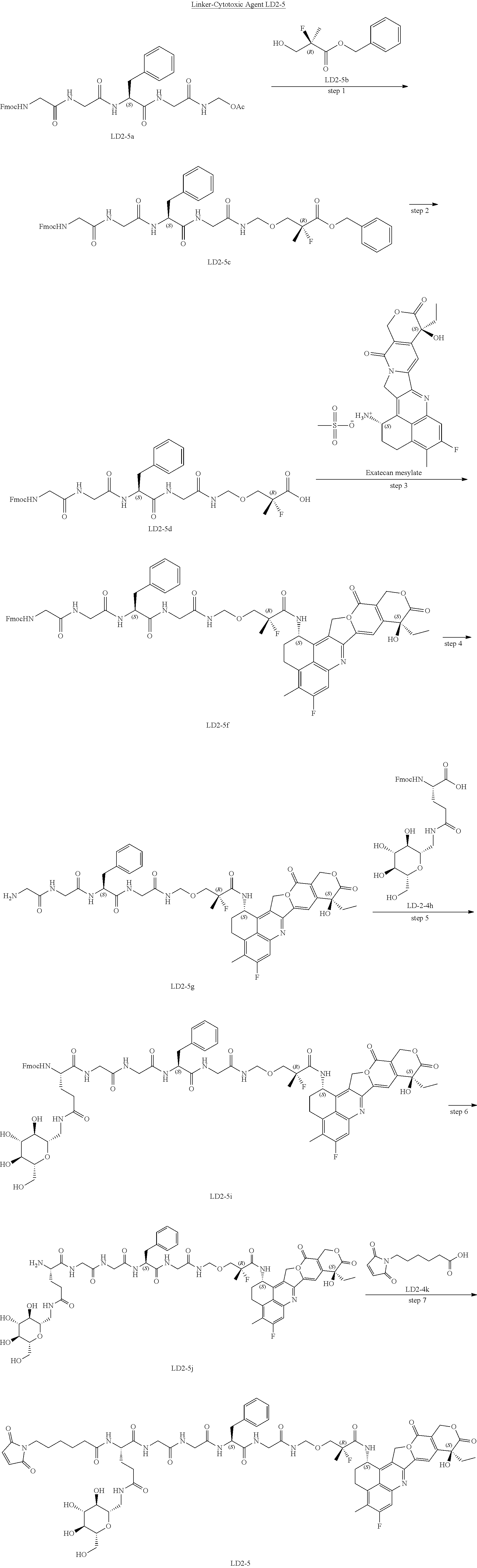

LD2-5 (30 mg, 50.7% yield) was synthesized according to the synthetic procedures of LD2-4.

MS (ESI) m/z: 1408.1 [M+Na]⁺.

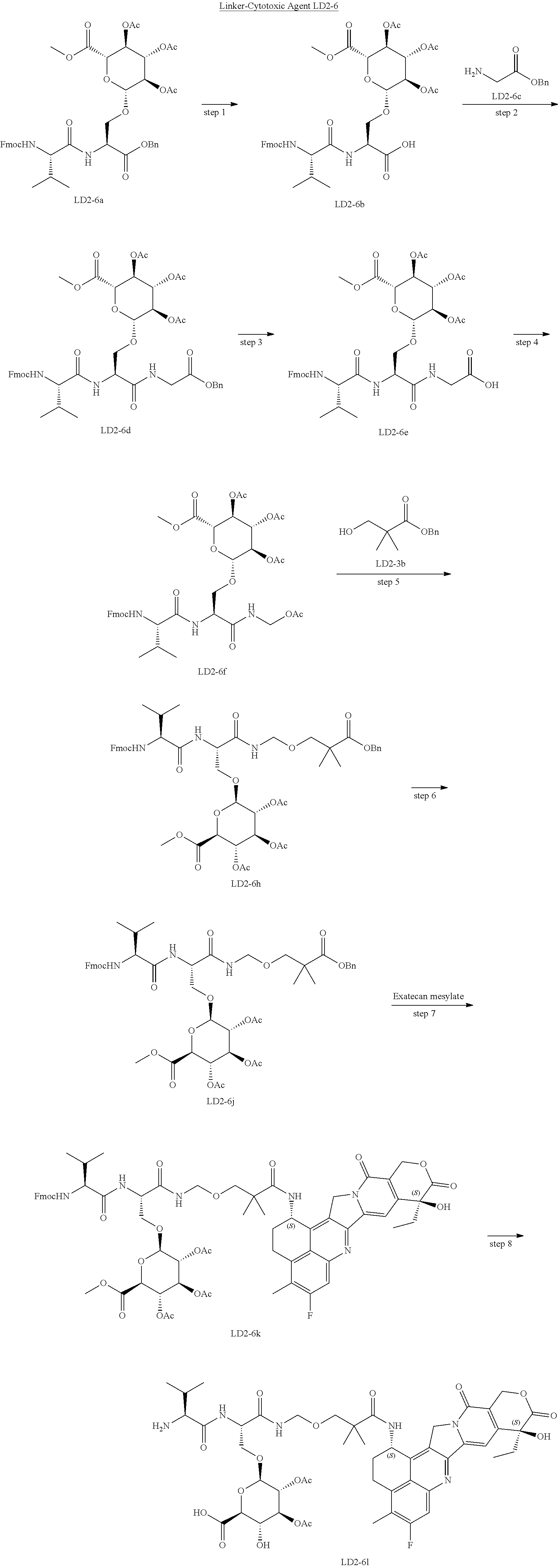

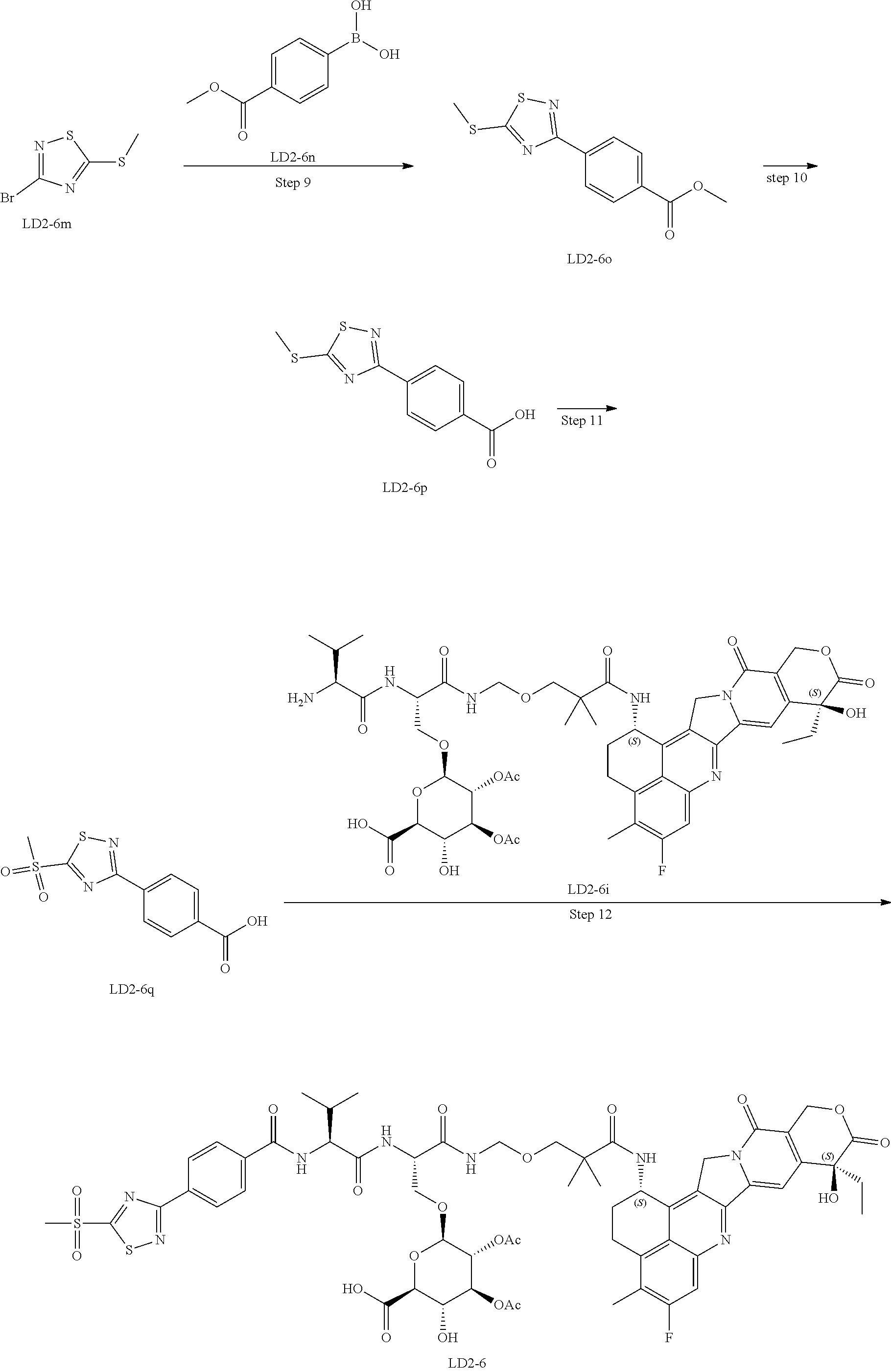

Step 1: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serine (LD2-6b). To a mixture of L1D2-6a (4.10 g, 4.92 mmol, purchase from MedChemExpress CO. LTD) in MeOH (50 mL), THF (100 mL) and DCM (20 mL) was added wet Pd/C (400 mg, 10% purity). The black suspension was purged with H₂ balloon for three times and then stirred at r.t. for 1 hr. The black suspension was filtered through a pad of celite, washed with MeOH (200 mL). Combined organic layers and concentrated under vacuum to give LD2-6b (3.65 g, 99.8% yield) as an off-white solid. MS (ESI) m/z: 743.6 [M+H]⁺.

Step 2: (2R,3R,4S,5S,6S)-2-((S)-2-((S)-2-((((9H-fluoren-9-yl)methoxy)carbonyl)amino)-3-methylbutanamido)-3-((2-(benzyloxy)-2-oxoethyl)amino)-3-oxopropoxy)-6-(methoxycarbonyl)tetrahydro-2H-pyran-3,4,5-triyl triacetate (LD2-6d). To a solution of LD2-6b (3.65 g, 4.92 mmol) and LD2-6c (1.66 g, 4.92 mmol) in DMF (50 mL) were added HATU (1.87 g, 4.92 mmol) and DIEA (1.59 g, 12.29 mmol). The mixture was stirred at r.t. for 30 min. The mixture was purified by FCC (MeOH/DCM=0%˜10⁰/), the fraction was concentrated under vacuum to give LD2-6d (3.80 g, 86.9% yield) as off-white foamed solid. MS (ESI) m/z: 890.7 [M+H]⁺.

Step 3: N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serylglycine (LD2-6e). To a mixture of LD2-6d (3.80 g, 4.27 mmol) in MeOH (150 mL) and DCM (50 mL) was added wet Pd/C (400 mg, 10% purity). The black suspension was purged with H₂ balloon for three times and then stirred at r.t. for 40 min. The black suspension was filtered through a pad of celite, washed with MeOH (150 mL). Combined organic layers and concentrated under vacuum to give LD2-6e (3.30 g, 96.6% yield) as an off-white solid. MS (ESI) m/z: 800.7 [M+H]⁺.

Step 4: (2R,3R,4S,5S,6S)-2-((S)-2-((S)-2-((((9H-fluoren-9-yl)methoxy)carbonyl)amino)-3-methylbutanamido)-3-((acetoxymethyl)amino)-3-oxopropoxy)-6-(methoxycarbonyl)tetrahydro-2H-pyran-3,4,5-triyl triacetate (LD2-6f). To a solution of LD2-6e (3.30 g, 4.13 mmol) in DMF (30 mL) were added Pb(OAc)₄ (2.74 g, 6.19 mmol), Cu(OAc)₂ (74.9 mg, 0.41 mmol) and HOAc (247.8 mg, 4.13 mmol). The resulted dark color mixture was purged with N₂ balloon for three times and then stirred at 65° C. for 40 min the mixture was turn to deep blue. The mixture was diluted with EtOAc (300 mL), washed with brine (100 mL*3), dried over Na₂SO₄, filtered and concentrated under vacuum to give a residue. It was purified by FCC (MeOH/DCM=0˜10%), the fraction was concentrated under vacuum to give LD2-6f (2.81 g, 83.4% yield) as a pale-yellow solid. MS (ESI) m/z: 836.6 [M+Na]⁺.

Step 5: (2R,3R,4S,5S,6S)-2-((S)-2-((S)-2-((((9H-fluoren-9-yl)methoxy)carbonyl)amino)-3-methylbutanamido)-3-(((3-(benzyloxy)-2,2-dimethyl-3-oxopropoxy)methyl)amino)-3-oxopropoxy)-6-(methoxycarbonyl)tetrahydro-2H-pyran-3,4,5-triyl triacetate (LD2-6h). A white suspension mixture of LD2-6f (300 mg, 0.37 mmol), LD2-3b (154 mg, 0.74 mmol) and 4 Å molecular sieve (200 mg) in anhydrous THF (10 mL) was stirred at r.t. for 10 min. Sc(OTf)₃ (218 mg, 0.44 mmol) was added and the resulted yellow suspension was stirred at r.t. for 4 hr. The yellow suspension mixture was filtered through a pad of celite and washed with EtOAc. Combined organic layers were washed with sat. NaHCO₃ (30 mL) and brine (30 mL), dried over Na₂SO₄, filtered and the filtrate was concentrated under vacuum to give a residue. It was purified by silica gel column (MeOH/DCM=0%-5%), fraction was concentrated under vacuum to give LD2-6h (275 mg, 77.5% yield) as a white foam solid. MS (ESI) m/z: 984.8 [M+Na]⁺.

Step 6: (5S,8S)-1-(9H-fluoren-9-yl)-5-isopropyl-14,14-dimethyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazapentadecan-15-oic acid (LD2-6j). To a solution of LD2-6h (275 mg, 0.29 mmol) in MeOH (10 mL) was added wet Pd/C (55 mg, 10% purity). The black suspension was purged with H₂ balloon for three times then stirred at r.t. for 2 hr. The mixture was filtered through syringe head and washed with MeOH (15 mL), concentrated under vacuum to give LD2-6j (230 mg, crude) as a white foam solid. MS (ESI) m/z: 894.6 [M+Na]⁺.

Step 7: (2R,3R,4S,5S,6S)-2-((S)-2-((S)-2-((((9H-fluoren-9-yl)methoxy)carbonyl)amino)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-2,2-dimethyl-3-oxopropoxy)methyl)amino)-3-oxopropoxy)-6-(methoxycarbonyl)tetrahydro-2H-pyran-3,4,5-triyl triacetate (LD2-6k). To a mixture of LD2-6j (230 mg, crude), Exatecan mesylate (140 mg, 0.26 mmol) and HATU (100 mg, 0.26 mmol) in DMF (5 mL) was added DIEA (102 mg, 0.79 mmol). The resulted brown mixture was stirred at r.t. for 1 hr. The mixture was diluted with EtOAc (20 mL), washed with brine (20 mL*3), dried over Na₂SO₄, filtered, and concentrated under vacuum to give a residue. It was purified by FCC (MeOH/DCM=0%˜3%), concentrated under vacuum to give LD2-6k (325 mg, 95.6% yield) as off-white foam solid. MS (ESI) m/z: 1289.9 [M+H]⁺.

Step 8: (2S,3S,4S,5R,6R)-6-((S)-2-((S)-2-amino-3-methylbutanamido)-3-(((3-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic acid (LD2-61). To a solution of LD2-6k (325 mg, 0.25 mmol) in DMF (5 mL) was added Et₂NH (523 mg, 5.06 mmol). The mixture was stirred at r.t. for 20 min. LCMS showed reaction was completed then concentrated under vacuum to give a crude product. Which was dissolved in MeOH (6 mL) was added K₂CO₃ (174.7 mg, 1.26 mmol) and stirred at r.t. for 10 min then H₂O (2 mL) was added to the mixture and stirred at r.t. for 30 min. The mixture was acidified with sat. KHSO₄ at 0° C. to pH=3. filtered and purified by prep-HPLC (0.1% FA), fraction was lyophilized to give LD2-61 (140 mg, 59.7% yield) as a pale yellow solid. MS (ESI) m/z: 927.4 [M+H]⁺. ¹H NMR (400 MHz, d₆-DMSO) δ 9.56 (s, 1H), 8.39 (s, 1H), 8.07 (d, J=8.4 Hz, 1H), 7.77 (d, J=11.2 Hz, 1H), 7.31 (s, 1H), 6.52 (s, 1H), 5.54 (dd, J=13.2, 7.2 Hz, 1H), 5.43 (s, 2H), 5.18 (dd, J=41.6, 18.8 Hz, 2H), 5.09-5.02 (m, 1H), 4.96 (s, 1H), 4.62 (dd, J=10.0, 6.8 Hz, 1H), 4.56-4.44 (m, 2H), 4.19 (d, J=7.6 Hz, 1H), 3.82 (dd, J=10.8, 6.8 Hz, 1H), 3.61 (dd, J=11.6, 6.4 Hz, 2H), 3.17-3.05 (m, 4H), 2.94 (t, J=8.0 Hz, 1H), 2.39 (s, 3H), 2.11 (dt, J=21.3, 7.6 Hz, 2H), 2.03-1.93 (m, 2H), 1.92-1.78 (m, 3H), 1.12 (d, J=8.0 Hz, 6H), 0.87 (dd, J=13.0, 6.6 Hz, 9H).

Step 9: methyl 4-(5-(methylthio)-1,2,4-thiadiazol-3-yl)benzoate (LD2-6o). To a solution of compound LD2-6m (100 mg, 0.47 mmol) in toluene (4 mL) and H₂O (1 mL) were added compound LD2-6n (110 mg, 0.57 mmol), K₂CO₃ (168 mg, 0.95 mmol) and Pd(dppf)Cl₂ DCM (35 mg, 0.047 mmol). The mixture was stirred at 110° C. for 3 h under N₂ atmosphere. The mixture was filtered through a pad of celite, diluted with EtOAc (100 mL), washed by brine (50 mL*4). The organic layer was dried over anhydrous Na₂SO₄, filtered and concentrated. The crude was purified by flash column chromatography (eluted with PE/EtOAc=0˜40%). Compound LD2-6o (56 mg, 44.4% yield) was obtained as off-white solid. MS (ESI) m/z: 267.1 [M+H]⁺.

Step 10: 4-(5-(methylthio)-1,2,4-thiadiazol-3-yl)benzoic acid (LD2-6p). To a solution of compound LD2-6o (54 mg, 0.20 mmol) in MeOH (3 mL) and H₂O (1 mL) was added LiOH (17 mg, 0.41 mmol). The mixture was stirred at r.t. for 2 h. The mixture was adjusted to pH 7 and purified by prep-HPLC (FA condition) to give compound LD2-6p (36 mg, 70.3% yield) as a white solid. MS (ESI) m/z: 253.1 [M+H]⁺.

Step 11: 4-(5-(methylsulfonyl)-1,2,4-thiadiazol-3-yl)benzoic acid (LD2-6q). To a solution of compound LD2-6p (35 mg, 0.14 mmol) in DCM (3 mL) and THF (3 mL) was added m-CPBA (96 mg, 0.55 mmol). The mixture was stirred at room temperature for 16 h. The mixture was concentrated and purified by prep-HPLC (Method: column: XBridge Prep C18 OBD Sum 19*150 mm; Mobile phase: A-water (0.1% TFA): B-acetonitrile; Flow rate: 20 mL/min). Compound LD2-6q (12 mg, 99% purity) was obtained as white solid. MS (ESI) m/z: 284.8 [M+H]⁺.

Step 12: (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benz′[d′]pyrano[3′,4′:6,7]indolizino[1,2-b]quinolin-1-yl)amino)-2,2-dimethyl-3-oxopropoxy)methyl)amino)-2-((S)-3-methyl-2-(4-(5-(methylsulfonyl)-1,2,4-thiadiazol-3-yl)benzamido)butanamido)-3-oxopropoxy)-3,4,5-trihydroxytetrahydro-2H-pyran-2-carboxylic acid (LD2-6). To a solution of compound LD2-6q (7.4 mg, 0.026 mmol) in DMF (2 mL) were added HATU (9. mg, 0.024 mmol) and DIEA (5.6 mg, 0.043 mmol). The mixture was stirred at r.t. for 30 min. Compound LD2-61 (20 mg, 0.022 mmol) was added into the mixture and stirred at r.t. for 15 min. The reaction was purified by prep-HPLC (Method: column: XBridge Prep C18 OBD Sum 19*150 mm; Mobile phase: A-water (0.1% TFA): B-acetonitrile; Flow rate: 20 mL/min) to give compound LD2-6 (7.6 mg, 29.5% yield) as a white solid. MS (ESI) m/z: 1193.5 [M+H]⁺.

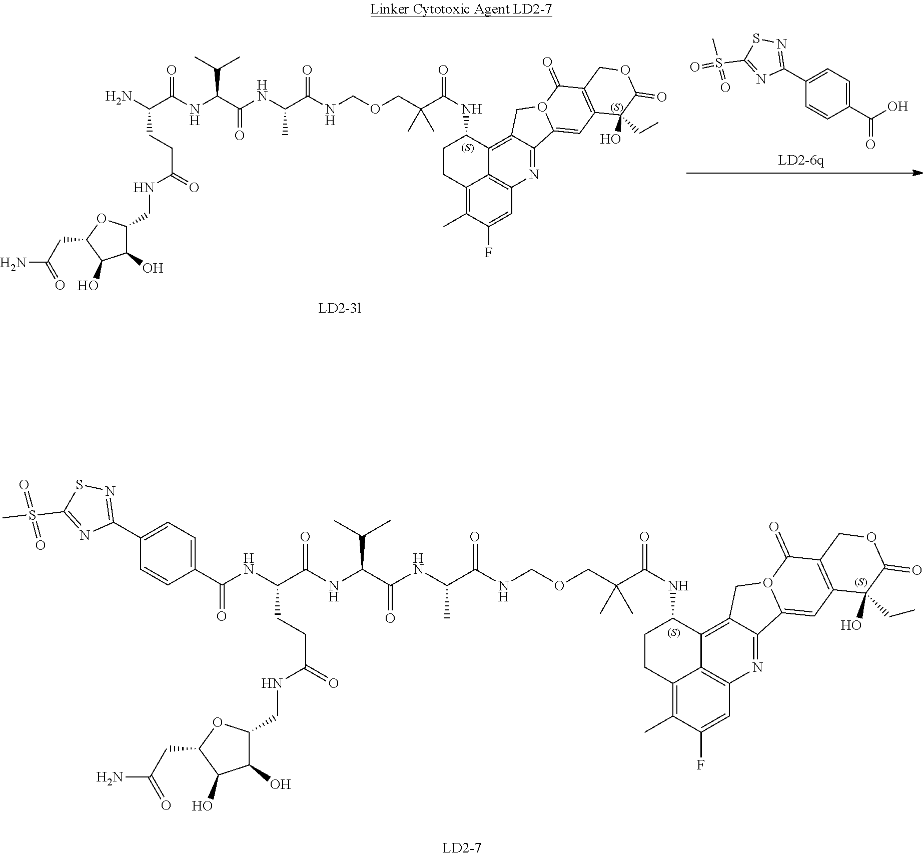

LD2-7 (32 mg, 50.9% yield) was synthesized according to the procedure of step 7 of LD2-3. MS (ESI) m/z: 1303.0 [M+H]⁺.

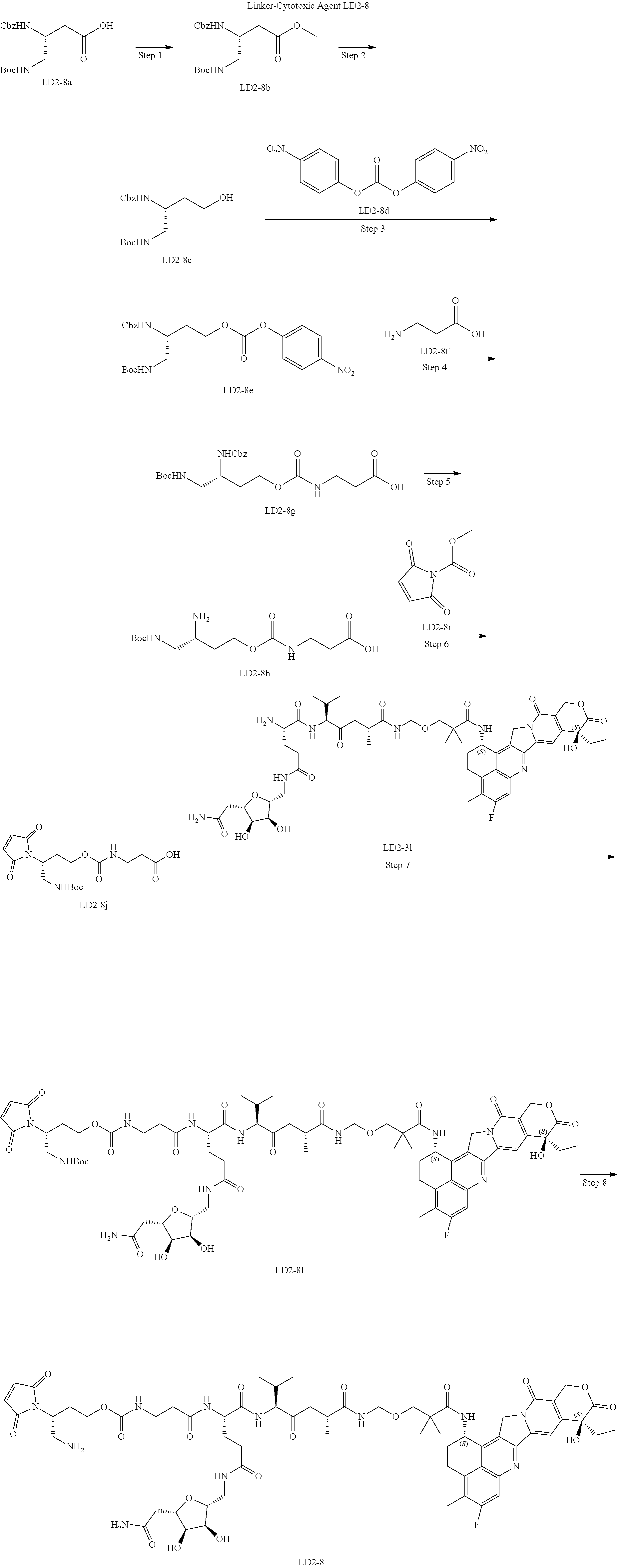

Step 1: Methyl (R)-3-(((benzyloxy)carbonyl)amino)-4-((tert-butoxycarbonyl)amino)butanoate (LD2-8b). LD2-8a (2.00 g, 5.68 mmol) and K₂CO₃ (863 mg, 6.24 mmol) were added into DMF (10 mL) followed by the dropwise addition of CH₃I (1.61 g, 11.35 mmol) at 0° C. The resulting mixture was stirred at 0° C. for 20 min and allowed to warm to 25° C. and further stirred at 25° C. for 60 min. The reaction process was monitored by TLC (PE/EA) and LCMS. After complete reaction, the reaction mixture was diluted with EA (80 mL) and washed with brine (30 mL*3) and H₂O (30 mL*2). The organic layer was dried over anhydrous Na₂SO₄, filtered, and concentrated under reduced pressure to afford the methyl ester of LD2-8b (2.08 g, quant.) as a light-yellow solid. MS (ESI) m/z: 267.2 [M-Boc+H]⁺.

Step 2: Benzyl tert-butyl (4-hydroxybutane-1,2-diyl)(R)-dicarbamate (LD2-8c). LD2-8b (1.00 g, 2.73 mmol) was dissolved in MeOH (15 mL) followed by the addition of LiBH₄ (2 M stock solution in THF, 6.80 mL) at 0° C. The resulting mixture was stirred at 25° C. for 2 h. Reaction process was monitored by LCMS and TLC. After complete reaction, saturated aqueous NH₄Cl (10 mL) was added to quench the reaction. The reaction mixture was diluted with H₂O (80 mL) and extracted with EA (50 mL*3). The combined organic layers were washed with brine (40 mL*2) and water (40 mL*2), dried over anhydrous Na₂SO₄, filtered, and concentrated under reduced pressure and further purified by flash column chromatography (PE/EA) to afford LD2-8c (760 mg, 82.3% yield) as a white solid. MS (ESI) m/z: 239.2 [M-Boc+H]+.

Step 3: Benzyl tert-butyl (4-(((4-nitrophenoxy)carbonyl)oxy)butane-1,2-diyl)(R)-dicarbamate (LD2-8e). LD2-8c (300 mg, 0.89 mmol) and LD2-8d (405 mg, 1.33 mmol) were dissolved in DMF (5 mL) followed by the addition of DIEA (229 mg, 1.77 mmol). The resulting mixture was stirred at 25° C. for 1.5 h. After complete reaction. The reaction mixture was diluted with EA (100 mL) and washed with brine (35 mL*2) and water (35 mL*2). The organic layer was dried over anhydrous Na₂SO₄, filtered, and concentrated under reduced pressure to afford LD2-8e as a white solid (371 mg, 83.1% yield). MS (ESI) m/z: 404.4 [M-Boc+H]⁺.

Step 4: (9H-fluoren-9-yl)methyl tert-butyl (4-(((3-(dimethylamino)-3-oxopropyl)carbamoyl)oxy)butane-1,3-diyl)(S)-dicarbamate (LD2-8g). LD2-8e (420 mg, 0.83 mmol) and LD2-8f (149 mg, 1.67 mmol) were dissolved in DMF (5 mL) followed by the addition of aqueous NaHCO₃ (1M, 5 mL). The resulting mixture was stirred at 25° C. for 2.5 h. After complete reaction, the reaction mixture was concentrated and purified by flash column chromatography (DCM/MeOH) to afford LD2-8g as a pale-yellow solid (365 mg, 96.5% yield). MS (ESI) m/z: 354.4 [M-Boc+H]⁺.

Step 5: (R)-7-(2,5-dioxo-2,5-dihydro-1H-pyrrol-1-yl)-2,2-dimethyl-4,11-dioxo-3,10-dioxa-5,12-diazapentadecan-15-oic acid (LD2-8h). LD2-8g (360 mg, 0.79 mmol) was dissolved in MeOH (18 mL) followed by the addition of Pd/C (wet base, 108 mg). The resulting mixture was stirred at r.t. under H₂ (15 psi) for 2 h. After complete reaction, the reaction mixture was filtered and concentrated under reduced pressure to afford LD2-8h as a clear syrup (252 mg, 99.4% yield). The crude product was used directly in next step without purification. MS (ESI) m/z: 320.3 [M+H]⁺.

Step 6: (R)-7-(2,5-dioxo-2,5-dihydro-1H-pyrrol-1-yl)-2,2-dimethyl-4,11-dioxo-3,10-dioxa-5,12-diazapentadecan-15-oic acid (LD2-8j). LD2-8h (250 mg, 0.78 mmol) and LD2-8i (243 mg, 1.57 mmol) were dissolved in a mixed solvent of ACN (8 mL) and aqueous NaHCO₃ (1M, 16 mL). The resulting mixture was stirred at 0° C. for 1 h and further stirred at 25° C. until the reaction was complete. Then the reaction mixture was acidified with aq. KHSO₄ (20 mL) and extracted with EA (35 mL*3). The combined organic layers were dried over anhydrous Na₂SO₄, filtered, and concentrated under reduced pressure to afford a yellow oil as the crude product which was purified by flash column chromatography to afford LD2-8j (280 mg, 89.6% yield) as a white solid. MS (ESI) m/z: 422.3 [M+Na]⁺. ¹H NMR (400 MHz, d₆-DMSO) δ 12.48 (s, 1H), 7.03-7.01 (m, 2H), 6.99 (s, 2H), 4.08-4.03 (m, 3H), 3.86-3.83 (m, 2H), 3.14-3.11 (m, 2H), 2.35 (t, J=7.2 Hz, 2H), 2.16-2.09 (m, 1H), 1.9LD2-8.84 (m, 1H), 1.32 (s, 9H).

Step 7: (R)-4-((tert-butoxycarbonyl)amino)-3-(2,5-dioxo-2,5-dihydro-1H-pyrrol-1-yl)butyl ((8S,11S,14S)-14-(3-((((2R,3S,4R,5S)-5-(2-amino-2-oxoethyl)-3,4-dihydroxytetrahydrofuran-2-yl)methyl)amino)-3-oxopropyl)-1-(((1S,9S)-9-ethyl-5-fluoro-9-hydroxy-4-methyl-10,13-dioxo-2,3,9,10,13,15-hexahydro-1H′12′-benzo[de]pyrano[3′,4′:6,7]indolizino[1,2-b]quinolin-1-yl)amino)-11-isopropyl-2,2,8-trimethyl-1,7,10,13,16-pentaoxo-4-oxa-6,9,12,15-tetraazaoctadecan-18-yl)carbamate (LD2-81). LD2-81 (32 mg, 50.9% yield) was synthesized according to the procedure of step 7 of example LD2-3. MS (ESI) m/z: 1418.1 [M+H]⁺.

Step 8: (R)-4-amino-3-(2,5-dioxo-2,5-dihydro-1H-pyrrol-1-yl)butyl ((8S,11S,14S)-14-(3-((((2R,3S,4R,5S)-5-(2-amino-2-oxoethyl)-3,4-dihydroxytetrahydrofuran-2-yl)methyl)amino)-3-oxopropyl)-1-(((1S,9S)-9-ethyl-5-fluoro-9-hydroxy-4-methyl-10,13-dioxo-2,3,9,10,13,15-hexahydro′1H′12H-benzo[de]pyrano[3′,4′:6,7]indolizino[1,2-b]quinolin-1-yl)amino)-11-isopropyl-2,2,8-trimethyl-1,7,10,13,16-pentaoxo-4-oxa-6,9,12,15-tetraazaoctadecan-18-yl)carbamate (LD2-8). LD2-81 (22 mg, 0.016 mmol) was dissolved in a mixed solvent of DCM (2 mL) followed by the addition of ZnBr2 (151 mg, 0.67 mmol). The resulting suspension was stirred at 40° C. for 12 h. After complete reaction, the reaction mixture was filtered and concentrated. The residue was diluted with a mixed solvent of CH3CN/aqueous 0.1% FA and purified by prep-HPLC (Method: column: XBridge Prep C18 OBD Sum 19*250 mm; Mobile phase: A-water (0.1% TFA): B-acetonitrile; Flow rate: 20 mL/min) to give compound LD2-8 (13 mg, 61.7% yield) as a white solid. MS (ESI) m/z: 1318.1 [M+H]⁺.

### Example 17. Antibody Drug Conjugate (ADC) Preparation and Evaluation Methods

Reference ADC BGA7650 (Table 18) preparation. Organic solvent (e.g.: DMSO, DMF, DMA, PG, acetonitrile, 0-25% v/v) and linker-payload (2-25 equiv, 10 mM stock in organic solvent) were added stepwise in reaction buffer (PBS buffer pH 6.0-9.0) with the anti-CEA antibody tusamitamab (1-20 mg/mL) under 0-37° C. for 0.5-48 h. The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

Drug-to-antibody ratio (DAR) determination: LCMS method. LC-MS analysis was carried out under the following measurement conditions:

LC-MS system: Vanquish Flex UHPLC and Orbitrap Exploris 240 Mass Spectrometer Column: MAbPacTm RP, 2.1*50 mm, 4 μm, 1,500 A, Thermo Scientific™ Column temperature: 80° C.
Mobile phase A: 0.1% formic acid (FA) aqueous solution; Mobile phase B: Acetonitrile solution containing 0.1% formic acid (FA); Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min) Injected sample amount: 2 μg; MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

DAR 8 antibody drug conjugate preparation. Antibody in conjugation buffer (with concentration 0.5-25 mg/mL, PBS buffer pH 6.0-8.5) was incubated under reduction temperature (0-40° C.) for 10 min and 8-15 equiv. TECP solution (5 mM stock in PBS buffer) was added into the reaction mixture and left the reduction reaction for 1-8 hours at reduction temperature. Organic solvent (e.g., DMSO, DMF, DMA, PG, acetonitrile, 0-25% v/v) and linker-payload stock (10-25 eq, 10 mM stock in organic solvent) were added stepwise after reduction mixture was cooled down to 0-25° C. Conjugation solution was left for 1-3 h at 0-25° C. and the reaction was quenched with N-acetyl cysteine (1 mM stock). The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

Maleimide hydrolysis after conjugation. After the conjugation step, the ADC underwent buffer exchange into ring opening buffer (pH 7.0-9.0, PBS, borate or Tris buffer) and the solution was left at 22 or 37° C. for 5-48 h. Ring opening process was monitored via reduced LCMS. Once the conjugated maleimide hydrolysis is completed, the resulting ADCs were buffer exchanged into basic Tris pH 8.0-8.5 buffer or acidic histidine-acetate pH 5.0-6.5 buffer via dialysis.

ADC characterization. ADC examples were prepared by following the above procedures with a DAR 8 profile. All ADCs were characterized via the following analytical methods. Drug to antibody ratio (DAR) of the ADCs were determined by LCMS method or hydrophobicity interaction column (HIC) method. SEC purity of constructed ADCs were all >95% pure.

### DAR Determination

LCMS Method. LC-MS analysis was carried out under the following measurement conditions:

LC-MS system: Vanquish Flex UHPLC and Orbitrap Exploris 240 Mass Spectrometer

### Column: MAbPac™ RP, 2.1*50 mm, 4 μm, 1,500 A, Thermo Scientific™

Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

HIC method. HPLC analysis was carried out under the following measurement conditions:

HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM Phosphate buffer, pH 7.0
Mobile phase B: 50 mM Phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### ADC Purity: SEC Method

HPLC analysis was carried out under the following measurement conditions:

HPLC system: Waters H-Class UPLC System
Detector: measurement wavelength: 280 nm

### Column: ACQUITY UPLC BEH200 SEC 1.7 um 4.6×150 mm, Waters

Column temperature: room temperature
Mobile phase A: 200 mM Phosphate buffer, 250 mM potassium chloride, 15% isopropyl alcohol, PH 7.0
Gradient program: under 10 min isocratic elutions with the flow rate of 0.3 mL/min
Injected sample amount: 20 μg

### ADC Hydrophobicity Evaluation: HIC

ADCs with greater hydrophobic properties appear at later retention times with HIC chromatography.

HPLC analysis was carried out under the following measurement conditions:

### Method 1

HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM Phosphate buffer, pH 7.0
Mobile phase B: 50 mM Phosphate buffer, 25% (V/V) Isopropanol, pH 7.0 Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### Method 2

HPLC system: Waters ACQUITY ARC HPLC System
Detector: measurement wavelength: 280 nm

### Column: MABPac HIC-10, 5 μm, 4.6×10 mm (Thermo)

Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B-20% B (0 min-1 min), 0% B-0% B (1 min-35 min), 20% B-20% B (35 min-40 min)
Flow rate: 0.5 mL/min
Sample preparation: The sample was diluted with initial mobile phase to 0.5 mg/mL.

TABLE 18
Constructed Conjugates

| ADC No. | Antibody | ADC structure |
|---|---|---|
| BGA7650 | Tusamitamab (Reference antibody) | 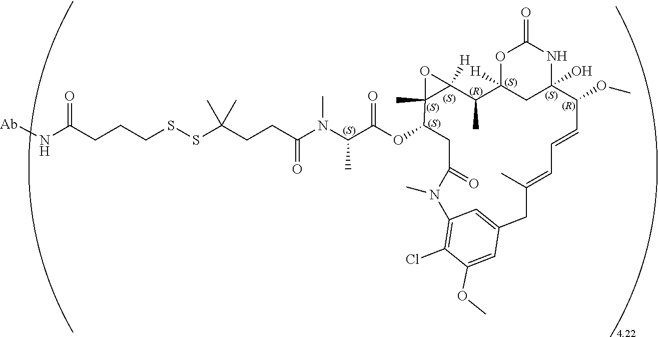 |
| BGA2588 | BGA5384 | 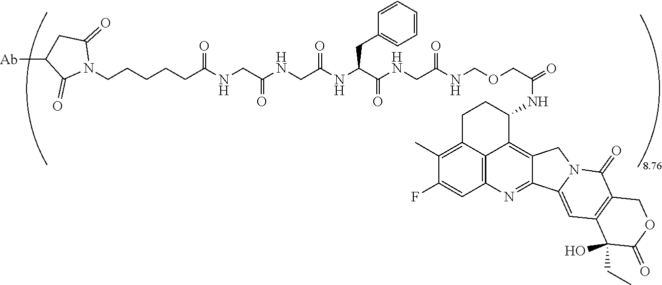 |
| BGA9962 | BGA5384 | 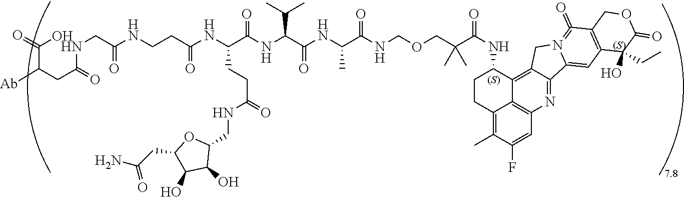 |
| BGA8357 | BGA5384 | 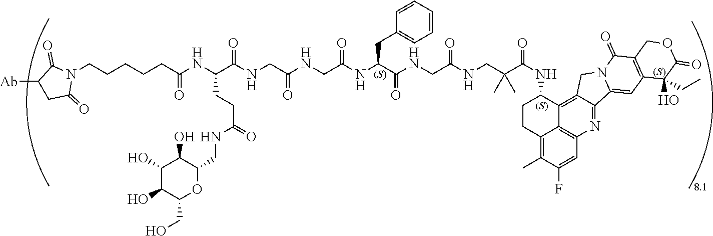 |
| BGA0084 | BGA5384 | 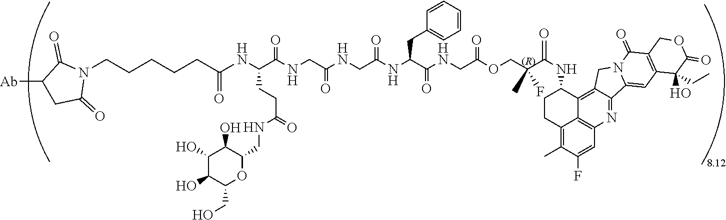 |
| BGA7413 | BGA5384 | 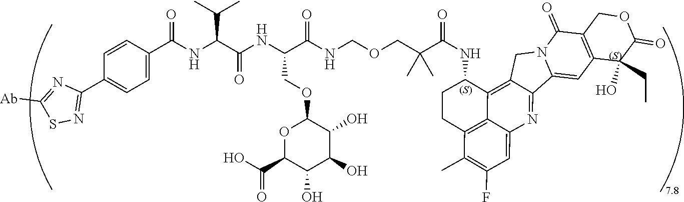 |
| BGA2490 | BGA5384 | 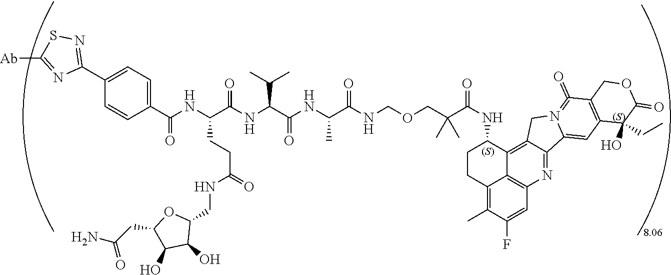 |
| BGA0179 | BGA5384 | 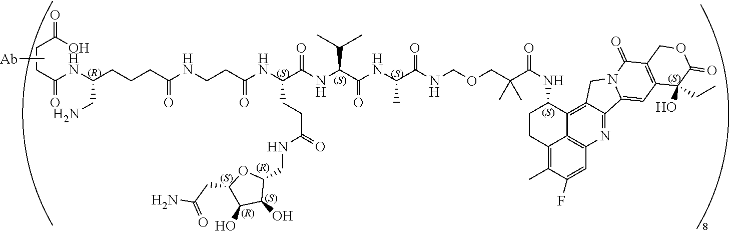 |

### Tusamitamab Sequence

>LC
(SEQ ID NO: 105)
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
>HC
(SEQ ID NO: 106)
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

### Cell Line Information

### MKN45 (JCRB, JCRB0254)

MKN45 is a cell line exhibiting round morphology. It loosely attaches to substratum that was isolated in 1998 from the stomach tissue of a 62-year-old female with stomach cancer. MKN45 was purchased from JCRB. The base medium for MKN45 is RPMI-1640 Medium, Gibco 22400089. To make the complete growth medium, the following components are added to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### SNU-16 (ATCC, CRL-5974)

SNU-16 is a cell line exhibiting epithelial morphology that was isolated in 1987 from ascites derived from a 33-year-old, female, Asian, stomach cancer patient prior to chemotherapy and SNU16 was purchased from ATCC. The base medium for SNU16 is RPMI-1640 Medium, Gibco 22400089. To make the complete growth medium, add the following components to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### NCI-H2122 (ATCC, CRL-5985)

NCI-H2122 cells are lymphoblasts that were isolated in 1989 from a pleural effusion metastasis derived from a 46-year-old female smoker and NCI-H2122 was purchased from ATCC. The base medium for NCI-H2122 is RPMI-1640 Medium, Gibco 22400089. To make the complete growth medium, the following components are added to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert” PLUS Mycoplasma Detection Kit (Lonza, LT07-710).

### LS174T (ATCC, CL-188)

LS 174T is a cell line that exhibits epithelial morphology that was isolated from the colon of a White, 58-year-old, female adenocarcinoma patient with colorectal cancer and LS174T was purchased from ATCC. The base medium for LS174T is ATCC-formulated Eagle's Minimum Essential Medium, 30-2003. To make the complete growth medium, the following components are added to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### MDA-MB-231 (ATCC, HTB-26)

MDA-MB-231 is an epithelial-like cell that was isolated from the mammary gland of a 40-year-old White female with adenocarcinoma and MDA-MB-231 was purchased from ATCC. The base medium for MDA-MB-231 is RPMI-1640 Medium, Gibco 22400089. To make the complete growth medium, the following components are added to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### HCT116 (ATCC, CCL-247)

The HCT116 cell line was isolated from the colon of an adult male with colon cancer, has a mutation in codon 13 of the ras proto-oncogene and was purchased from ATCC. The base medium for MDA-MB-231 is RPMI-1640 Medium, Gibco 22400089. To make the complete growth medium, the following components are added to the base medium: fetal bovine serum to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### Additional Cell Lines

SW1463 cells were derived from a human colorectal adenocarcinoma and express a moderate level of CEA.

NCI-N87 express low levels of CEA and were derived from a gastric carcinoma.

HT29 cells are low to negative for CEA expression and were derived from a human colorectal adenocarcinoma.

TABLE 19
Cell Lines and Their CEA Expression Levels

| | | | CEA Level (Molecular |
|---|---|---|---|
| Cell Line | Vendor | Cat. No. | No. (Dako kit)) |
| MKN45 | JCRB | JCRB0254 | ~200K |
| SNU16 | ATCC | CRL-5974 ™ | ~100K |
| Ls174T | ATCC | CL-188 ™ | ~13K |
| NCI-H2122 | ATCC | CRL-5985 ™ | ~18K |
| MDA-MB-231 | ATCC | HTB-26 ™ | 0 |
| HCT116 | ATCC | CCL-247 ™ | 0 |

### Example 18. In Vitro Cell Killing by CEA Antibodies Conjugated with Various Payloads

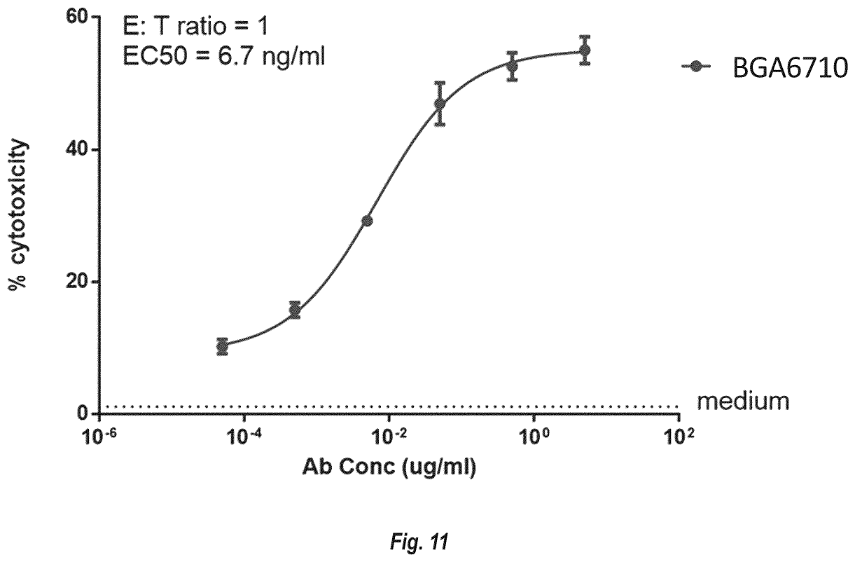

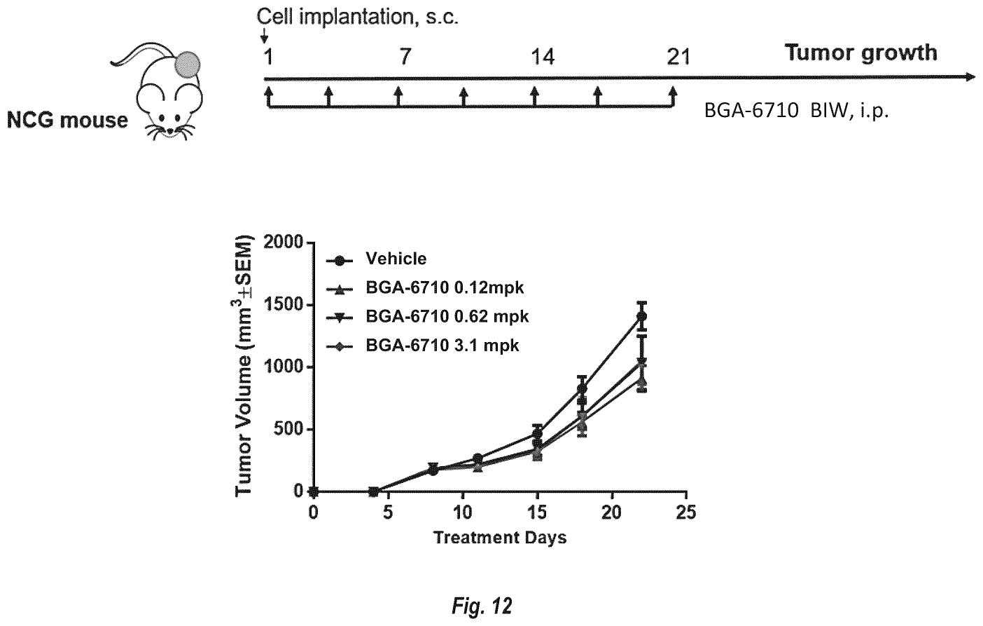

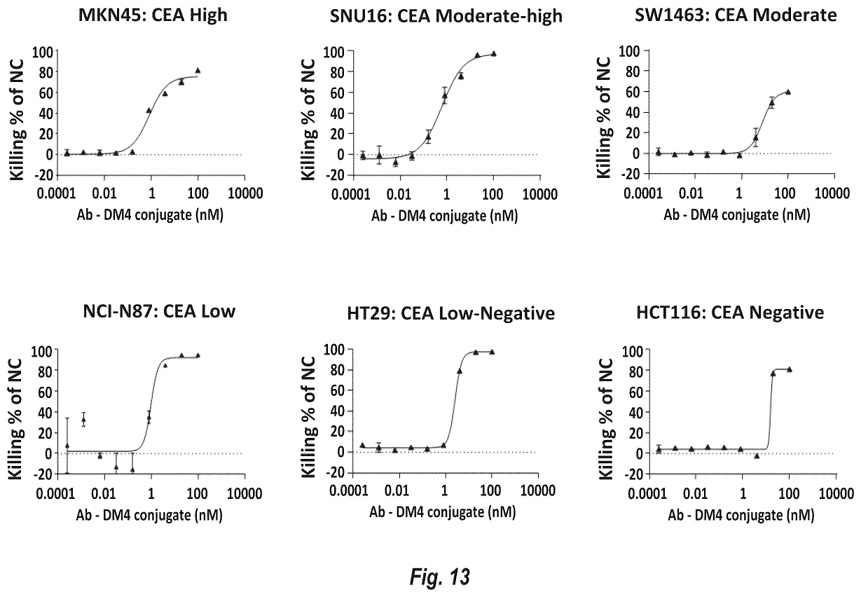

BGA5384 antibodies (Table 20) were conjugated using an in-house generated linker and various payloads according to Example 17. BGA5384 was conjugated with the maytansinoid DM4 (FIG. 13), the auristatin MMAE (FIG. 14), and the topoisomerase DXD (BGA2588) (FIG. 15).

TABLE 20
Amino acid sequences of BGA5384

| | | |
|---|---|---|
| SEQ ID | QVQLVQSGAEVKKPGASVKVSCKASGYIFTSYYLHWVRQAPGQGLEWIGYIN | BGA5384 |
| NO: 99 | PQTGKTSYAQKFQGRVTMTRDTSTSTVYMELSSLRSEDTAVYYCAREYGNYN | (BGA7592-1K) |
| | YPLDYWGQGTLVTVSSASTKGPSVFPLAPSSKSTSGGTAALGCLVKDYFPEP | heavy |
| | VTVSWNSGALTSGVHTFPAVLQSSGLYSLSSVVTVPSSSLGTQTYICNVNHK | chain |
| | PSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSRDELTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPGK | |
| SEQ ID | DIQMTQSPSSLSASVGDRVTITCRASENQYGYLAWYQQKPGKVPKLLIYNYK | BGA5384 |
| NO: 100 | NLVEGVPSRFSGSGSGTDETLTISSLQPEDVATYYCQHHLGTPYTFGQGTKV | (BGA7592-1K) |
| | EIKRTVAAPSVFIFPPSDEQLKSGTASVVCLLNNFYPREAKVQWKVDNALQS | light |
| | GNSQESVTEQDSKDSTYSLSSTLTLSKADYEKHKVYACEVTHQGLSSPVTKS | chain |
| | ENRGEC | |

To determine the amount of cell killing by each ADC, cells from lines with varying CEA expression levels (Example 17; Table 20) were seeded in 96-well plates and incubated at 37° C. overnight. Serial diluted ADC was added, and the cells were then cultured for 6 days and subjected to a cell viability assay. As shown in FIGS. 13-15, all of the CEA antibody drug conjugates showed good cell killing in high to moderate CEA expressing cells at a low concentration of CEA ADC. In low to low-negative to negative expressing CEA cells, high concentrations of CEA ADC were necessary to show cell killing. These data indicate that the BGA5384 antibody can be conjugated to various payloads and achieve cell killing in a range of CEA expressing cells.

### Example 19. Payload Sensitivity: MKN45

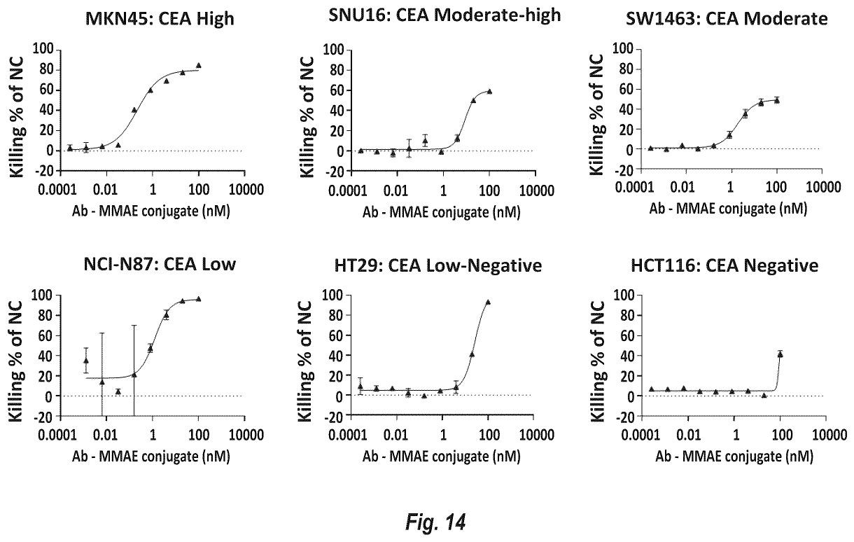

The effects of payloads on MKN45 cells are shown in FIG. 16. At Day 0, cells were harvested with 0.25% trypsin-EDTA, plated at 5,000 cells/well in 96-well plates (655090, Greiner), and incubated at 37° C., 5% CO₂ overnight. At Day 1, payload compounds were added (5× dilution) into plates. Cell and compound mixtures were incubated at 37° C., 5% CO₂ for 6 days. Survival cell signaling was collected at Day 6 with 100 μL of detection reagent (G7573, Promega®); signal was read by Tecan Spark®. Data were analyzed by GraphPad Prism 9.0.0. All killing concentrations were duplicated or triplicated (Table 21 and FIG. 16). “SABC” refers to specific antibody binding capacity.

TABLE 21
Effects of payloads on MKN45 cells

| | MKN45 | |
|---|---|---|
| Payload | Emax | EC₅₀(nM) |
| P1 | 97.6 | 1.16 |
| P2 | 98.1 | 2.08 |
| P3 | 97.0 | 0.89 |

### Example 20. ADC Direct Cellular Killing Assays

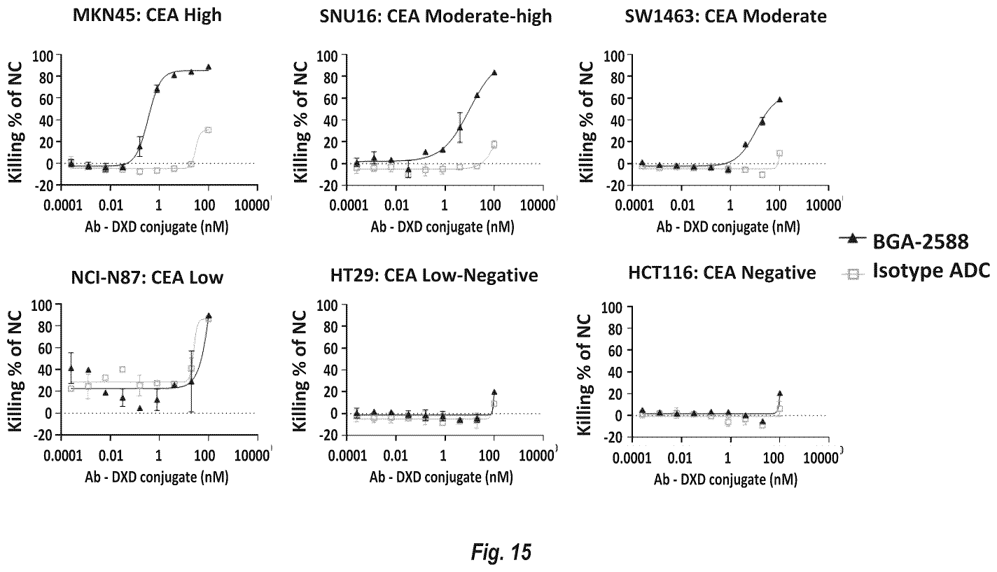

FIGS. 17-20 show the cellular activities of 8 of the different constructed ADCs (see Table 18) in MKN45 (stomach cancer), H2122 (lung adenocarcinoma), LS174T (colorectal adenocarcinoma), and MB-231 (breast adenocarcinoma) patient-derived cell lines, respectively.

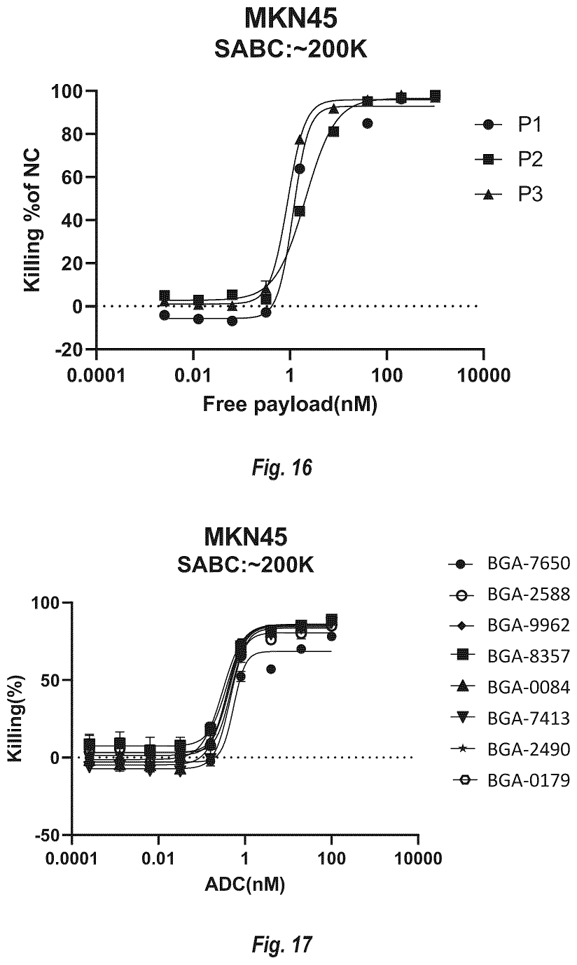

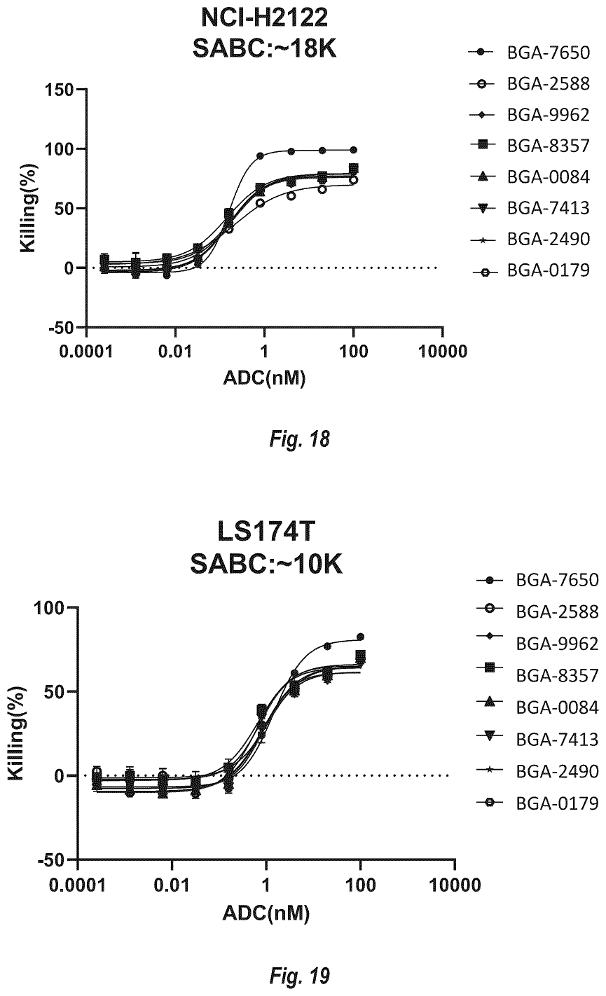

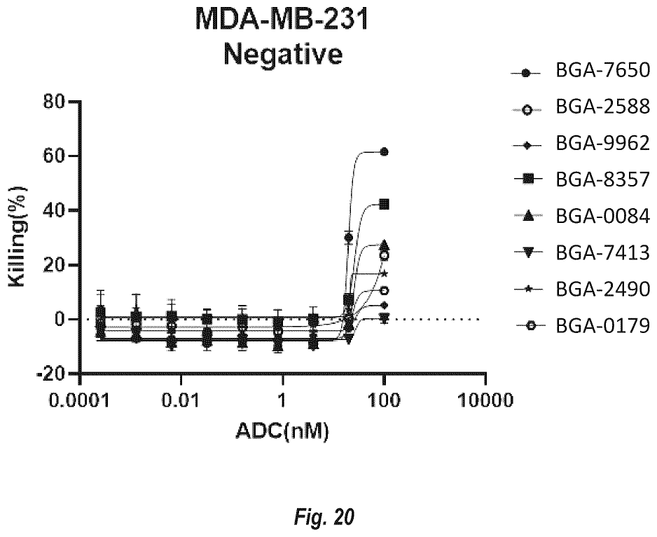

At Day 0, cells were harvested with 0.25% Trypsin-EDTA, plated at 5,000 cells/well (MKN45 or Ls174T) or 2,000 cells/well (NCI-H2122 or MDA-MB-231) in 96-well plates (655090, Greiner), and incubated at 37° C., 5% CO₂ overnight. At Day 1, ADCs were added (5× dilution) into plates. The cell and ADC mixture was incubated at 37° C., 5% CO₂ for 6 days. Survival cell signaling was collected at Day 6 with 100 μL of detection reagent (G7573, Promega®); the signal was read by Tecan Spark*. Data were analyzed by GraphPad Prism 9.0.0. All of the killing concentrations were duplicated or triplicated. Results are presented for each of MKN45 (Table 22 and FIG. 17), NCI-H2122 (Table 23 and FIG. 18), Ls174T (Table 24 and FIG. 19), and MDA-MB0231 (Table 25 and FIG. 20) cells.

TABLE 22
ADC cellular activities on MKN45 cell line

| | MKN45 | |
|---|---|---|
| ADC | Emax | EC₅₀(nM) |
| BGA7650 | 78.2 | 0.55 |
| BGA2588 | 85 | 0.46 |
| BGA9962 | 87.1 | 0.39 |
| BGA8357 | 89.1 | 0.38 |
| BGA0084 | 88.8 | 0.38 |
| BGA7413 | 87.2 | 0.46 |
| BGA2490 | 89 | 0.44 |
| BGA0726 | 89.1 | 0.3 |

TABLE 23
ADC cellular activities on NCI-H2122 cell line

| | NCI-H2122 | |
|---|---|---|
| ADC | Emax | EC₅₀(nM) |
| BGA7650 | 99 | 0.16 |
| BGA2588 | 74 | 0.22 |
| BGA9962 | 80 | 0.17 |
| BGA8357 | 83.9 | 0.14 |
| BGA0084 | 83.8 | 0.19 |
| BGA7413 | 81.5 | 0.16 |
| BGA2490 | 82.8 | 0.19 |
| BGA0726 | 81.2 | 0.16 |

TABLE 24
ADC cellular activities on Ls147T cell line

| | LS174T | |
|---|---|---|
| ADC | Emax | EC₅₀(nM) |
| BGA7650 | 82.5 | 1.44 |
| BGA2588 | 69.7 | 1.12 |
| BGA9962 | 66.7 | 0.83 |
| BGA8357 | 71.8 | 0.67 |
| BGA0084 | 69.9 | 0.85 |
| BGA7413 | 66.9 | 0.88 |
| BGA2490 | 70.3 | 0.76 |
| BGA0726 | 69.7 | 0.62 |

TABLE 25
ADC cellular activities on MDA-MB-231 cell line

| | MDA-MD-231 | |
|---|---|---|
| ADC | Emax | EC₅₀(nM) |
| BGA7650 | 61.6 | 20 |
| BGA2588 | 23.6 | >100 |
| BGA9962 | 5.2 | 27 |
| BGA8357 | 42.4 | 26 |
| BGA0084 | 27.4 | 25 |
| BGA7413 | 0.4 | 28 |
| BGA2490 | 16.9 | 21 |
| BGA0726 | 10.7 | 24 |

### Example 21. Efficacy of ADCs BGA7650 and BGA9962 in Cell-Line Derived Xenograft Models

Cell-line derived xenograft (CDX) models using MKN-45 (CEA high expression) (gastric adenocarcinoma), SW-1463 (CEA moderate) (rectal adenocarcinoma), and NCI-H2122 (CEA low) (lung adenocarcinoma) were generated as described below.

Cells were cultured in RPMI-1640 medium, supplemented with 10% (v/v) fetal bovine serum, 100 U/ml penicillin, and 100 μg/mL streptomycin. On the day of implantation, cells were collected and re-suspended in cold (4° C.) serum-free RPMI-1640 medium. Cell density was adjusted to 2 (MKN-45), 1.5 (SW1463), or 4 (NCI-H2122)×107 cells/mL, and the cells were placed on ice prior to inoculation.

Six- to eight-week-old female mice were purchased and housed in ventilated cages, given food and water ad libitum, and allowed to acclimate for approximately 1 week prior to inoculation. MKN-45, SW-1463, and NCI-H2122 tumors were induced on the right flank by a subcutaneous injection of 2.0 (MKN-45), 3.0 (SW1463), or 8.0 (NCI-H2122)×10⁶ cells in NCG, NCG, and Balb/c nude mice, respectively.

Experiments were performed approximately 2-3 weeks following injection of the cancer cells. For tumor volume measurements, all tumors were measured with calipers and tumor volumes were calculated using the formula V=0.5 (a×b²), in which “a” is the tumor length and “b” is the tumor width and/or height. When tumor volume reached approximately 200 mm; in size, mice were randomized into 5 groups with 8, 9, and 9 animals in vehicle, BGA7650, and BGA9962 groups on Day 0, respectively. After ensuring all cohorts had approximately equal average tumor volumes to start, animals were intravenously administered vehicle, BGA7650 (1.3 mg/kg or 4 mg/kg; or “mpk”), and BGA9962 (2 mg/kg or 6 mg/kg) on treatment Day 1. Animal body weight and tumor volume were measured twice weekly. Data is presented as mean tumor volume f standard error of the mean (SEM). Tumor growth inhibition (TGI) was calculated using the following formula:

$$\% TGI = [ 1 - ( treated Tt - treated T 0 ) / ( vehicle Tt - vehicle T 0 ) ] \times 100 \%$$

treated Tt=mean tumor volume of a dosing group on Day t
treated T0=mean tumor volume of a dosing group on Day 0
vehicle Tt=mean tumor volume of vehicle group on Day t
vehicle T0=mean tumor volume of vehicle group on Day 0

### Results

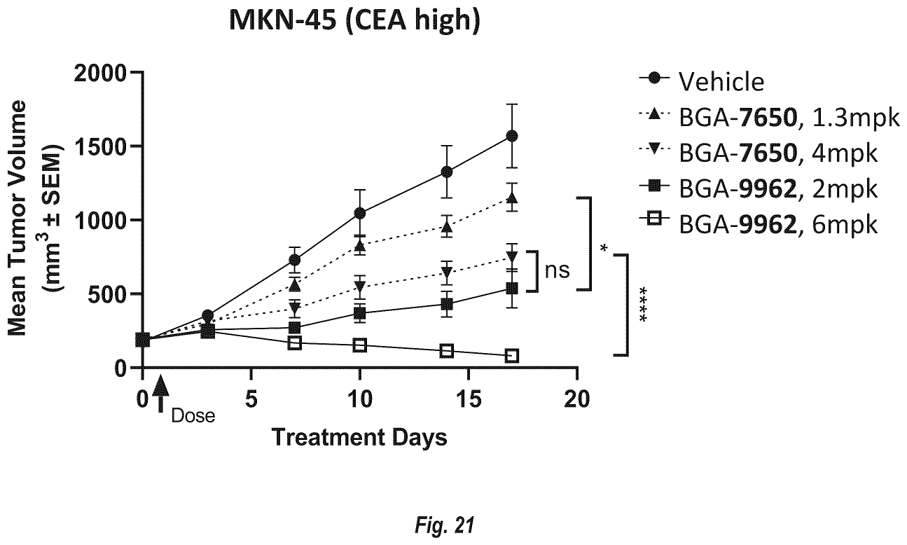

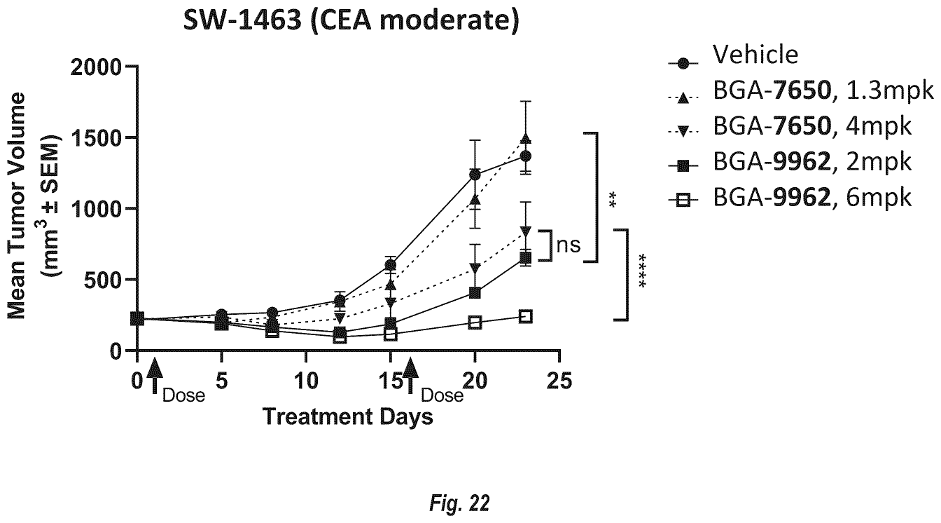

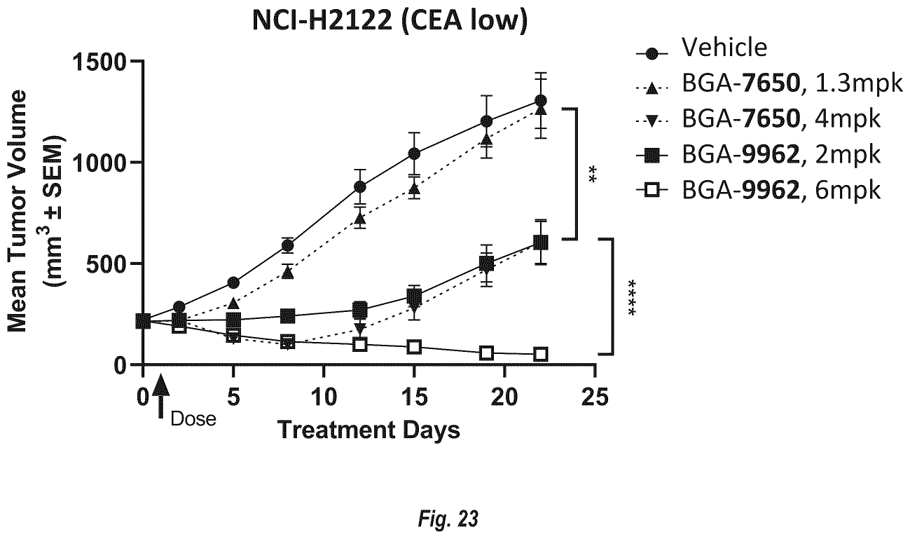

In cell-line derived xenograft (CDX) models using MKN-45 (FIG. 21); SW-1463 (FIG. 22); and NCI-H2122 (FIG. 23), BGA9962 and BGA7650 treatment groups slowed tumor growth compared to the vehicle group.

In these three CDX models, both BGA9962 and BGA7650 showed dose-dependent efficacy (FIGS. 21, 22, and 23). BGA7650 at 4 mg/kg, but not 1.3 mg/kg, induced significant anti-tumor efficacy. BGA9962 at 2 mg/kg demonstrated superior anti-tumor efficacy compared to BGA7650 at 1.3 mg/kg and comparable efficacy to BGA7650 at 4 mg/kg. In addition, BGA9962 at 6 mg/kg significantly reduced tumor growth and demonstrated a higher anti-tumor effect than BGA7650 at both dosages (1.3 and 4 mg/kg). All animals tolerated treatment well with no significant body weight decrease or abnormal clinical observations.

### Example 22. Efficacy of BGA7650 and BGA9962 in Human Patient-Derived Gastric Cancer Xenograft Model

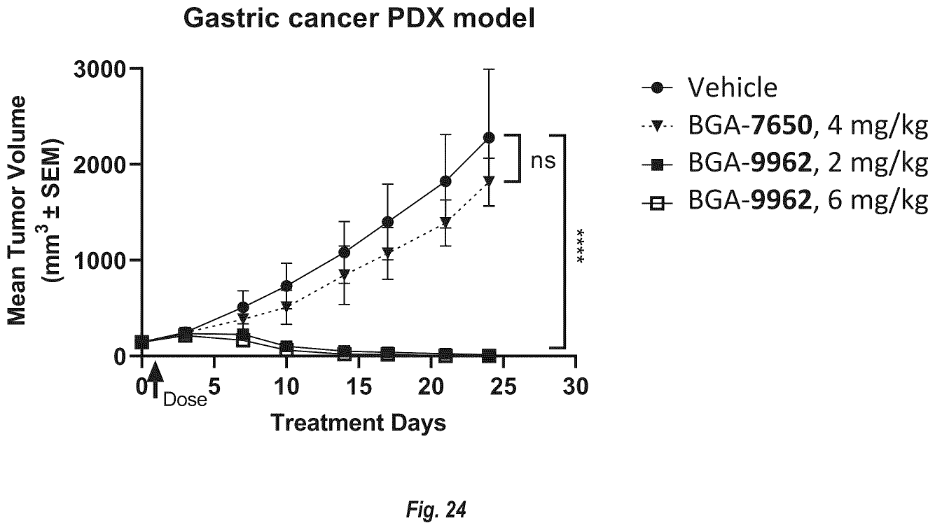

The anti-tumor effects of BGA7650 and BGA9962 were evaluated in a human patient-derived gastric cancer (“GC”) xenograft model (FIG. 24) initiated as described in Example 21. Single dose treatment with 4 mg/kg BGA7650, 2 mg/kg BGA9962, or 6 mg/kg BGA9962 induced 22% (P=0.8444), 106% (P<0.0001), and 107% (P<0.0001) tumor growth inhibition (TGI) rate on treatment day 24, respectively. BGA9962 at both dosages (2 mg/kg and 6 mg/kg) demonstrated significantly higher anti-tumor activity than BGA7650 at 4 mg/kg (FIG. 24 and Table 26). All animals tolerated treatment well with no significant body weight decrease or abnormal clinical observations.

TABLE 26
TGI measurement of BGA7650 and
BGA9962 at treatment Day 24.

| ADC | TGI (%) at T24 |
|---|---|
| BGA7650 4 mg/kg | 22 |
| BGA9962 2 mg/kg | 106 |
| BGA9962 6 mg/kg | 107 |

### Example 23. Single-Dose PK in Non-Tumor-Bearing Mice at 3 mg/kg (i.v.)

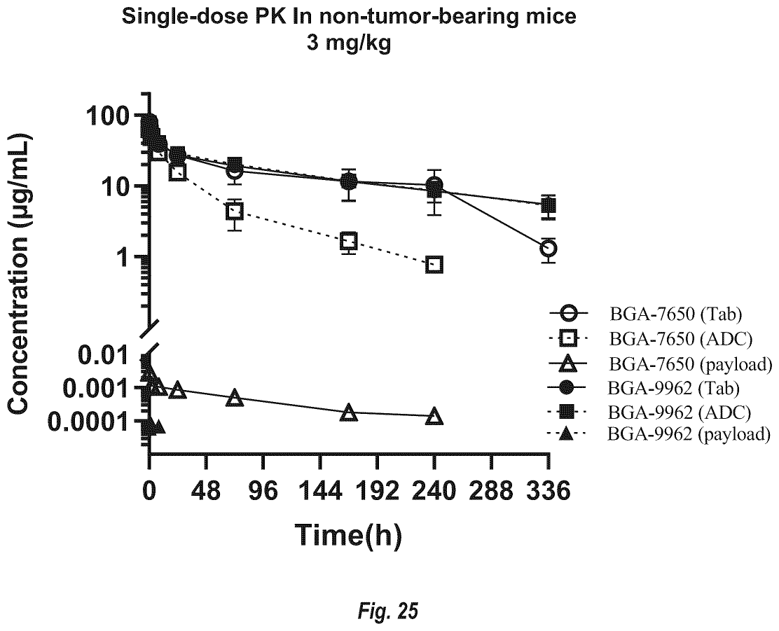

A single dose of BGA9962 or BGA7650 at 3 mg/kg (i.v.) was administered to Balb/c nude (non-tumor-bearing) mice as described in Example 21 and pharmacokinetics were evaluated. BGA9962 showed a strong pharmacokinetic (PK) profile in Balb/c nude (non-tumor-bearing) mice (n=3) compared to BGA7650 (FIG. 25). BGA9962 demonstrated superior PK against comparator BGA7650. BGA9962 (triangles linked by dashed lines) resulted in lower serum-free payload in plasma compared to BGA7650 (triangles linked by solid lines).

A separation was observed between TAb (total antibody) and ADC for the comparator BGA7650 (solid line linking unfilled circles) but not for BGA9962 (solid lines linking unfilled squares). These results confirm a stable DAR over time for BGA9962 compared to BGA7650. In vitro, BGA9962 is stable in mouse and human plasma with no DAR change after 336 hours of incubation (data not shown).

### Example 24. In Vivo DAR Quantification and Comparative Stability

In vivo drug-to-antibody ratio (DAR) for BGA7650 and BGA9962 was analyzed by intact LC-MS. A serum sample (from Balb/c nude mice treated with a single i.v. dose of ADC at 3 mg/k, n=3 per group) was processed into payload-conjugated peptides from the heavy chain (HC) and light chain (LC) of the ADC. DAR mass spectra were analyzed to calculate average DAR of each chain, and average DAR of the intact ADC was calculated using the following formula: DAR=(DAR(LC)+DAR(HC))×2.

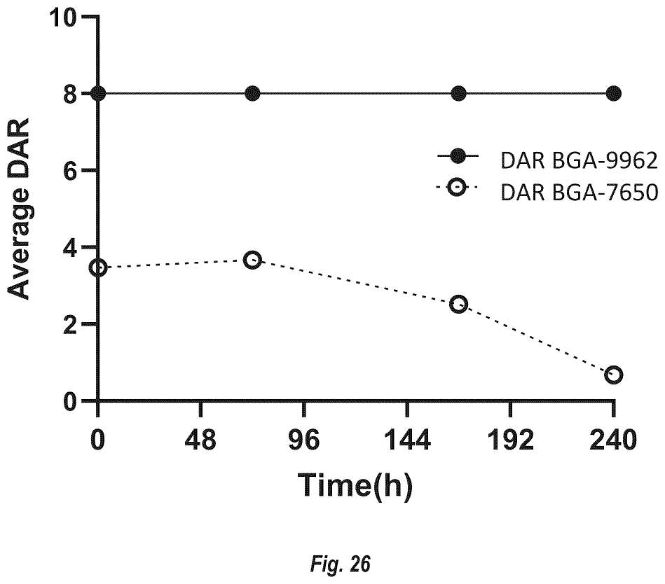

As BGA7650 possesses payload conjugation through lysine at no specific positions, it was difficult to process for intact LC-MS DAR analysis. Instead, the in vivo DAR for BGA7650 was measured indirectly by analyzing the ratio of total conjugated payload to total antibody concentration using immunocapture followed by anti-Fc (for total antibody detection) and LC-MS (for conjugated payload detection). Thus, FIG. 26 shows that BGA9962 maintained stable DAR of 8 in vivo with minimum deconjugation, while BGA7650 deconjugates steadily over time.

1. An antibody drug conjugate of the formula Ab4C-L-(D)ₘ)ₙ, or a pharmaceutically acceptable salt, solvate, or hydrate thereof, wherein:
Ab is an antibody or antigen-binding fragment thereof, which binds to human CEA and which comprises:
(i) three heavy chain CDRs:
HCDR1 comprising an amino acid sequence as set forth in SEQ ID NO:24,
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:23; or
(ii) three heavy chain CDRs:
HCDR1 comprising an amino acid sequence as set forth in SEQ ID NO:7,
HCDR2 comprising an amino acid sequence as set forth in SEQ ID NO:8,
HCDR3 comprising an amino acid sequence as set forth in SEQ ID NO:9, and
three light chain CDRs:
LCDR1 comprising an amino acid sequence as set forth in SEQ ID NO:10,
LCDR2 comprising an amino acid sequence as set forth in SEQ ID NO:11,
LCDR3 comprising an amino acid sequence as set forth in SEQ ID NO:6; or
(iii) three heavy chain CDRs:
HCDR1 comprising an amino acid sequence as set forth in SEQ ID NO:41,
HCDR2 comprising an amino acid sequence as set forth in SEQ ID NO:42,
HCDR3 comprising an amino acid sequence as set forth in SEQ ID NO:43, and
three light chain CDRs:
LCDR1 comprising an amino acid sequence as set forth in SEQ ID NO:44,
LCDR2 comprising an amino acid sequence as set forth in SEQ ID NO:45,
LCDR3 comprising an amino acid sequence as set forth in SEQ ID NO:40;
C is a conjugator;
L is a linker;
D is a cytotoxic agent:
m is an integer from 1 to 8; and
n is from 1 to 10.

2. (canceled)

3. The antibody drug conjugate of claim 1, wherein m is 1.

4. The antibody drug conjugate of claim 1, wherein n is from 3 to 10.

5. The antibody drug conjugate of claim 4, wherein n is about 8.

6. The antibody drug conjugate of claim 1, wherein C is a formula selected from (C-I), (C-Ia), (C-Ib), (C-II), (C-III), (C-IIIa), or (C-IV): 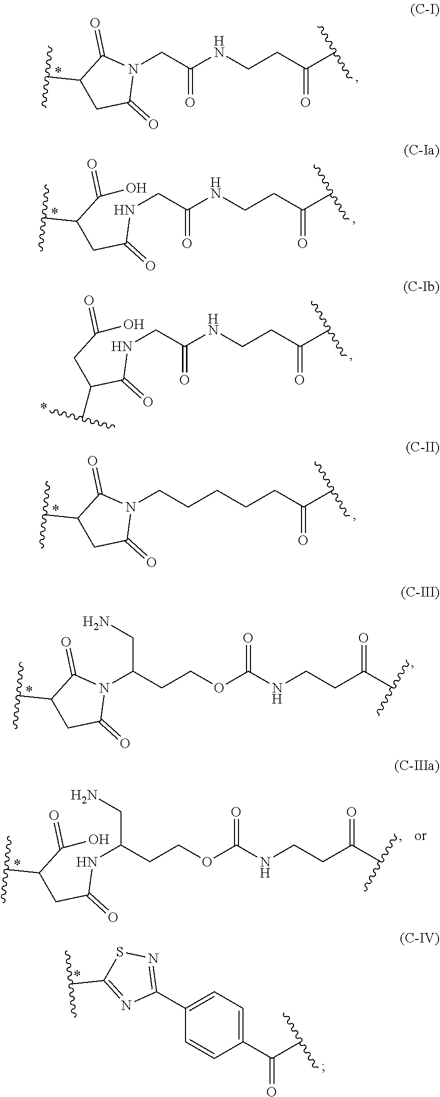
and
* marks the bond where C connects to Ab.

7. The antibody drug conjugate of claim 6, wherein C is

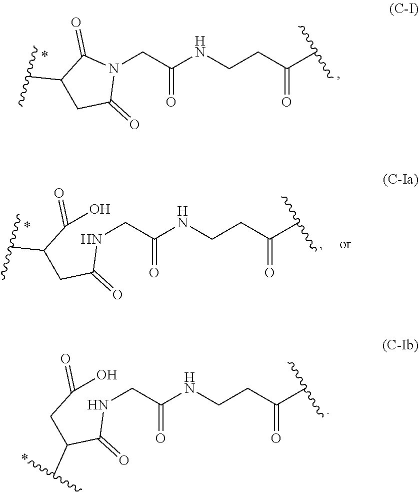

8. The antibody drug conjugate of claim 1, wherein
L is a formula selected from (L-I), (L-II), or (L-III): 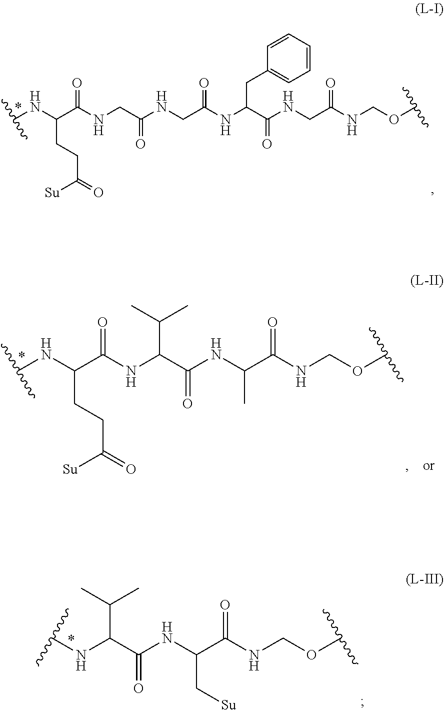
wherein Su is a hydrophilic residue; and
* marks the bond where L connects to C.

9. The antibody drug conjugate of claim 8, wherein Su is

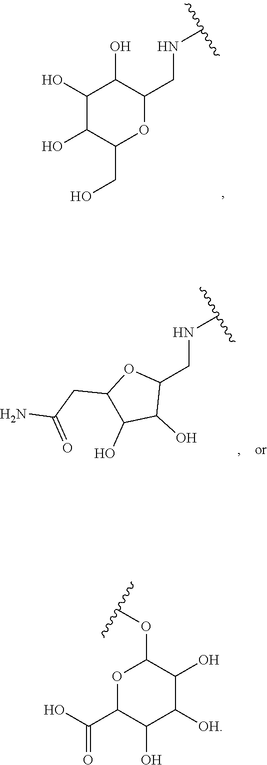 [SMILES: *NCC1OC(CO)C(O)C(O)C1O ; *NCC1OC(CC(N)=O)C(O)C1O ; *OC1OC(C(=O)O)C(O)C(O)C1O]

10. The antibody drug conjugate of claim 9, wherein Su is

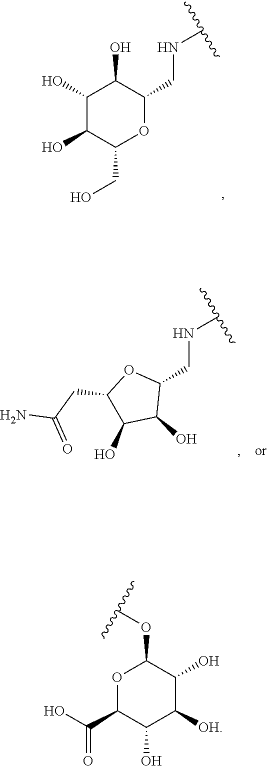 [SMILES: *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

11. The antibody drug conjugate of claim 1, wherein L is 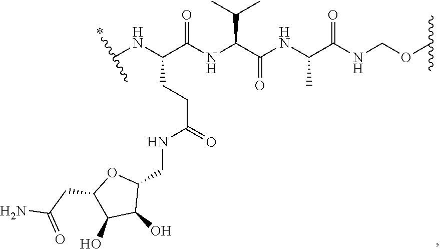
wherein * marks the bond where L connects to C.

12. The antibody drug conjugate of claim 1, wherein the cytotoxic agent is a topoisomerase inhibitor.

13. The antibody drug conjugate of claim 1, wherein D is: 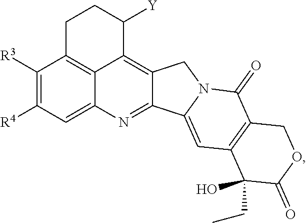
wherein
Y is -A-B-C′-D′-*, wherein * marks the bond where D connects to L;
A is a bond, CR¹R², or N-R¹;
B is a bond, —C(═O)—, or —C(═O)O—;
C′ is a bond or a divalent group, wherein the divalent group is unsubstituted or substituted C₁₋₈ alkyl, unsubstituted or substituted cycloalkyl, unsubstituted or substituted heterocyclyl, unsubstituted or substituted aryl, or unsubstituted or substituted heteroaryl;
D¹ is a bond, NH, or O;
each of R¹ and R² is, independently, hydrogen, halogen, substituted or unsubstituted alkyl, or substituted or unsubstituted alkoxyl; or R¹ and R² together with the atom to which they are attached, form unsubstituted or substituted cycloalkyl, unsubstituted or substituted heterocyclyl, unsubstituted or substituted aryl, or unsubstituted or substituted heteroaryl;
each of R³ and R⁴ is, independently, hydrogen, halogen, substituted or unsubstituted alkyl, or substituted or unsubstituted alkoxyl; or R³ and R⁴ together with the atoms to which they are attached, form unsubstituted or substituted cycloalkyl, unsubstituted or substituted heterocyclyl, unsubstituted or substituted aryl, or unsubstituted or substituted heteroaryl.

14. The antibody drug conjugate of claim 1, wherein D is: 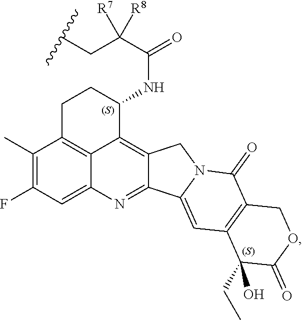
wherein R⁷ and R⁸ are each independently hydrogen, halogen, or alkyl.

15. The antibody drug conjugate of claim 1, wherein D is selected from:

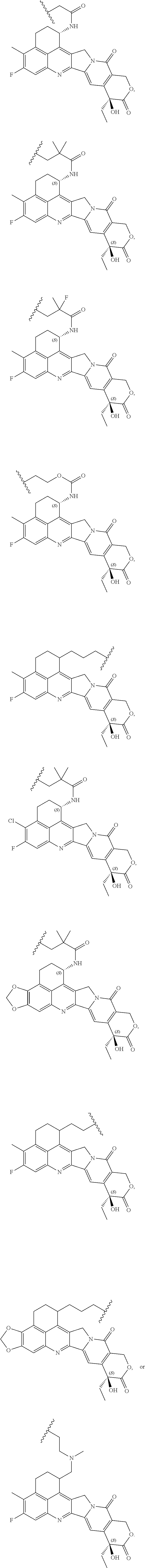

16. The antibody drug conjugate of claim 15, wherein D is

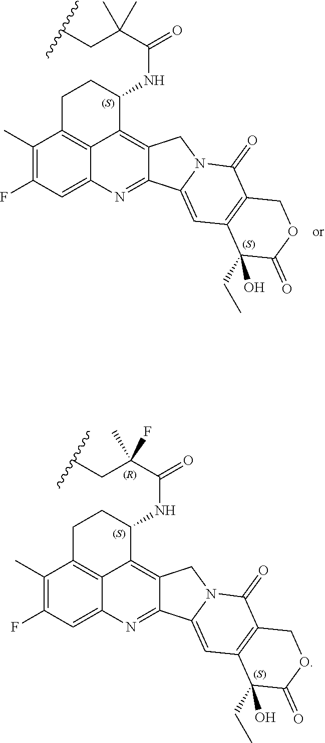

17. The antibody drug conjugate of claim 1, wherein C-L-(D)ₘ is:

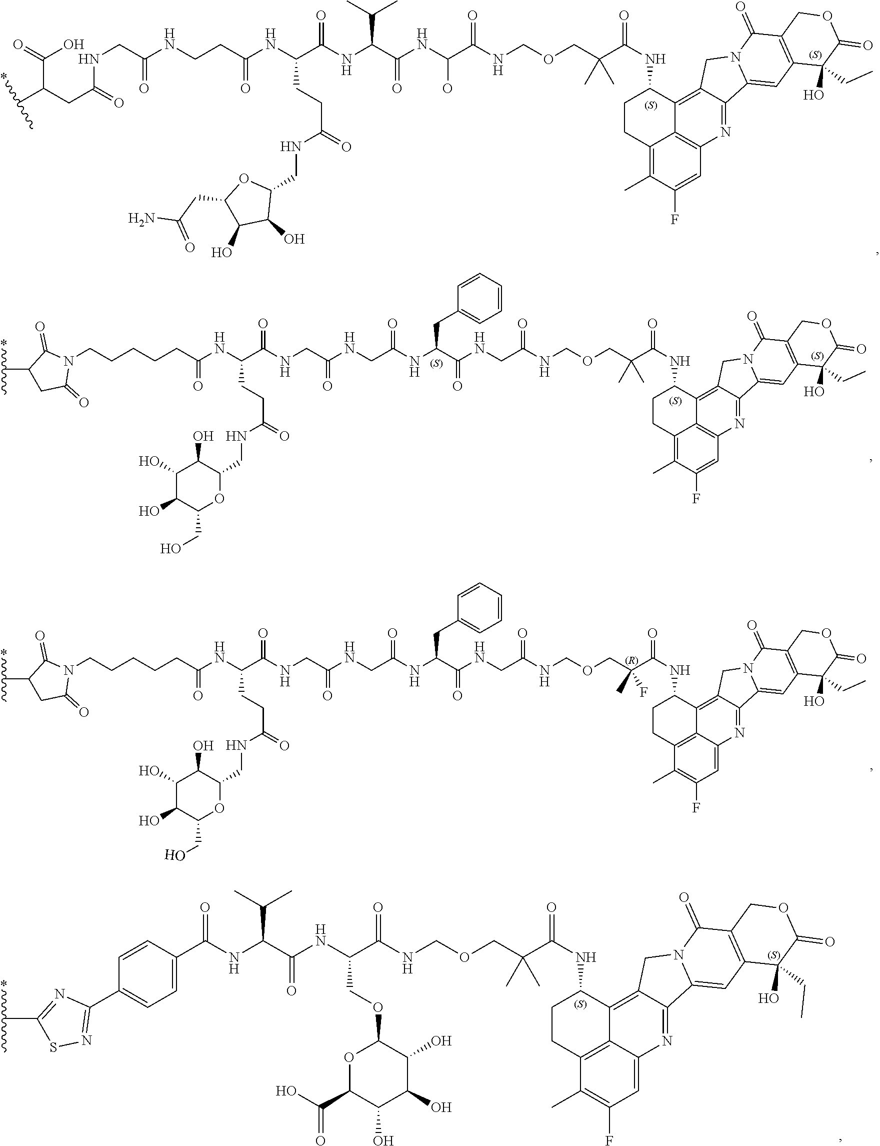

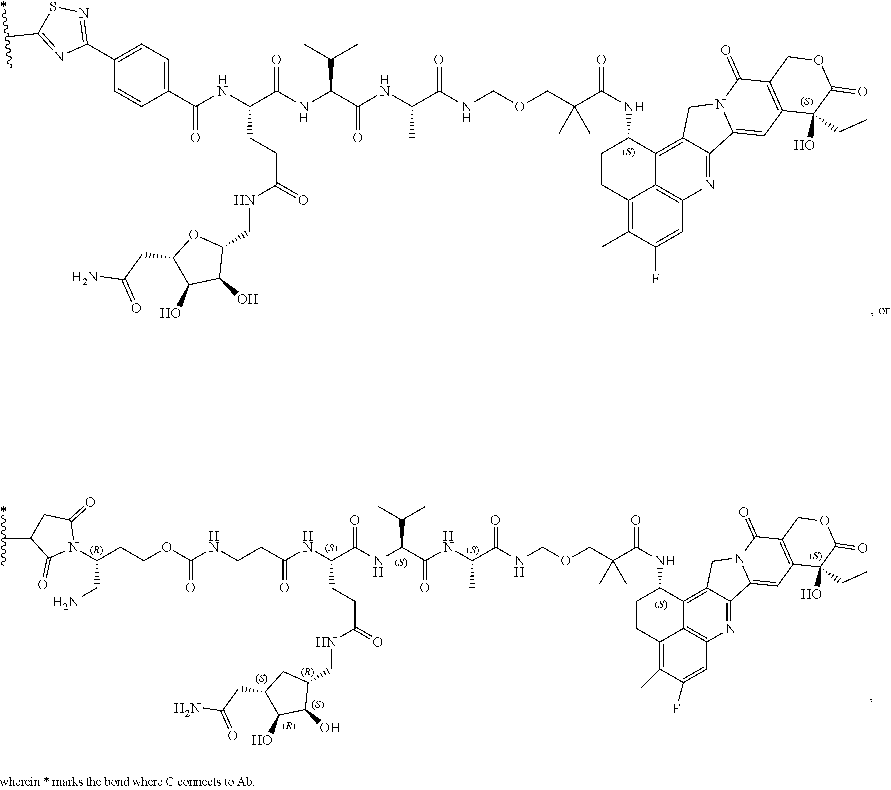

18. The antibody drug conjugate of claim 1, wherein C-L-(D)ₘ is:

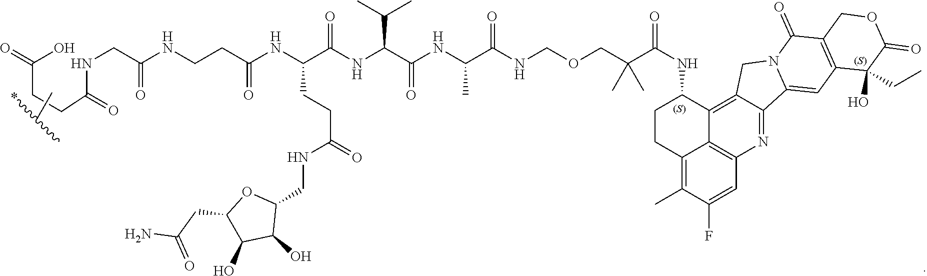

19. The antibody drug conjugate of claim 1, or a tautomer, pharmaceutically acceptable salt, solvate, or hydrate thereof, wherein the antibody drug conjugate has one of the following formulas:

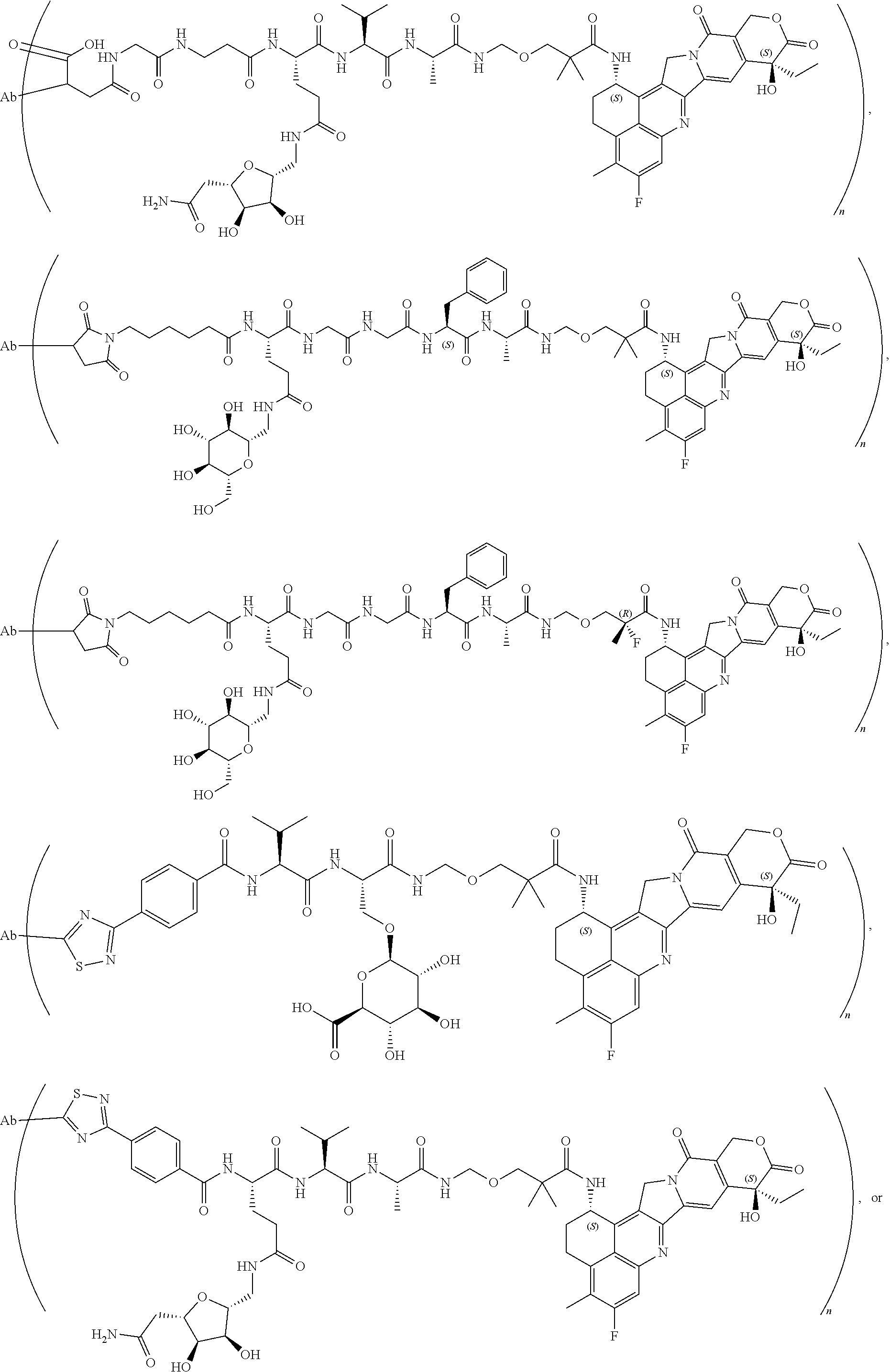

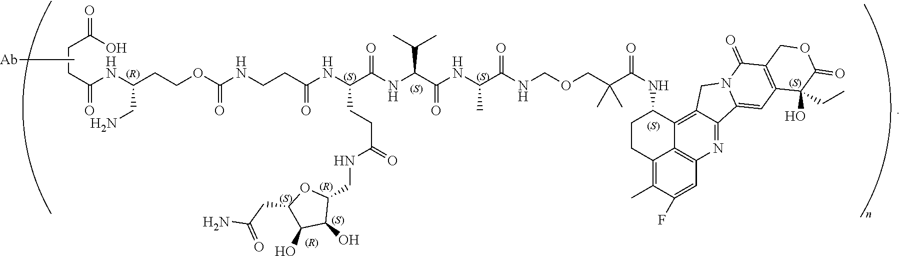

20. The antibody drug conjugate of claim 1, wherein the antibody or antigen-binding fragment comprises:
(i) a heavy chain variable region comprising SEQ ID NO. 31, and a light chain variable region comprising SEQ ID NO:32;
(ii) a heavy chain variable region comprising SEQ ID NO:48, and a light chain variable region comprising SEQ ID NO:49; or
(iii) a heavy chain variable region comprising SEQ ID NO:14, and a light chain variable region comprising SEQ ID NO:15.

21-24. (canceled)

25. The antibody drug conjugate of claim 1, wherein the antibody or antigen-binding fragment thereof comprises a heavy chain constant region of the subclass of IgG1, and a light chain constant region of the type of kappa.

26. An antibody drug conjugate of the formula: 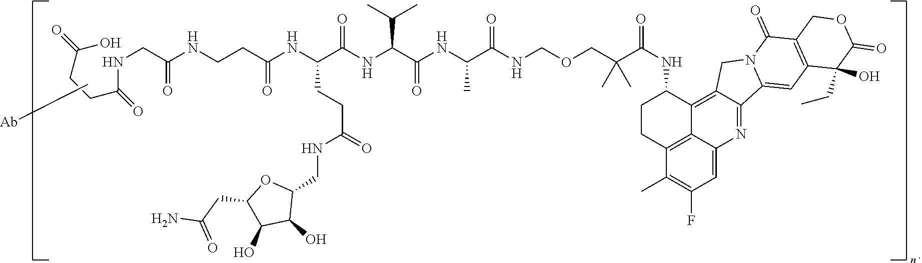
or a tautomer, pharmaceutically acceptable salt, solvate, or hydrate thereof, wherein:
n is from 4 to 10; and
Ab is an antibody or antigen-binding fragment thereof that binds CEA and comprises:
(i) three heavy chain CDRs:
HCDR1 comprising an amino acid sequence as set forth in SEQ ID NO:7,
HCDR2 comprising an amino acid sequence as set forth in SEQ ID NO:8,
HCDR3 comprising an amino acid sequence as set forth in SEQ ID NO:9, and
three light chain CDRs:
LCDR1 comprising an amino acid sequence as set forth in SEQ ID NO:10,
LCDR2 comprising an amino acid sequence as set forth in SEQ ID NO:11,
LCDR3 comprising an amino acid sequence as set forth in SEQ ID NO:6; or
(ii) three heavy chain CDRs:
HCDR1 comprising an amino acid sequence as set forth in SEQ ID NO:24,
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:23; or
(iii) three heavy chain CDRs:
HCDR1 comprising an amino acid sequence as set forth in SEQ ID NO:41,
HCDR2 comprising an amino acid sequence as set forth in SEQ ID NO:42,
HCDR3 comprising an amino acid sequence as set forth in SEQ ID NO:43, and
three light chain CDRs:
LCDR1 comprising an amino acid sequence as set forth in SEQ ID NO:44,
LCDR2 comprising an amino acid sequence as set forth in SEQ ID NO:45,
LCDR3 comprising an amino acid sequence as set forth in SEQ ID NO:40.

27. An antibody drug conjugate, or a tautomer, pharmaceutically acceptable salt, solvate, or hydrate thereof, wherein
the antibody drug conjugate has the following formula: 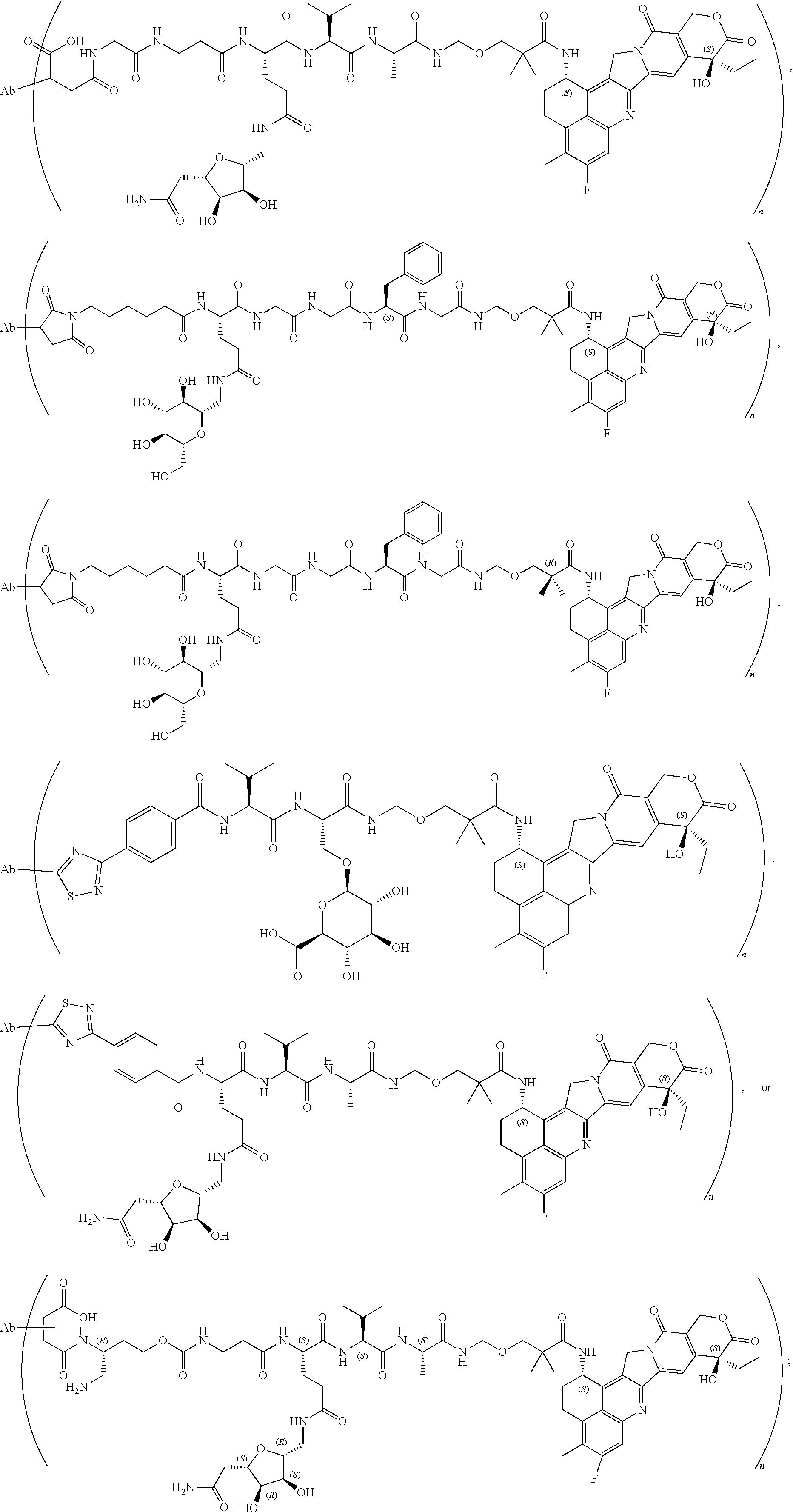
n is from 4 to 10; and
Ab is an antibody or antigen-binding fragment thereof that binds CEA and comprises:
(i) three heavy chain CDRs:
HCDR1 comprising an amino acid sequence as set forth in SEQ ID NO:7,
HCDR2 comprising an amino acid sequence as set forth in SEQ ID NO:8,
HCDR3 comprising an amino acid sequence as set forth in SEQ ID NO:9, and
three light chain CDRs:
LCDR1 comprising an amino acid sequence as set forth in SEQ ID NO:10,
LCDR2 comprising an amino acid sequence as set forth in SEQ ID NO:11,
LCDR3 comprising an amino acid sequence as set forth in SEQ ID NO:6; or
(ii) three heavy chain CDRs:
HCDR1 comprising an amino acid sequence as set forth in SEQ ID NO:24,
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:23; or
(iii) three heavy chain CDRs:
HCDR1 comprising an amino acid sequence as set forth in SEQ ID NO:41,
HCDR2 comprising an amino acid sequence as set forth in SEQ ID NO:42,
HCDR3 comprising an amino acid sequence as set forth in SEQ ID NO:43, and
three light chain CDRs:
LCDR1 comprising an amino acid sequence as set forth in SEQ ID NO:44,
LCDR2 comprising an amino acid sequence as set forth in SEQ ID NO:45,
LCDR3 comprising an amino acid sequence as set forth in SEQ ID NO:40.

28. A pharmaceutical composition comprising the antibody drug conjugate of claim 27, or a tautomer, pharmaceutically acceptable salt, solvate, or hydrate thereof and a pharmaceutically acceptable carrier.

29. A method of treating a CEA-expressing cancer comprising administering to a subject in need thereof an effective amount of the antibody drug conjugate of claim 1, or a tautomer, pharmaceutically acceptable salt, solvate, or hydrate thereof.

30-33. (canceled)

34. A method of producing the antibody drug conjugate of claim 1, comprising:
(i) culturing a host cell that has been transformed by an isolated nucleic acid comprising a sequence encoding the antibody or antigen-binding fragment thereof, wherein the antibody or antigen-binding fragment thereof comprises a heavy chain comprising an amino acid sequence of SEQ ID NO:99 and a light chain comprising an amino acid sequence of SEQ ID NO:100;
(ii) expressing the antibody or antigen-binding fragment thereof;
(iii) recovering the expressed antibody or antigen-binding fragment thereof; and
(iv) conjugating the cytotoxic agent to the antibody or fragment thereof using a linker, such that the antibody drug conjugate is formed.

35. (canceled)

36. A pharmaceutical composition comprising the antibody drug conjugate of claim 1, or a tautomer, pharmaceutically acceptable salt, solvate, or hydrate thereof, and a pharmaceutically acceptable carrier.

37. The antibody drug conjugate of claim 1, wherein the antibody drug conjugate has the following formula: 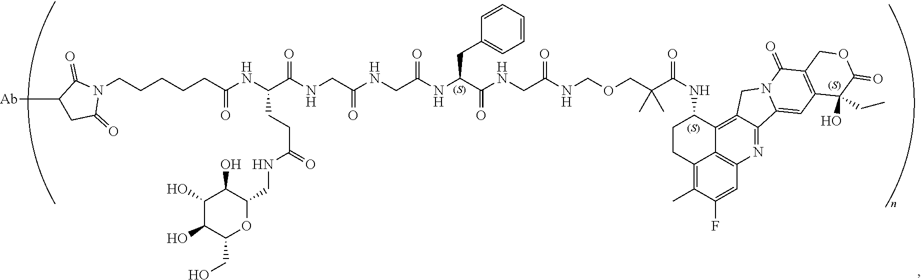
or a tautomer, pharmaceutically acceptable salt, solvate, or hydrate thereof, wherein n is from 4 to 10.

38. The antibody drug conjugate of claim 37, wherein Ab comprises:
three heavy chain CDRs:
HCDR1 comprising an amino acid sequence as set forth in SEQ ID NO:24,
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:23.

39. The antibody drug conjugate of claim 37, wherein Ab comprises:
three heavy chain CDRs:
HCDR1 comprising an amino acid sequence as set forth in SEQ ID NO:41,
HCDR2 comprising an amino acid sequence as set forth in SEQ ID NO:42,
HCDR3 comprising an amino acid sequence as set forth in SEQ ID NO:43, and
three light chain CDRs:
LCDR1 comprising an amino acid sequence as set forth in SEQ ID NO:44,
LCDR2 comprising an amino acid sequence as set forth in SEQ ID NO:45,
LCDR3 comprising an amino acid sequence as set forth in SEQ ID NO:40.

40. The antibody drug conjugate of claim 37, wherein Ab comprises:
three heavy chain CDRs:
HCDR1 comprising an amino acid sequence as set forth in SEQ ID NO:7,
HCDR2 comprising an amino acid sequence as set forth in SEQ ID NO:8,
HCDR3 comprising an amino acid sequence as set forth in SEQ ID NO:9, and
three light chain CDRs:
LCDR1 comprising an amino acid sequence as set forth in SEQ ID NO:10,
LCDR2 comprising an amino acid sequence as set forth in SEQ ID NO: 11,
LCDR3 comprising an amino acid sequence as set forth in SEQ ID NO:6.

41. The antibody drug conjugate of claim 37, wherein the antibody or antigen-binding fragment comprises:
(i) a heavy chain variable region comprising a sequence having at least 90% identity to SEQ ID NO:31, and a light chain variable region comprising a sequence having at least 90% identity to SEQ ID NO:32;
(ii) a heavy chain variable region comprising a sequence having at least 90% identity to SEQ ID NO:48, and a light chain variable region comprising a sequence having at least 90% identity to SEQ ID NO:49; or
(iii) a heavy chain variable region comprising a sequence having at least 90% identity to SEQ ID NO:14, and a light chain variable region comprising a sequence having at least 90% identity to SEQ ID NO:15.

42. The antibody drug conjugate of claim 37, wherein the antibody or antigen-binding fragment comprises a heavy chain having an amino acid sequence of SEQ ID NO:99 and a light chain having an amino acid sequence of SEQ ID NO:100.

43. The antibody drug conjugate of claim 1, wherein the antibody drug conjugate has the following formula: 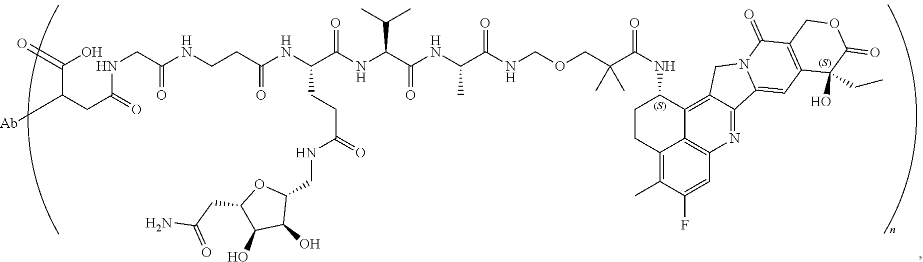
or a tautomer, pharmaceutically acceptable salt, solvate, or hydrate thereof, wherein n is from 4 to 10.

44. The antibody drug conjugate of claim 43, wherein Ab comprises:
three heavy chain CDRs:
HCDR1 comprising an amino acid sequence as set forth in SEQ ID NO:24,
HCDR2 comprising an amino acid sequence as set forth in SEQ ID NO:25,
HCDR3 comprising an amino acid sequence as set forth in SEQ ID NO:26, and
three light chain CDRs:
LCDR1 comprising an amino acid sequence as set forth in SEQ ID NO:27,
LCDR2 comprising an amino acid sequence as set forth in SEQ ID NO:28,
LCDR3 comprising an amino acid sequence as set forth in SEQ ID NO:23.

45. The antibody drug conjugate of claim 43, wherein Ab comprises:
three heavy chain CDRs:
HCDR1 comprising an amino acid sequence as set forth in SEQ ID NO:41,
HCDR2 comprising an amino acid sequence as set forth in SEQ ID NO:42,
HCDR3 comprising an amino acid sequence as set forth in SEQ ID NO:43, and
three light chain CDRs:
LCDR1 comprising an amino acid sequence as set forth in SEQ ID NO:44,
LCDR2 comprising an amino acid sequence as set forth in SEQ ID NO:45,
LCDR3 comprising an amino acid sequence as set forth in SEQ ID NO:40.

46. The antibody drug conjugate of claim 43, wherein Ab comprises:
three heavy chain CDRs:
HCDR1 comprising an amino acid sequence as set forth in SEQ ID NO:7,
HCDR2 comprising an amino acid sequence as set forth in SEQ ID NO:8,
HCDR3 comprising an amino acid sequence as set forth in SEQ ID NO:9, and
three light chain CDRs:
LCDR1 comprising an amino acid sequence as set forth in SEQ ID NO:10,
LCDR2 comprising an amino acid sequence as set forth in SEQ ID NO:11,
LCDR3 comprising an amino acid sequence as set forth in SEQ ID NO:6.

47. The antibody drug conjugate of claim 43, wherein the antibody or antigen-binding fragment comprises:
(i) a heavy chain variable region comprising a sequence having at least 90% identity to SEQ ID NO:31, and a light chain variable region comprising a sequence having at least 90% identity to SEQ ID NO:32;
(ii) a heavy chain variable region comprising a sequence having at least 90% identity to SEQ ID NO:48, and a light chain variable region comprising a sequence having at least 90% identity to SEQ ID NO:49; or
(iii) a heavy chain variable region comprising a sequence having at least 90% identity to SEQ ID NO:14, and a light chain variable region comprising a sequence having at least 90% identity to SEQ ID NO:15.

48. The antibody drug conjugate of claim 43, wherein the antibody or antigen-binding fragment comprises a heavy chain having an amino acid sequence of SEQ ID NO:99 and a light chain having an amino acid sequence of SEQ ID NO:100.
